(19) United States
(12) Patent Application Publication
(10) Pub. No.: US 2026/0275028 A1
(43) Pub. Date: Sep. 17, 2026

(54) DHNT MONOMERS AND POLYMER DYES WITH MODIFIED PHOTOPHYSICAL PROPERTIES

(71) Applicant: Beckman Coulter, Inc., Brea, CA (US)

(72) Inventors: Massimiliano TOMASULO, Miami, FL (US); Arunkumar EASWARAN, Miami, FL (US); Sergei GULNIK, San Jose, CA (US)

(73) Assignee: Beckman Coulter, Inc., Brea, CA (US)

(21) Appl. No.: 18/871,787

(22) PCT Filed: Aug. 24, 2023
(86) PCT No.: PCT/US2023/031069

§ 371 Date: Dec. 4, 2024

Related U.S. Application Data
(60) Provisional application No. 63/401,458, filed on Aug. 26, 2022.

(51) Int. Cl. C08G 61/12; C09B 69/10; C09K 11/06; G01N 33/58

(52) U.S. Cl. CPC C08G 61/126; C09B 69/10; C09K 11/06; G01N 33/582; C08G 2261/124; C08G 2261/1422; C08G 2261/145; C08G 2261/314; C08G 2261/3243; C08G 2261/412; C09K 2211/1416; C09K 2211/1458

(57) ABSTRACT

The present disclosure relates to dihydronaphthothiophene (DHNT), dihydrobenzo dithiophene (DHBDT), and dihydronapthothienothiophene (DHNTT) monomer units and fluorescent polymers and co-polymers thereof. DHNT, DHNTT, and/or DHBDT containing water-soluble fluorescent co-polymers, water-soluble tandem fluorescent co-polymers, water-soluble fluorescent co-polymer complexes, water-soluble tandem fluorescent co-polymer complexes, and their use in methods for detecting analytes in a biological sample are also provided.

This application is being filed on Aug. 24, 2023, as a PCT International Patent application and claims the benefit of and priority to U.S. Provisional patent application 63/401,458, filed on Aug. 26, 2022, the entire disclosure of which is incorporated by reference herein in its entirety.

### FIELD OF THE DISCLOSURE

The present disclosure relates to dihydronaphthothiophene (DHNT), dihydrobenzodithiophene (DHBDT), and dihydronapthothienothiophene (DHNTT) monomer units and fluorescent polymers and co-polymers thereof. The disclosure provides DHNT, DHNTT, and/or DHBDT containing water-soluble fluorescent co-polymers, water-soluble tandem fluorescent co-polymers, water-soluble fluorescent co-polymer complexes, water-soluble tandem fluorescent co-polymer complexes, and their use in methods for detecting analytes in a biological sample. The present disclosure also provides a kit comprising a polymer comprising a dihydronaphthothiophene (DHNT), dihydrobenzodithiophene (DHBDT), and/or dihydronapthothienothiophene (DHNTT) monomer, optionally with a conjugation tag.

### BACKGROUND

Polymer dyes are of interest because of very high extinction coefficients obtained when longer polymers are obtained.

There is an increasing demand for a variety of fluorescent dyes for use in current flow cytometers as well as in spectral flow instruments. Fluorescent compounds and their conjugates can be used in a variety of biological applications by generating signals which can be monitored in real time and provide simple and rapid methods for the detection of biological targets and events, e.g., in diagnostic kits, in microscopy, in cytometry, or in drug screening.

Molecular recognition involves the specific binding of two molecules. Molecules which have binding specificity for a target biomolecule find use in a variety of research and diagnostic applications, such as the labelling and separation of analytes, flow cytometry, in situ hybridization, enzyme-linked immunosorbent assays (ELISAs), western blot analysis, magnetic cell separations and chromatography. Target biomolecules may be detected by labelling with a fluorescent compound.

Current polyfluorene (e.g., Sirigen U.S. Pat. No. 8,362,193B2) and dihydrophenanthrene based polymer dyes having strong excitation in the ultraviolet and violet regions of 350-450 nm are known. U.S. Pat. No. 11,208,527 describes water soluble fluorene and 9,10-dihydrophenanthrene (DHP) based fluorescent polymer dyes, for example, exhibiting excitation maxima between 395-415 nm with emitted light between about 415-475 nm. U.S. Pat. No. 11,584,825 describes water soluble dihydrophenanthrene (DHP) based violet excitable polymers and tandem dyes.

Water solubility of the DHP polymers is obtained by, for example, introduction of polyethylene glycol (PEG) units on the DHP structure before polymerization. These polymers show very intense absorption centered at about 414 nm and an emission centered on about 427 nm which overlap the Pacific blue channel in flow cytometers. Conjugation of these DHP polymer dyes with antibodies results in bright conjugates excitable with the 405 nm laser line for, e.g., flow cytometry applications.

Since the polymeric nature of these fluorophores is responsible for the overall brightness, preparation of novel polymer dyes with modified absorption and emission characteristics is desirable so other useful laser lines (e.g., 355, 488, 563, 638, and 808 nm) and detection channels can be employed for flow cytometry applications, thereby expanding the number of colors in flow cytometry panels.

Parameters considered by a user in choosing a fluorescent dye may include excitation wavelength maximum, the emission wavelength maximum, brightness of the dye, and the fluorescence lifetime. Brightness of a dye is an overall contribution from the extinction coefficient (ε, measure of the amount of light absorbed at a particular wavelength) and fluorescence quantum yield (Φ, measure of the light emitted in the form of radiation from its singlet excited state).

Bright water-soluble polymer dyes that can be excited using UV, violet, blue, yellow, green, red, and near infrared (NIR) wavelengths are desirable.

### SUMMARY

The present disclosure generally provides novel bright water-soluble fluorescent polymers comprising at least one co-monomer M¹ according to the disclosure, fluorescent tandem dyes according to the disclosure, labeled specific binding partners according to the disclosure, and methods for detecting analytes in a sample. The polymers, tandem dyes, and labeled specific binding partners include one or more monomeric units based on dihydronaphthothiophene (DHNT), dihydronapthothienothiophene (DHNTT), or dihydrobenzodithiophene (DHBDT) structures. The polymers, tandem dyes, and labeled specific binding partners according to the present disclosure may be water soluble, and may be useful in biological applications, including for the detection of target analytes. The present disclosure also provides for kits comprising the polymers, tandem dyes and/or labeled specific binding partners, according to the present disclosure, optionally having a conjugation tag. The polymers, tandem dyes, and labeled specific binding partners according to the present disclosure that are included in the kit may be water soluble, and may be useful in biological applications, including for the detection of target analytes.

The present disclosure provides the synthesis of novel structures including 4,5-dihydronaphtho[1,2-b]thiophene (DHNT) monomers, dihydronapthothienothiophene monomers (DHNTT), and dihydrobenzodithiophene (DHBDT) monomers which, when co-polymerized with DHP monomers produces polymers with red shifted emission. Novel structures based on 5,6,12,13-tetrahydronaphtho[1,2-b]naphtho[2′,1′:4,5]thieno[2,3-d]thiophene-5,6,12,13-tetraol and 5,6-dihydronaphtho[1,2-b]thieno[2,3-d]thiophene-5,6-diol (each described as DHNTT), and 2,7-dibromo-4,5-dimethyl-4,5-dihydrobenzo[2,1-b:3,4-b′]dithiophene-4,5-diol (DHBDT) monomers are disclosed.

The disclosure provides a water-soluble polymer dye comprising at least one co-monomer M¹ having a structure according to Formula (Ia):

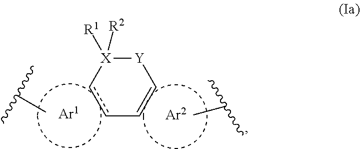

wherein
Ar¹ and Ar² are each independently substituted or unsubstituted aryl or heteroaryl groups and at least one of Ar¹ and Ar² comprises at least one fused thienyl ring;
each X is independently selected from the group consisting of C and Si;
each Y is independently selected from the group consisting of a bond, CR¹R², CHR¹, CHR², SiHR², SiHR¹, and SiR¹R², and when Y is a bond X is directly bonded to both rings. In some cases, when Y is a bond, Ar¹ and Ar² are not both thienyl. In some cases, Ar¹ and Ar² are different. In some cases, Ar¹ and Ar² are the same.

Each R¹ may be independently selected from the group consisting of a water-solubilizing moiety, a linker moiety, alkyl, alkene, alkyne, cycloalkyl, hydroxy, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, phosphonate,

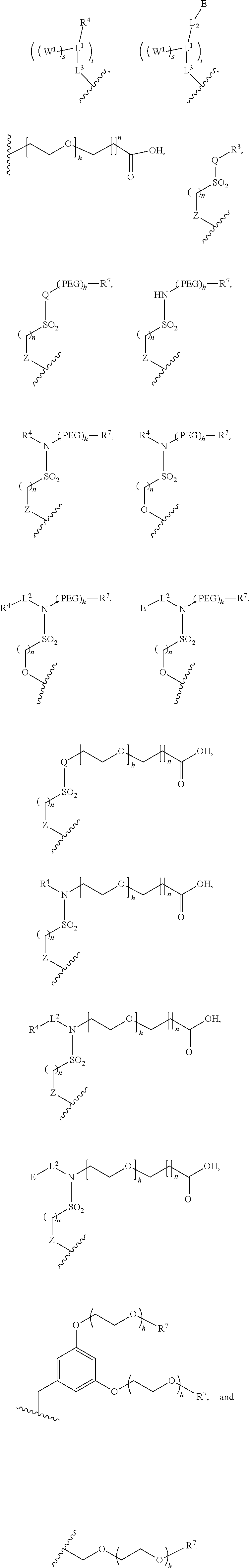

Each R² may be independently selected from the group consisting of a water-solubilizing moiety, a linker moiety, H, alkyl, alkene, alkyne, cycloalkyl, halogen, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, phosphonate,

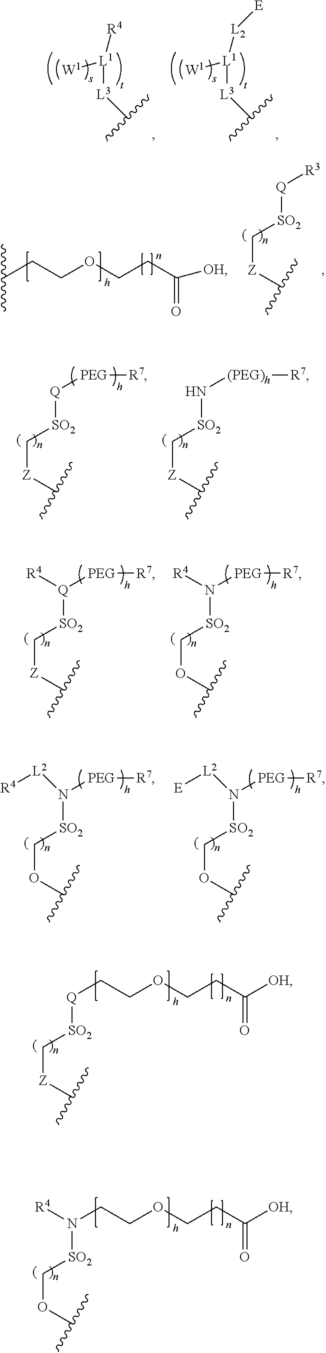

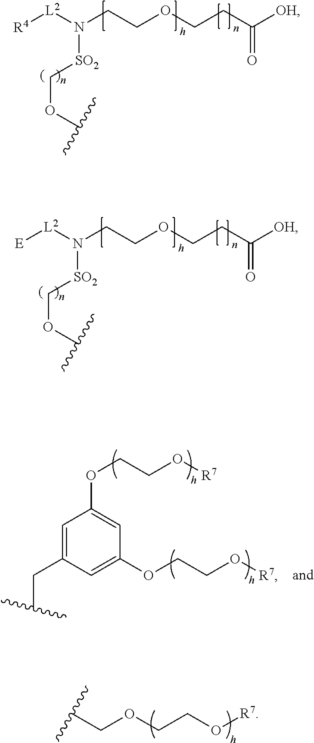

Each R³ may be independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, and a PEG group.

Each Z may be independently selected from the group consisting of CH₂, CHR⁴, O, NR⁴, and NH.

Each Q may be independently selected from the group consisting of a bond, NH, NR⁴, C₁-C₁₂ alkylene, CHR⁴, and CH₂.

Each R⁴ may be independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, an amine, hydroxyl, alkylamino, substituted or unsubstituted C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₈ alkyl, x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester. N-hydroxysuccinimidyl, hydrazine, hydrazone, azide, aldehyde, thiol, and protected groups thereof.

Each W¹ may be independently a water-solubilizing moiety.

L¹, L², and L³ each may be independently selected linker moieties.

Each E may be independently selected from the group consisting of a chromophore, a functional moiety, a substrate, and a binding partner.

Each R⁷ may be independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester and —OC₁-C₁₂hydroxy.

At least one of R¹, R², R³, or R⁴ comprises a water-solubilizing moiety.

Each h may be independently an integer from 0 to 50. Each n may be independently an integer from 1 to 20.

Each s may be independently 1 or 2.

Each t may be independently 0, 1, 2, or 3.

The at least one co-monomer M may have a structure selected from the group consisting of Formula (IIa), (IIb), (IIc), (IId), (IIe), (IIf), and (IIg):

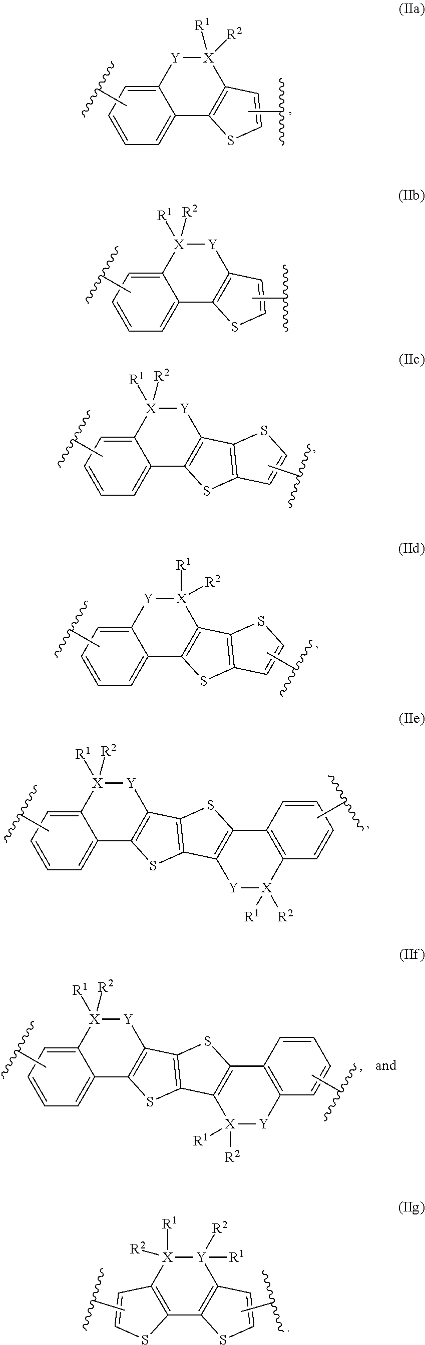

The at least one co-monomer M¹ may have a structure selected from the group consisting of Formula (IIaa), (IIbb), (IIcc), (IIdd), (IIee), (IIff), (IIgg), (IIaaa), (IIbbb), (IIccc), (IIddd), (IIeee), (IIfff), and (IIggg):

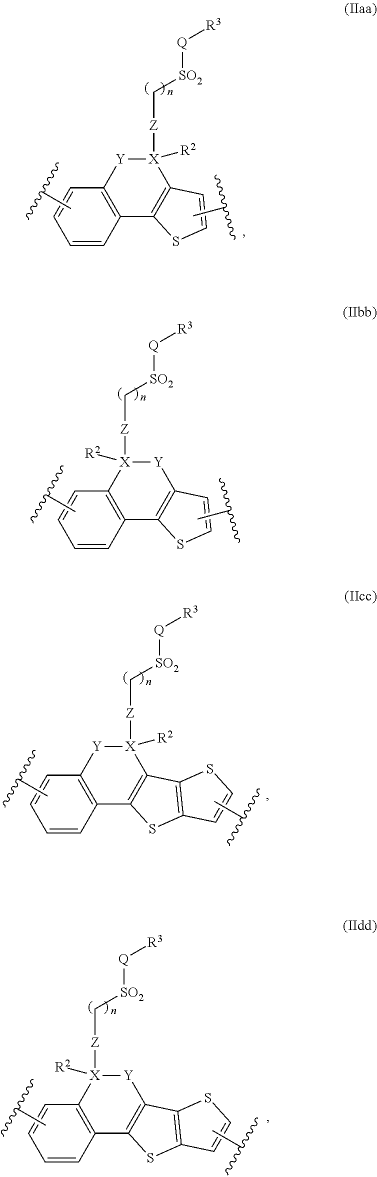

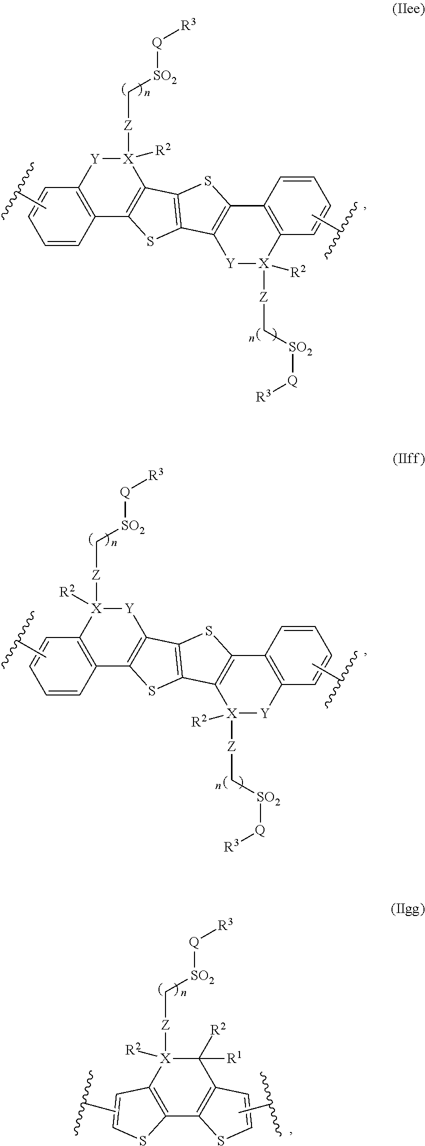

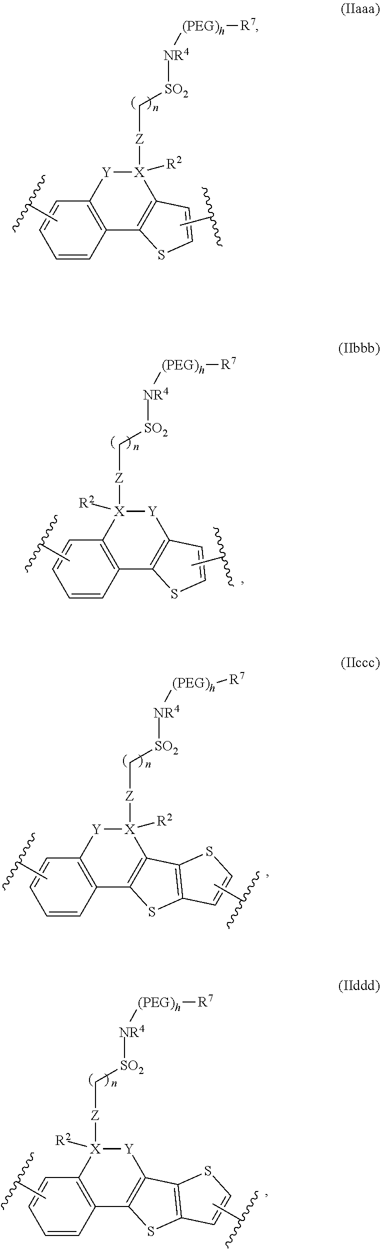

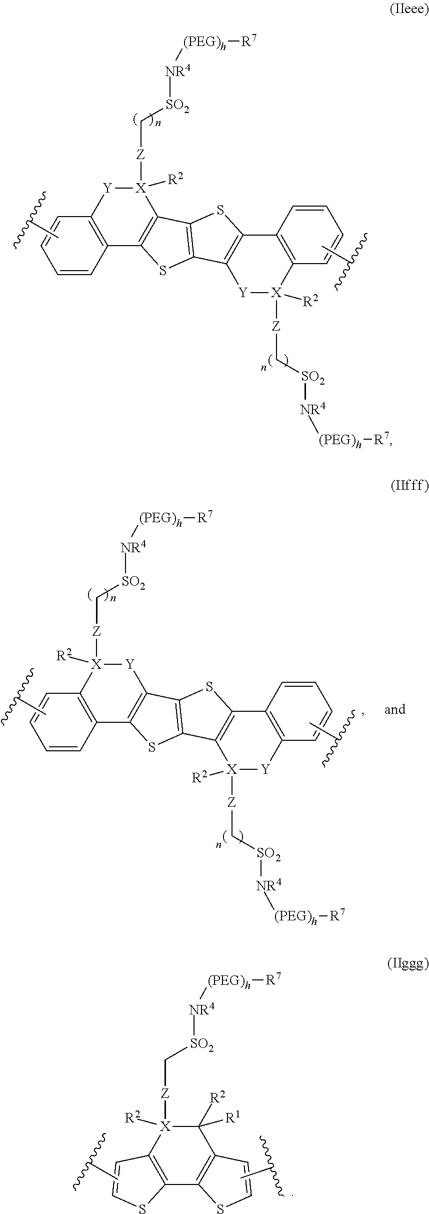

The disclosure provides a water-soluble polymer dye, comprising the structure of Formula (Ib):

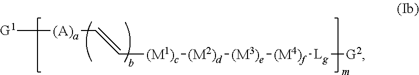

wherein
each A is selected from a substituted or unsubstituted aryl or heteroaryl group;
each M¹ is a co-monomer comprising a structure according to Formula (Ia):

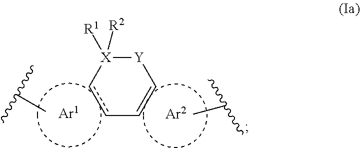

each optional linker L is independently a linker moiety;
G¹ and G² are each independently selected from the group consisting of an unmodified polymer terminus and a modified polymer terminus, optionally conjugated to E;
a, c, d, e, f and g define the mol % of each unit within the structure which each can be evenly or randomly repeated along the polymer main chain and where a is a mol % from 10 to 100%, c is a mol % from >0 to 90%, each d, e, and f is a mol % from 0 to 90%, and each g is a mol % from 0 to 25%;
each b is independently 0 or 1;
m is an integer from 1 to about 10,000;
each optional M², M³, and M⁴ is independently selected from the group consisting of an aryl moiety, heteroaryl moiety, or co-monomer M¹ having a structure of Formula (Ia), wherein
M¹, M², M³, and M⁴ are evenly or randomly distributed along the polymer main chain, and
each M¹ or optional M², M³, or M⁴ may be optionally substituted, and may be optionally terminated with a functional group selected from amine, carbamate, carboxylic acid, carboxylate, alkyl carboxylate, carboxylic amine, carbamate, carboxylate ester, maleimide, halogenated maleimide, activated ester, N-hydroxysuccinimidyl, imido ester, halogen, boronic ester, boronic acid, hydrozonyl, hydrazine, hydrazide, hydrazone, azide, alkyne, cyclooctyne, aldehyde, tetrazine, alkene, cyclooctene, dienes, dienophiles, thiol, amide, sulfonamide, alkyl sulfonate, ether, thioether, thiocarbamate, sulfonyl fluoride, hydroxyl, iodoacetyl, iodoacetamide, hydrazido, hydrazino, aldehyde, ketone, phosphine, epoxide, urea, thiourea, thioester, imine, disulfides, and protected groups thereof, each optionally conjugated to a substrate, chromophore, or binding partner, and
X, Y, R¹, R², Ar¹ and Ar² are defined herein.

In some cases, m is an integer from 1 to about 10,000, 2 to 8,000, 3 to 5,000, 4 to 1,000, 5 to 500, 6 to 100, or 10 to 50.

In some cases, each co-monomer M¹ is as defined herein. In some cases, each co-monomer M¹ may independently comprise a structure selected from the group consisting of Formula (IIa), (IIb), (IIc), (IId), (IIe), (IIf), and (IIg). In some cases, each co-monomer M¹ may independently comprise a structure selected from the group consisting of Formula (IIaa), (IIbb), (IIcc), (IIdd), (IIee), (IIff), (IIgg), (IIaaa), (IIbbb), (IIccc), (IIddd), (IIeee), (IIfff), and (IIggg).

In some cases, each optional M², M³, and M⁴ may independently comprise a structure selected from the group consisting of Formulas (IIa), (IIb), (IIc), (IId), (IIe), (IIf), (IIg), (IIaa), (IIbb), (IIcc), (IIdd), (IIee), (IIff), (IIgg), (IIaaa), (IIbbb), (IIccc), (IIddd), (IIeee), (IIfff), and (IIIggg). In some examples, M¹, M², M³ and M⁴ are evenly or randomly distributed along the polymer main chain.

Each of X, Y, Z, Q, R¹, R², R³, R⁴, R⁷, W¹, L, L¹, L², and L³, may be independently as described herein. In some cases, at least one of, at least two of, or at least three of R¹, R², R³, or R⁴ comprises a water-solubilizing moiety.

Each E may be independently selected from the group consisting of a chromophore, a functional moiety, a substrate, and a binding partner.

Each h may be independently an integer from 0 to 50, 10 to 20, or 11 to 18.

Each n may be independently an integer from 1 to 20, 2 to 18, 3 to 15, 4 to 12, or 5 to 10.

Each s may be independently 1 or 2.

Each t may be independently 0, 1, 2, or 3.

In some cases, A is selected from the group consisting of

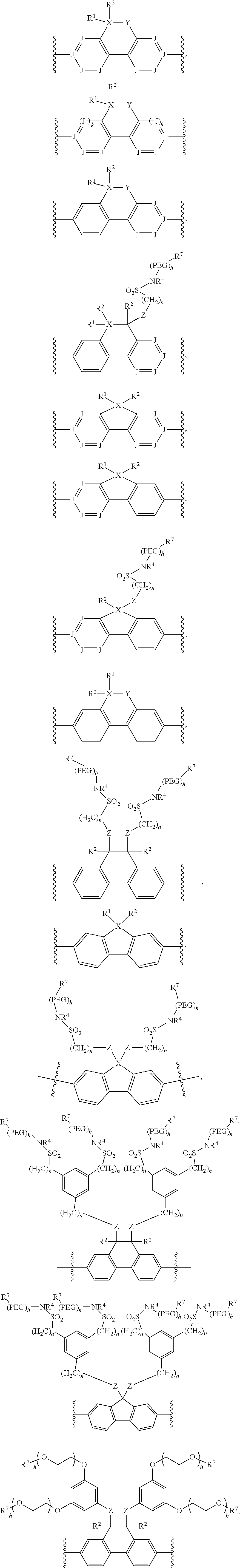

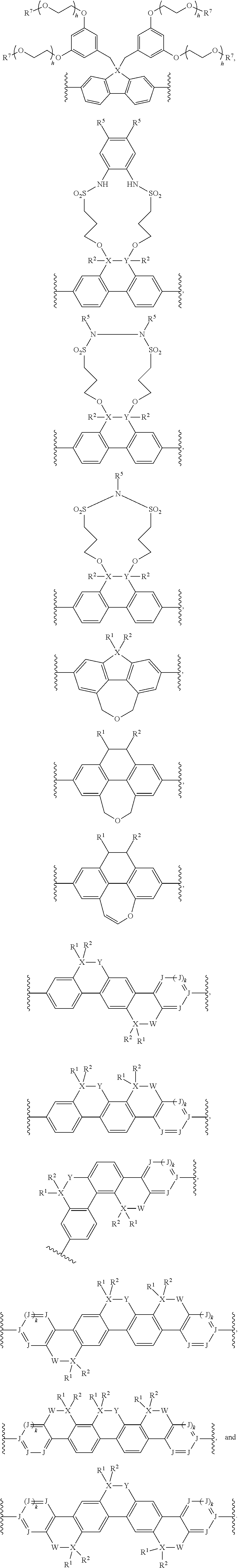

Each R⁵ may be independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50.

Each J may be independently selected from the group consisting of C, C(R²), N, N(R²), P, S, O, Se, and Si(R²). In some cases, J can be C or C(R²). In some cases, J can be N or N(R²). J can be P. J can be S. J can be O. J can be Se. J can be Si(R²).

Each W may be independently selected from the group consisting of a bond and Y; when W is a bond X is directly bonded to both rings.

Each k may independently be 0, 1, or 2.

The disclosure provides a polymer dye according to Formula (Ib) comprising a structure selected from the group consisting of Formula (IIIa), (IIIb), (IV), (V), (VI), (VIIa), (VIIb), (VIIc), (VIId), (VIIe), (VIIIa), (VIIIb), (XII), (XIIIa), (XIIIb), (XIIIc), (XIIId), and (XIIIe):

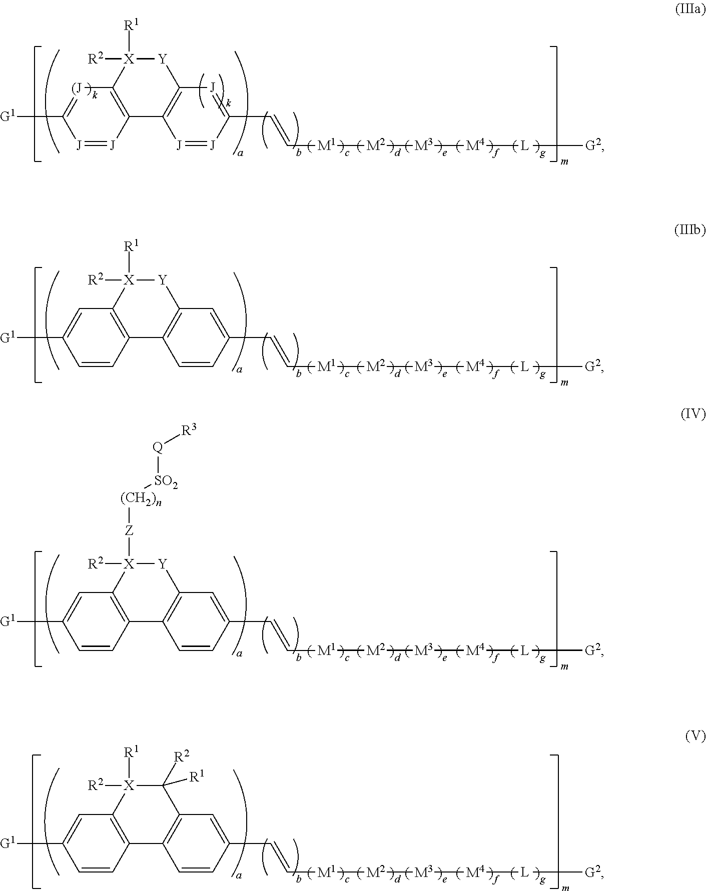

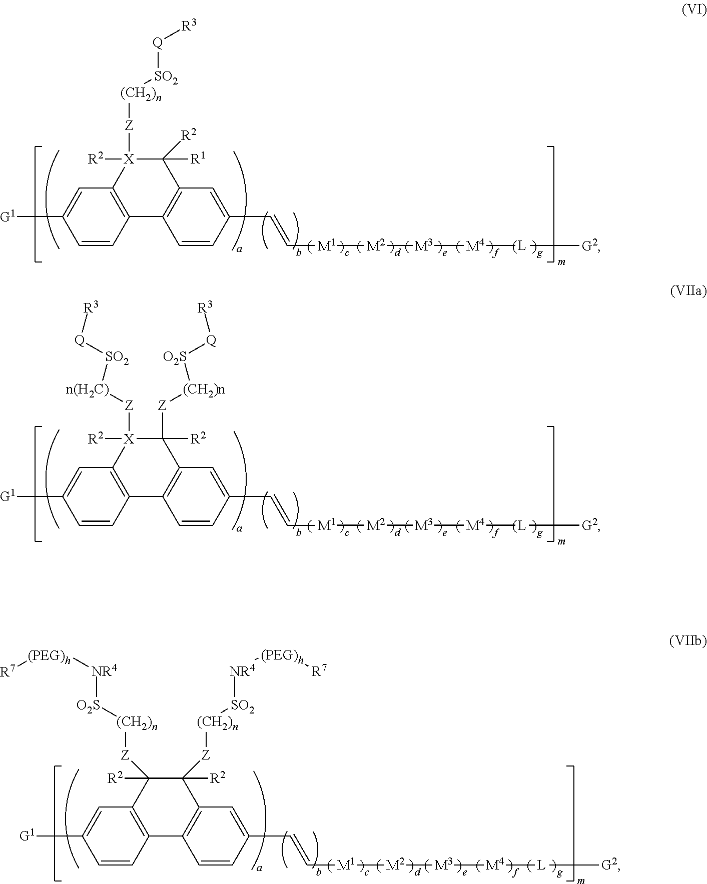

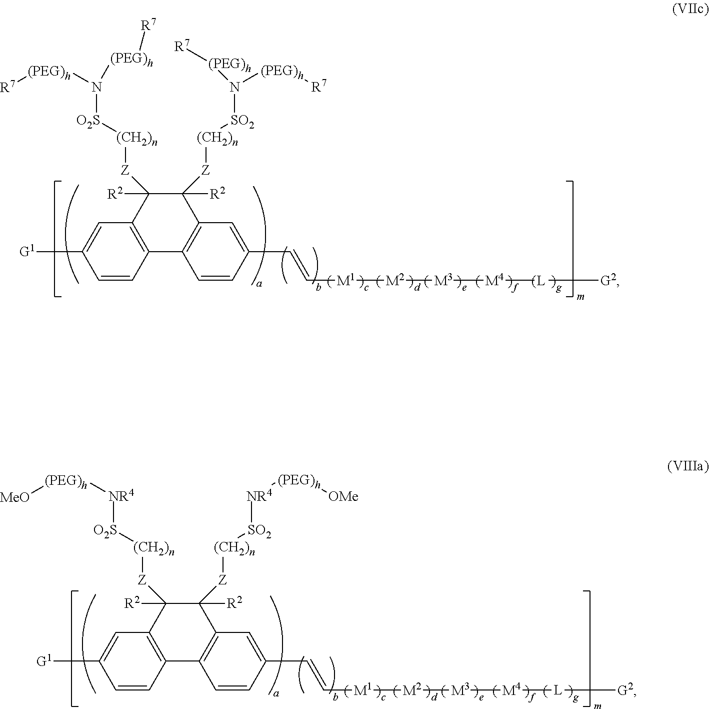

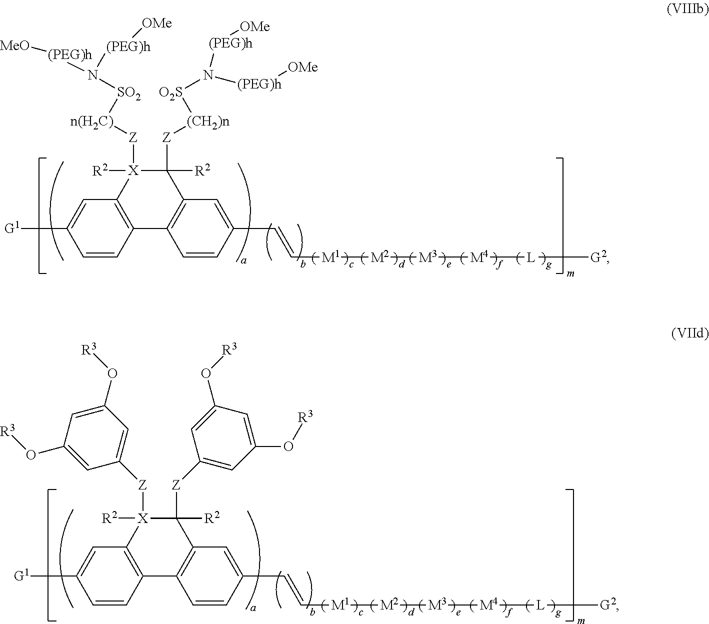

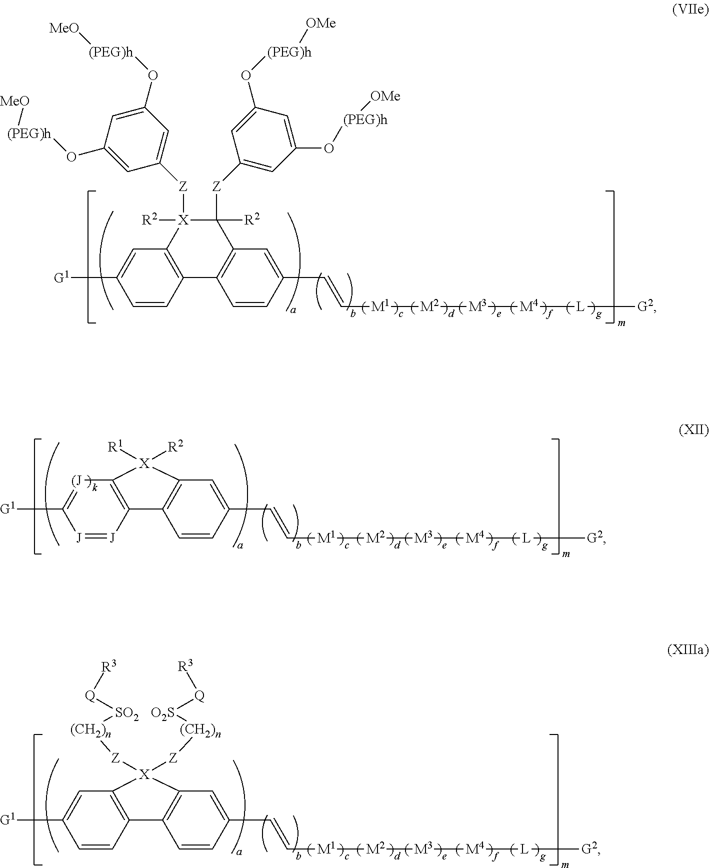

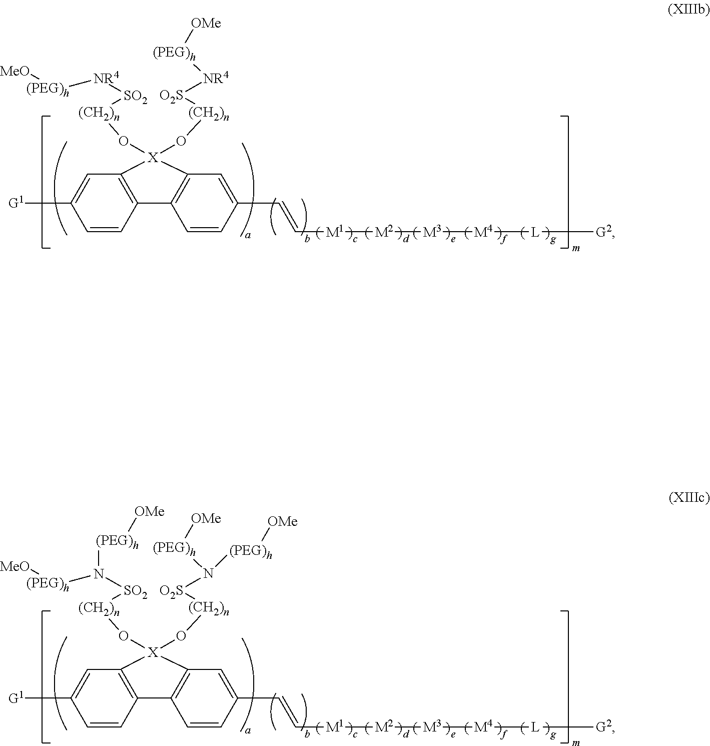

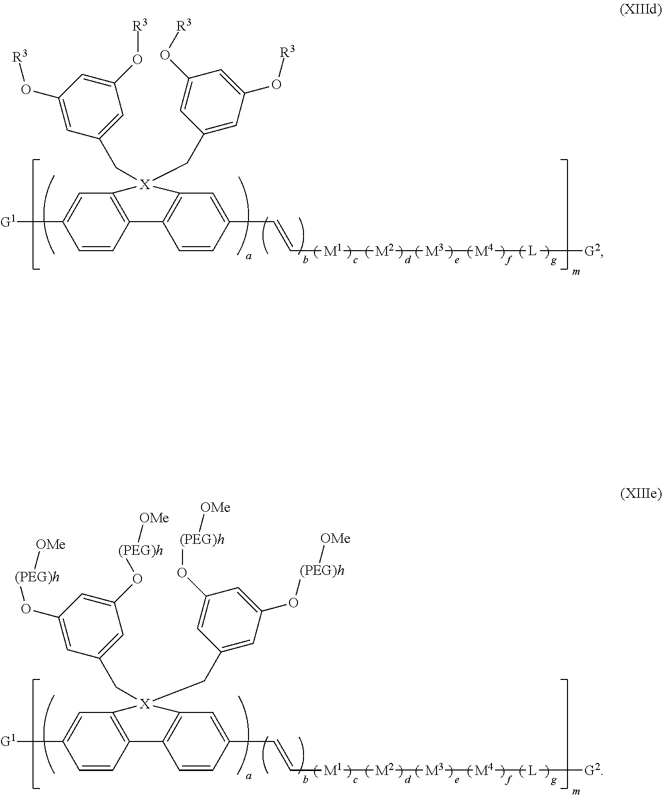

A polymer dye is provided according to Formula (Ib), wherein the dye comprises a structure selected from the group consisting of Formula (IXa), (IXb), (IXc), (Xa), (Xb), and (XI):

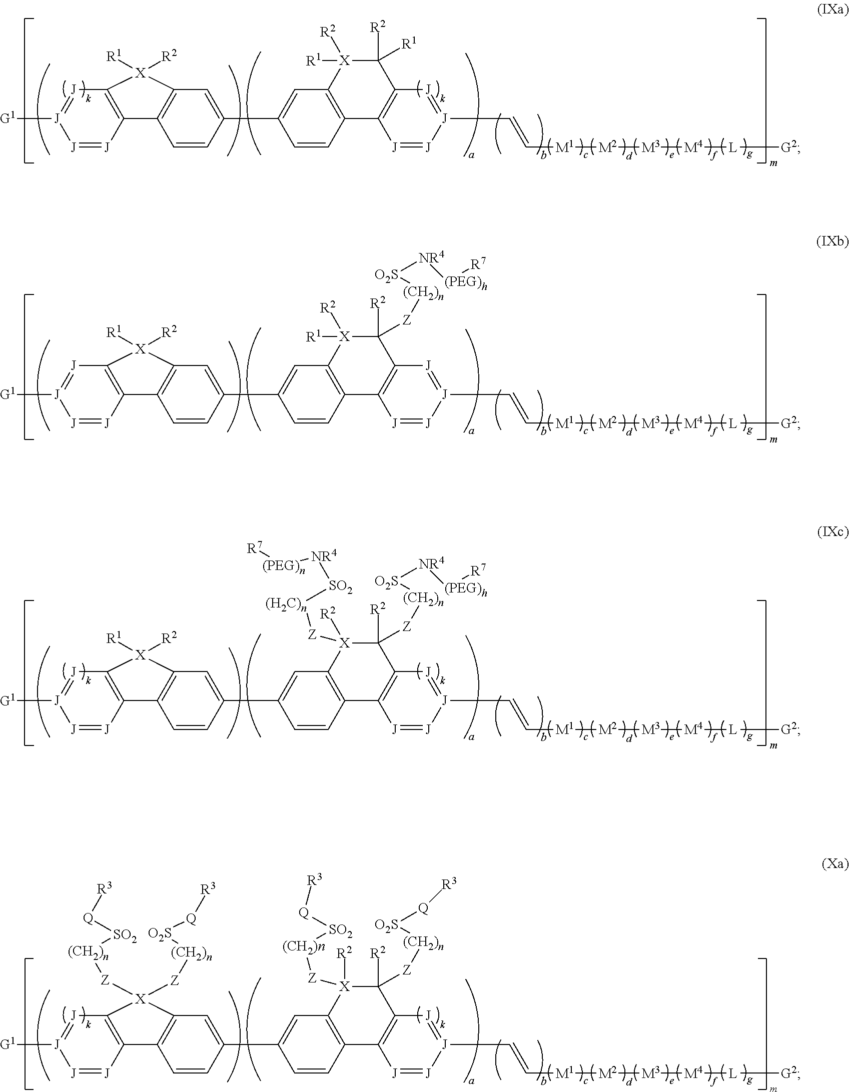

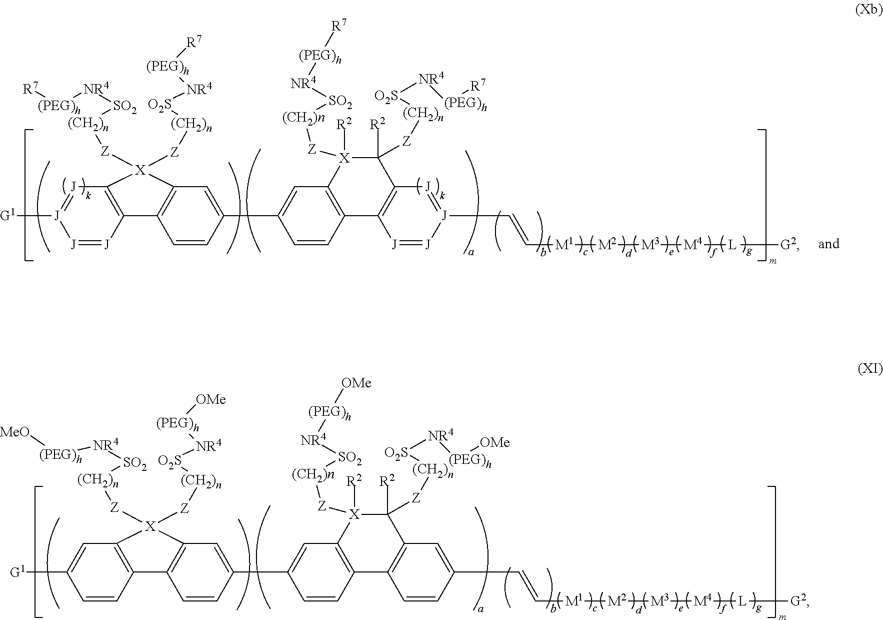

wherein a and i together is a mol % from 10 to 100%.

The disclosure provides a polymer dye comprising a structure selected from the group consisting of Formula (XIVa), (XIVb), (XIVc), (XIVd), (XIVe), (XIVf), and (XIVg):

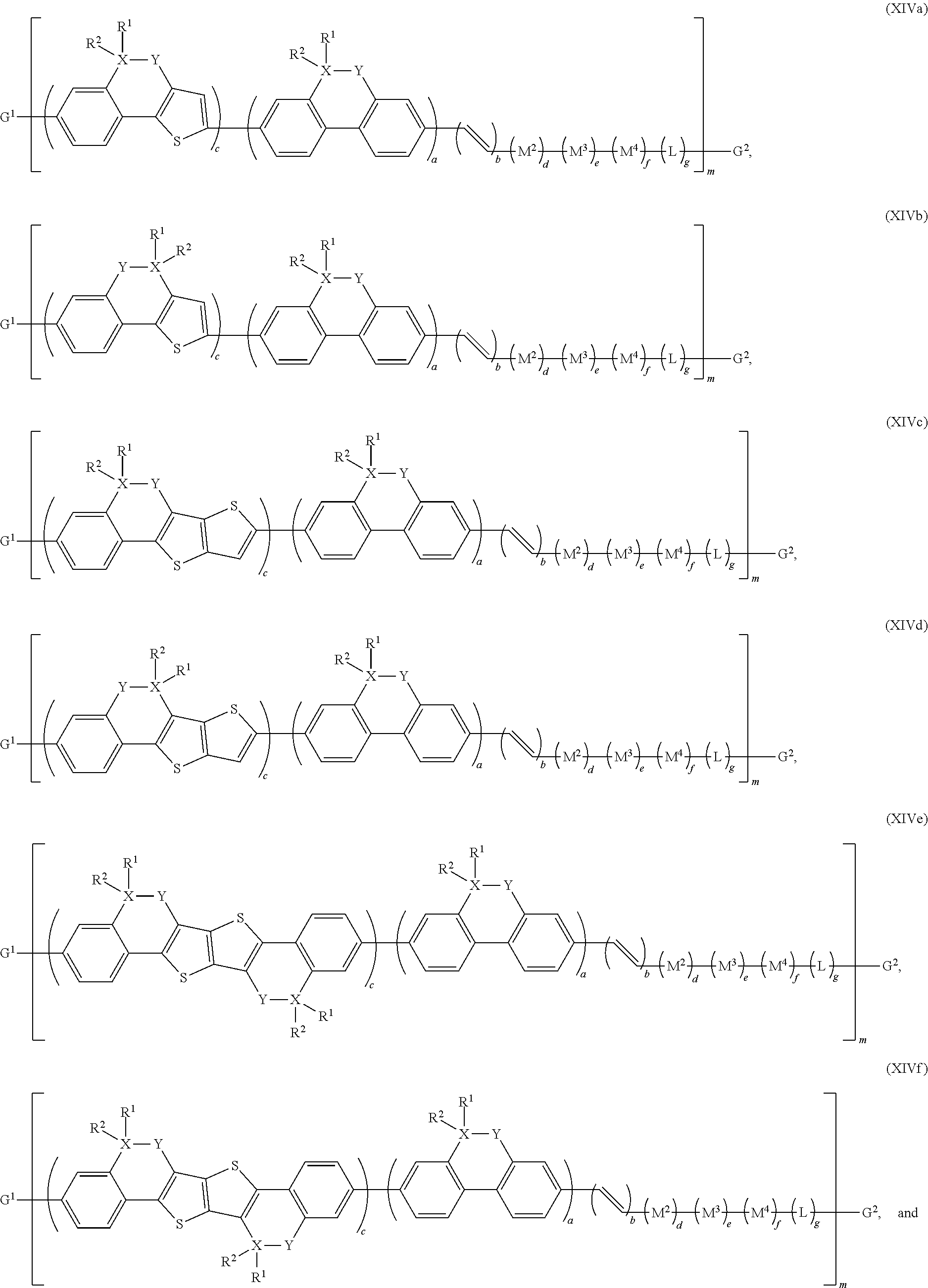

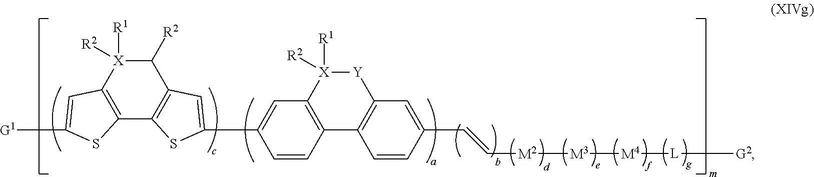

wherein each X, Y, Z, Q, R¹, R², R³, R⁴, R⁷, W¹, L, L¹, L², L³, E, G¹ and G² are each as described herein.

G¹ and G² may each independently selected from the group consisting of an unmodified polymer terminus and a modified polymer terminus, optionally conjugated to E. G¹ and G² may each independently comprise a conjugation tag.

a, c, d, e, f and g define the mol % of each unit within the structure which each can be evenly or randomly repeated along the polymer main chain and where a is a mol % from 10 to 100%, c is a mol % from >0 to 90%, each d, e, and f is a mol % from 0 to 90%, and each g is a mol % from 0 to 25%. Each b may be independently 0 or 1. m may be an integer from 1 to about 10,000. Each optional M², M³ and M⁴ may be independently an aryl or hetero-aryl moiety. Each h may be independently an integer from 0 to 50. Each n may be independently an integer from 1 to 20. Each s may be independently 1 or 2. Each t may be independently 0, 1, 2, or 3.

The disclosure provides a polymer dye comprising a structure selected from the group consisting of Formula (XVa), (XVb), (XVb), (XVb), (XVb), (XVb), (XVg), (XVIa), (XVIb), (XVIc), (XVId), (XVIIa), (XVIIb), (XVLIc), and (XVJId):

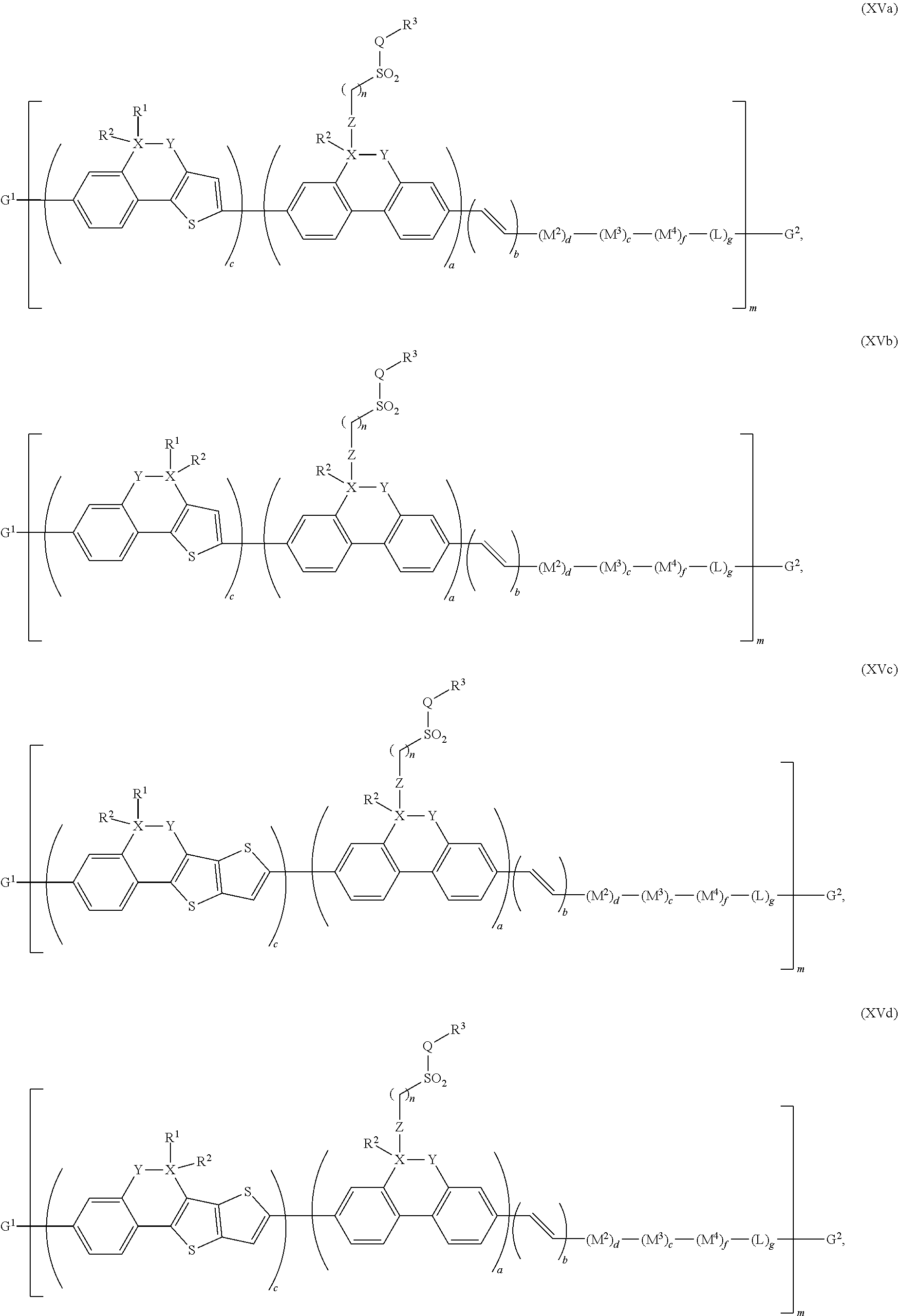

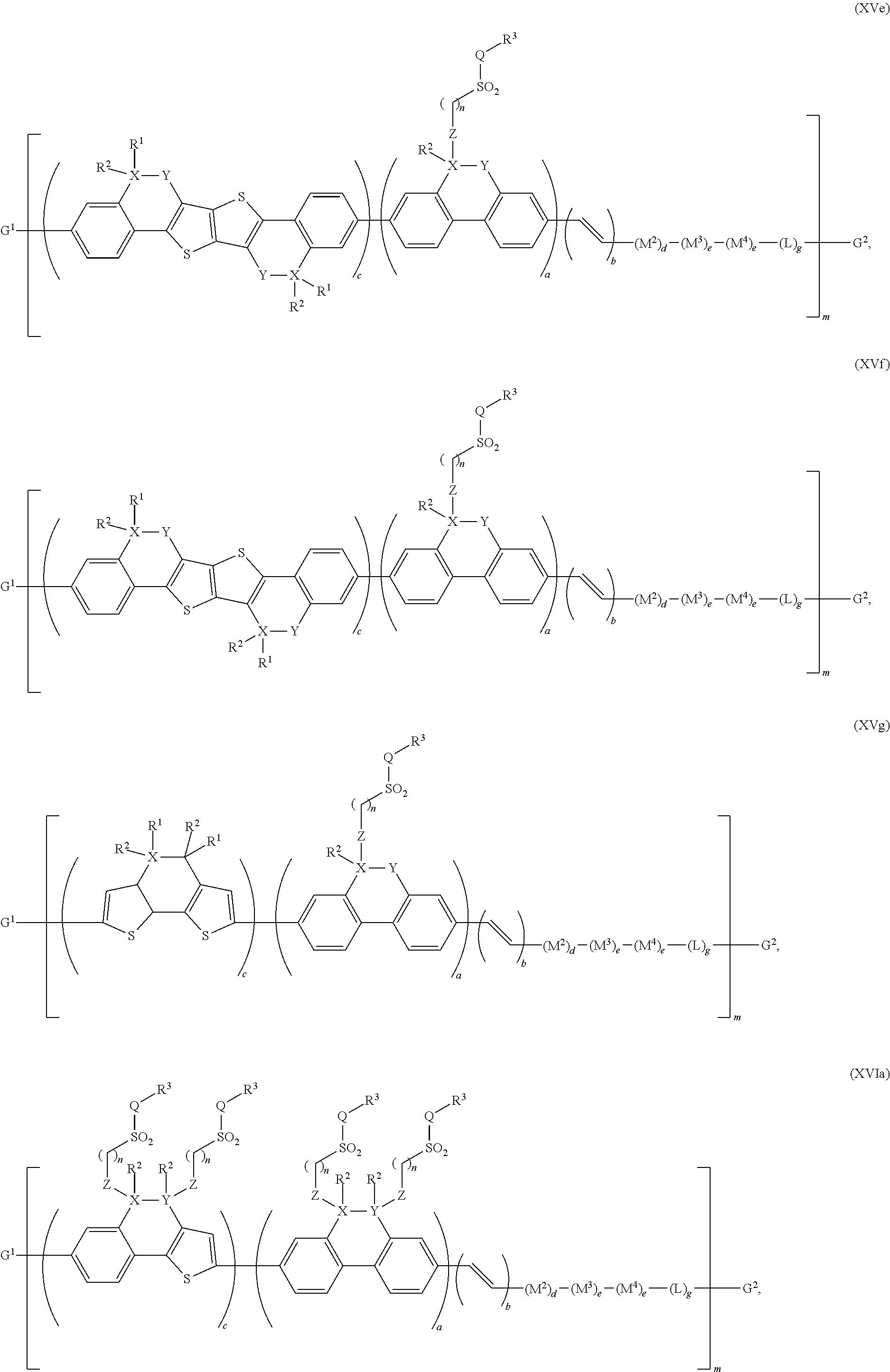

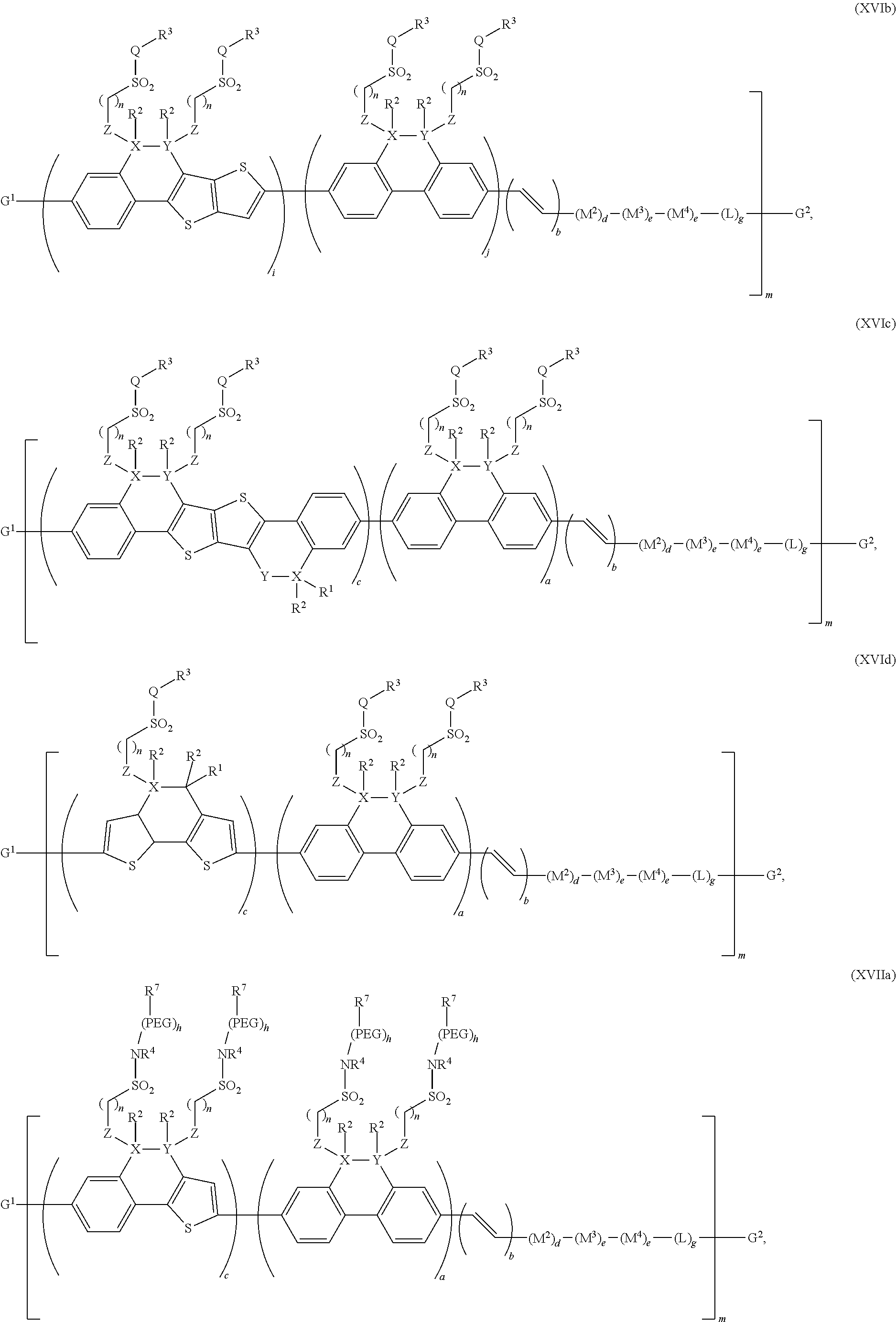

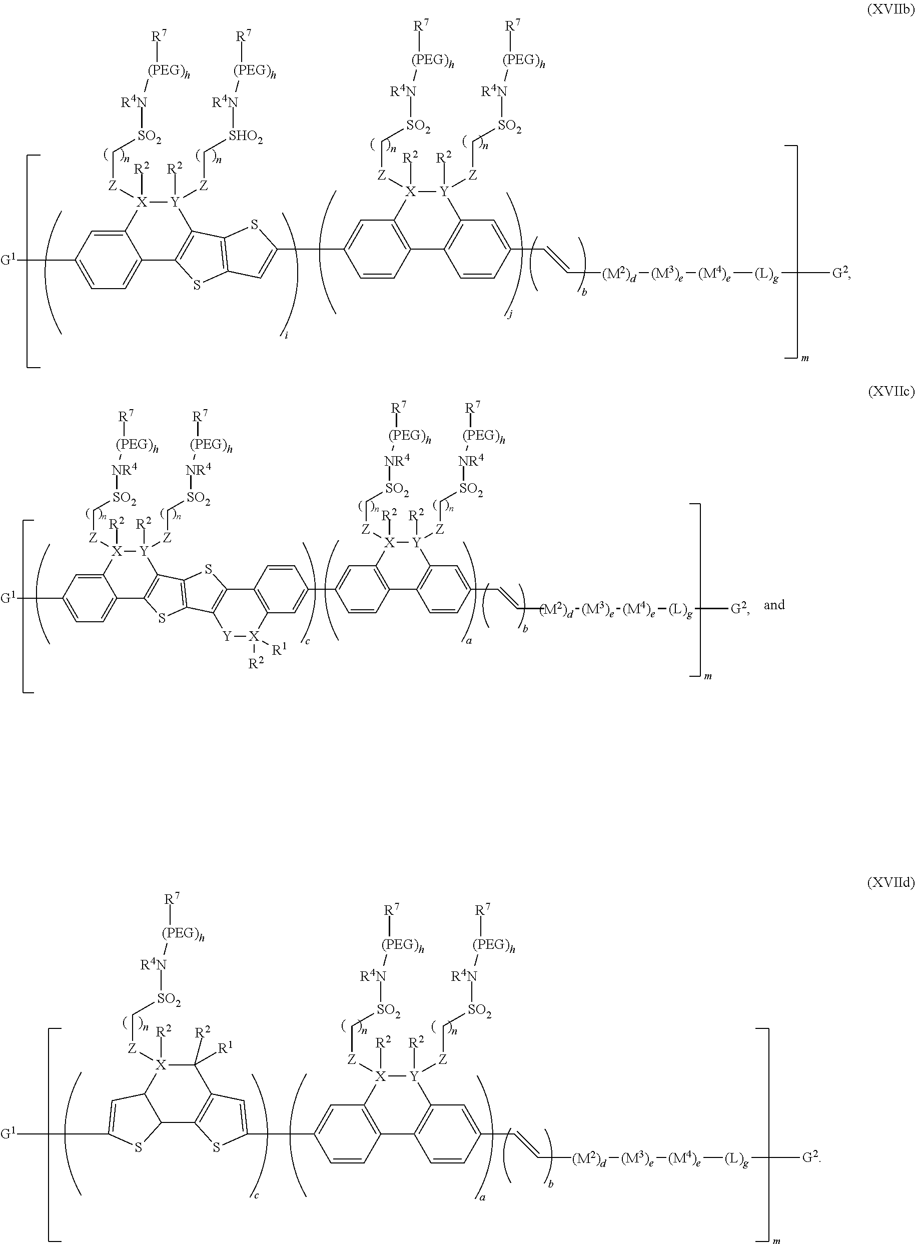

In some embodiments, each optional M², M³, or M⁴ may be independently a co-monomer having a structure selected from the group consisting of:

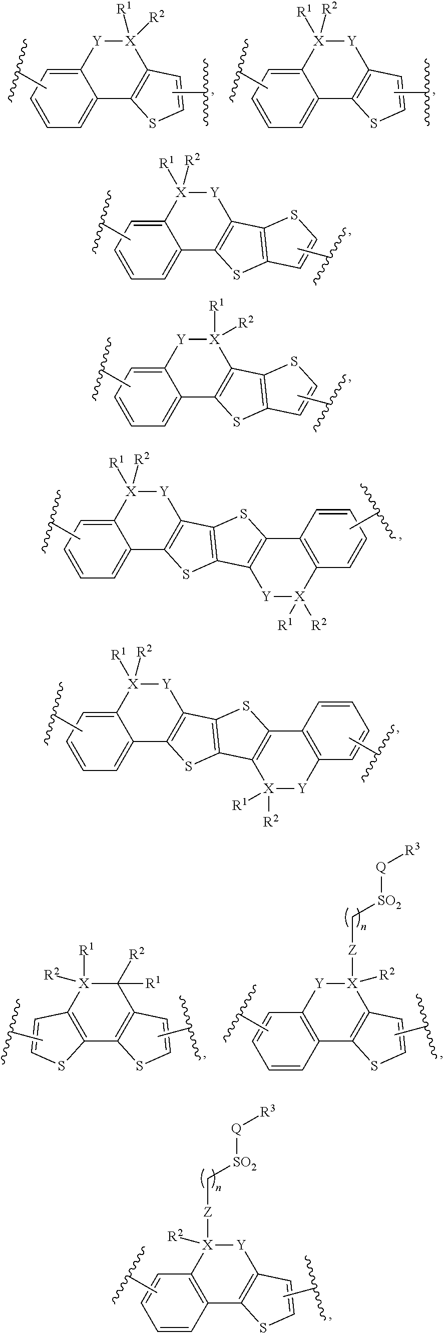

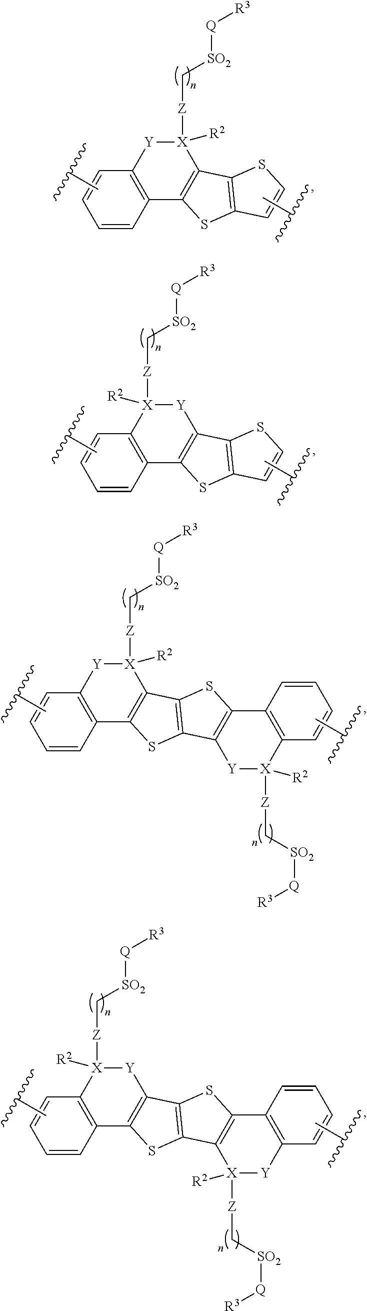

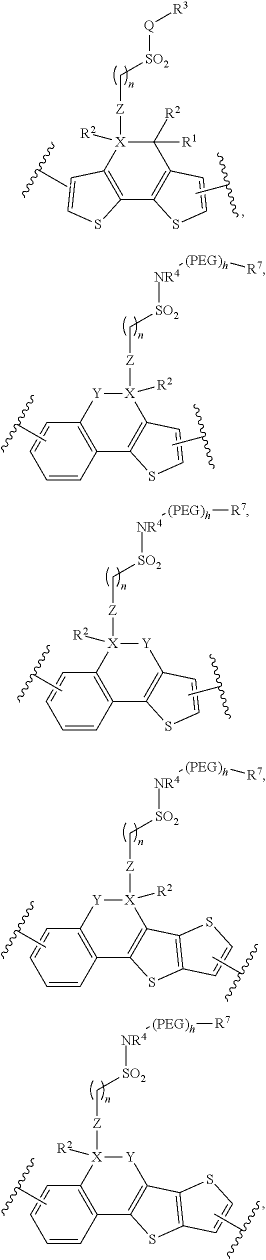

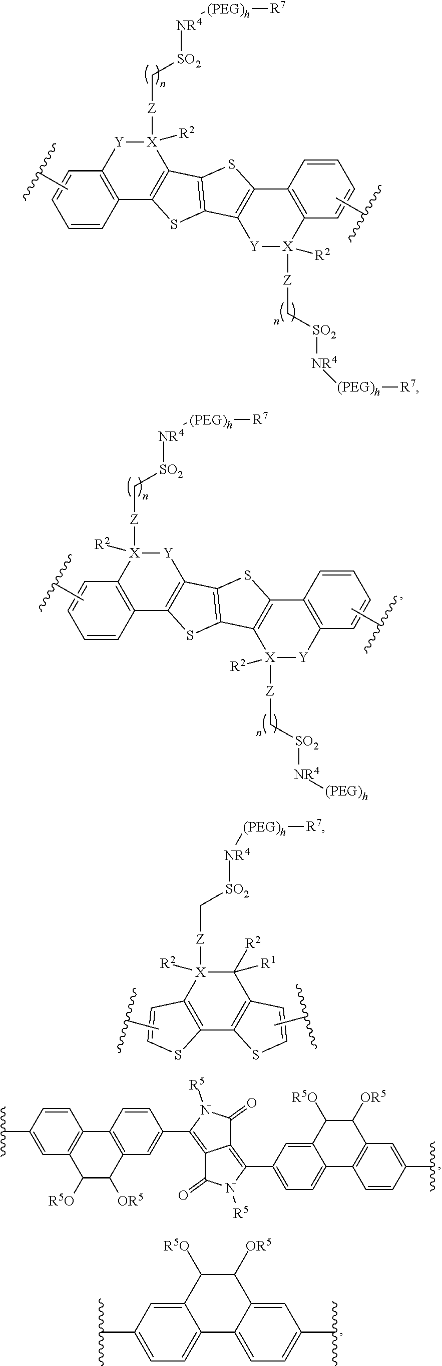

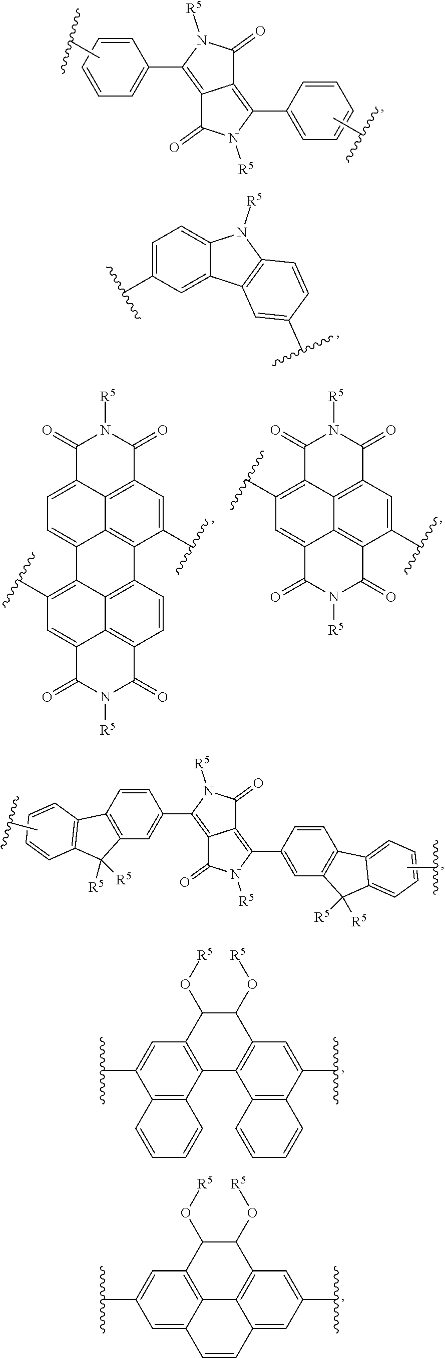

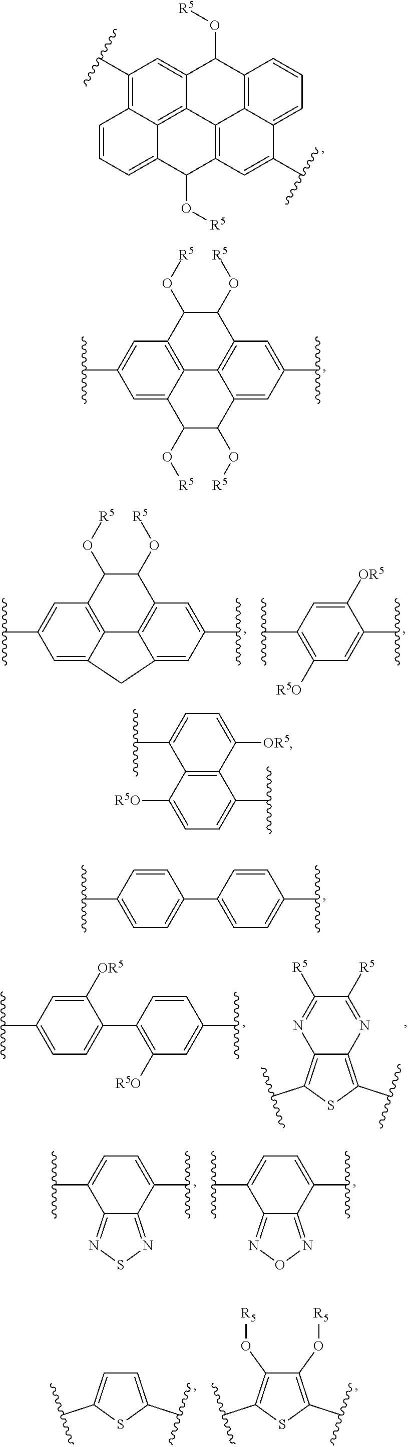

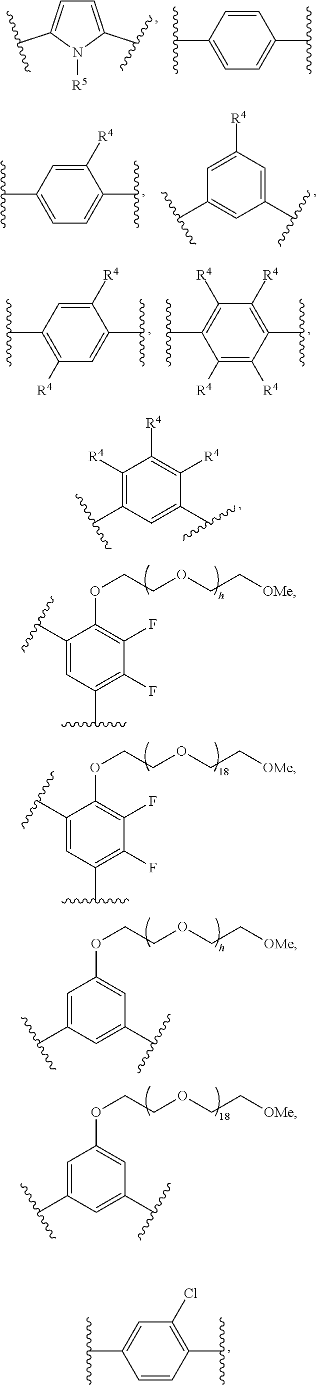

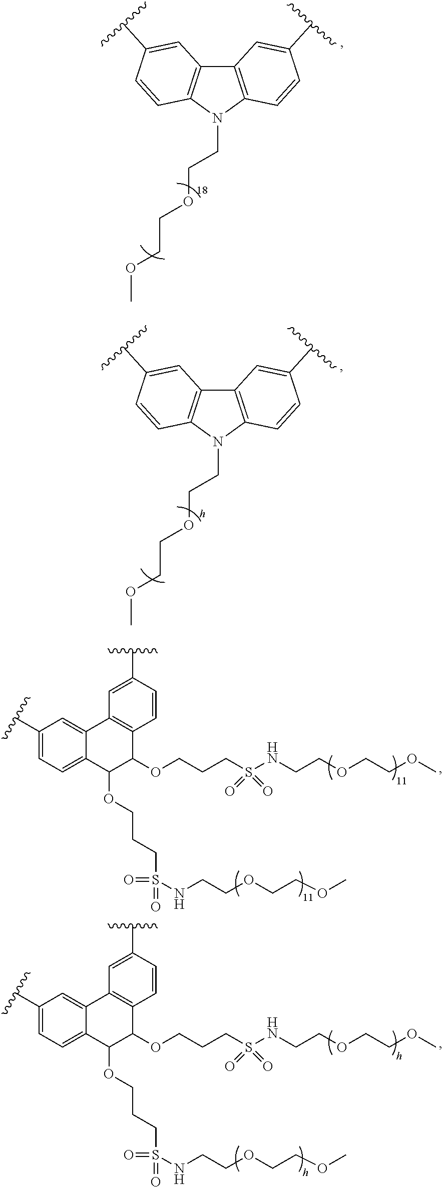

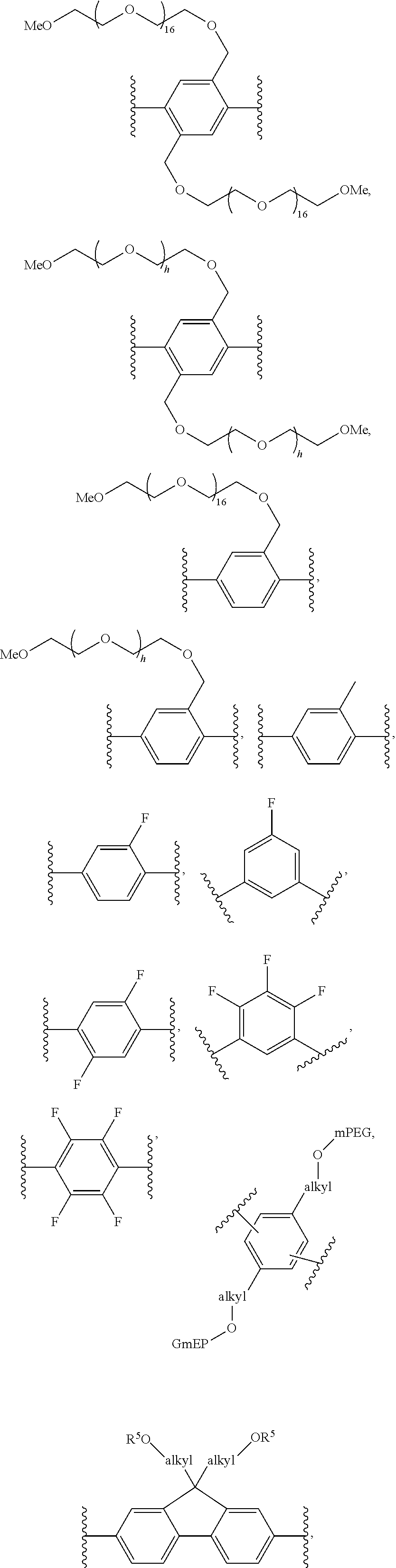

an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substitute, wherein
each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50, optionally conjugated to E.

In some cases, the optional linker L may be independently selected from the group consisting of an aryl or heteroaryl group evenly or randomly distributed along the polymer main chain and that is substituted with one or more pendant chains terminated with a functional group selected from amine, carbamate, carboxylic acid, carboxylate, alkyl carboxylate, carboxylic amine, carbamate, carboxylate ester, maleimide, halogenated maleimide, activated ester, N-hydroxysuccinimidyl, imido ester, halogen, boronic ester, boronic acid, hydrozonyl, hydrazine, hydrazide, hydrazone, azide, alkyne, cyclooctyne, aldehyde, tetrazine, alkene, cyclooctene, dienes, dienophiles, thiol, amide, sulfonamide, alkyl sulfonates, ether, thioether, thiocarbamate, sulfonyl fluoride, hydroxyl, iodoacetyl, iodoacetamide, hydrazido, hydrazino, aldehyde, ketone, phosphine, epoxide, urea, thiourea, thioester, imine, disulfides, and protected groups thereof, and protected groups thereof, optionally conjugated to E.

In some embodiments, L may be independently selected from the group consisting of

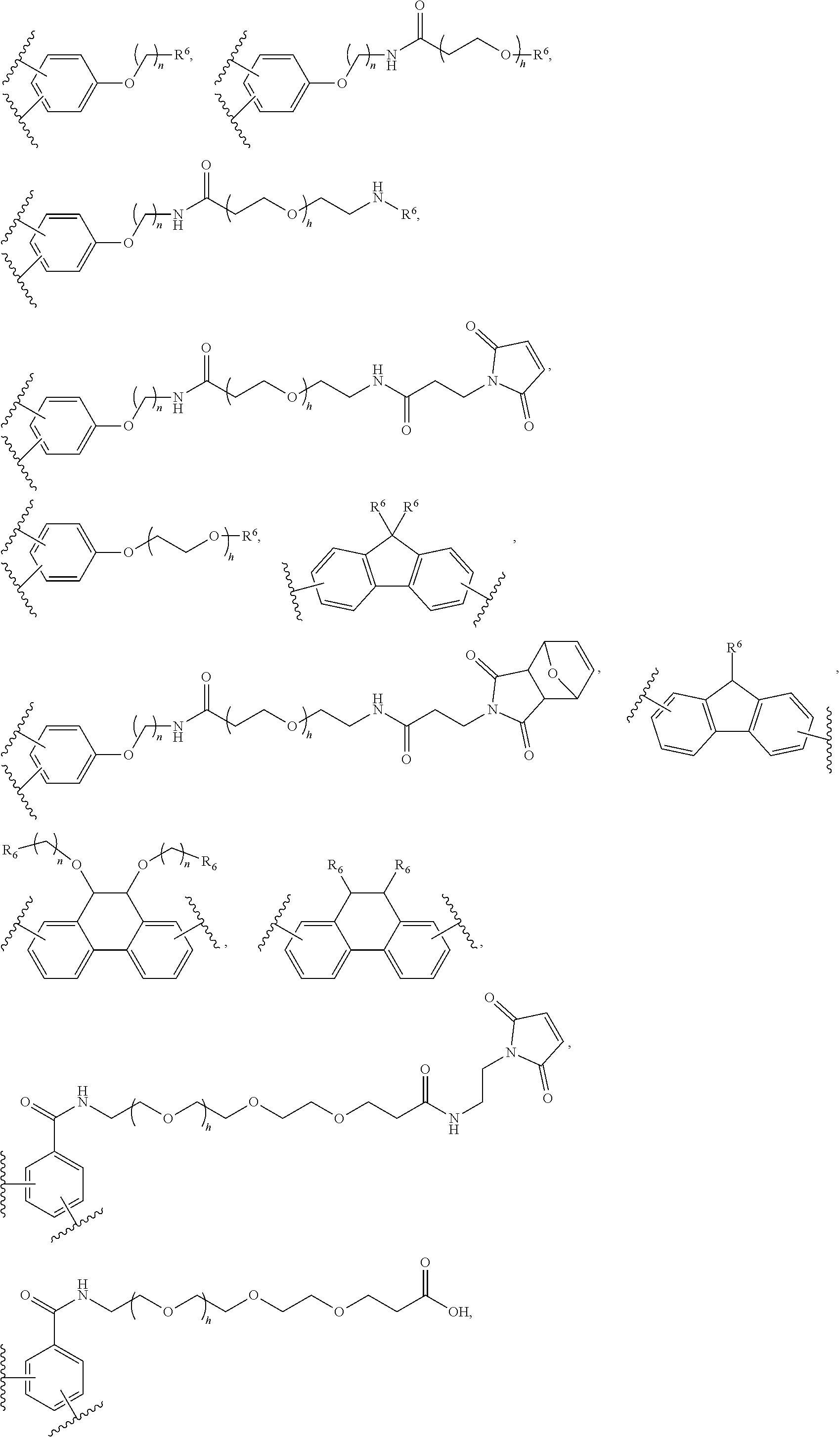

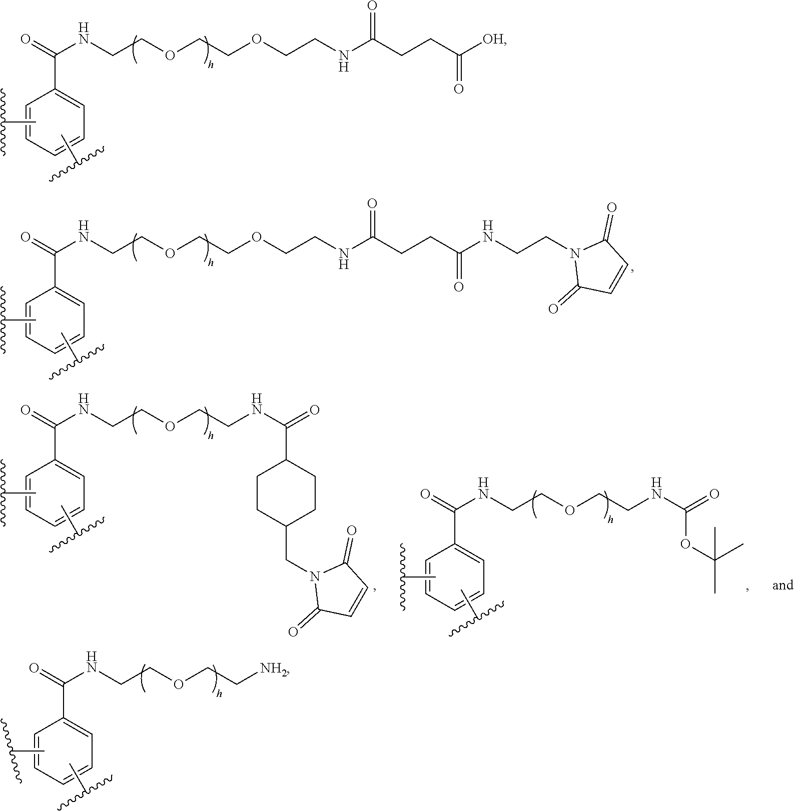

wherein
each R⁶ is independently selected from the group consisting of H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂ halo alkyl, C₁-C₁₂ (hetero)aryl, C₁-C₁₂ (hetero)arylamino, benzyl optionally substituted with one or more halogen, hydroxyl, C₁-C₁₂ alkoxy, or (OCH₂CH₂)_{f}OCH₃,

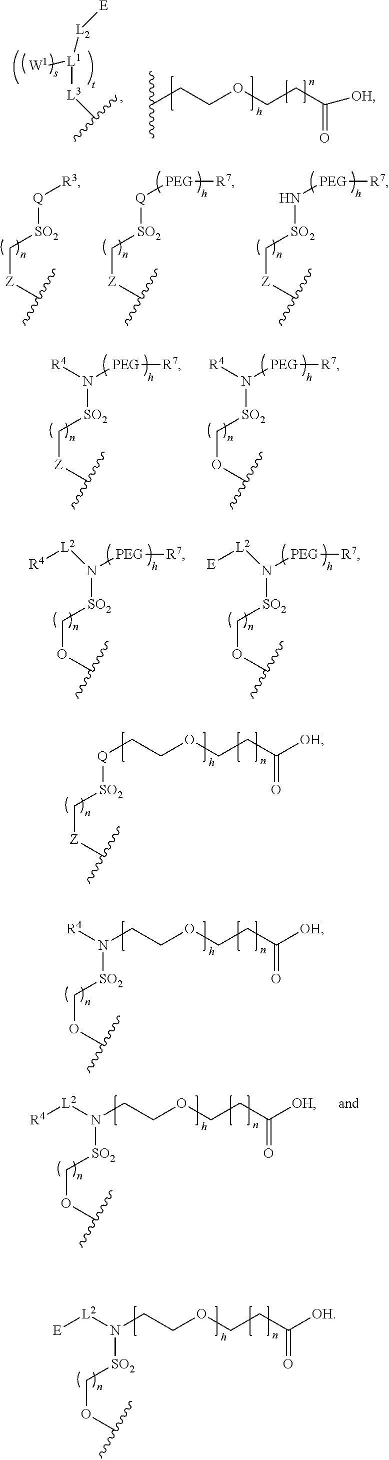

In some cases, each n is independently an integer from 1 to 20, 2 to 18, 3 to 15, 4 to 12, or 5 to 10.

In some embodiments, G¹ and G² may be each independently selected from the group consisting of hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), and optionally substituted fluorene, optionally substituted tetrahydropyrene (THP), wherein the substituted aryl, heteroaryl, fluorene, DHP or THP are substituted with one or more pendant chains terminated with a functional group, optionally conjugated to E.

In some cases, G¹ and G² may be each independently selected from the group consisting of.

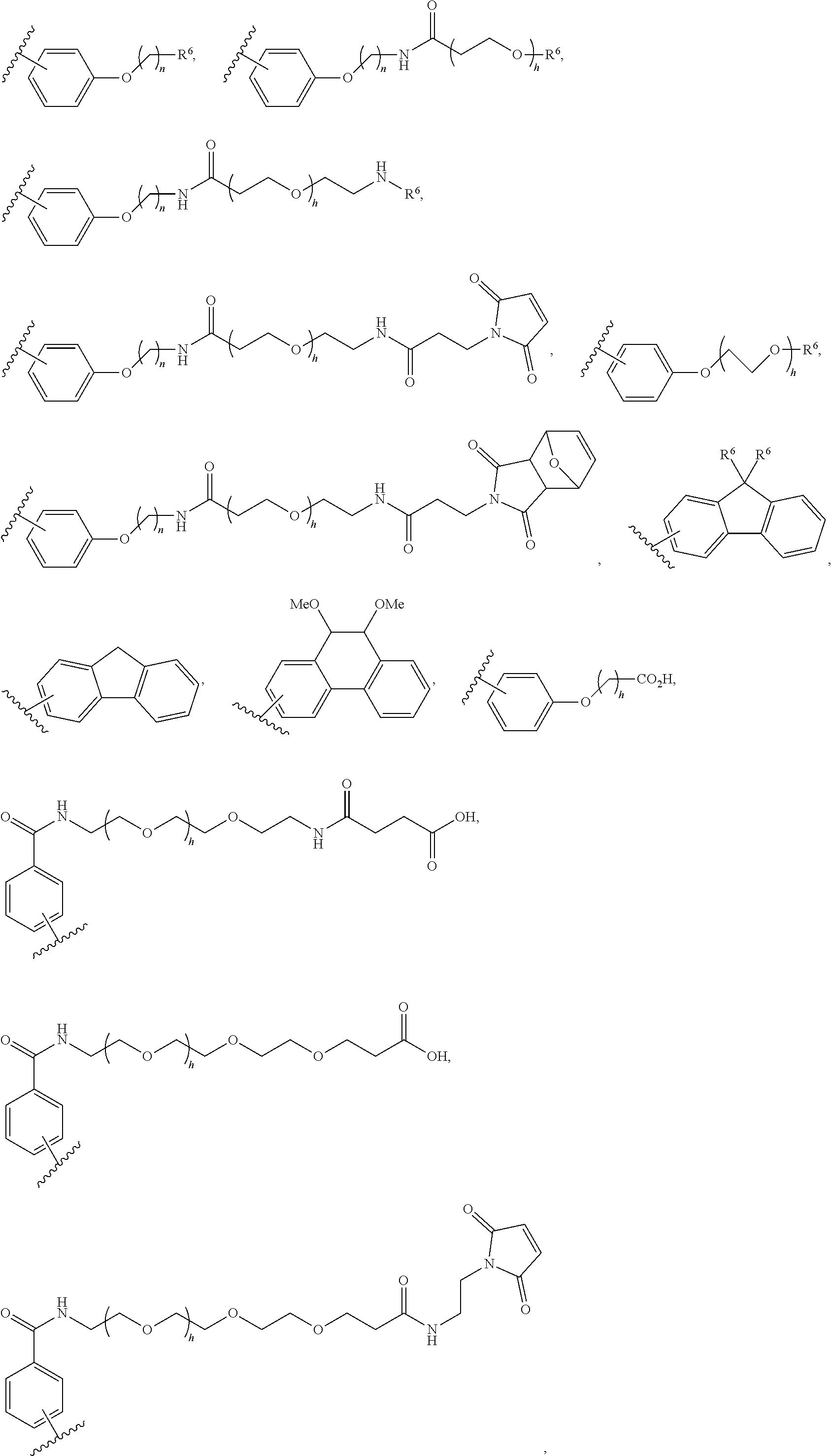

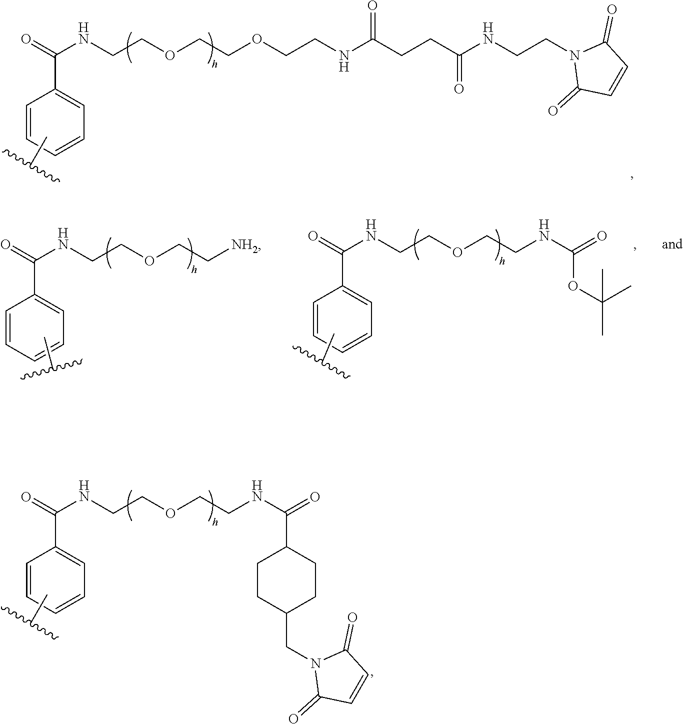

wherein
each R⁶ is independently selected from the group consisting of H, OH, SH, NHCOO-t-butyl, (CH₂)ₙCOOH, (CH₂)ₙCOOCH₃, (CH₂)ₙ(CH₂CH₂O)_{f}COOH, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino,

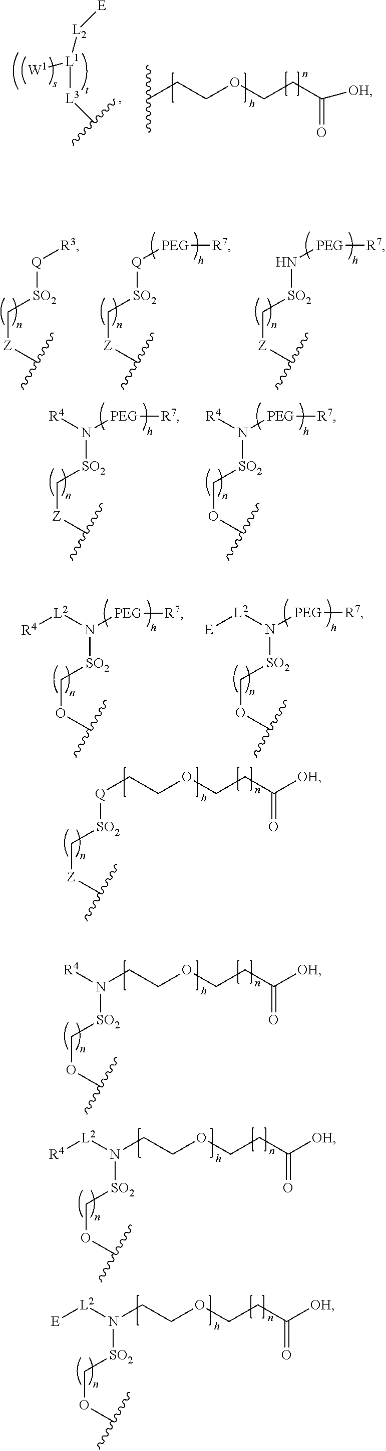

benzyl optionally substituted with one or more halogen, hydroxyl, C₁-C₁₂ alkoxy, or (OCH₂CH₂)_{f}OCH₃, and one or more pendant chains terminated with a functional group, optionally conjugated to E. In some cases, n is independently an integer from 1 to 20, 2 to 18, 3 to 15, 4 to 12, or 5 to 10.

The polymer dye according to the disclosure may be a water-soluble fluorescent polymer dye. The water-soluble polymer dye may comprise one or more water-solubilizing moieties. The water-solubilizing moieties may be each independently selected from the group consisting of a PEG group, carboxylic acid, carboxylate, polyvinyl alcohol, glycol, peptide, polyphosphate, polyalcohol, sulfonate, phosphonate, boronate, amine, ammonium, sulfonium, phosphonium, alcohol, polyol, oxazoline, zwitterionic derivative, carbohydrate, nucleotide, polynucleotide, substituted PEG group, substituted carboxy group, substituted carboxylic acid, substituted carboxylate, substituted glycol, substituted peptide, substituted polyphosphate, substituted polyalcohol, substituted sulfonate, substituted phosphonate, substituted boronate, substituted amine, substituted ammonium, substituted sulfoniums substituted phosphonium, substituted zwitterionic derivative, substituted carbohydrate, substituted nucleotide, and substituted polynucleotide, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide,

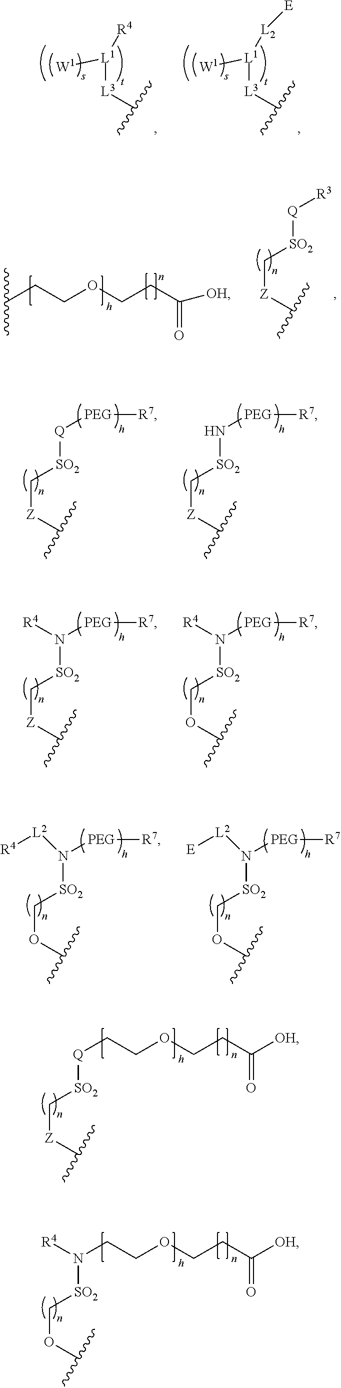

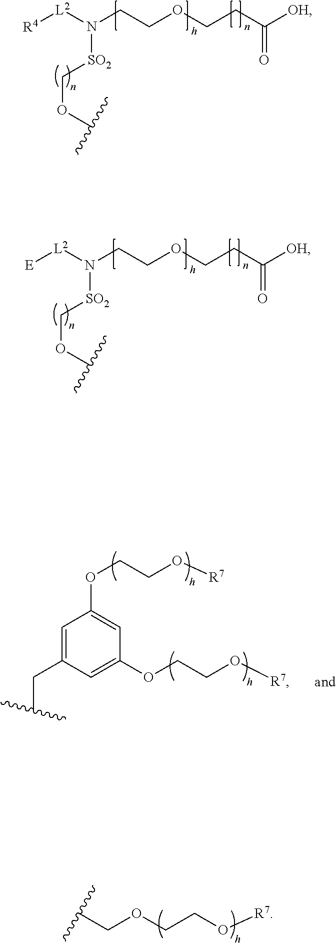

A polymer tandem dye is provided comprising a polymer dye according to the present disclosure; and a signaling chromophore (also referred to as an “acceptor dye”) covalently linked to the polymer dye in energy-receiving proximity therewith.

A labeled specific binding partner is provided, comprising a polymer dye or polymer tandem dye according to the present disclosure; and a specific binding partner capable of specific binding to a target analyte covalently linked to the polymer dye or polymer tandem dye. The specific binding partner may be selected from the group consisting of a protein, peptide, affinity ligand, antibody, antibody fragment, carbohydrate, lipid, nucleic acid, and an aptamer. The specific binding partner may be an antibody or an antigen-binding fragment thereof. In some cases, the specific binding partner may be covalently linked to a G¹ and/or G² moiety of the polymer dye or polymer tandem dye.

A method for detecting a target analyte in a sample is provided, the method comprising: providing a sample that is suspected of containing the target analyte; and contacting the sample with a labeled specific binding partner according to the present disclosure to form a fluorescent polymer dye conjugate complex with the target analyte if the target analyte is present; applying a light source to the sample that can excite the at least one fluorescent polymer dye conjugate complex; and detecting light emitted from the fluorescent polymer dye conjugate complex. The emitted light may have a wavelength greater than about 380 nm, or within a range of about 380 nm and about 1000 nm, or about 380 nm and about 800 nm.

The sample may be any biological sample. The sample may be selected from the group consisting of blood, bone marrow, spleen cells, lymph cells, bone marrow aspirates, urine, serum, saliva, cerebral spinal fluid, urine, amniotic fluid, interstitial fluid, feces, mucus, tissue, or is from a cell culture. The biological sample may be a blood sample. The biological sample may be a whole blood sample. The biological sample may be one or more cells of whole blood. The biological sample may be one or more cells such as erythrocytes, leukocytes, lymphocytes, phagocytes, monocytes, macrophages, granulocytes, basophils, neutrophils, eosinophils, platelets, and the like. The biological sample may be any cell with one or more detectable markers. The biological sample may be from a cell culture.

A kit is provided comprising at least one polymer dye, polymer tandem dye, or labeled specific binding partner according to the disclosure. The polymer dye, polymer tandem dye, or labeled specific binding partner according to the disclosure may comprise a conjugation tag for attachment of a signaling chromophore or specific binding partner.

### BRIEF DESCRIPTION OF THE DRAWINGS

FIG. 1 shows general structures of dihydronaphthothiophene (DHNT) and dihydrobenzodithiophene (DHBDT) monomer units.

FIG. 2 shows general structures of dihydronapthothienothiophene (DHNTT) monomer units.

FIG. 3 shows an exemplary synthetic method to provide DHNT molecule 6: 2,7-dibromo-4,5-dihydronaphtho[1,2-b]thiophene-4,5-diol.

FIG. 4 shows an exemplary synthetic method to provide DHNTT molecule 12: 3,10-dibromo-5,6,12,13-tetrahydronaphtho[1,2-b]naphtho[2′,1′:4,5]thieno[2,3-d]thiophene-5,6,12,13-tetraol.

FIG. 5 shows an exemplary synthetic method to provide DHNTT molecule 19: (3,8-dibromo-5,6-dimethyl-5,6-dihydronaphtho[1,2-b]thieno[2,3-d]thiophene-5,6-diol).

FIG. 6A shows an exemplary synthetic method to provide DHBDT molecule 21: 2,7-dibromo-4,5-dimethyl-4,5-dihydrobenzo[2,1-b:3,4-b′]dithiophene-4,5-diol.

FIG. 6B shows an exemplary synthetic method to provide PEGylated DHNT monomers and boronic esters. Similar methods were employed to prepare PEGylated DHNTT and DHBDT monomers and boronic esters.

FIG. 7 shows normalized excitation/emission spectra of DHP-DHP violet polymer A (green traces (a), SN427) emission max=427 nm vs DHP-DHNT co-polymer B emission max 457 nm. (red traces (b), λex=364 nm, Abs=0.05 in PBS 1×).

FIG. 8 shows an exemplary synthetic method to provide DHP-DINT co-polymer B from DHNT molecule 6.

FIG. 9 shows exemplary synthetic method to provide DHP-DHBDT co-polymer C from DHBDT molecule 21.

FIG. 10 shows Table 1 with photophysical data of DHP-DHNT co-polymer B and DHP-DHBDT co-polymer C, including Mn, PD, Abs max, emission max, extinction coefficient, and quantum yield.

FIG. 11 shows normalized UV-Vis spectra of violet polymer (a, black), DHP-DHNT co-polymer B (b, blue) and DHP-DHBDT co-polymer C (c, red) in PBS 1×.

FIG. 12 shows normalized excitation and emission spectra of violet polymer (a, black), DHP-DHNT co-polymer B (b, blue) and DHP-DHBDT co-polymer C (c, red) in PBS 1×.

FIG. 13 shows an exemplary method for preparing water-soluble polymer-antibody conjugates according to the present disclosure.

FIG. 14 shows an exemplary method for preparing a tandem polymer dye and antibody conjugation from water-soluble fluorescent polymers according to the present disclosure.

### DETAILED DESCRIPTION OF THE INVENTION

Water-soluble fluorescent polymers can be used in a variety of biological applications by generating signals in response to laser light excitation which can be monitored in real time and provide simple and rapid methods for the detection of biological targets and events.

The present disclosure provides novel fluorescent polymers, tandem polymers, labeled specific binding partners, and methods for detecting analytes in a sample using labeled specific binding partners comprising fluorescent polymers or polymer tandem dyes conjugated to binding partners. The present disclosure also provides kits comprising at least one polymer, tandem dye, and/or labeled specific binding partner of the disclosure, wherein the at least one polymer, tandem dye, and/or labeled specific binding partner may optionally include a conjugation tag (functional group). The fluorescent polymers of the present disclosure demonstrate water solubility, desirable brightness, and can be excited using violet, blue, yellow, green, red, and near infrared (NIR) wavelengths. Polymers according to the disclosure exhibit excitation maxima in a range of from about 300-810 nm, or 350-650 nm, or 350-565 nm, or 300-500 nm, or 320-420 nm.

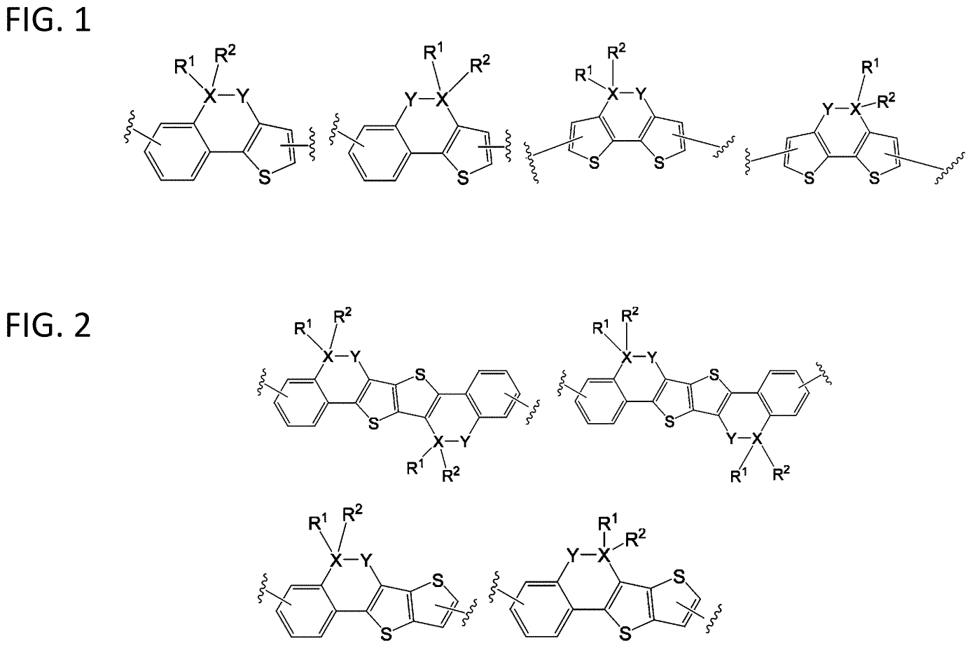

The present disclosure provides polymer and co-polymer dyes in which at least one phenyl ring of the dihydrophenanthrene (DHP) structure was replaced by a thiophene ring to obtain a dihydronaphthothiophene (DHNT) structure. In some examples of the present disclosure, both phenyl rings of the DHP structure were replaced by thiophene rings to obtain a dihydrobenzodithiophene (DHBDT) structure. The general structures of DHNT and DHBDT monomer units are shown in FIG. 1.

In some examples of the present disclosure, one phenyl ring of the DHP structure was replaced by thienothiophene rings to obtain a dihydronapthothienothiophene (DHNTT) structure. The general structure of DHNTT monomer units is shown in FIG. 2.

These monomeric compounds clearly show different absorption wavelengths when compared to DHP and upon polymerization produce polymers with red shifted emission spectra. The present disclosure provides the synthesis of DHNT, DHBDT, and DHNTT based monomers and their polymerization. Results shows that polymers with red shifted photophysical properties were obtained.

In some embodiments, the polymers according to the present disclosure exhibit longer Stokes shifts compared to prior art and control polymers. For example, DHP-DHNT co-polymer B exhibited excitation absorbance maxima of ~362 nm and fluorescence emission max of ~459 nm (Stokes shift of ~97 nm), and DHP-DHBDT exhibited excitation absorbance max of 472 nm and emission max of ~576 nm (Stokes shift ~104 nm). In contrast, DHP-DHP polymers and prior art fluorene-thiophene polymers can exhibit shorter Stokes shifts.

For example, DHP-DHP control polymer SN427 exhibited excitation abs max at about 414 nm and fluorescence emission max of 427 nm, (Stokes shift ~13 nm), and fluorene-thiophene polymers of U.S. Pat. No. 10,920,082, for example, having excitation absorbance max of 471 nm and emission max of 516 nm (Stokes shift ~45 nm).

A polymer dye exhibiting longer Stokes shift can be advantageous. For example, a single laser source can be used to activate fluorophores that emits in separate detectors of the flow cytometer, and this increases the number of markers that can be resolved in flow cytometry experiments. This is what tandem dyes also do, however, in tandem dyes often the acceptor dye can absorb laser source other than the donor activating source (collateral excitation) creating compensation issues when analyzing flow cytometry results. Generating dyes with long Stokes shift may help to minimize or eliminate the issue of collateral excitation.

In some embodiments, the polymers according to the present disclosure exhibit a Stokes shift of at least about 50 nm, at least about 60 nm, at least about 70 nm, at least about 80 nm, or at least about 90 nm, or in a range of from about 50 nm to about 130 nm, about 60 nm to about 120 nm, about 70 nm to about 110 nm.

### Definitions

The abbreviations used herein have their conventional meaning within the chemical and biological arts.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the present disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “reactive group” refers to a functional group that can selectively react with another compatible functional group to form a covalent bond, in some cases, after optional activation of one of the functional groups. Chemoselective functional groups of interest include, but are not limited to, thiols, maleimides, halogenated maleimides, iodoacetamides, amines, amide, sulfonamide, alkyl carboxylates, activated esters alkyl sulfonates, carboxylic amines, carbamate, carboxylate esters, N-hydroxysuccinimidyl (NHS), imido ester, halogen, boronic esters, boronic acids, hydrazonyl, carboxylic acids or active esters thereof, as well as groups that can react with one another via Click chemistry, e.g., azide and alkyne groups (e.g., cyclooctyne groups), tetrazine and alkene groups (e.g., cyclooctene groups), dienes and dienophiles, sulfur (VI) fluoride exchange chemistry (SuFEX), sulfonyl fluoride, as well as hydroxyl, hydrazido, hydrazino, aldehyde, ketone, azido, alkyne, phosphine, epoxide, urea, thiourea, thioester, imine, disulfides, and protected groups thereof and the like, or protected groups thereof. The reactive group may be a conjugation tag. The chemoselective functional group may be protected or unprotected. Additionally, orthogonal “functional group(s)” can be included that can be used for either bioconjugation of a binding partner to or the attachment of acceptor signaling chromophores in donor acceptor tandem dyes.

The term “amine-reactive group” refers to any group that forms a chemical bond with a primary amine. Amine-reactive groups of interest include, but are not limited to, isothiocyanates, isocyanates, acyl azides, NHS esters, imidoesters, sulfonyl chlorides, aldehydes, glyoxals, epoxides, oxiranes, carbonates, aryl halides, imidoesters, carbodiimides, anhydrides, and fluorophenyl esters. The amine-reactive group can be a NHS ester or imidoesters.

In some cases, non-covalent linking may involve specific binding between two moieties of interest (e.g., two affinity moieties such as a hapten and an antibody or a biotin moiety and a streptavidin, etc.). In certain cases, non-covalent linking may involve absorption to a substrate.

The term “symmetric” in reference to a compound of the present disclosure refers to wherein each terminal heterocyclic ring system is the same, and substituents may be the same or different. In some symmetric compounds, n=2.

The term “asymmetric” in reference to a compound of the present disclosure refers to wherein each terminal heterocyclic ring system is different. In some asymmetric compounds, n=3.

The term “counterion” refers to an ion that is charge balancing to the fluorescent compound according to the disclosure. The counter ion may be a cation. The counterion may be an anion. In some cases the counterion may be a halogen ion, perchlorate ion, PF⁶⁻, phosphate ion, sulfate ion, and the like. The counterion may be, F⁻, Cl⁻, Br⁻, I⁻, ClO₄⁻, CF₃CO₂⁻, CH₃CO₂⁻, PO₄³⁻, SO₄²⁻, BF₄⁻, and the like. In some cases the counterion may be Na⁺, K⁺, Mg⁺⁺, Ca⁺⁺, and the like.

The term “organic group” as used herein refers to any carbon-containing functional moiety. Examples can include an oxygen-containing group such as an alkoxy group; aryloxy group; aralkyloxy group; oxo(carbonyl) group; an amine group, including alkyl amine amine esters, and sulfonamide groups; a carboxyl group including a carboxylic acid, carboxylate, and a carboxylate ester; a sulfur-containing group such as an alkyl and aryl sulfide group, thiol, thiol reactive group, and sulfone group; maleimide; iodoacetamide; azide group; alkyne group; and other heteroatom-containing groups. Non-limiting examples of organic groups include OR, OOR, OC(O)N(R)₂, CN, CF₃, OCF₃, R, C(O)R, methylenedioxy, ethylenedioxy, N(R)₂, N₃, S(H)R, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “heteroatom” as used herein refers to any appropriate atom that is not carbon, such as, for example, N, O, S, Se, P, B, Al, Si, and Ge, inserted between adjacent carbon atoms in an organic group. The organic group may be a cyclic, aryl, or straight or branched chain group (e.g., alkyl or alkene). More than one heteroatom (e.g., 1, 2, 3, 4 or 5 heteroatoms) may be inserted between adjacent carbon atoms. The heteroatoms can be oxidized to form moieties such as, but not limited to, —S(O)— and S(O)₂—, sulfinate, sulfonamide.

The term “substituted” as used herein in conjunction with a molecule or an organic group as defined herein refers to the state in which one or more hydrogen atoms contained therein are replaced by one or more non-hydrogen atoms, such as, for example an alkyl, aryl, or a functional group. The “substituted” group may include one or more groups selected from halogen, hydroxy, amino, alkylamino, amido, acyl, nitro, cyano, and alkoxy. In some instances, the “substituted” group is selected from R¹, R², R³, R^{3′}and/or R⁴ groups as defined herein, In some instances the “substituted group, is selected from R¹, R², aryl, heteroaryl, H, alkyl, alkene, alkyne, cycloalkyl, haloalkyl, (hetero)aryloxy, (hetero)arylamino, halogen, a water-solubilizing moiety, functional moiety, a PEG group, —B(R³)₂, O-alkyl, O-aryl, NR′R′, NHR′, NH₂, —S—R′, SO₃H, —SO₂R′, —SO₂NR′, —PR′₃, POR′₃, —SiR′₃, -ammonium, alkylammonium, and arylammonium, where each R′ is independently selected from the group consisting of substituted or unsubstituted alkyl, substituted or unsubstituted aryl, optionally substituted with one or more PEG groups, water solubilizing moieties, linking moieties, linked water-solubilizing moiety, a linker moiety, a linked E, a reactive group, a linked reactive group, binding partner, linked binding partner, a functional group, a linked functional group.

The term “Stokes shift” refers to the difference between the positions of the band maxima of the absorption and emission spectra of the same electronic transition.

The term “functional group,” “functional moiety,” or “substituent” as used herein refers to a group that can be or is substituted onto a molecule or onto an organic group. Examples of substituents or functional groups include, but are not limited to, a halogen (e.g., F, Cl, Br, and I); alkene; cycloalkene; alkyne; cycloalkyne; an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, imides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl. The subject fluorescent compounds can include one or more “chemoselective functional group(s)” (also referred to as a “conjugation tag”) that provide for bioconjugation to a dye having the corresponding “chemoselective functional group” or “conjugation tag”. In some cases, such functionality may be used to covalently attach an acceptor or donor dye, or a biomolecule or binding partner such as a protein, peptide, affinity ligand, antibody, antibody fragment, polynucleotide, or aptamer. In some cases the functional group or conjugation tag may be selected from the group consisting of amine, carbamate, carboxylic acid, carboxylate, maleimide, activated ester, N-hydroxysuccinimidyl, hydrazine, hydrazide, hydrazone, isothiocyanato, azide, alkyne, cycloalkyne (e.g., alkene, cycloalkene (e.g., cyclooctene), tetrazine, aldehyde, thiol, and protected groups thereof for conjugation to a substrate, acceptor dye, functional moiety, or binding partner. The functional group may be protected or unprotected. The functional group may be a reactive or chemoselective functional group that can react with another group via copper-free click chemistry, including strain-promoted azide-alkyne cycloaddition (SPAAC) and inverse-electron-demand Diels-Alder (iEDDA) reactions that enable fast and specific chemical conjugation. See Kim et al., Chem. Sci., 2019, 10, 7835; and Davis et al., J. Org. Chem. 2016, 81, 6816-6819, both incorporated herein by reference in their entireties. The functional group or conjugation tag can be, for example, cycloalkene (e.g., cyclooctene); alkyne; cycloalkyne (e.g., cyclooctyne group, such as, for example, bicyclo[6.1.0] nonyne (BCN)), dibenzocyclooctyne (DBCO)); cycloalkene (e.g., cyclooctene group, such as, for example, trans-cyclooctene (TCO)); an azide group; or a tetrazine group.

As used herein, the term “activated ester” or “active esters” by itself or as part of another substituent refers to carboxyl-activating groups employed in peptide chemistry to promote facile condensation of a carboxyl group with a free amino group of an amino acid derivative. Descriptions of these carboxyl-activating groups are found in general textbooks of peptide chemistry, for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

As used herein, the term “ammonium” by itself or as part of another substituent refers to a cation having the formula NHR₃⁺where each R group, independently, is hydrogen or a substituted or unsubstituted alkyl, aryl, aralkyl, or alkoxy group. Preferably, each of the R groups is hydrogen.

As used herein, the term “oligoether” is understood to mean an oligomer containing structural repeat units having an ether functionality. As used herein, an “oligomer” is understood to mean a molecule that contains one or more identifiable structural repeat units of the same or different formula.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein some or all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

As used herein, the term “alkyl” by itself or as part of another substituent refers to a straight or branched, saturated, aliphatic radical having the number of carbon atoms indicated. Alkyl groups can be optionally substituted alkyl groups. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together. Alkyl can be optionally substituted or unsubstituted. Unless otherwise specified, “substituted alkyl” groups can be substituted with one or more groups selected from halo, hydroxy, amino, alkylamino, amido, acyl, nitro, cyano, and alkoxy. The alkyl group is typically monovalent, but can be divalent, such as when the alkyl group links two moieties together.

As used herein, the term “alkylene” refers to an alkyl group, as defined above, linking at least two other groups (i.e., a divalent alkyl radical). The two moieties linked to the alkylene group can be linked to the same carbon atom or different carbon atoms of the alkylene group. As used herein, the term “cycloalkyl” by itself or as part of another substituent refers to a saturated or partially unsaturated, monocyclic, fused bicyclic or bridged polycyclic ring assembly containing from 3 to 12 ring atoms, or the number of atoms indicated monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbomane. Cycloalkyl can be optionally substituted or unsubstituted. Heterocycloalkyl can be cycloalkyl wherein one or more carbon atoms are replaced with one or more heteroatoms.

As used herein, the term “haloalkyl” by itself or as part of another substituent refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, flouromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “halogen” by itself or as part of another substituent refers to fluorine, chlorine, bromine, and iodine.

As used herein, the term “alkoxy” by itself or as part of another substituent refers to an alkyl group, as defined above, having an oxygen atom that connects the alkyl group to the point of attachment. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

As used herein, the term “alkene” or “alkenyl” by itself or as part of another substituent refers to either a straight chain, branched chain, or cyclic hydrocarbon, having at least one double bond between two carbon atoms. Examples of alkene groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkene group is typically monovalent, but can be divalent, such as when the alkenyl group links two moieties together. The alkene can optionally be substituted or unsubstituted.

As used herein, the term “alkyne” or “alkynyl” by itself or as part of another substituent refers to either a straight chain or branched hydrocarbon, having at least one triple bond between two carbon atoms. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. The alkynyl group is typically monovalent, but can be divalent, such as when the alkynyl group links two moieties together. The alkyne can optionally be substituted or unsubstituted.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acyl group can optionally also include heteroatoms within the meaning herein. Examples of acyl groups include, but are not limited to, a nicotinoyl group (pyridyl-3-carbonyl) acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

As used herein, the term “aryl” by itself or as part of another substituent refers to cyclic aromatic hydrocarbon groups that do not contain heteroatoms in the aromatic ring assembly. “Aryl” groups can be a monocyclic or fused bicyclic, tricyclic or greater, aromatic ring assembly containing 6 to 16 ring carbon atoms. For example, aryl may be, but is not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, benzyl or naphthyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as herein before defined; or 1- or 2-naphthyl; or 1- or 2-phenanthrenyl. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. An example for oxy-C₂-C₃-alkylene-phenyl is 2,3-dihydrobenzofuran-5-yl. Substituted aryl groups include, e.g., but are not limited to, naphthyl or phenyl, optionally mono- or disubstituted by alkoxy, phenyl, halogen, alkyl or trifluoromethyl, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂alkoxy, C₂-C₁₈ (hetero)aryloxy, C₂-C₁₈ (hetero)arylamino, carboxylate, carboxylic acid, C₂-C₁₂ alkyl carboxylic acid, C₂-C₁₂ alkyl carboxylate, C₂-C₁₂ alkyl carboxylate ester, C₁-C₁₂ alkoxy, a water-solubilizing group (WSG), a functional group, sulfonic acid, sulfonate, C₁-C₁₂ alkyl sulfonate. In some cases, the substituted aryl group, such as for example naphthyl or phenyl, may be mono- or disubstituted by a functional group, a WSG, optionally a WSG comprising a functional group, alkoxy, halogen or trifluoromethyl. The WSG can be a branched WSG, optionally comprising a functional group, such as, for example, a WSG comprising PEG and a functional group.

Preferred as aryl is naphthyl, phenyl, or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

The term “polycyclic aryl” refers to an unsubstituted or substituted polycyclic ring system comprising 2 to 9, 2 to 8, or 2 to 6 aryl rings with or without fused cycloalkyl or cycloalkenyl rings. Examples of polycyclic aryl groups may include fluorene, 9H-fluorene, phenanthrene, dihydrophenanthrene, 9,10-dihydrophenanthrene, naphthalene, anthracene, tetracene, pentacene, and the like.

The term “Arylene” refers to a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. An example for oxy-C₂-C₃-alkylene-phenyl is 2,3-dihydrobenzofuran-5-yl. The polycyclic heteroaryl may be substituted or unsubstituted.

As used herein, the term “aryloxy” by itself or as part of another substituent refers to a O-aryl group, wherein aryl is as defined above. An aryloxy group can be unsubstituted or substituted with one or two suitable substituents. The term “phenoxy” refers to an aryloxy group wherein the aryl moiety is a phenyl ring. The term “(hetero)aryloxy” as used herein means an —O-heteroaryl group, wherein heteroaryl is as defined below. The term “(hetero)aryloxy” is used to indicate the moiety is either an aryloxy or (hetero)aryloxy group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

As used herein, the term “heteroaryl” by itself or as part of another substituent refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by, e.g., alkyl, nitro or halogen. Pyridyl represents 2-, 3- or 4-pyridyl, advantageously 2- or 3-pyridyl. Thienyl represents 2- or 3-thienyl. Heteroaryl may be thienyl, benzothienyl, thienothienyl, benzothienothienyl, dihydronaphthothienothienyl, thieno[3,2-b]thienyl, benzo[b]thieno[2,3-d]thienyl, or 5,6-dihydronaphtho[1,2-b]thieno[2,3-d]thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

In some embodiments, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′, —NH—C(NH₂)═NH, —NR′C(NH₂)═NH, —NH—C(NH₂)=NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X′—(CH₂)ₜ—, where s and t are independently integers of from 0 to 3, and X′ is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

The term “polycyclic heteroaryl” refers to an unsubstituted or substituted polycyclic ring system comprising 2 to 9, 2 to 8, or 2 to 6 aryl rings with or without fused cycloalkyl or cycloalkenyl rings, and wherein the polycyclic ring system comprises one or more, two or more, three or more, or four or more heteroatoms. Non-limiting examples of polycyclic heteroaryl systems may include quinoline, benzoxazole, benzothiazole, benzimidazole, indole, benzindole, pyridinium, benzopyrylium, thiopyrylium, 6,8-dihydro-5H-naphtho[2,1-f]indole, 4,5-dihydro-3H-naphtho[2,1-e]indole, 6,7-dihydro-3H-naphtho[2,1-g]indole, 5,6-dihydrophenanthro[3,2-d]thiazole, 4,5-dihydrophenanthro[2,1-d]thiazole, 6,7-dihydrophenanthro[4,3-d]thiazole, 5,6-dihydrophenanthro[3,2-d]oxazole, 4,5-dihydrophenanthro[2,1-d]oxazole, 6,7-dihydrophenanthro[4,3-d]oxazole, 5,6-dihydronaphtho[2,1-g]quinoline, 7,8-dihydronaphtho[2,1-h]quinoline, 5,6-dihydronaphtho[2,1-f]quinoline, 5,6-dihydro-812-phenanthro[3,2-d]imidazole, 4,5-dihydro-312-phenanthro[2,1-d]imidazole, 6,7-dihydro-312-phenanthro[4,3-d]imidazole, 5,6-dihydronaphtho[1,2-g]quinoxaline, 5,6-dihydronaphtho[2,1-f]quinoxaline, 7,8-dihydronaphtho[1,2-f]quinoxaline, 5,6-dihydropentapheno[3,2,1-cd:10,11,12-c′d′]diindole, 3,8-dihydrophenanthro[2,3-e:7,6-e′]diindole, 3,5,6,8-tetrahydrophenanthro[2,3-e:7,6-e′]diindole, 1,2,3,5,6,8-hexahydrophenanthro[2,3-e:7,6-e′]diindole-1,10-diium salt, 3,5,6,8-tetrahydrophenanthro[2,3-e:7,6-e′]diindole-ethane (1/1), 5,6-dihydropentapheno[3,4-d:10,9-d′]bis(oxazole), 1,2,5,6,9,10-hexahydropentapheno[3,4-d:10,9-d′]bis(thiazole)-1,10-diium, pentapheno[3,4-d:10,9-d′]bis(thiazole), 1,2,9,10-tetrahydropentapheno[3,4-d:10,9-d′]bis(thiazole)-1,10-diium, 6,7-dihydrophenanthro[2,3-f:7,6-f]diquinoline-1,12-diium, 6,7-dihydrophenanthro[2,3-f:7,6-f]diquinoline, 2,6,7,11-tetrahydrobenzo[1,2-g:4,3-g′]dichromene, 2,11-dihydrobenzo[1,2-g:4,3-g′]dichromene, 5,10-dihydro-6H-naphtho[2,1-g]chromene, 10H-naphtho[2,1-g]chromene, 2,6,7,11-tetrahydrobenzo[1,2-g:4,3-g′]bis(thiochromene), 2,11-dihydrobenzo[1,2-g:4,3-g′]bis(thiochromene), and the like. The term “monocyclic heteroaryl” refers to an unsubstituted or substituted heteroaryl ring system comprising 1 aryl ring, and wherein the monocyclic ring system comprises one or more, two or more, three or more, or four or more heteroatoms. Nonlimiting examples of a monocyclic heteroaryl group are substituted or unsubstituted pyridinyl, pyranyl, thiophenyl, furanyl, pyrrolyl, pyrazolyl, imidazolyl, thiazolyl, oxazolyl.

As used herein, the terms “heteroalkyl” or “heteroalkoxy” by itself or as part of another substituent refers to an alkyl or alkoxy group, preferably a C₁-C₁₂ alkyl group or C₁-C₁₂ alkoxy group where a C is substituted by a heteroatom such as N, O or S. For example, heteroalkyl or heteroalkoxy can include ethers, thioethers and alkyl-amines. Additional heteroatoms can also be useful, including, but not limited to, B, Al, Si, or P. The heteroatoms can be oxidized to form moieties such as, but not limited to, —S(O)—, —S(O)₂—, sulfinate, sulfonamide. The heteroatom portion of the heteroalkyl can replace a hydrogen atom of the alkyl group to form a hydroxy, thio or amino group. Alternatively, the heteroatom portion can be the connecting atom, or be inserted between two carbon atoms.

As used herein, the term “heteroalkylene” refers to a heteroalkyl group, as defined above, linking at least two other groups (i.e., a divalent heteroalkyl radical). The two moieties linked to the heteroalkylene group can be linked to the same atom or different atoms of the heteroalkylene group.

As used herein, the term “cycloalkylene” refers to a cycloalkyl group, as defined above, linking at least two other groups (i.e., a divalent cycloalkyl radical). The two moieties linked to the cycloalkylene group can be linked to the same atom or different atoms of the cycloalkylene group.

As used herein, the terms “polyethylene glycol”, “PEG”, “PEG group”, P“polyethylene oxide” or “PEO” refer to the family of biocompatible water-solubilizing linear polymers based on the ethylene glycol monomer unit described by the formula —(CH₂—CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, alkoxy, aryl, hydroxyl, amino, acyl, carboxylic acid, carboxylate ester, acyloxy, and amido terminal and/or substituent groups. As used herein, PEG groups include, but are not limited to, PEG, modified PEG, linked PEG, amide-PEG, sulfonamide-PEG, phosphoramide-PEG, alkyl sulfonamide-PEG and alkoxy sulfonamide-PEG. It is understood that the PEG polymeric moiety may be of any convenient length and may include a variety of terminal groups and/or further substituent groups, including but not limited to, alkyl, aryl, hydroxyl, amino, acyl, acyloxy, and amido terminal and/or substituent groups. PEG groups that may be adapted for use with the subject compounds include those PEGs described by S. Zalipsky in “Functionalized poly(ethylene glycol) for preparation of biologically relevant conjugates”, Bioconjugate Chemistry 1995, 6 (2), 150-165; by Zhu et al in “Water-Soluble Conjugated Polymers for Imaging, Diagnosis, and Therapy”, Chem. Rev., 2012, 112 (8), pp 4687-4735; by J. M. Harris in “Poly(ethylene glycol) Chemistry: Biotechnical and Biomedical Applications”, Plenum Press, New York, N.Y. (1992); and by J. M. Harris and S Zalipsky in “Poly(ethylene glycol) Chemistry and Biological Applications”, ACS (1997). In some instances, PEG and modified PEG moieties can be, for example, those taught in International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964 U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966 each of which are incorporated herein by reference).

As used herein, the term “amine” by itself or as part of another substituent as used herein refers to an alkyl groups as defined within, having one or more amino groups. The amino groups can be primary, secondary or tertiary. The alkyl amine can be further substituted with a hydroxy group. Amines useful in the present disclosure include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present disclosure.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “amide” refers to a functional group having a carbonyl group attached to an amine group, having the general formula RC(═O)NR′R″, where R, R′, and R″ represent organic groups or hydrogen atoms. The term “amido” refers to a substituent containing an amide group.

As used herein, the term “(hetero)arylamino” by itself or as part of another substituent refers an amine radical substituted with an aryl group (e.g., —NH-aryl). An arylamino may also be an aryl radical substituted with an amine group (e.g., -aryl-NH₂). Arylaminos may be substituted or unsubstituted.

As used herein, the term “carbamate” by itself or as part of another substituent refers to the functional group having the structure —NR″CO₂R′, where R′ and R″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, V-(2-pivaloylamino)-1,1-dimethylethyl carbamate, NpSSPeoc.

As used herein, the term “carboxylic acid” by itself or as part of another substituent refers to a structure R—COOH where R is a carbon-containing group of atoms.

As used herein, the term “carboxylate” by itself or as part of another substituent refers to the conjugate base of a carboxylic acid, which generally can be represented by the formula RCOO⁻. For example, the term “magnesium carboxylate” refers to the magnesium salt of the carboxylic acid The term “carboxylate ester” as used herein by itself or as part of another substituent refers to a compound derived from a carboxylic acid, which generally can be represented by the formula RCOOR′ where R′ can be an alkyl, alkene, alkyne, haloalkyl, heteroalkyl, cycloalkyl, aryl, heteroaryl, (unsubstituted aryl)alkyl, and (unsubstituted aryl)oxy-alkyl or other carbon-containing group of atoms. R′ can optionally contain functional groups.

As used herein, the term “sulfonate functional group” or “sulfonate” either by itself or as part of another substituent refers to both the free sulfonate anion (—S(═O)₂O—) and salts thereof. Therefore, the term sulfonate encompasses sulfonate salts such as sodium, lithium, potassium and ammonium sulfonate.

As used herein, the term “sulfonamido” by itself or as part of another substituent refers to a group of formula —SO₂NR— where R can be, for example, a water solubilizing moiety, hydrogen, alkyl, alkene, alkyne, cycloalkyl, haloalkyl, aryl, or functional group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “sulfonamide” by itself or as part of another substituent refers to a group of formula —SO₂NR₂ where each R can independently be, for example, a water solubilizing moiety, hydrogen, alkyl, alkene, alkyne, cycloalkyl, haloalkyl, aryl, or functional group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “sulfinamide” by itself or as part of another substituent refers to a group of formula —SONR₂ where each R can independently be, for example, a water solubilizing moiety, hydrogen, alkyl, alkene, alkyne, cycloalkyl, haloalkyl, aryl, or functional group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “activated ester” or “active esters” by itself or as part of another substituent refers to carboxyl-activating groups employed in peptide chemistry to promote facile condensation of a carboxyl group with a free amino group of an amino acid derivative. Descriptions of these carboxyl-activating groups are found in general textbooks of peptide chemistry, for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

As used herein, the terms “hydrazine” and “hydrazide” by themselves or as part of another substituent refer to compounds that contain singly bonded nitrogens, one of which is a primary amine functional group. For example, the term “hydrazine” refers to a moiety having the structure —NHNH₂.

As used herein, the term “aldehyde” by itself or as part of another substituent refers to a chemical compound that has a —CHO group.

As used herein, the term “thiol” by itself or as part of another substituent refers to a compound that contains the functional group composed of a sulfur-hydrogen bond. The general chemical structure of the thiol functional group is R—SH, where R represents an alkyl, alkene, aryl, or other carbon-containing group of atoms.

As used herein, the term “silyl” by itself or as part of another substituent refers to Si(R^{z})₃ wherein each R independently is alkyl, aryl or other carbon-containing group of atoms.

As used herein, the term “diazonium salt” by itself or as part of another substituent refers to a group of organic compounds with a structure of R—N₂⁺X⁻, wherein R can be any organic group (e.g., alkyl or aryl) and X is an inorganic or organic anion (e.g., halogen).

As used herein, the term “triflate” by itself or as part of another substituent also referred to as trifluoromethanesulfonate, is a group with the formula CF₃SO₃.

As used herein, the term “boronic acid” by itself or as part of another substituent refers to a structure —B(OH)₂. It is recognized by those skilled in the art that a boronic acid may be present as a boronate ester at various stages in the synthesis of the quenchers. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. In some embodiments, the boronic ester moiety is a 5-membered ring. In some other embodiments, the boronic ester moiety is a 6-membered ring. In some other embodiments, the boronic ester moiety is a mixture of a 5-membered ring and a 6-membered ring.

As used herein, the term “maleimide” by itself or as part of another substituent refers a structure

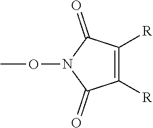

where R can be, for example, a water solubilizing moiety, hydrogen, halogens, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “hydrazone” by itself or as part of another substituent refers to a structure

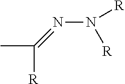

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “azide” by itself or as part of another substituent refers to a structure —N₃.

As used herein, the term “tetrazine” by itself or as part of another substituent refers to a compound having the molecular formula C₂H₂N₄ that consists of a six-membered heteroaromatic ring structure having 4 nitrogen atoms. The term “tetrazine” includes all of its isomers, namely 1,2,3,4-tetrazines, 1,2,3,5-tetrazines, and 1,2,4,5-tetrazines.

As used herein, the term “N-hydroxysuccinimidyl” by itself or as part of another substituent refers to a structure

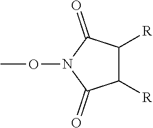

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphoramide” by itself or as part of another substituent refers to a structure

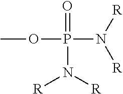

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphonamidate” by itself or as part of another substituent refers to a structure

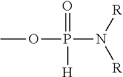

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphinamide” by itself or as part of another substituent refers to a structure

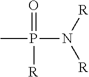

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphonate” by itself or as part of another substituent refers to the structure

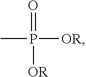

where R can be, for example, alkyl, aryl, hydrogen, or other group.

The term “fluorescent” as used herein refers to a compound which, when irradiated by light of a wavelength that the compound absorbs, emits light of a (typically) different wavelength. Fluorescence is the emission of light by a substance that has absorbed light or other electromagnetic radiation. In most cases, the emitted light has a longer wavelength than the absorbed light.

The term “absorbance maxima” or “Abs 1 max” or “max 1 abs” refer to wavelength of maximum absorbance measured by UV Vis spectroscopy.

The term “excitation wavelength” or “1 ex” refers to the wavelength where the compound can be excited to induce fluorescence emission, it does not necessarily have to be at the Abs 1 max.

The term “chromophore” refers to a molecular entity or a portion thereof consisting of an atom or a group of atoms in which the electronic transition responsible for a given spectral band is approximately localized. In some instances, the “chromophore” may itself be fluorescent. The terms “chromophore” and “fluorophore” are used interchangeably herein and may refer to an acceptor dye. As used herein, the terms “fluorescent chromophore” and “fluorescent dye” are used interchangeably and refer to a compound which has a structure capable of harvesting light with a particular absorption maximum wavelength and converting it to emitted light at a longer emission maximum wavelength. A chromophore may have a reactive group (e.g., a carboxylate moiety, an amino moiety, a haloalkyl moiety, or the like) that can be covalently bonded. Examples of suitable chromophores include, but are not limited to, those described in U.S. Pat. Nos. 7,687,282; 7,671,214; 7,446,202; 6,972,326; 6,716,979; 6,579,718; 6,562,632; 6,399,392; 6,316,267; 6,162,931; 6,130,101; 6,005,113; 6,004,536; 5,863,753; 5,846,737; 5,798,276; 5,723,218; 5,696,157; 5,658,751; 5,656,449; 5,582,977; 5,576,424; 5,573,909; and 5,187,288, which patents are incorporated herein by reference in their entirety.

The term “moiety” refers to a group as a portion of a molecule, which may be a functional group, or a portion of a molecule with multiple groups which share common structural and/or functional aspects. Examples of group or moiety include but are not limited to a linker moiety, a functional group, a water-solubilizing moiety, a PEG moiety, according to the present disclosure.

The term “linker,” “linked” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some embodiments, the linker is a branching linker that refers to a linking moiety that connects three or more groups. In some embodiments, the linker backbone includes a linking functional group, such as an ether, thioether, amino, amide, carbonyl, acyl, sulfonamide, a sulfinamide, a disulfonamide, a disulfinamide, a sultam, an amide, a secondary amine, a phosphonamide, a phosphinamide, a phosphonamidate, a selenonamide, a seleninamide, carbamate, thiocarbamate, urea, thiourea, ester, thioester or imine. In some embodiments, the linker backbone includes a linking functional group, such as an amino, amide, carbonyl, sulfonamide, sulfinamide, a disulfonamide, a disulfinamide, a sultam, an amide, a secondary amine, a phosphonamide, a phosphinamide, a phosphonamidate, a selenonamide, or a seleninamide. In certain cases, one, two, three, four or five or more carbon atoms of a linker backbone may be optionally substituted with a sulfur, nitrogen or oxygen heteroatom. In some embodiments, the linker backbone includes a linking functional group, such as an ether, thioether, amino, amide, carbonyl, acyl, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol, ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3, or 4 atoms, of the cyclic group are included in the backbone. A linker may be cleavable or non-cleavable.

A linker moiety can be attached to a polymer according to the disclosure, as taught in U.S. Pat. No. 11,584,825 B2, which is incorporated herein by reference in its entirety. A linker moiety can comprise covalent bond, an alkoxy, sulfonamide, disulfonamide, a selenomide, a sulfinamide, a sultam, a disulfinamide, an amide, carbonyl, a seleninamide, a phosphonamide, a phosphinamide, a phosphonamidate, or a secondary amine.

As described in U.S. Pat. No. 11,584,825 B2, and as each pertains to a linker moiety, the term “sulfonamide,” refers to a moiety —S(O)₂NR—; the term “disulfonamide,” refers to a moiety —S(O)₂NRS(O)₂—; the term “selenonamide,” refers to a moiety —Se(O)₂NR—; the term “sulfinamide,” refers to a moiety —S(O)NR₂; the term “disulfinamide,” refers to a moiety —S(O)NRS(O)—; the term “seleninamide,” refers to a moiety —Se(O)NR—; the term “phosphonamide,” refers to a moiety —NR—PR(O)NR—; the term “phosphinamide,” refers to a moiety —PR(O)NR—; and the term “phosphonamidate,” refers to a moiety —O—PR(O)NR—; and the term “sultam” refers to a cyclic sulfonamide (e.g., wherein the R group is bonded to the sulfur atom via an alkylene moiety); wherein for each term the R group is independently H, alkyl, haloalkyl, or aryl.

The term “quantum yield” (“QY”) (φ) or “fluorescence quantum yield” refers to the ratio of the number of photons emitted to the number of photons absorbed. The quantum yield is independent of instrument settings and describes how efficiently a fluorophore converts the excitation energy into fluorescence. Experimentally the relative fluorescence quantum yields can be determined by measuring fluorescence of a fluorophore of known quantum yield with same experimental parameters (excitation wavelength, slit widths, photomultiplier voltage, etc.) as the test dye. The quantum yield may be determined by any method known in the art. For example, the QY may be determined per manufacturer's instructions in a fluorescence spectrofluorometer or fluorescence spectrometer at a selected excitation wavelength. For example, Quantum yield (QY) may be determined on a Shimadzu Rf-6000 Fluorescence Spectrofluorometer by measuring emission intensity at an emission wavelength maxima, e.g., in a range of λₑₘ425-600 nm; 440-580 nm, e.g., 428 nm, 459 nm, or 576 nm from a diluted PBS solution of a staining buffer compared with absorbance at an absorbance wavelength maxima, e.g., in a range of λₘₐₓ340-500 nm; 360-475 nm, e.g., 362 nm, 472 nm, or for example, 405 nm=0.05 (excitation at 405 nm, ex slit 1.5, em slit 3.0, 1 cm quartz cuvette). The quantum yield may be calculated, for example, by comparing the intensity measured from the sample and the intensity measured from an appropriate control compound, e.g., NHS Pacific Blue (QY=0.78 in PBS 1×) solution under the same experimental conditions. In some embodiments, the QY may be determined, for example, according to the method of Lawson-Wood et al., Application Note-Fluorescence Spectroscopy, Determination of relative fluorescence quantum yield using the FL5600 fluorescence spectrometer, 2018, PerkinElmer, Inc. The selected excitation wavelength may be any appropriate excitation wavelength in a range of, e.g., λₘₐₓ340-500 nm; 360-475 nm, e.g.,

362 nm, 405 nm, or 472 nm. The “brightness” of a fluorescent molecule depends on the extinction coefficient (how strongly a fluorescent molecule absorbs light at a particular wavelength), and fluorescence quantum yield (how efficiently the absorbed light is converted to emitted light). In some cases, the brightness=extinction coefficient×fluorescence quantum yield. For example, DHP-DHBDT co-polymer C polymer brightness=1,700,000 cm⁻¹M⁻¹×0.065=110,500 (or 110.5 K). To obtain the “brightness” of a polymer dye in K, it may be calculated as QY*Extinction/1000. For example, DHP-DHNT co-polymer B brightness=150,000 cm⁻¹M⁻¹×0.1/1000=15 K.

The subject water-soluble fluorescent polymers feature termini on the conjugated polymer chains that can include a functional group that provides for bioconjugation. In some cases, such functionality is referred to as an end linker or end group. With these end linkers, a covalent bond can be formed to attach a biomolecule such as a protein, peptide, affinity ligand, antibody, antibody fragment, polynucleotide, or aptamer. For example, polymeric dye-labeled antibodies find use in flow cytometry as reagents exhibiting high brightness. Additionally, orthogonal functional groups can be installed along the conjugated polymer chain that can be used for either bioconjugation or the attachment of acceptor signaling chromophores in donor acceptor polymeric tandem dyes.

The phrase “conjugated water-soluble fluorescent polymer” refers to a water-soluble fluorescent polymer having a binding partner conjugated thereto.

“ [symbol] ” represents either a single or double bond.

The phrase “binding partner” refers to any molecule or complex of molecules capable of specifically binding to a target analyte. A binding partner of the present disclosure includes for example, a protein (e.g., an antibody or an antibody fragment), a small organic molecule, a carbohydrate (e.g., a polysaccharide), an oligonucleotide, a polynucleotide, a lipid, an affinity ligand, an aptamer, or the like. In some embodiments, the binding partner is an antibody or fragment thereof. Specific binding in the context of the present disclosure refers to a binding reaction which is determinative of the presence of a target analyte in the presence of a heterogeneous population. Thus, under certain assay conditions, the specified binding partners bind preferentially to a particular protein or isoform of the particular protein and do not bind in a significant amount to other proteins or other isoforms present in the sample.

In some cases, the antibody includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. Aspects of IVIG are described, for example, in US. Pat. Appl. Pub. Nos. 2010/0150942; 2004/0101909; 2013/0177574; 2013/0108619; and 2013/0011388.

In some cases, the antibody is a monoclonal antibody of a defined sub-class (e.g., IgG1, IgG2, IgG3, or IgG4). If combinations of antibodies are used, the antibodies can be from the same subclass or from different subclasses. For example, the antibodies can be IgG1 antibodies. In some embodiments, the monoclonal antibody is humanized.

The phrase “water-soluble fluorescent polymer complex” refers to a water-soluble fluorescent polymer of the present disclosure conjugated with a binding partner.

The phrase “protected group” (also referred to as “protecting group”) refers to a reversibly formed derivative of an existing functional group in a molecule attached to decrease reactivity so that the protected functional group does not react under synthetic conditions to which the molecule is subjected. Examples of amine protecting groups include, but are not limited to, benzyloxycarbonyl; 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihvdrobenzofuran-5-sulfonyl (Pbf); mesityl-2-sulfonyl (Mts); 4-methoxy-2,3,6-trimethylphenylsulfonyl (Mtr); acetamido; phthalimido; and the like. These and other protecting groups for amines, carboxylic acids, alcohols, and further functional groups can be added to and removed from compounds of the present disclosure using known techniques as described, for example, by Green and Wuts (Protective Groups in Organic Synthesis, 4^{th} Ed. 2007, Wiley-Interscience, New York).

The term “substrate” refers to a solid material having a variety of configurations. The substrate can be, for example, a sheet, bead, or other structure, such as a plate with wells, a polymer, particle, a semiconductor surface, nanotubes, a fibrous mesh, hydrogels, porous matrix, a pin, a microarray surface, a chromatography support, and the like. In some instances, the substrate is selected from the group consisting of a particle, a planar solid substrate, a fibrous mesh, a hydrogel, a porous matrix, a pin, a microarray surface and a chromatography support.

The term “sample” refers to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants, or fungi.

The term “water” as used herein refers to any aqueous solution that is primarily water and is compatible with physiological conditions. In some instances, the aqueous solution contains more than 50% water, such as more than 60% water, more than 70% water, more than 80% water, more than 90% water, or more than 95% water. The term “water” includes, for example, biological buffers and other aqueous solutions that may contain additives such as salts, detergents, stabilizers, and other water-soluble components, for example, sugars, proteins, amino acids, and nucleotides. In some instances, “water” may be an aqueous solution containing up to 10% miscible organic solvent (e.g., up to 10% DMSO in water). The term “water” does not include pure solvents or solvent combinations different from water, such as pure alcohols, for example pure methanol or ethanol, pure ethers, for example pure diethyl ether or tetrahydrofuran, or any other pure solvent either miscible or not miscible with water.

The term “water solubilizing moiety” or “water-solubilizing group” (WSG or W¹) as used herein by itself or part of another group refers to any hydrophilic group that is well solvated in aqueous environments, for example such as under physiological conditions, and is capable of increasing the water solubility of the polymer to which it is attached. The increase in water solubility of the molecule can vary depending upon the moiety attached. In some instances, the increase in water solubility (as compared to the solubility of the polymer without the moiety attached) is 2 fold or more, 5 fold or more, 10 fold or more, 25 fold or more, 50 fold or more, or 100 fold or more. “Water-solubilizing moiety” includes moieties, such as, but not limited to, PEG groups, carboxy groups including but not limited to carboxylic acids and carboxylates, polyvinyl alcohol, glycols, peptides, polyphosphates, polyalcohols, sulfonates, phosphonates, boronates, amines, ammoniums, sulfoniums, phosphonium, alcohols, polyols, oxazolines, zwitterionic derivatives, carbohydrates, nucleotides, polynucleotides, substituted PEG groups, substituted carboxy groups including but not limited to substituted carboxylic acids and substituted carboxylates, substituted glycols, substituted peptides, substituted polyphosphates, substituted polyalcohols, substituted sulfonates, substituted phosphonates, substituted boronates, substituted amines, substituted ammoniums, substituted sulfoniums, substituted phosphonium, alcohols, substituted zwitterionic derivatives, substituted carbohydrates, substituted nucleotides, substituted polynucleotides, and combinations thereof.

Any convenient WSG may be included in the dyes described herein to provide for increased water-solubility. A water-solubilizing moiety can increase the solubility of a compound in a predominantly aqueous solution, as compared to a control compound which lacks the water-solubilizing moiety. The water-solubilizing moiety may be any convenient hydrophilic moiety that is well solvated in aqueous environments. In some instances, the water-solubilizing moiety can be capable of imparting solubility in water (e.g., aqueous buffer) >1 mg/mL, >2 mg/mL, >3 mg/mL, >4 mg/mL, >5 mg/mL, >6 mg/mL, >7 mg/mL, >8 mg/mL, >9 mg/mL, or >10 mg/mL. In some instances, the water-solubilizing moiety can be capable of imparting solubility in water of >10 mg/mL, >20 mg/mL, >30 mg/mL, >40 mg/mL, >50 mg/mL, >60 mg/mL, >70 mg/mL, >80 mg/mL, >90 mg/mL or >100 mg/mL.

The increase in water solubility of the molecule can vary depending upon the moiety attached. In some instances, the increase in water solubility (as compared to the solubility of the molecule without the moiety attached) is 2 fold or more, 5 fold or more, 10 fold or more, 25 fold or more, 50 fold or more, or 100 fold or more. In some cases, the water-solubilizing moiety is charged, e.g., a positively or negatively charged hydrophilic moiety. In some instances, the water-solubilizing moiety is a neutral hydrophilic moiety. In some instances, the water-solubilizing moiety is branched (e.g., as described herein). In some instances, the water-solubilizing moiety is linear. Water-solubilizing moieties include, but are not limited to, those taught in US Patent Publication No. 2022/0348770 which is incorporated herein by referenced in its entirety.

A “water-soluble polymer” may exhibit solubility in water (e.g., aqueous buffer) of >1 mg/mL, >2 mg/mL, >3 mg/mL, >4 mg/mL, >5 mg/mL, >6 mg/mL, >7 mg/mL, >8 mg/mL, >9 mg/mL, or >10 mg/mL at ambient room temperature. In some instances, the water-soluble polymer can exhibit solubility in water of >10 mg/mL, >20 mg/mL, >30 mg/mL, >40 mg/mL, >50 mg/mL, >60 mg/mL, >70 mg/mL, >80 mg/mL, >90 mg/mL and/or >100 mg/mL at ambient room temperature.

Any convenient water-solubilizing moiety or WSG may be included in the dyes described herein to provide for increased water-solubility. WSGs may be, but are not limited to, carboxylate, phosphonate, phosphate, sulfonate, sulfate, sulfinate, sulfonium, ester, polyethylene glycols (PEG) and modified PEGs, linear PEG groups, branched PEG groups, hydroxyl, amine, amino acid, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups, glycols, including, polyethers, a zwitterionic derivative, a peptide sequence, nucleotides (DNA and RNA), a peptoid, a carbohydrate, an oxazoline, a polyol, a dendron, a dendritic polyglycerol, a cellulose, a chitosan, —COOM′, —SO₃M′, —PO₃M′, —NR³₊, Y′, (CH₂CH₂O)ₚR and mixtures thereof, where Y′ can be any halogen, sulfate, sulfonate, or oxygen containing anion, p can be 1 to 500, each R can be independently H or an alkyl (such as methyl) and M′ can be a cationic counterion or hydrogen, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, and polyethylene glycol, wherein yy is selected from 1 to 1000, X is selected from O, S, and NR^{ZZ}, and R^{ZZ} and R^{YY} are independently selected from H and C₁₋₃ alkyl, and combinations or derivatives thereof. In some instances, WSGs include, but are not limited to, PEG, a modified PEG, a peptide sequence, a peptoid, a carbohydrate, an oxazoline, a polyol, a dendron, a dendritic polyglycerol, a cellulose, a chitosan, or a derivative thereof. WSGs may be unsubstituted or substituted.

In some instances, the water-solubilizing moiety or WSGs may be a hydrophilic polymer. For example, hydrophilic polymers that can be utilized in the WSG include, but are not limited to, polyalkylene oxide based polymers comprising an ethylene oxide repeat unit of the formula —(CH₂—CH₂—O)ₙ— or —(O—CH₂—CH₂)ₙ—, such as, for example, PEG, polyamide alkylene oxide, or derivatives thereof. Further examples of polymers of interest include a polyamide having a molecular weight greater than 1,000 Daltons of the formula —[C(O)—X—C(O)—NH—Y—NH]ₙ— or —[NH—Y—NH—C(O)—X—C(O)]ₙ, where X and Y are divalent radicals that may be the same or different and may be branched or linear, and n is a discrete integer from 2-100, such as from 2 to 50, and where either or both of X and Y comprises a biocompatible, substantially non-antigenic water-soluble repeat unit that may be linear or branched. The number of such water-soluble repeat units can vary significantly, with the number of such units being from 2 to 500, 2 to 400, 2 to 300, 2 to 200, 2 to 100, 6-100, for example from 2 to 50 or 6 to 50. An example of an embodiment is one in which one or both of X and Y is selected from: —((CH₂)ₙ₁—(CH₂—CH₂—O)ₙ₂—(CH₂)— or —((CH₂)ₙ₁—(O—CH₂—CH₂)ₙ₂—(CH₂)ₙ₁—), where n1 is 1 to 6, 1 to 5, 1 to 4, or 1 to 3, and where n2 is 2 to 50, 2 to 25, 2 to 15, 2 to 10, 2 to 8, or 2 to 5. In some instances, the water-soluble polymer is a group of 1-50 monomeric units, such as 1-40, 1-30, 1-20, 2-24, 2-20, 2-10 or 2-6 monomeric units. A further example of an embodiment is one in which X is —(CH₂—CH₂)—, and where Y is —(CH₂—(CH₂—CH₂—O)₃—CH₂—CH₂—CH₂)— or —(CH₂—CH₂—CH₂—(O—CH₂—CH₂)₃—CH₂)—. In certain instances, any one of the formulae described herein may be substituted with a water-soluble moiety that is a dendron, as known in art.

In some instances, hydrophilic polymers can be, for example, PEG, a peptide sequence, a peptoid, a carbohydrate, an oxazoline, a polyol, a dendron, a dendritic polyglycerol, a cellulose, a chitosan, or a derivative thereof.

In some cases, a water-solubilizing moiety or WSG is (CH₂)ₓ(OCH₂CH₂)_{y}OCH₃ where each x is independently an integer from 0-20, each y is independently an integer from 0 to 50. In some instances, the water-soluble polymer is a PEG group or modified PEG polymer of 6-24 monomeric units, such as 10-30, 10-24, 10-20, 12-24, 12-20, 12-16 or 16-20 monomeric units.

In some cases, the water-solubilizing moiety or WSG includes a non-ionic polymer (e.g., a PEG polymer) substituted at the terminal with an ionic group (e.g., a sulfonate). In some embodiments of the formulae, the WSG includes a substituent selected from (CH₂)ₓ(OCH₂CH₂)_{y}OCH₃ where each x is independently an integer from 0-20, each y is independently an integer from 0 to 50; and a benzyl optionally substituted with one or more halogen, hydroxyl, C₁-C₁₂ alkoxy, or (OCH₂CH₂)_{z}OCH₃ where each z is independently an integer from 0 to 50. In some instances, the WSG is (CH₂)₃(OCH₂CH₂)₁₁OCH₃. In some embodiments, one or more of the substituents is a benzyl substituted with at least one WSG groups (e.g., one or two WSG groups) selected from (CH₂)ₓ(OCH₂CH₂)_{y}OCH₃ where each x is independently an integer from 0-20 and each y is independently an integer from 0 to 50. It is understood that hydroxy-terminated polymer chains (e.g., PEG chains) instead of methoxy-terminated polymer chains (e.g., PEG chains) may be utilized in any of the water-solubilizing moieties.

The term modified polymer, such as a modified PEG, refers to water soluble polymers that have been modified or derivatized at either or both terminals, e.g., to include a terminal substituent (e.g., a terminal alkyl, substituted alkyl, alkoxy or substituted alkoxy, etc.) and/or a terminal linking functional group (e.g., an amino or carboxylic acid group suitable for attachment via amide bond formation) suitable for attached of the polymer to a molecule of interest (e.g., to a light harvesting chromophore via a branching group). The subject water-soluble polymers can be adapted to include any convenient linking groups. It is understood that in some cases, the water-soluble polymer can include some dispersity with respect to polymer length, depending on the method of preparation and/or purification of the polymeric starting materials. In some instances, the water-soluble polymers are monodisperse.

The water-soluble polymer can include one or more spacers or linkers. Examples of spacers or linkers include linear or branched moieties comprising one or more repeat units employed in a water-soluble polymer, diamino and or diacid units, natural or unnatural amino acids or derivatives thereof, as well as aliphatic moieties, including alkyl, aryl, heteroalkyl, heteroaryl, alkoxy, and the like, which can contain, for example, up to 18 carbon atoms or even an additional polymer chain.

The water-soluble polymer moiety, or one or more of the spacers or linkers of the polymer moiety when present, may include polymer chains or units that are biostable or biodegradable. For example, polymers with repeat linkages have varying degrees of stability under physiological conditions depending on bond lability. Polymers with such bonds can be categorized by their relative rates of hydrolysis under physiological conditions based on known hydrolysis rates of low molecular weight analogs, e.g., from less stable to more stable, e.g., polyurethanes (—NH—C(O)—O—)>polyorthoesters (—O—C((OR)(R′))—O—)>polyamides (—C(O)—NH—). Similarly, the linkage systems attaching a water-soluble polymer to a target molecule may be biostable or biodegradable, e.g., from less stable to more stable: carbonate (—O—C(O)—O—)>ester (—C(O)—O—)>urethane (—NH—C(O)—O—)>orthoester (—O—C((OR)(R′))—O—)>amide (—C(O)—NH—). In general, it may be desirable to avoid use of a sulfated polysaccharide, depending on the lability of the sulfate group. In addition, it may be less desirable to use polycarbonates and polyesters. These bonds are provided by way of example, and are not intended to limit the types of bonds employable in the polymer chains or linkage systems of the water-soluble polymers useful in the WSGs disclosed herein.

In some instances, the water-solubilizing moieties include, but are not limited to, hydroxy, alkoxy, (hetero)aryloxy, (hetero)arylamino, PEG, linked PEG, amide-PEG, sulfonamide-PEG, phosphoramide-PEG, ammonium alkyl salt, ammonium alkyloxy salt, ammonium oligoether salt, carbonyl, acyl, sulfonate, alkyl sulfonate, alkyl carboxylate, sulfonate alkyl salt, sulfonate alkoxy salt, sulfonate oligoether salt, sulfonamido oligoether, sulfonamide, sulfinamide, phosphonamidate, phosphinamide, phosphonate, alkoxy sulfonamide PEG, alkylcarboxylate, alkylamide, alkoxy sulfonate, alkyl sulfonate, alkyl sulfonate salt,

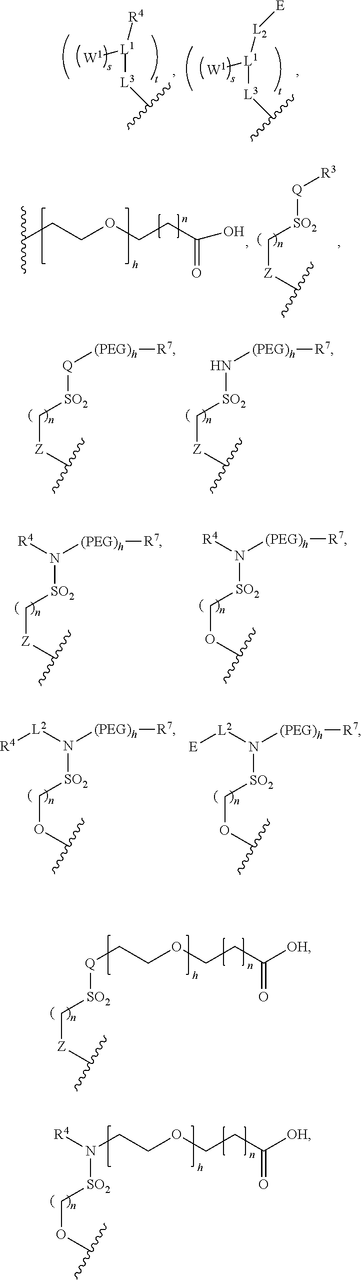

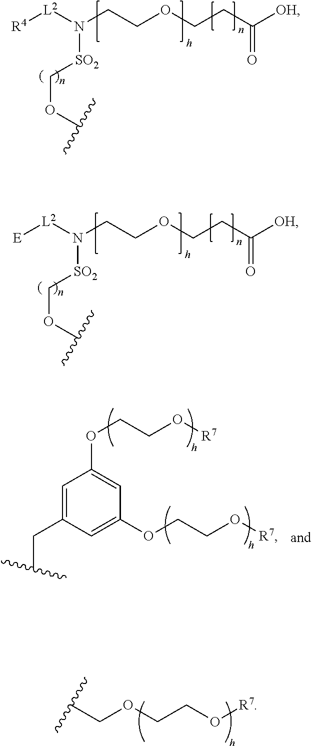

In some instances, the subject compounds may comprise multiple water-solubilizing moieties or WSG attached at a single location in the subject compounds, for example, via a branching linker, such as, for example, an aralkyl substituent further di-substituted with water solubilizing groups. As such, in some cases, the branching linker group is a substituent of the dye that connects the dye to two or more water solubilizing groups. In some instances, multiple water-solubilizing moieties may be attached to the subject compounds via groups having, for example, the following formulas:

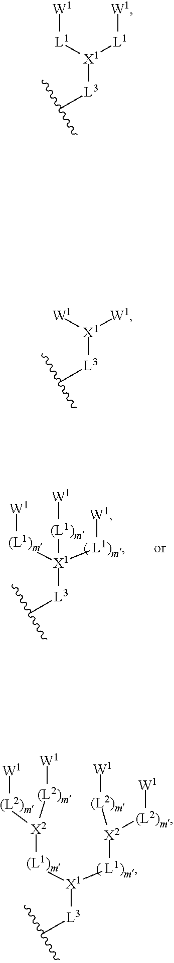

wherein X¹, X² are branching points, L¹, L², L³ are linkers, m′ is an integer from 1, 2, or 3; W¹ is a water-solubilizing moiety.

In some instances, one or more water-solubilizing moieties may be attached to the subject polymers via a group comprising linkers according to the disclosure, for example, as taught in US Published Application No. 2020/0190253A1, which is incorporated herein by reference in its entirety. A linker moiety can be attached to the at the X, Y,

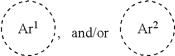

groups of the fluorescent compounds of the instant disclosure. A linker may be cleavable or non-cleavable.

One or more water-solubilizing moieties can also be attached to the subject polymers via a group comprising linkers, such as, for example, but not limited to, the following linker formula (VIe):

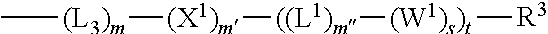

wherein:
each optional L¹ and L³ is an independently selected linker moiety;
X¹, optionally present, is a branching point;
W¹ is a water-soluble moiety, including, but not limited to, a water-soluble polymer comprising 2-50, 4-30, or 6-24 monomeric units;
each m is independently 0 or 1; each m′ is independently 0 or 1; each m″ is independently 0 or 1;
each s is independently 1 or 2;
each t is independently 0, 1, 2, or 3; and
R³ is as defined herein.

In some instances, L¹, L³, and X are absent and W¹ is a water-solubilizing moiety, for example, a water-soluble polymer comprising 2-50, 4-30, or 6-24 monomeric units, such as 10-30, 10-24, 10-20, 12-24, 12-20, 12-16 or 16-20 monomeric units. In some cases, the water-solubilizing moiety may be a linear water-solubilizing moiety. For example, L¹ and X may be absent, L³ is a linker (e.g., as disclosed herein), and W¹ is a water-solubilizing moiety.

In some cases, at least one of, at least two of, or all three of L¹, L² and/or L³ may be selected from an alkyl or substituted alkyl linker, an alkenyl or substituted alkenyl linker, an alkynyl or substituted alkynyl linker, an alkoxy or substituted alkoxy linker, a PEG linker, a sulfonamido-alkyl or substituted sulfonamido-alkyl linker, an amido-alkyl or substituted amido-alkyl linker and an alkyl-amido-alkyl or substituted alkyl-amido-alkyl linker. In certain cases, the linker comprises a carbonyl group. A linker moiety can be a covalent bond, an alkoxy, sulfonamide, disulfonamide, a selenomide, a sulfinamide, a sultam, a disulfinamide, an amide, carbonyl, a seleninamide, a phosphonamide, a phosphinamide, a phosphonamidate, or a secondary amine.

In some instances, L² and L³ may be linker moieties each independently selected from the group consisting of a covalent bond, C₁₋₈ alkylene, 2- to 8-membered heteroalkylene, and a chain of between 2 and 200 backbone atoms in length, wherein the chain comprises a linear chain, a branched chain, and/or a cyclic moiety.

In some instances, L¹ can be a sulfonamide, a sulfinamide, a disulfonamide, a disulfinamide, a sultam, an amide, a secondary amine, a phosphonamide, a phosphinamide, a phosphonamidate, a selenonamide, or a seleninamide.

In some instances, L³ can be a linker having a backbone of 20 atoms or less in length and W¹ is a water-solubilizing moiety (e.g., as described herein). In some instances, L³ can be selected from an alkyl or substituted alkyl linker, an alkenyl or substituted alkenyl linker, an alkynyl or substituted alkynyl linker, an acyl or substituted acyl, an alkoxy or substituted alkoxy linker, a PEG linker, a sulfonamido-alkyl or substituted sulfonamido-alkyl linker, an amido-alkyl or substituted amido-alkyl linker and an alkyl-amido-alkyl or substituted alkyl-amido-alkyl linker. In some instances, L³ can be a bond. In some instances, L³ can be an alkyl or substituted alkyl linker, an alkenyl or substituted alkenyl linker, an alkynyl or substituted alkynyl linker, an alkoxy or substituted alkoxy linker and X can be an aryl group.

In some instances, L¹ and L³ are each independently selected from a C₁-C₁₂ alkyl or substituted alkyl linker, a C₁-C₁₂ alkenyl or substituted alkenyl linker, a C₁-C₁₂ alkynyl or substituted alkynyl linker, a C₁-C₁₂ acyl or substituted acyl linker, a C₁-C₁₂ alkoxy or substituted alkoxy linker, a C₁-C₁₂ amido-alkyl or substituted amido-alkyl linker, a C₁-C₁₂ alkyl-amido-alkyl or substituted alkyl-amido-alkyl linker, a sulfonamide, a sulfinamide, a disulfonamide, a disulfinamide, a sultam, an amide, a secondary amine, a phosphonamide, a phosphinamide, a phosphonamidate, a selenonamide, and a seleninamide. In certain cases, L³ comprises a carbonyl group or alkoxy group, and L¹ is a C₁-C₁₂ alkyl or substituted alkyl, a sulfonamide, a sulfinamide, a disulfonamide, a disulfinamide, a sultam, an amide, a secondary amine, a phosphonamide, a phosphinamide, a phosphonamidate, a selenonamide, and a seleninamide. In some instances, L³ can be an alkoxy or substituted alkoxy linker, X can be absent, and L¹ can be a sulfonamide, a sulfinamide, a disulfonamide, a disulfinamide, a sultam, an amide, a secondary amine, a phosphonamide, a phosphinamide, a phosphonamidate, a selenonamide, or a seleninamide.

In some instances, the branching point X¹ is selected from N, CR′, C(═O)N, SO₂N, a tri-substituted aryl moiety (e.g., a 1,3,5-phenyl), a tetra-substituted aryl moiety (e.g., a 1, 3, 4, 5-phenyl), and a tri-substituted heteroaryl group. In certain instances, the branching point X¹ is a nitrogen atom. In other instances, the branching point X¹ is CR′, where R′ is selected from hydrogen, alkyl, substituted alkyl, or -L³-W¹ (e.g., as described herein).

The term “water soluble” when referring to a polymer as used herein refers to a polymer having solubility in “water” as used herein of 1 mg/mL or more, such as 3 mg/mL or more, 10 mg/mL or more, 20 mg/mL or more, 30 mg/mL or more, 40 mg/mL or more, 50 mg/mL or more, 60 mg/mL or more, 70 mg/mL or more, 80 mg/mL or more, 90 mg/mL or more, 100 mg/mL or more, or even more. It is understood that water soluble polymers may, under certain conditions, form discrete water-solvated nanoparticles in aqueous systems and can be resistant to aggregation. The fluorescent polymers of the disclosure can be water soluble

### Polymers

The disclosure provides water-soluble polymer dyes comprising at least one co-monomer M¹ having a structure according to Formula (Ia):

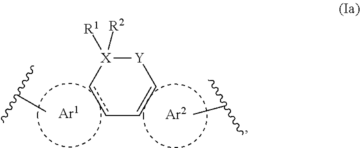

wherein
Ar¹ and Ar² are each independently substituted or unsubstituted aryl or heteroaryl groups and at least one of Ar¹ and Ar² comprises at least one fused thienyl ring;
each X is independently selected from the group consisting of C and Si; and
each Y is independently selected from the group consisting of a bond, CR¹R², CHR¹, CHR², SiHR², SiHR¹, and SiR¹R², and when Y is a bond X is directly bonded to both rings. In some cases, when Y is a bond, Ar¹ and Ar² are not both thienyl. In some cases, Ar¹ and Ar² are different. In some cases, Ar¹ and Ar² are the same. In some cases, Ar¹ and Ar² can be substituted with a water-solubilizing group or linked water-solubilizing group. In some cases, Ar¹ and Ar² can be substituted with a functional group or linked functional group.

Each X may be independently selected from the group consisting of C and Si. In some instances, X is C. X can be Si. Each X can be different. Each X can be the same.

Each Y may be independently selected from the group consisting of a bond, CR¹R², CHR¹, CHR², SiHR², SiHR¹, and SiR¹R², and when Y is a bond X is directly bonded to both rings. In some cases, when Y is a bond, Ar¹ and Ar² are not simultaneously thienyl. In some cases, each Y may be independently selected from the group consisting of CR¹R², CHR¹, CHR². Y can be CH₂. Y can be a bond. Y can be CR¹R². Y can be CHR². Y can be CHR². Y can be SiR¹R². Y can be SiHR². Y can be SiHR¹. Each Y can be different. Each Y can be the same.

Each R¹ may be independently selected from the group consisting of a water-solubilizing moiety, a linker moiety, alkyl, alkene, alkyne, cycloalkyl, hydroxy, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, phosphonate,

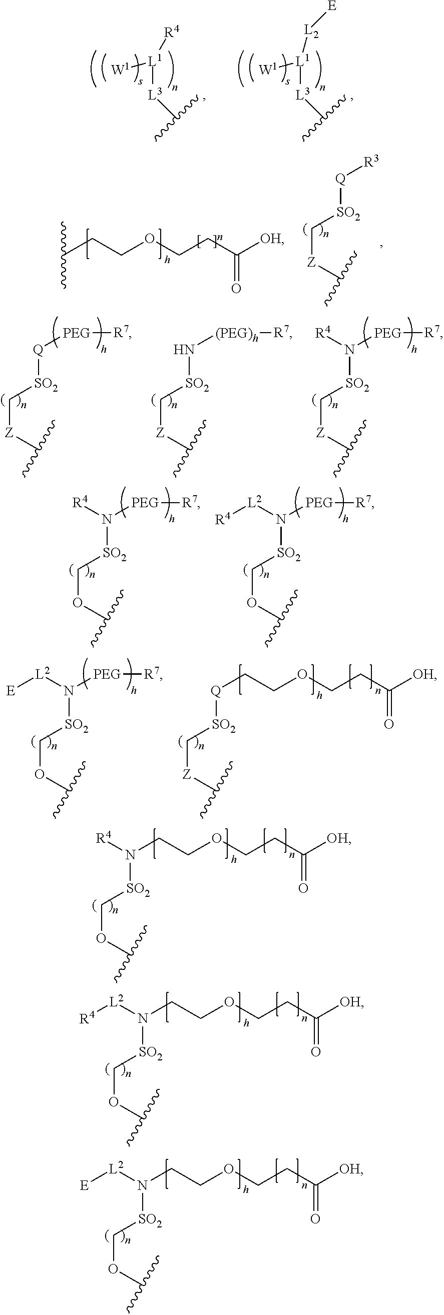

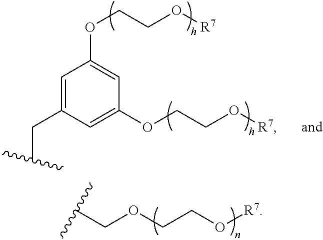

In some cases, each R¹ may be independently selected from the group consisting of

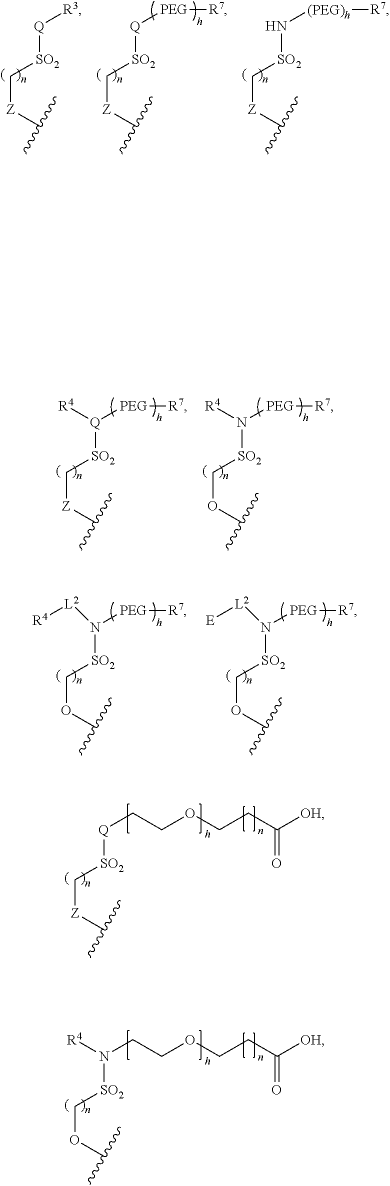

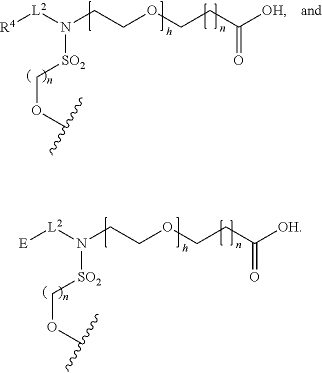

In some cases, each R¹ may be independently selected from the group consisting of a water-solubilizing moiety, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, phosphonate,

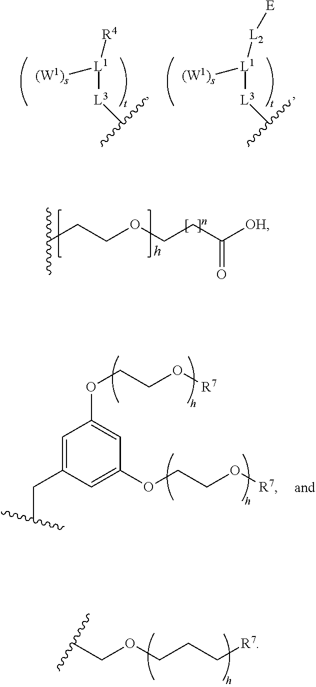

In some cases, each R¹ may be independently selected from the group consisting of a water-solubilizing moiety, a linker moiety, alkyl, alkene, alkyne, cycloalkyl, hydroxy, haloalkyl, (hetero)aryloxy, (hetero)arylamino, aryl, and heteroaryl.

R¹ can be a water-solubilizing moiety. R¹ can be a linker moiety, R¹ can be alkyle. R1 can be alkene. R¹ can be C₁₋₁₀-alkene. R¹ can be methene, ethene, n-propene, i-propene, n-butene, i-butene, or t-butene. R¹ can be alkyne. R¹ can be C₁₋₁₀-alkyne. R¹ can be methyne, ethyne, n-propyne, i-propyne, n-butyne, i-butyne, or t-butyne. R¹ can be cycloalkyl. R¹ can be haloalkyl. R¹ can be (hetero)aryloxy. R⁵ can be (hetero)arylamino. R¹ can be PEG. R¹ can be carboxylic acid. R¹ can be ammonium alkyl salt. R¹ can be ammonium alkyloxy salt. R¹ can be ammonium oligoether salt. R¹ can be sulfonate alkyl salt. R¹ can be sulfonate alkoxy salt. R¹ can be sulfonate oligoether salt. R¹ can be sulfonamido oligoether. R¹ can be sulfonamide. R¹ can be sulfinamide. R¹ can be phosphonamidite. R¹ can be phosphinamide. R¹ can be phosphonate.

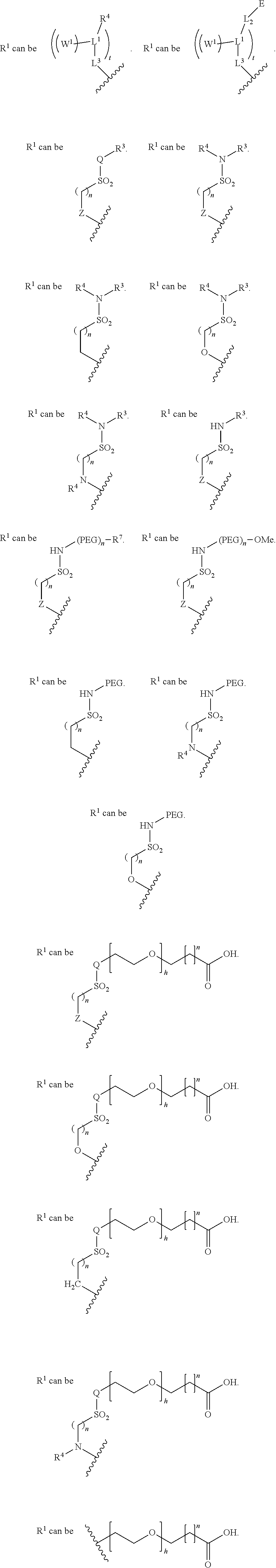

Each instance of R¹ can be different. All instances of R¹ can be the same.

Each R² may be independently selected from the group consisting of a water-solubilizing moiety, a linker moiety, H, alkyl, alkene, alkyne, cycloalkyl, halogen, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, phosphonate,

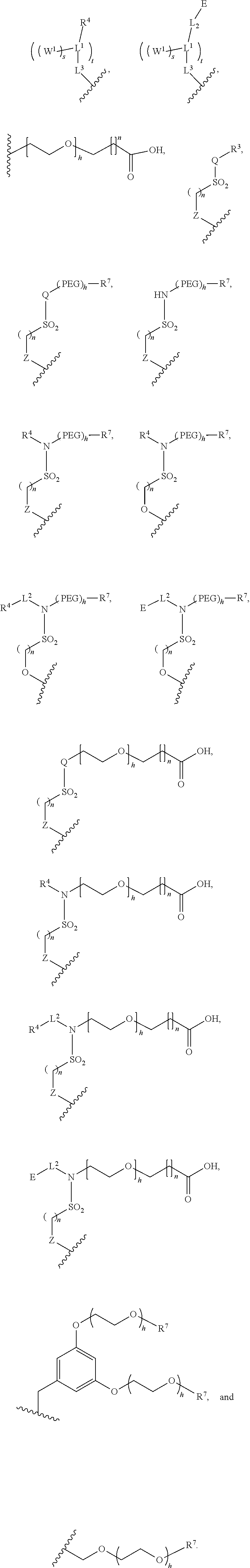

In some cases, R² may be H.

R² can be a water-solubilizing moiety. R² can be a linker moiety. R² can be C₁₋₁₀-alkyl. R² can be methyl, ethyl, n-propyl, i-propyl, n-butyl, i-butyl, or t-butyl. R² can be alkene. R² can be C₁₋₁₀-alkene. R² can be methene, ethene, n-propene, i-propene, n-butene, i-butene, or t-butene. R² can be alkyne. R² can be C₁₋₁₀-alkyne. R² can be methyne, ethyne, n-propyne, i-propyne, n-butyne, i-butyne, or t-butyne. R² can be cycloalkyl. R² can be haloalkyl. R² can be alkoxy. R² can be (hetero)aryloxy. R² can be aryl. R² can be (hetero)arylamino. R² can be PEG. R² can be sulfonamide-PEG. R² can be phosphoramide-PEG. R² can be ammonium alkyl salt. R² can be ammonium alkyloxy salt. R² can be ammonium oligoether salt. R² can be sulfonate alkyl salt. R² can be sulfonate alkoxy salt. R² can be sulfonate oligoether salt. R² can be sulfonamido oligoether. R² can be sulfonamide. R² can be sulfinamide. R² can be phosphonamidite. R² can be phosphinamide. R² can be phosphonate. R² can be

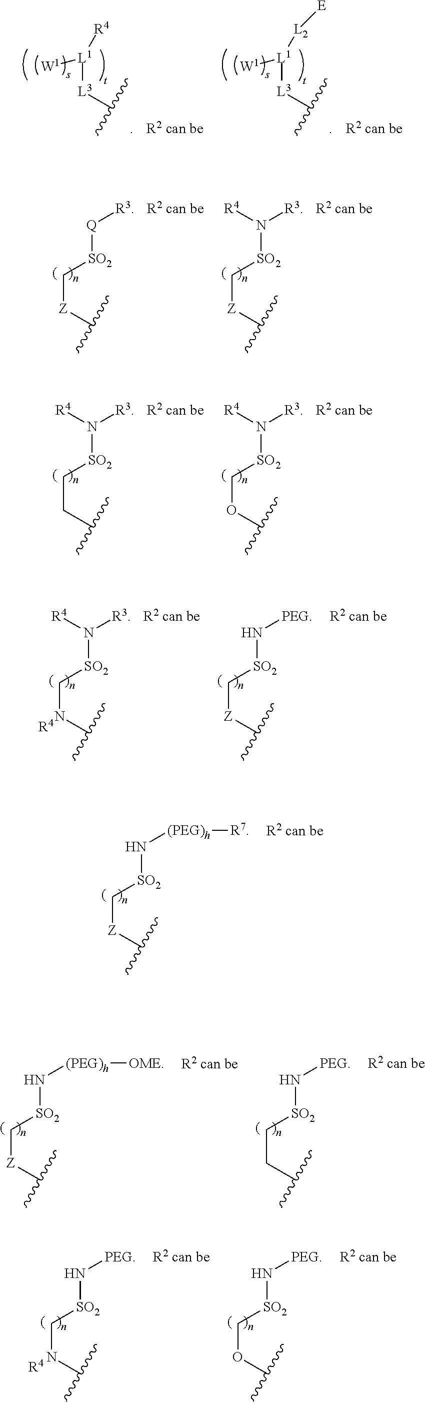

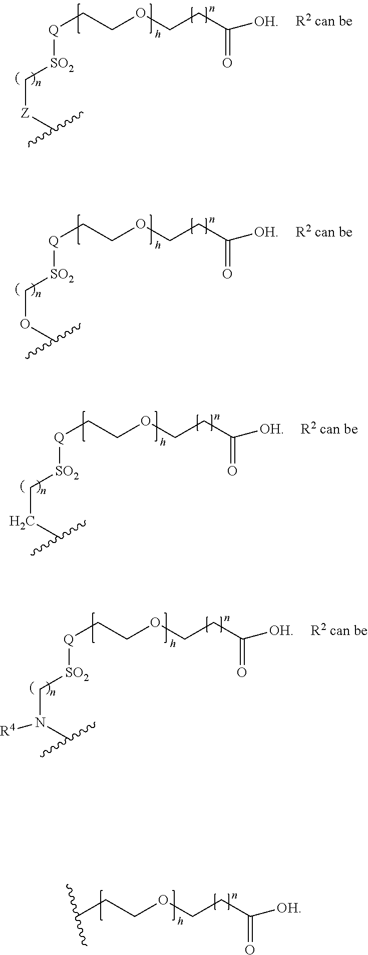

Each instance of R² can be different. All instances of R² can be the same.

Each R³ may be independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, and a PEG group. In some cases, each R³ may be independently selected from the group consisting of a water-solubilizing moiety and a PEG group.

R³ can be a water solubilizing moiety. R³ can be PEG or modified PEG polymer. The modified PEG polymer can be of 6-30 monomeric units, such as 6-24 or 10-30, 10-24 or 10-20, 12-24, 12-20, 12-16 or 16-20 monomeric unit. The modified PEG polymer can be terminated with a carboxlic acid or carboxylate ester.

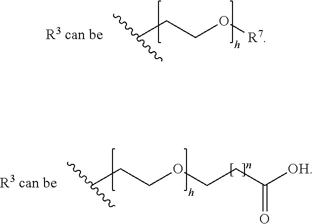

R³ can be alkyl. R³ can be a polymer comprising 6-24 monomeric units.

Each Z may be independently selected from the group consisting of CH₂, CHR⁴, O, NR⁴, and NH. In some cases, Z may be O. In some cases, each Z may be independently selected from CH₂ and CHR⁴. In some cases each Z may be independently selected from NR⁴ and NH. Z can be CH₂. Z can be CHR⁴. Z can be O. Z can be NR⁴.

Each Q may be independently selected from the group consisting of a bond, NH, NR⁴, C₁-C₁₂ alkylene, CHR⁴, and CH₂. In some cases, each Q may be independently selected from the group consisting of NH and NR⁴. In some cases, each Q may be independently selected from the group consisting of a bond, C₁-C₁₂ alkylene, CHR⁴, and CH₂. Q can be a bond. Q can be NR⁴. Q can be CHR⁴. Q can be —CH₂.

Each R⁴ may be independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, an amine, hydroxyl, alkylamino, substituted or unsubstituted C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₈ alkyl, x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof.

In some cases, each R⁴ may be independently selected the group consisting of a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, and (CH₂)ₓ—(OCH₂—CH₂)_{y′}OR⁹, wherein each R⁹ is C₁-C₈ alkyl, x′ is independently an integer from 0-20 and each y′ is independently an integer from 0-50, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof. In some cases, each R⁴ may be independently selected the group consisting of H, a linker moiety, a chromophore, a linked chromophore, a functional group, a linked functional group, a substrate, a linked substrate, a binding partner, a linked binding partner, a quenching moiety, L²-E, halogen, an amine, hydroxyl, alkylamino, substituted or unsubstituted C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, and C₂-C₁₈ (hetero)arylamino.

In some cases, R⁴ can be H. R⁴ can be alkyl. R⁴ can be PEG. R⁴ can be a water-solubilizing moiety. R⁴ can be a linker moiety. R⁴ can be a chromophore. R⁴ can be carboxylic amine. R⁴ can be amine. R⁴ can be carbamate. R⁴ can be carboxylic acid. R⁴ can be carboxylate. R⁴ can be maleimide. R⁴ can be activated ester. R⁴ can be N-hydroxysuccinimidyl, hydrazine. R⁴ can be L²-E. R⁴ can be hydrazide. R⁴ can be hydrazone. R⁴ can be azide. R⁴ can be alkyne. R⁴ can be aldehyde. R⁴ can be thiol. R⁴ can be cycloalkyne. R⁴ can be cycloalkene. R⁴ can be tetrazine. Each R⁴ can optionally have protected groups.

Each W¹ may be independently a water-solubilizing moiety.

L¹, L², and L³ are each independently selected linker moieties. Linking moieties L¹, L², and L³ may independently be, but are not limited to, a covalent bond, C₁₋₈ alkylene, 2- to 8-membered heteroalkylene. In some embodiments, the linker is a single atom, a linear chain, a branched chain, a cyclic moiety. In some embodiments, the linker is chain of between 2 and 100 backbone atoms (e.g., carbon atoms) in length, such as between 2 and 50 backbone atoms in length or between 2 and 20 atoms backbone atoms in length. In certain cases, one, two, three, four or five or more carbon atoms of a linker backbone can be optionally replaced with sulfur, nitrogen, or oxygen. The bonds between backbone atoms can be saturated or unsaturated; typically, not more than one, two, or three unsaturated bonds will be present in a linker backbone. The linker can include one or more substituent groups (e.g., an alkyl group or an aryl group). A linker can include, without limitation, oligo(ethylene glycol); ethers; thioethers; tertiary amines; and alkylene groups (i.e., divalent alkyl radicals), which can be straight or branched. The linker backbone can include a cyclic group, for example, a divalent aryl radical, a divalent heterocyclic radical, or a divalent cycloalkyl radical, where 2 or more atoms, e.g., 2, 3, or 4 atoms, of the cyclic group are included in the backbone.

In some cases, L¹ comprises a sulfonamide, a sulfonimide, a sultam, a disulfinamide, an amide, a phosphonamide, a phosphonamidate, a phosphinamide, a selenoonamide, a seleninamde, or a secondary amine. In some embodiments, L¹ comprises a sulfonamide, an amide, a phosphonamide, or a secondary amine. In some cases, L¹ is a linker moiety optionally terminated with L²-E. In some cases, L² comprises a linear or branched, saturated or unsaturated C₁₋₃₀ alkylene group; wherein one or more carbon atoms in the C₁₋₃₀ alkylene group is optionally and independently replaced by O, S, NR^{a}; wherein two or more groupings of adjacent carbon atoms in the C₁₋₃₀ alkylene are optionally and independently replaced by —NR^{a}(CO)— or —(CO)NR^{a}—; and wherein each R^{a} is independently selected from H and C₁₋₆ alkyl.

In some cases, L² is a linker moiety optionally terminated with a functional moiety selected from amine, carbamate, carboxylic acid, carboxylate, maleimide, activated ester, N-hydroxysuccinimidyl, hydrazine, hydrazide, hydrazone, azide, alkyne, aldehyde, thiol, alkene, cycloalkene, cycloalkyne, tetrazine, and protected groups thereof, for conjugation to a chromophore, substrate, or a binding partner;

In some embodiments, L³ is selected from the group consisting of a covalent bond, C₁₋₈ alkylene, 2- to 8-membered heteroalkylene (e.g., a divalent alkoxy linker such as —O-alkyl), C₃₋₈ cycloalkylene, C₆₋₁₀ arylene, 5- to 12-membered heteroarylene, 5- to 12-membered heterocyclylene, an amine, —NHC(O)L^{a}-, —C(O)NHL^{a}-, —C(O)L^{a}-, and combinations thereof, wherein L^{a} is selected from the group consisting of C₁₋₈ alkylene and 2- to 8-membered heteroalkylene.

In some cases, L¹, L², and L³ together form the following:

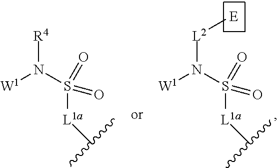

or wherein L^{1a} is a linker moiety.

In some cases, L^{1a} is selected from the group consisting of a covalent bond, C₁₋₈ alkylene, 2- to 8-membered heteroalkylene (e.g., a divalent alkoxy linker), C₃₋₈ cycloalkylene, C₆₋₁₀ arylene, 5- to 12-membered heteroarylene, 5- to 12-membered heterocyclylene, —NHC(O)L^{a}-, —C(O)NHL^{a}-, —C(O)L^{a}-, and combinations thereof. In some embodiments, L^{1a} is selected from the group consisting of a covalent bond, C₁₋₈ alkylene, 2- to 8-membered heteroalkylene, —NHC(O)L^{a}-, —C(O)NHL^{a}-, and —C(O)L^{a}-.

In some embodiments, L³ is a trivalent arylalkyl moiety having: a first point of attachment to a first L¹ moiety (or a first Lia moiety); a second point of attachment to a second L¹ moiety (or a second Lia moiety); and a third point of attachment to an A monomer. For example, some embodiments of the disclosure provide conjugated polymers having two or more E groups, such as chromophores, attached as shown in Formula (L³-1):

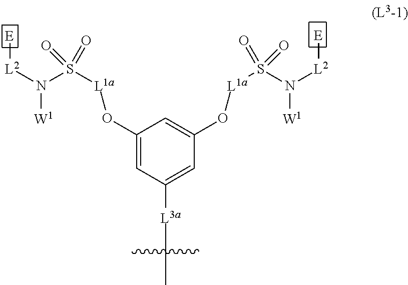

wherein, L³a is selected from the group consisting of a covalent bond, C₁₋₈ alkylene, 2- to 8-membered heteroalkylene, —NHC(O)L^{a}-, —C(O)NHL^{a}-, and —C(O)L^{a}-; L^{1a} is C₁₋₈ alkylene or 2- to 8-membered heteroalkylene; and the wavy line is the point of the attachment to the A monomer.

Each E may be independently selected from the group consisting of a chromophore, a functional moiety, a substrate, and a binding partner. In some cases, each E may be independently selected from the group consisting of a chromophore, and a functional moiety. In some cases, E may a chromophore. In some cases, each E may be independently selected from the group consisting of a functional moiety, and a substrate, and a binding partner. In some cases, each E may be a binding partner. In some cases, each E may be a substrate. In some cases, each E may be a functional moiety.

Each R⁷ may be independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester and —OC₁-C₁₂hydroxy. In some cases, each R⁷ may be independently selected from the group consisting of H, hydroxyl, and C₁-C₁₂ alkoxy. R⁷ can be H. R⁷ can be C₁-C₁₂ alkyl. R⁷ can be C₂-C₁₂ alkene. R⁷ can be C₂-C₁₂ alkyne. R⁷ can be C₃-C₁₂ cycloalkyl. R⁷ can be C₁-C₁₂haloalkyl. R⁷ can be C₁-C₁₂ alkoxy. R⁷ can be C₂-C₁₈ (hetero)aryloxy. R⁷ can be C₂-C₁₈ (hetero)arylamino. R⁷ can be C₂-C₁₂ carboxylic acid. R⁷ can be C₂-C₁₂ carboxylate ester. R⁷ can be C₁-C₁₂ alkoxy.

In some cases, at least one of R¹, R², R³, or R⁴ comprises a water-solubilizing moiety. In some cases, at least one of, at least two of, or at least three of R¹, R², R³, or R⁴ comprises a water-solubilizing moiety.

Each h may be independently an integer from 0 to 50. In some cases, each h is an integer from 0 to 50, 1 to 50, 2 to 40, 3 to 30, or 5 to 25.

Each n may be independently an integer from 1 to 20;

Each s is independently 1 or 2.

Each t may be independently 0, 1, 2, or 3.

In some cases, one or both [symbol] in the monomer or co-monomer is a site for covalent attachment to the unsaturated backbone of the fluorescent polymer.

In some cases, Ar¹ and Ar² may be independently selected from the group consisting of phenyl, thienyl, thienothiophene, benzothienothiophene, dihydronaphthothienothiophene, benzo[b]thieno[2,3-d]thiophene, 5,6-dihydronaphtho[1,2-b]thieno[2,3-d]thiophene, and thieno[3,2-b]thiophene, with at least one of Ar¹ and Ar² comprising at least one fused thienyl ring.

In some cases, the polymer dye comprises at least one co-monomer having a structure selected from the group consisting of Formula (IIa), (IIb), (IIc), (IId), (IIe), (IIf), and (IIg):

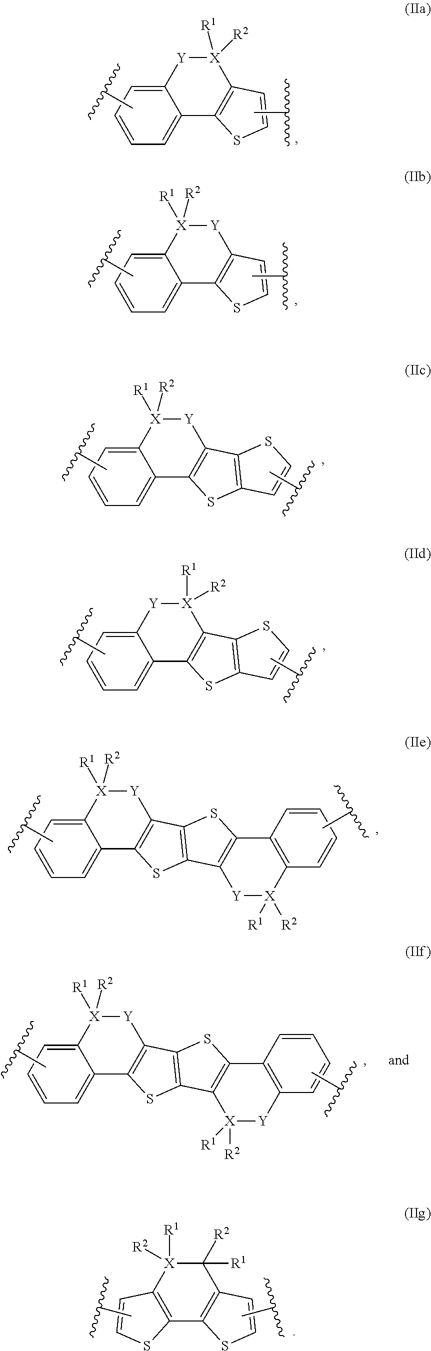

In some cases, the polymer dye comprises at least one co-monomer having the structure of any one of Formula (IIaa), (IIbb), (IIcc), (IIdd), (IIee), (IIff), or (IIgg):

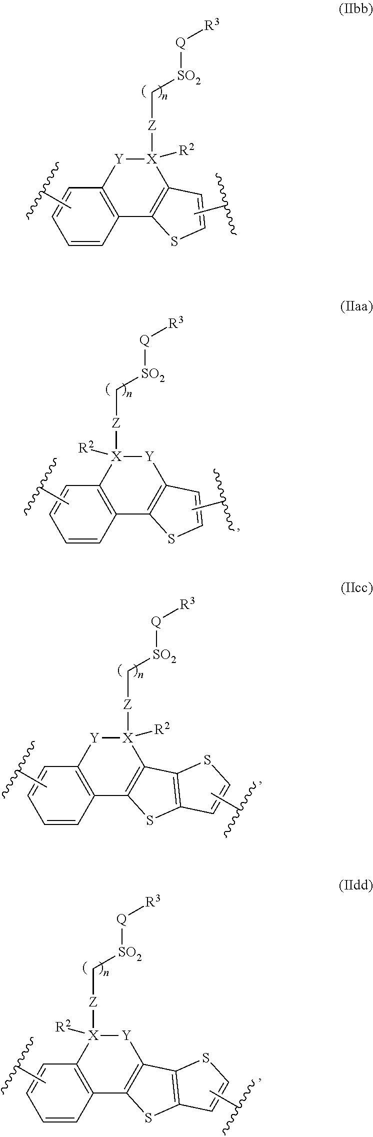

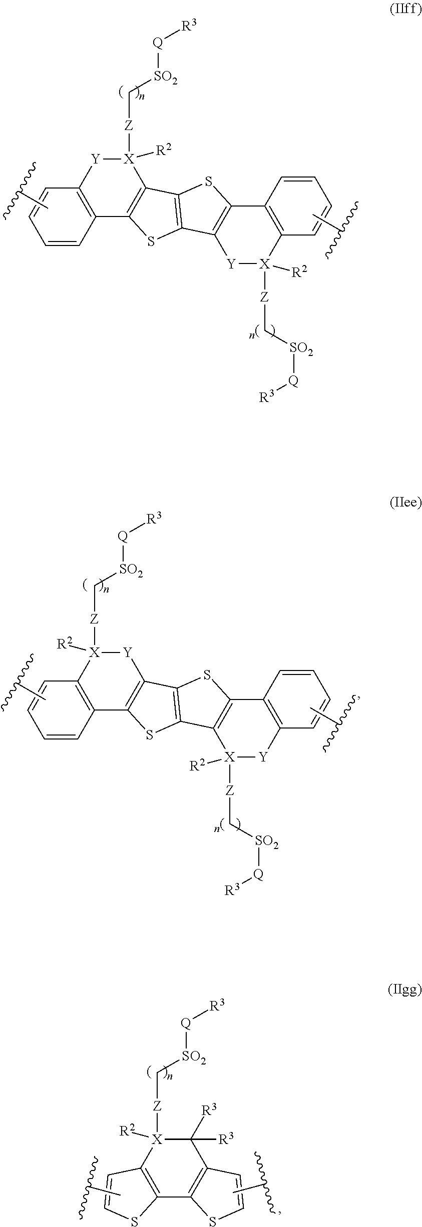

wherein X, Y, Z, Q, R¹, R², R³, and n are as described herein.

The polymer dye may comprise at least one co-monomer having the structure of any one of Formula (IIaaa), (IIbbb), (IIccc), (IIddd), (IIeee), (IIfff), or (IIggg).

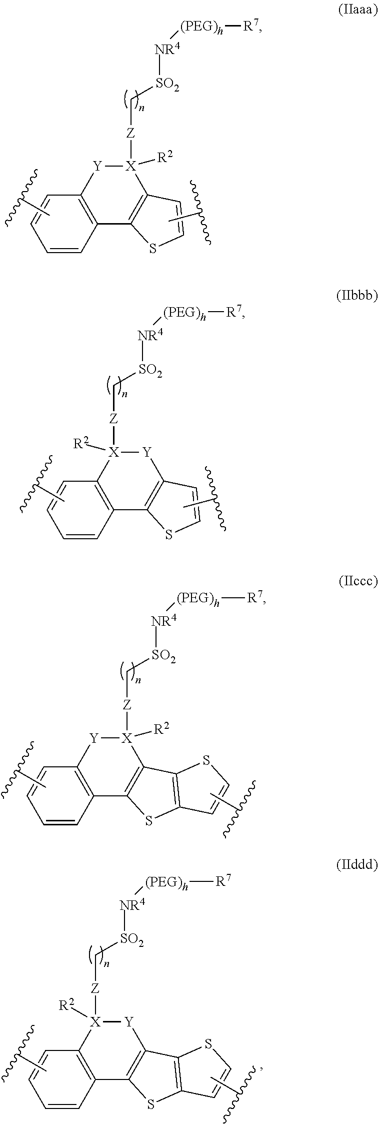

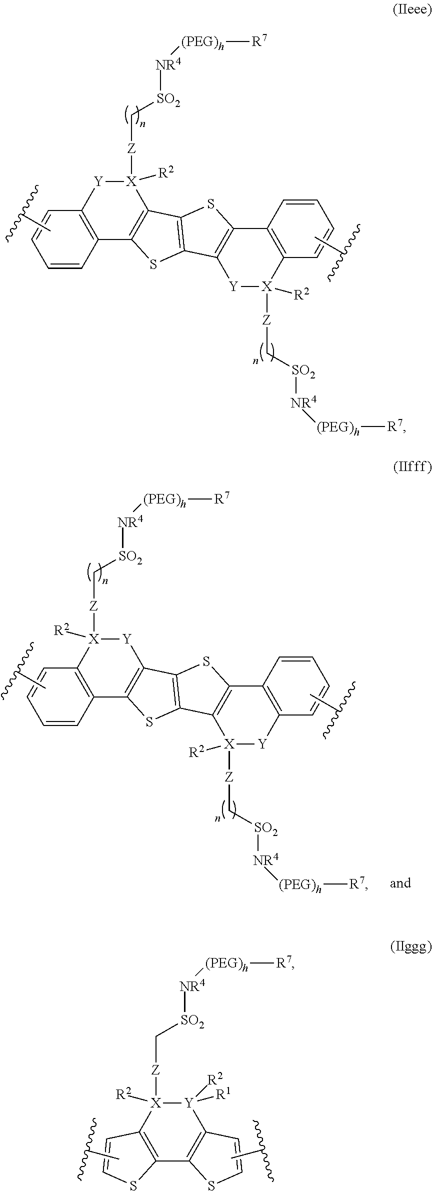

wherein X, Y, Z, R¹, R², R⁴, R⁷, n, and h are as described herein.

M¹ can have a structure according to Formula (Ia). M¹ can have a structure according to Formula (IIa). M¹ can have a structure according to Formula (IIb). M¹ can have a structure according to Formula (IIc). M¹ can have a structure according to Formula (IId). M¹ can have a structure according to Formula (IIe). M¹ can have a structure according to Formula (IIf). M¹ can have a structure according to Formula (IIg). M¹ can have a structure according to Formula (IIaa). M¹ can have a structure according to Formula (IIbb). M¹ can have a structure according to Formula (IIcc). M¹ can have a structure according to Formula (IIdd). M¹ can have a structure according to Formula (IIee). M¹ can have a structure according to Formula (IIff). M¹ can have a structure according to Formula (IIgg). M¹ can have a structure according to Formula (IIaa). M¹ can have a structure according to Formula (IIbbb). M¹ can have a structure according to Formula (IIccc). M¹ can have a structure according to Formula (IIddd). M¹ can have a structure according to Formula (IIeee). M¹ can have a structure according to Formula (IIfff). M¹ can have a structure according to Formula (IIggg).

The disclosure provides a polymer dye comprising a structure of Formula (Ib):

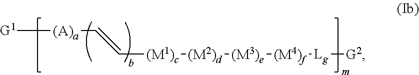

wherein
each A is selected from a substituted or unsubstituted aryl or heteroaryl group;
each optional linker L is independently a linker moiety;
G¹ and G² are each independently selected from the group consisting of an unmodified polymer terminus and a modified polymer terminus, optionally conjugated to E;
a, c, d, e, f and g define the mol % of each unit within the structure which each can be evenly or randomly repeated along the polymer main chain and where a is a mol % from 10 to 100%, c is a mol % from >0 to 90%, each d, e, and f is a mol % from 0 to 90%, and each g is a mol % from 0 to 25%;
each b is independently 0 or 1;
m is an integer from 1 to about 10,000;
each M¹ is a co-monomer comprising a structure according to Formula (Ia):

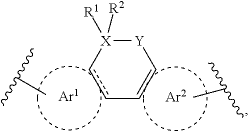

wherein Ar¹ and Ar² are each independently substituted or unsubstituted aryl or heteroaryl groups and at least one of Ar¹ and Ar² comprises at least one fused thienyl ring;
each optional M², M³ and M⁴ is independently selected from the group consisting of an aryl moiety, heteroaryl moiety, or co-monomer having a structure of Formula (Ia), wherein
M¹, M², M³ and M⁴ are evenly or randomly distributed along the polymer main chain, and
each M¹ or optional M², M³ or M⁴ is optionally substituted, and optionally terminated with a functional group selected from amine, carbamate, carboxylic acid, carboxylate, alkyl carboxylate, carboxylic amine, carbamate, carboxylate ester, maleimide, halogenated maleimide, activated ester, N-hydroxysuccinimidyl, imido ester, halogen, boronic ester, boronic acid, hydrozonyl, hydrazine, hydrazide, hydrazone, azide, alkyne, cyclooctyne, aldehyde, tetrazine, alkene, cyclooctene, dienes, dienophiles, thiol, amide, sulfonamide, alkyl sulfonate, ether, thioether, thiocarbamate, sulfonyl fluoride, hydroxyl, iodoacetyl, iodoacetamide, hydrazido, hydrazino, aldehyde, ketone, phosphine, epoxide, urea, thiourea, thioester, imine, disulfides, and protected groups thereof, each optionally conjugated to a substrate, chromophore, or binding partner, wherein each M¹, Ar¹, Ar², X, Y, E, Z, Q, W¹, L¹, L², L³, R¹, R², R³, R⁴, R⁷, R⁹, h, n, s, and t are as defined herein, and at least one of R¹, R², R³, or R⁴ comprises a water-solubilizing moiety.

In some cases, each m is an integer from 1 to about 10,000, 2 to 8,000, 3 to 5,000, 4 to 1,000, 5 to 500, 6 to 100, or 10 to 50.

In some cases, the disclosure provides a polymer dye comprising a structure as provided herein comprising a linker L.

Each optional L can be evenly or randomly distributed along the polymer main chain. L can be —(CH₂)ₚ—O— wherein p is from 1 to 12, e.g., 1 to 6. L can be —O—(CH₂)ₚ— wherein p is from 1 to 12, e.g., 1 to 6. L can be —(CH₂)ₚ— wherein p is from 1 to 12, e.g., 1 to 6. L can be —O—. L can be C₁-C₁₂-alkyl linker, e.g., a C₁-C₆-alkyl linker, wherein one or more backbone atoms are optionally substituted with a heteroatom. L can be an aryl group. L can be a heteroaryl group. When L is an aryl or heteroaryl group, it can be substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, cycloalkyne, cycloalkene, tetrazine, and protected groups thereof for conjugation to another substrate, acceptor dye or chromophore, molecule or binding partner.

In some cases, each optional linker L can be independently selected from the group consisting of:

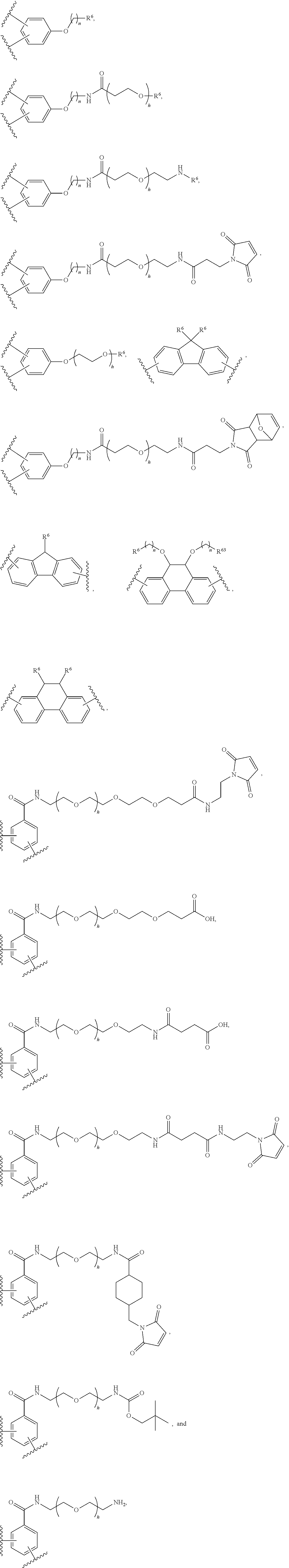

each R⁶ is independently selected from the group consisting of H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino, benzyl optionally substituted with one or more halogen, hydroxyl, C₁-C₁₂ alkoxy, or (OCH₂CH₂)_{f}OCH₃,

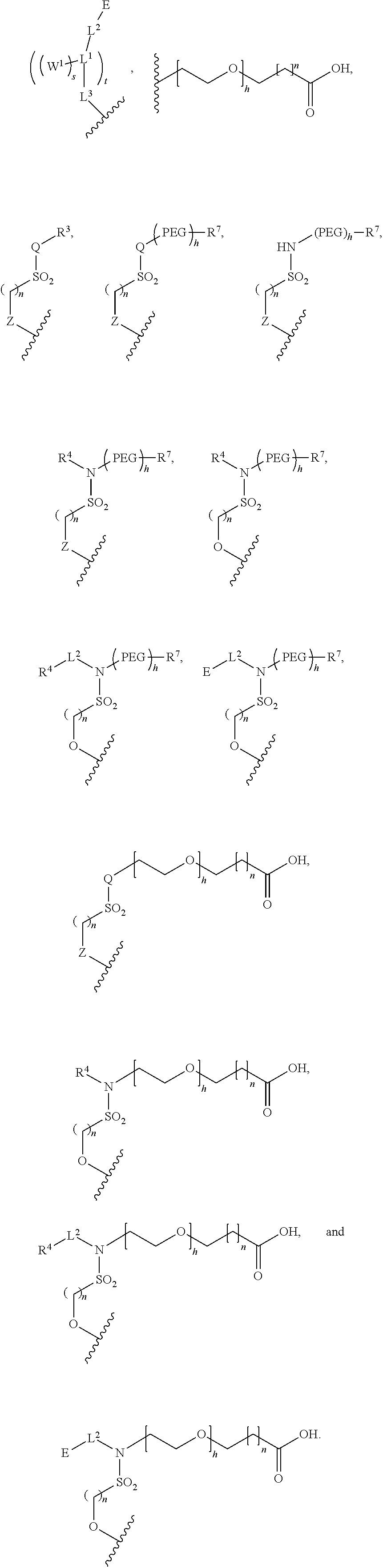

R⁶ can be H. R⁶ can be OH R⁶ can be SH. R⁶ can be NHCOO-t-butyl. R⁶ can be (CH₂)ₙCOOH. R⁶ can be (CH₂)ₙ(CH₂CH₂O)_{f}COOH. R⁶ can be —(CH₂)ₙCOOCH₃. R⁶ can be —(CH₂)ₙNH₂. R⁶ can be —(CH₂)ₙNH(CH₂)ₙCH₃. R⁶ can be —(CH₂)ₙNHCOOH. R⁶ can be —(CH₂)ₙNHCO(CH₂)ₙCO(CH₂)ₙCH₃. R⁶ can be —(CH₂)ₙNHCOO(CH₂)ₙCH₃. R⁶ can be —(CH₂)ₙNHCOOC(CH₃)₃. R⁶ can be —(CH₂)ₙNHCO(C₃-C₁₂)cycloalkyl. R⁶ can be —(CH₂)ₙNHCO(CH₂CH₂O)_{f} (C₁-C₆)alkyl. R⁶ can be —(CH₂)ₙNHCO(CH₂)ₙCOOH. R⁶ can be —(CH₂)ₙNHCO(CH₂)ₙCOO(CH₂)ₙCH₃. R⁶ can be —(CH₂)ₙ(OCH₂CH₂)_{f}OCH₃. R⁶ can be N-maleimide. R⁶ can be halogen, C₂-C₁₂ alkene. R⁶ can be C₂-C₁₂ alkyne. R⁶ can be C₃-C₁₂ cycloalkyl. R⁶ can be C₁-C₁₂halo alkyl. R⁶ can be C₁-C₁₂ (hetero)aryl. R⁶ can be C₁-C₁₂ (hetero)arylamino. R⁶ can be benzyl optionally substituted with one or more halogen, hydroxyl, C₁-C₁₂ alkoxy, or (OCH₂CH₂)_{f}OCH₃. R⁶ can be carboxylic acid. R⁶ can be carboxylate ester.

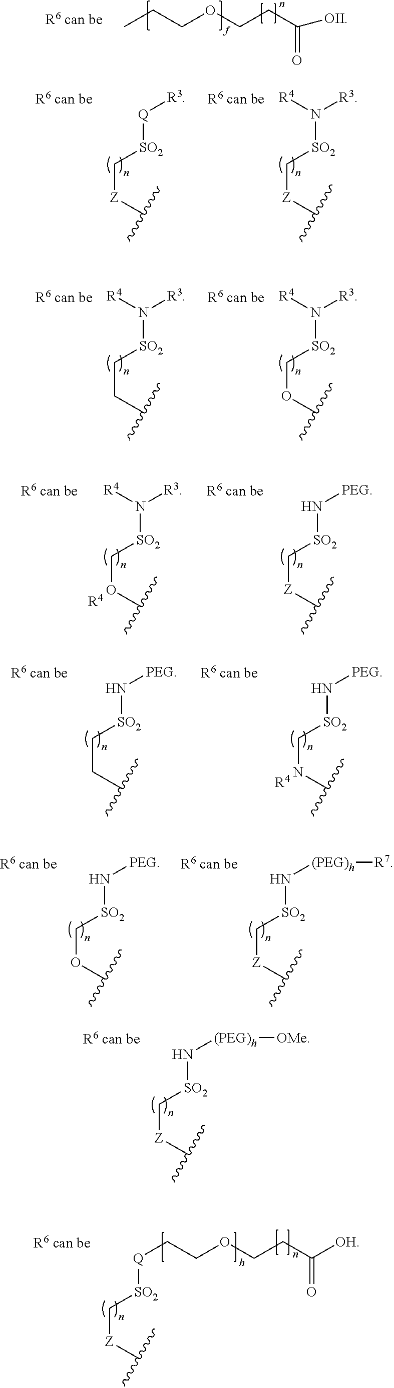

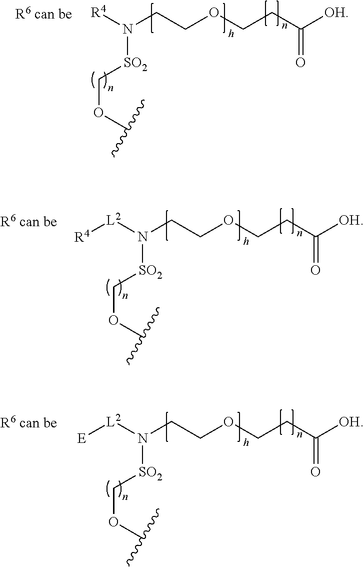

R³, R⁴, h, and n can be as described above.

The fluorescent polymers of the present disclosure also contain capping units represented in Formula (Ib) as each G¹ and G². In some cases, the disclosure provides a polymer dye comprising a structure as provided herein, wherein G¹ and G² are each independently selected from the group consisting of hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), and optionally substituted fluorene, optionally substituted tetrahydropyrene (THP), wherein the substituted aryl, heteroaryl, fluorene, DHP or THP are substituted with one or more pendant chains terminated with a functional group, optionally conjugated to E.

G¹ can be hydrogen. G¹ can be halogen. G¹ can be alkyne. G¹ can be optionally substituted aryl. G¹ can be optionally substituted heteroaryl. G¹ can be halogen. G¹ can be substituted aryl. G¹ can be silyl. G¹ can be diazonium salt. G¹ can be triflate. G¹ can be acetyloxy. G¹ can be azide. G¹ can be sulfonate. In some aspect, G¹ can be phosphate. G¹ can be boronic acid substituted aryl. G¹ can be boronic ester substituted aryl. G¹ can be boronic ester. G¹ can be boronic acid. G can be optionally substituted tetrahydropyrene (THP). G¹ can be optionally substituted dihydrophenanthrene (DHP). G¹ can be optionally substituted fluorene. In some aspect, G¹ can be aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate, or a binding partner.

In some embodiments, G² can be hydrogen. G² can be halogen. G² can be alkyne. G² can be optionally substituted aryl. G² can be optionally substituted heteroaryl. G² can be halogen. G² can be substituted aryl. G² can be silyl. G² can be diazonium salt. G² can be triflate. G² can be acetyloxy. G² can be azide. G² can be sulfonate. G² can be phosphate. G² can be boronic acid substituted aryl. G² can be boronic ester substituted aryl. G² can be boronic ester. G² can be boronic acid. G² can be optionally substituted tetrahydropyrene (THP). G² can be optionally substituted fluorene. G² can be optionally substituted dihydrophenanthrene (DHP). G² can be aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate, or a binding partner.

In some cases, the disclosure provides a polymer dye comprising a structure as provided herein, wherein G¹ and G² are each independently selected from the group consisting of

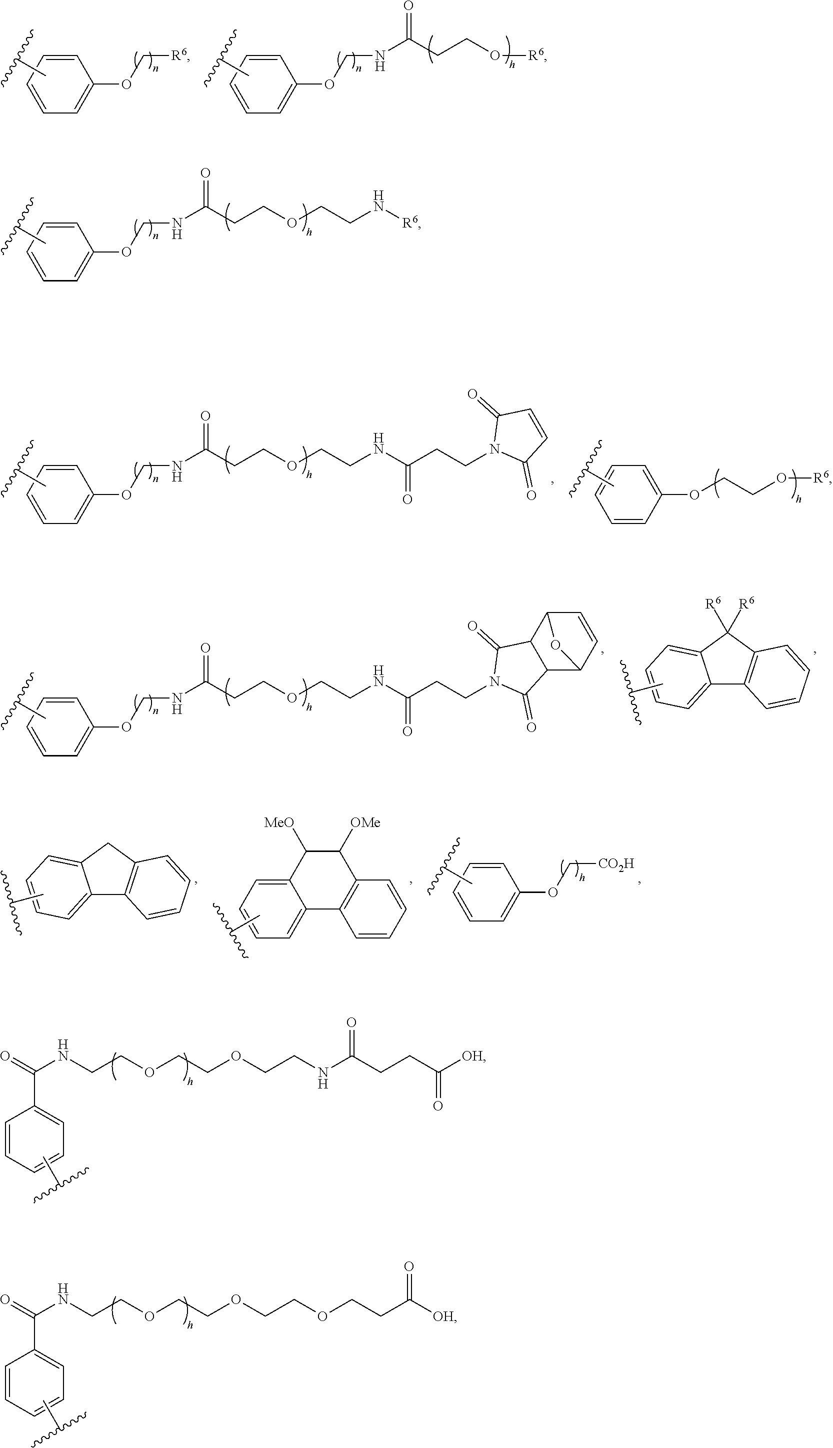

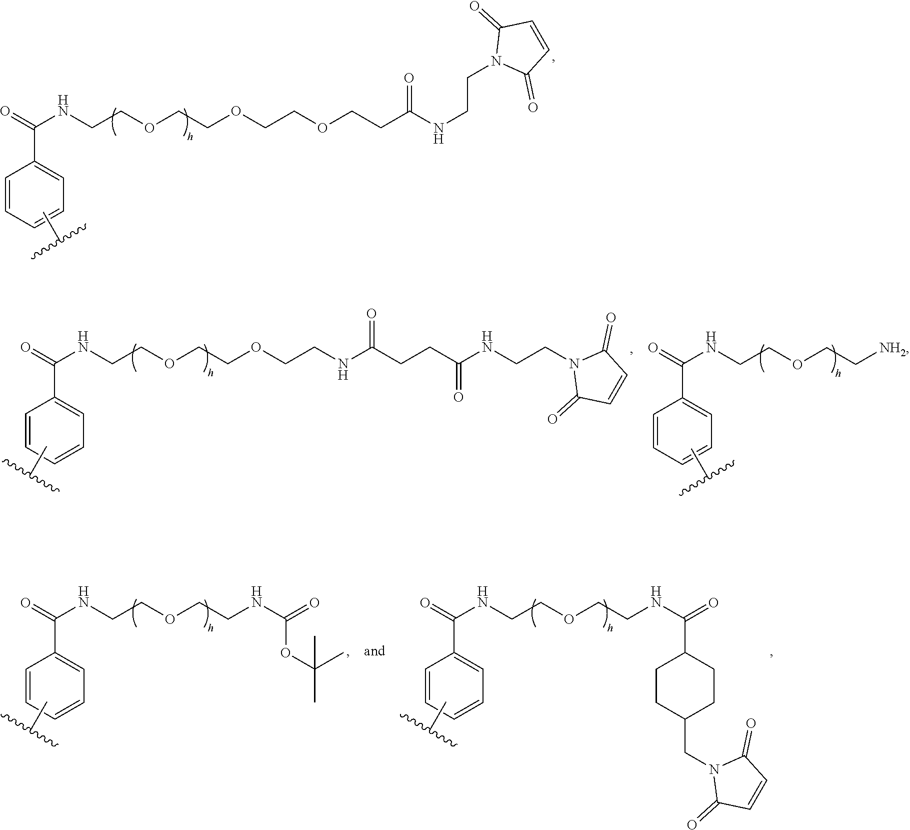

wherein
each R⁶ is independently selected from the group consisting of H, OH, SH, NHCOO-t-butyl, (CH₂)ₙCOOH, (CH₂)ₙCOOCH₃, (CH₂)ₙ(CH₂CH₂O)_{f}COOH, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino,

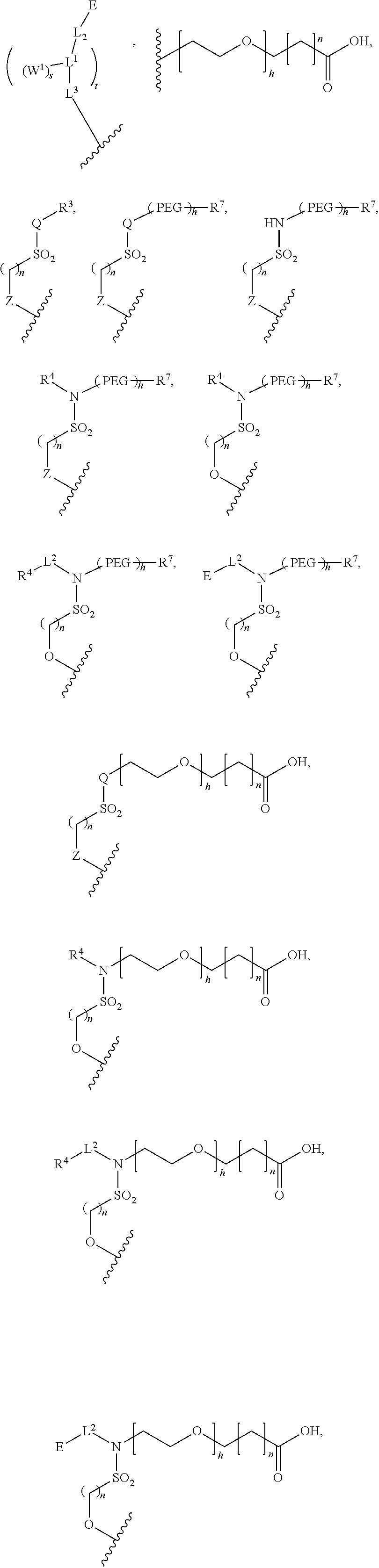

benzyl optionally substituted with one or more halogen, hydroxyl, C₁-C₁₂ alkoxy, or (OCH₂CH₂)_{f}OCH₃, and one or more pendant chains terminated with a functional group, optionally conjugated to E.

In some cases, the functional group is selected from the group consisting of amine, carbamate, carboxylic acid, carboxylate, maleimide, activated ester, N-hydroxylsuccinimidyl, hydrazine, tetrazine, cycloalkene, hydrazide, hydrazone, azide, alkyne, cycloalkyne, aldehyde, thiol, and protected groups thereof.

G¹ and G² can each independently be optionally substituted dihydrophenanthrene (DHP). G¹ and G² can each independently be optionally substituted fluorene. G¹ and G² can each independently be aryl substituted with one or more pendant chains terminated with a functional group. G¹ and G² can each independently be a heteroaryl substituted with one or more pendant chains terminated with a functional group.

G¹ and G² can each independently be

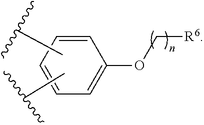

G¹ and G² can each independently be

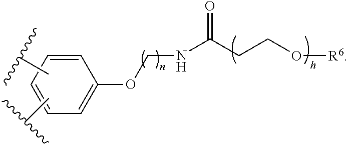

G¹ and G² can each independently be

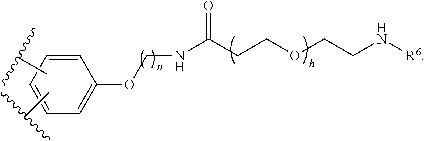

G¹ and G² can each independently be

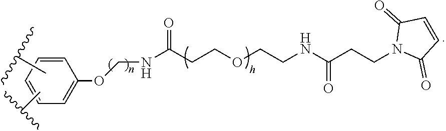

G¹ and G² can each independently be

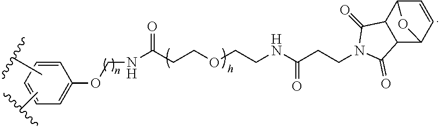

G¹ and G² can each independently be

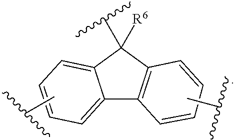

G¹ and G² can each independently be

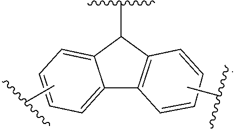

G¹ and G² can each independently be

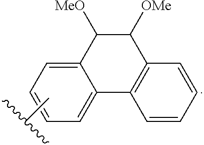

G¹ and G² can each independently be

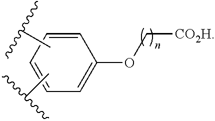

R⁶, h and n can be as described above.

In some cases, when Y is a bond, Ar¹ and Ar² are not both thienyl.

In some cases, Ar¹ and Ar² are different. In some cases, Ar¹ and Ar² are the same.

In some cases, the polymer according to Formula (Ib) comprises co-monomer M¹ according to Formula (Ia) having a structure selected from the group consisting of Formula (IIa), (IIb), (IIc), (IId), (IIe), and (IIg):

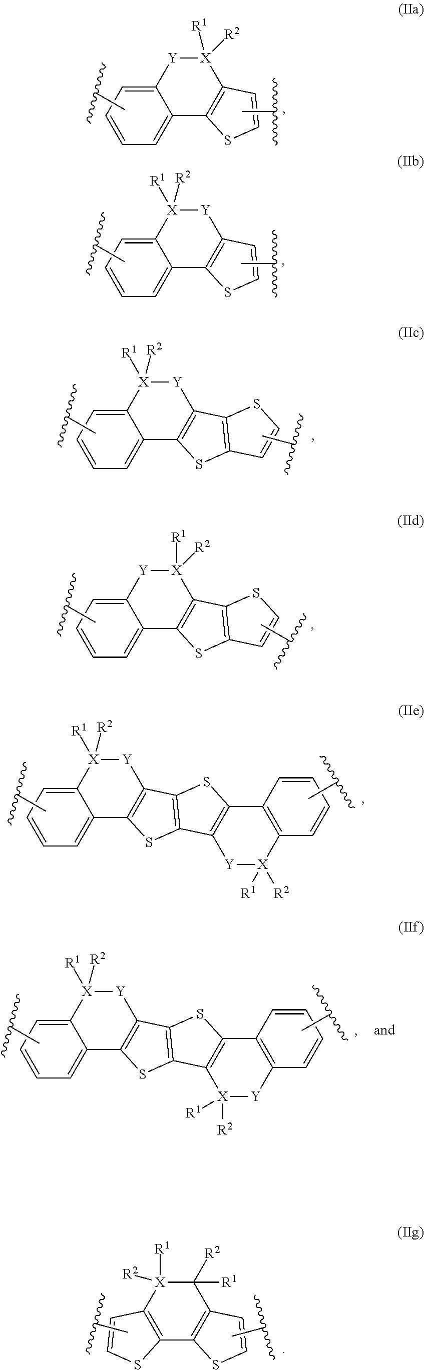

In some cases, the co-monomer M¹ according to Formula (Ia) comprises a structure selected from the group consisting of Formula (IIaa), (IIbb), (IIcc), (IIdd), (IIee), (IIff), (IIgg):

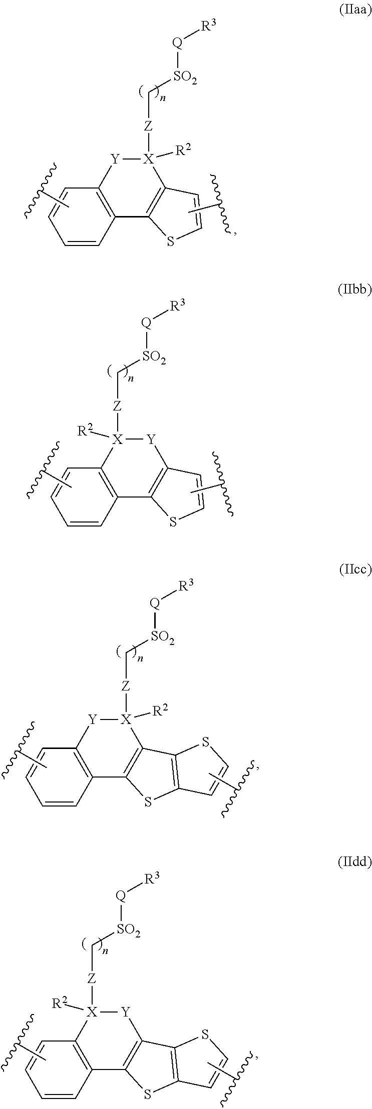

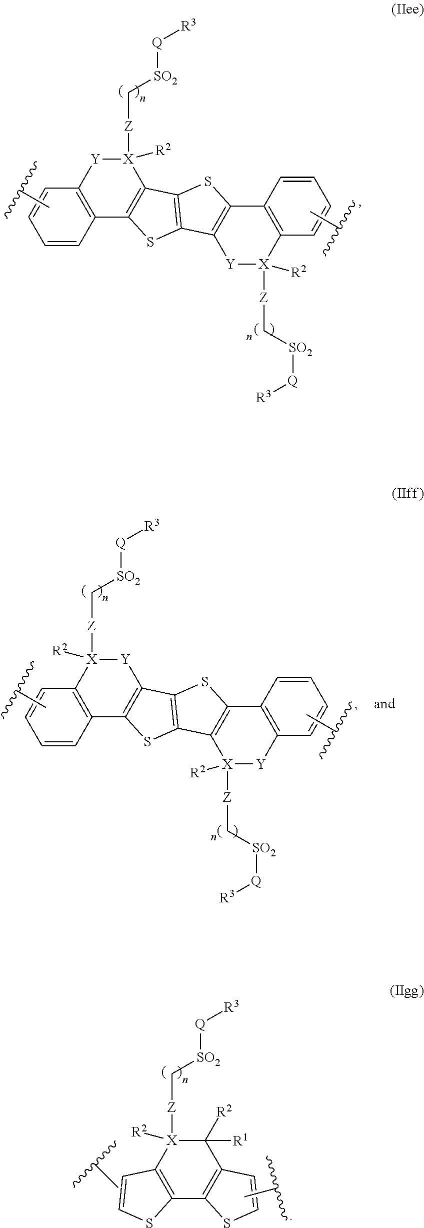

In some cases, the co-monomer M¹ according to Formula (Ia) comprises a structure selected from the group consisting of Formula (IIaaa), (IIbbb), (IIccc), (IIddd), (IIeee), (IIfff), and (IIggg):

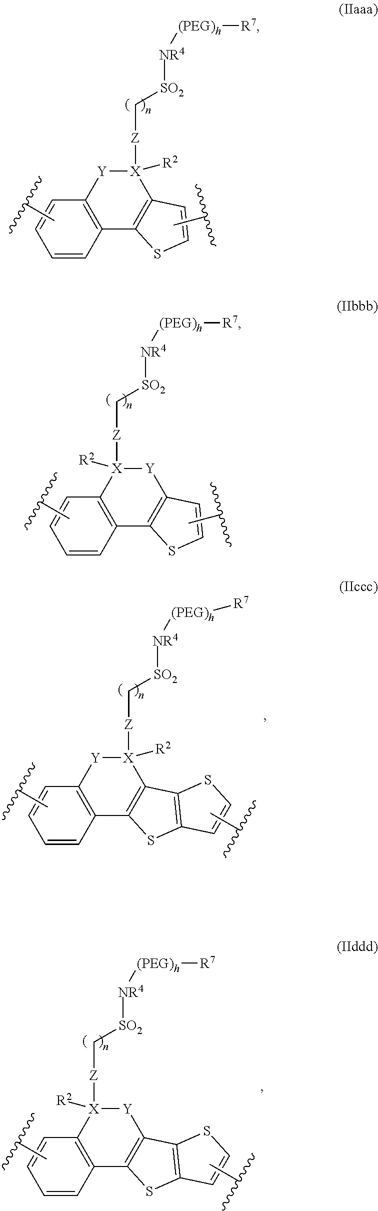

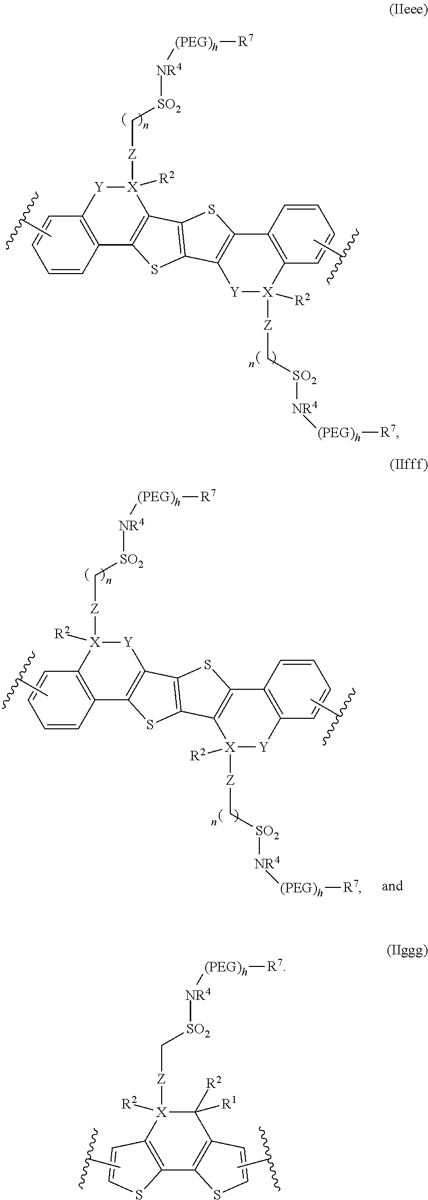

In some cases, each optional M², M³ and M⁴ may independently comprise a structure according to Formula (Ia), (IIa), (IIb), (IIc), (IId), (IIe), (IIf), or (IIg). In some cases, each optional M², M³ and M⁴ may independently comprise a structure according to Formula (IIaa), (IIbb), (IIcc), (IIdd), (IIee), (IIff), (IIgg). In some cases, each optional M², M³ and M⁴ may independently comprise a structure according to Formula (IIaaa), (IIbbb), (IIccc), (IIddd), (IIeee), (IIff), or (IIggg).

The fluorescent polymers according to the present disclosure can additionally comprise a co-monomer as known in the art. The co-monomer can be a π-conjugated co-monomer. The π-conjugated co-monomer can be selected from the group consisting of optionally substituted fluorene monomers, optionally substituted DHP monomers, optionally substituted tetrahydropyrene (THP) monomers, optionally substituted fluorenooxepine monomers, optionally substituted fused DHP monomers, or optionally substituted benzene monomers. The co-monomer can be any monomer disclosed in WO 2017/180998A2, U.S. application Ser. No. 18/207,983, and U.S. Pat. Nos. 7,629,448, 8,158,444, 8,362,193, 8,575,303, 8,802,450, 8,969,509, 9,371,559, or 9,383,353, the disclosures of each of which are incorporated herein in their entireties.

In some cases, the polymer dye according to Formula (Ib) comprises an A co-monomer selected from the group consisting of:

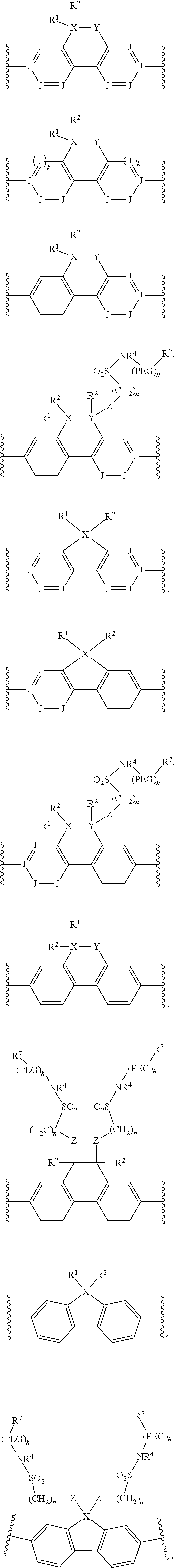

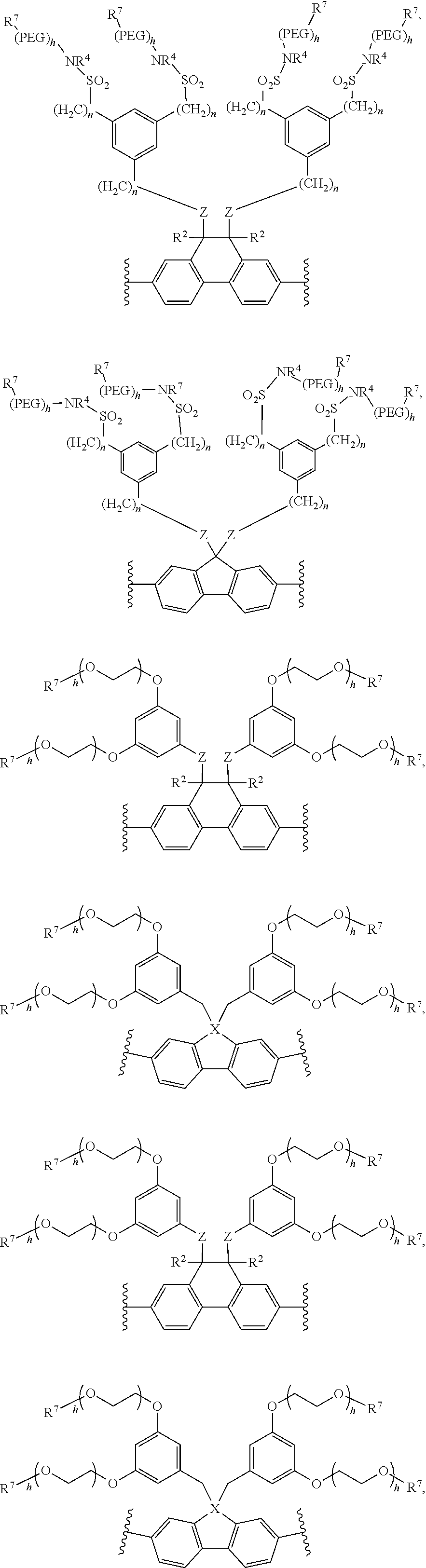

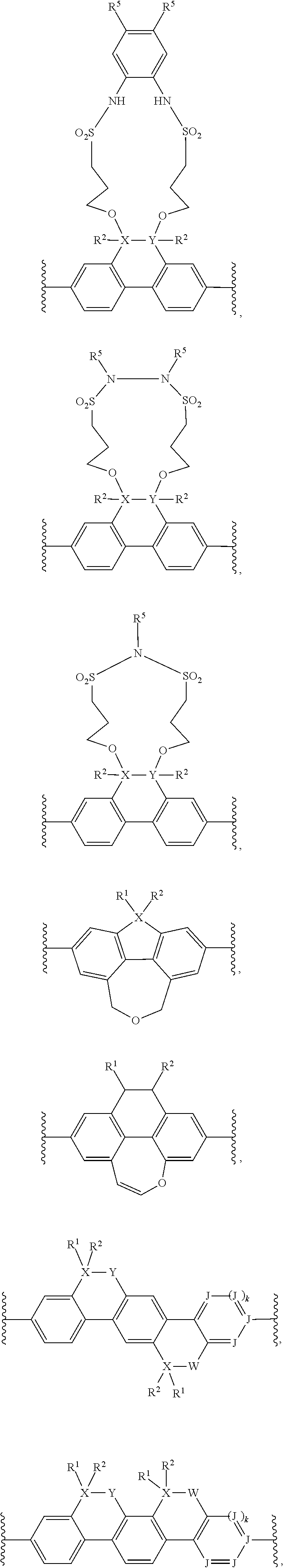

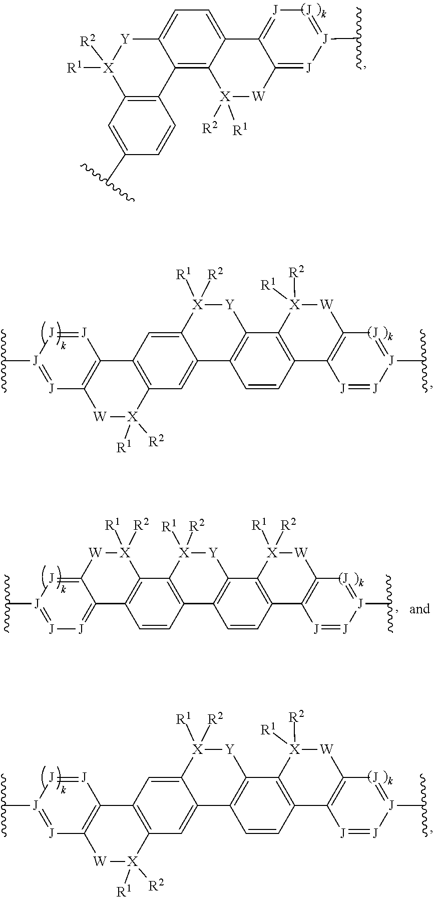

wherein
each R^{S} is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50;
each J is independently selected from the group consisting of C, C(R²), N, N(R²), P, S, Se, O, and Si(R²);
each W is independently selected from the group consisting of a bond and Y; when W is a bond X is directly bonded to both rings; and
k is independently selected from 0, 1, or 2.

k can be 0. k can be 1. k can be 2.

h can independently be an integer from 0 to 50.

n can independently be an integer from 1 to 20.

s can be 1. s can be 2.

t can be 0. t can be 1. t can be 2. t can be 3.

In some cases, the disclosure provides a polymer dye comprising a structure according to Formula (IIIa) or (IIIb):

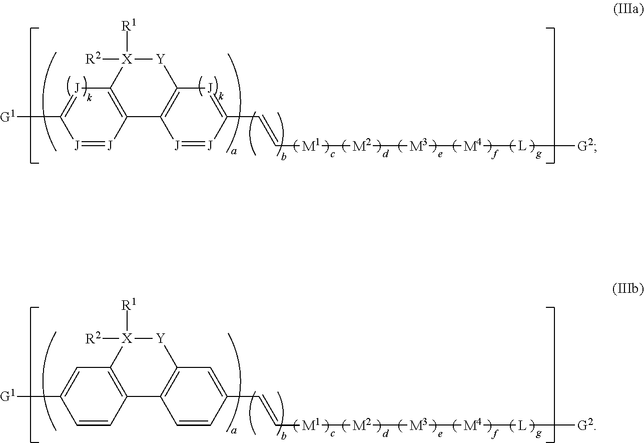

In some cases, the disclosure provides a polymer dye comprising a structure of Formula (IV):

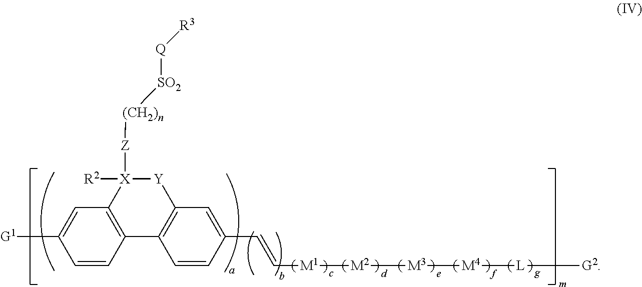

In some cases, the disclosure provides a polymer dye comprising a structure of Formula (V):

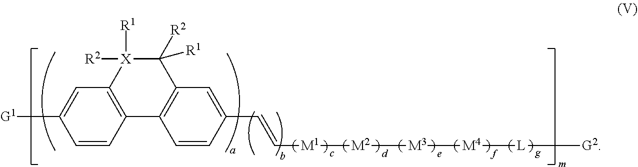

In some cases, the disclosure provides a polymer dye comprising a structure of Formula (VI):

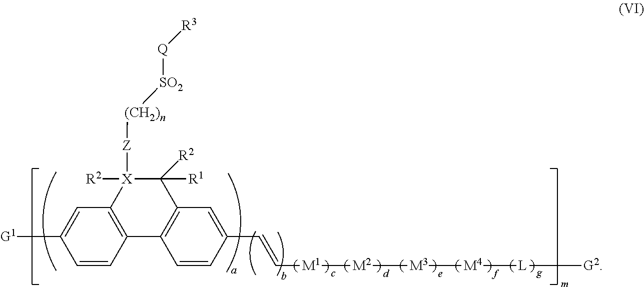

In some cases, the disclosure provides a polymer dye comprising a structure of Formula (VIIa):

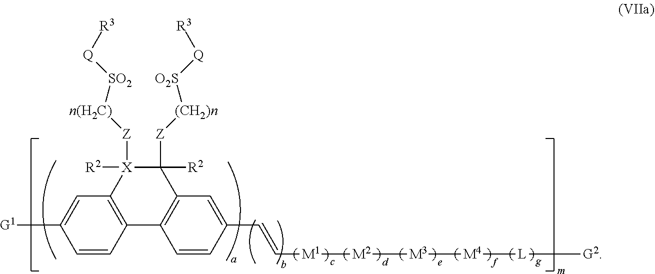

In some cases, the disclosure provides a polymer dye comprising a structure of Formula (VIIb):

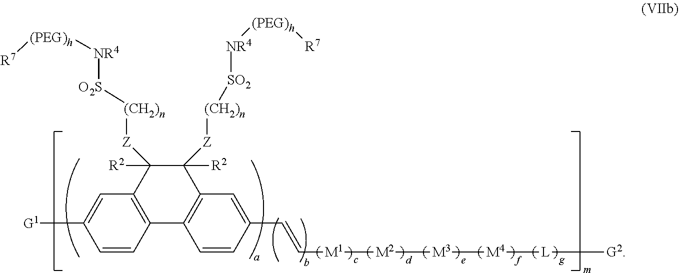

In some cases, the disclosure provides a polymer dye comprising a structure of Formula (VIIc):

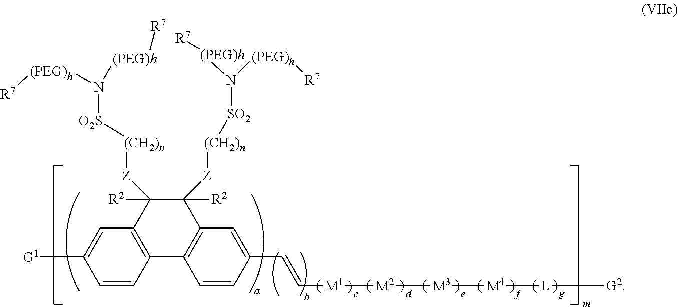

In some cases, the disclosure provides a polymer dye comprising a structure of Formula (VIIIa):

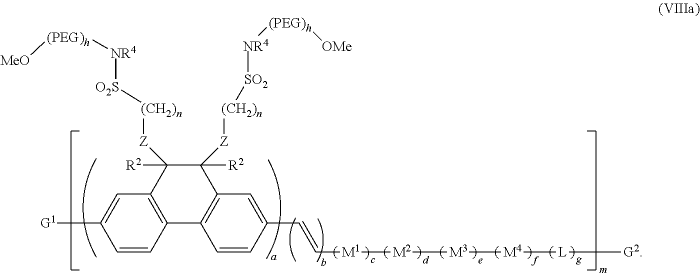

In some cases, the disclosure provides a polymer dye comprising a structure of Formula (VIIIb):

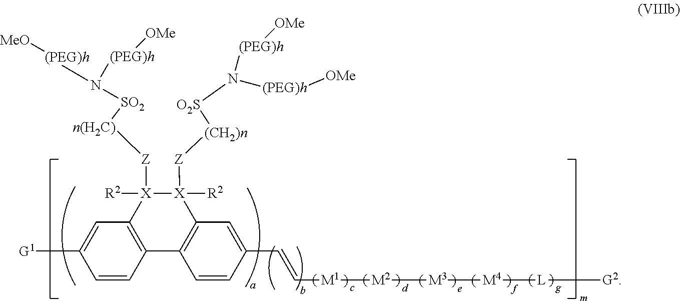

In some cases, the disclosure provides a polymer dye comprising a structure of Formula (VIId):

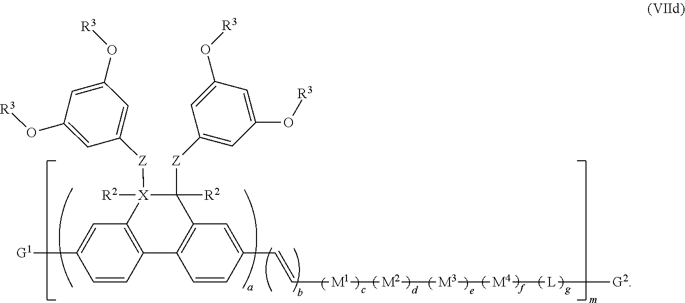

In some cases, the disclosure provides a polymer dye comprising a structure of Formula (VIIe):

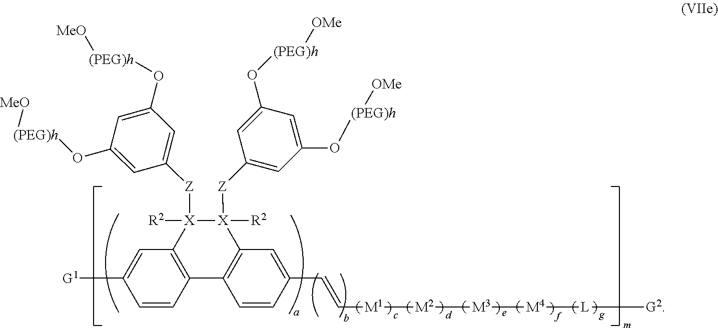

In some cases, the disclosure provides a polymer dye comprising a structure of Formula (IXa):

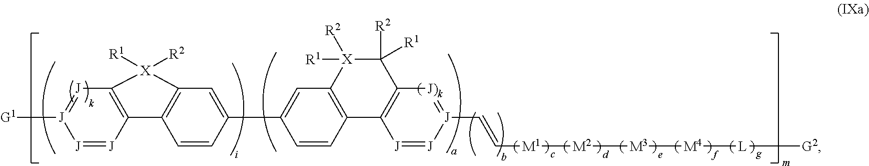

wherein, a and i together is a mol % from 10 to 100%.

In some cases, the disclosure provides a polymer dye comprising a structure of Formula (IXb):

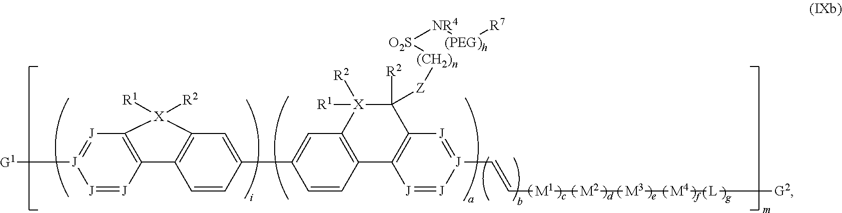

wherein, a and i together is a mol % from 10 to 100%.

In some cases, the disclosure provides a polymer dye comprising a structure of Formula (IXc):

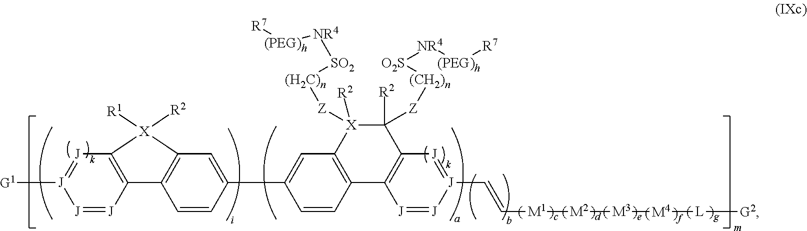

wherein, a and i together is a mol % from 10 to 100%.

In some cases, the disclosure provides a polymer dye comprising a structure of Formula (Xa):

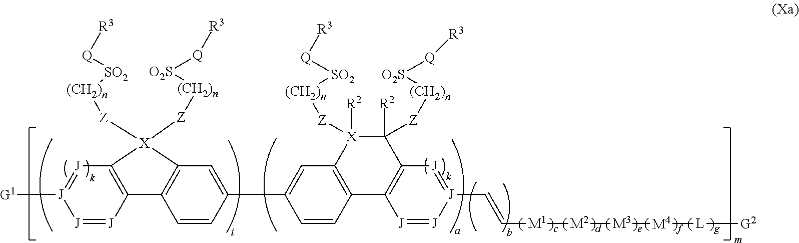

wherein, a and i together is a mol % from 10 to 100%.

In some cases, the disclosure provides a polymer dye comprising a structure of Formula (Xb):

wherein, a and i together is a mol % from 10 to 100%.

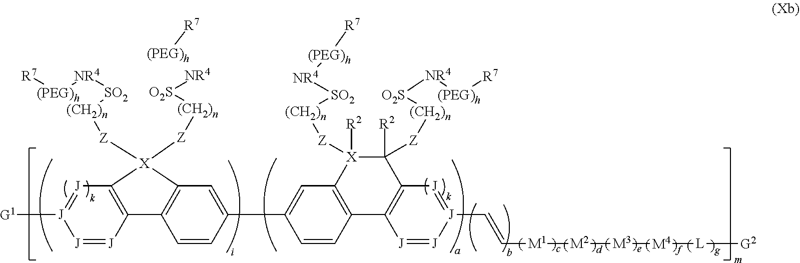

In some cases, the disclosure provides a polymer dye comprising a structure of Formula (XI):

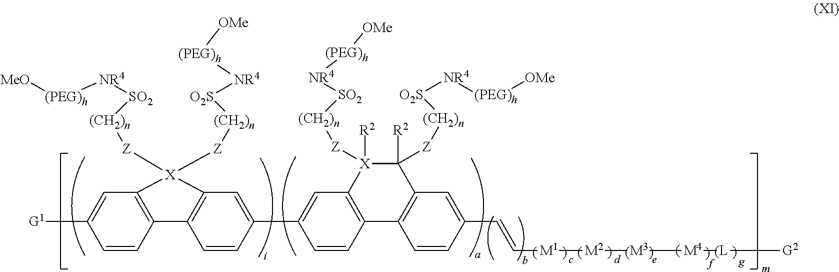

wherein, a and i together is a mol % from 10 to 100%.

In some cases, the disclosure provides a polymer dye comprising a structure of Formula (XII):

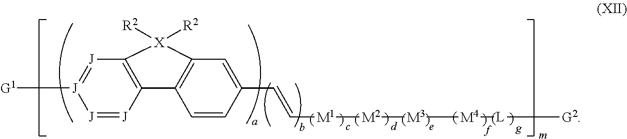

In some cases, the disclosure provides a polymer dye comprising a structure of Formula (XIIIa):

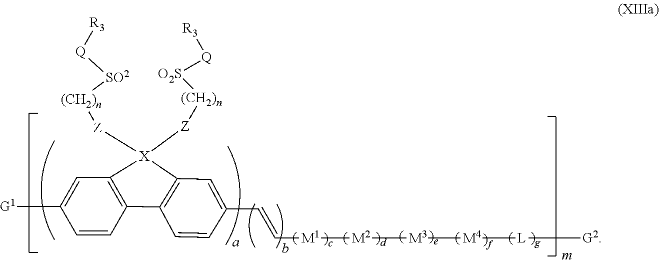

In some cases, the disclosure provides a polymer dye comprising a structure of Formula (XIIIb):

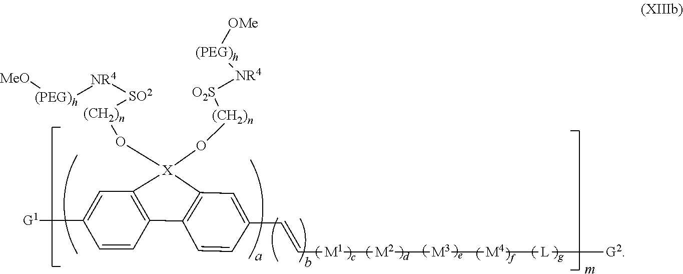

In some cases, the disclosure provides a polymer dye comprising a structure of Formula (XIIIc):

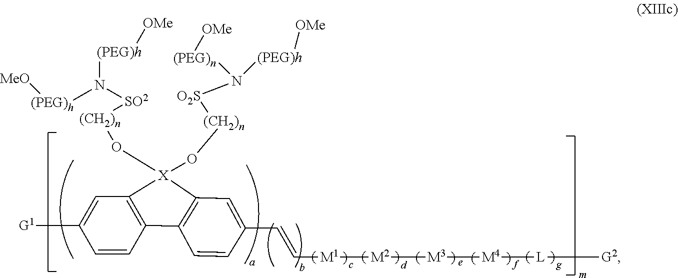

In some cases, the disclosure provides a polymer dye comprising a structure of Formula (XIIId):

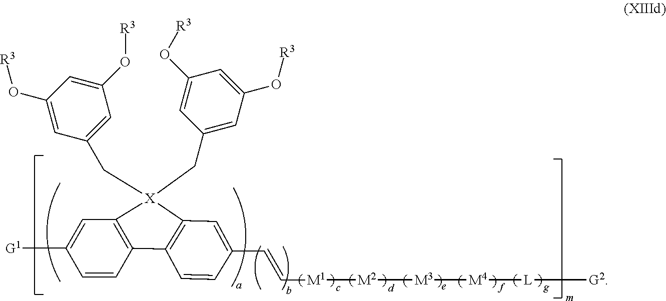

In some cases, the disclosure provides a polymer dye comprising a structure of Formula (XIIIe):

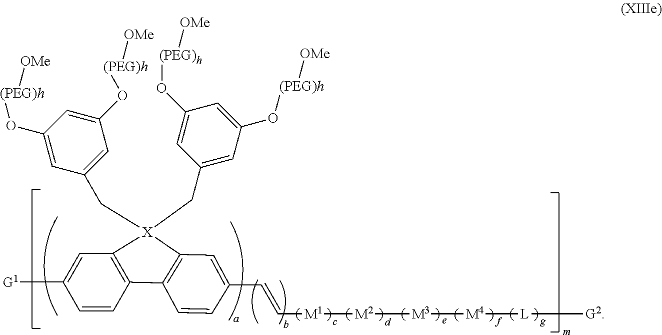

In some cases, the disclosure provides a polymer dye comprising a structure of Formula (XIVa):

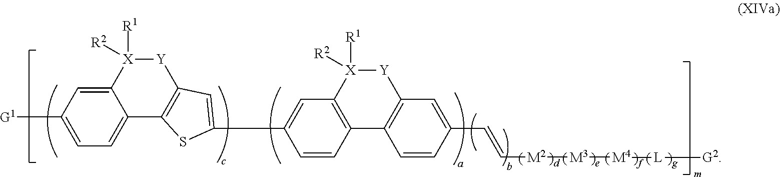

In some cases, the disclosure provides a polymer dye comprising a structure of Formula (XIVb):

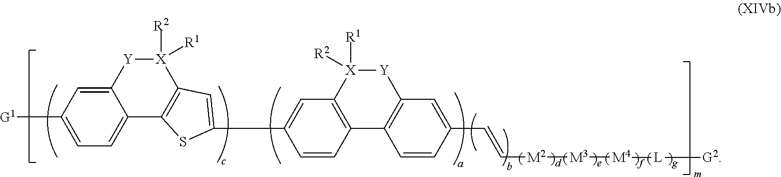

In some cases, the disclosure provides a polymer dye comprising a structure of Formula (XIVc):

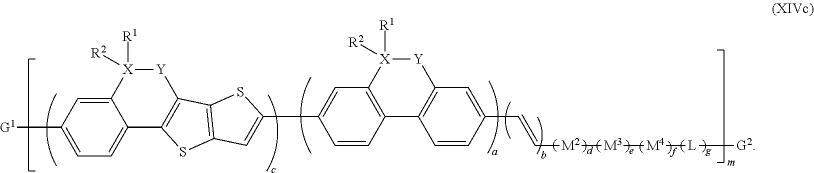

In some cases, the disclosure provides a polymer dye comprising a structure of Formula (XIVd):

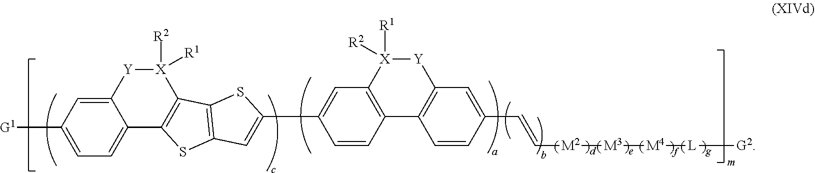

In some cases, the disclosure provides a polymer dye comprising a structure of Formula (XIVe):

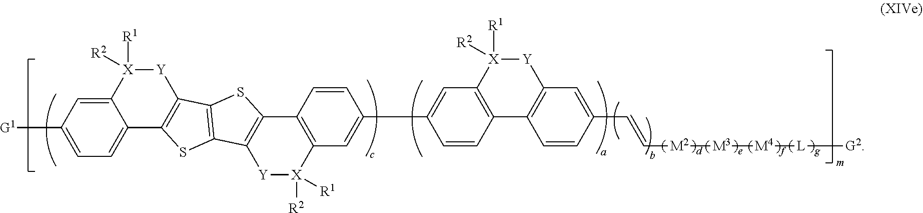

In some cases, the disclosure provides a polymer dye comprising a structure of Formula (XIVf):

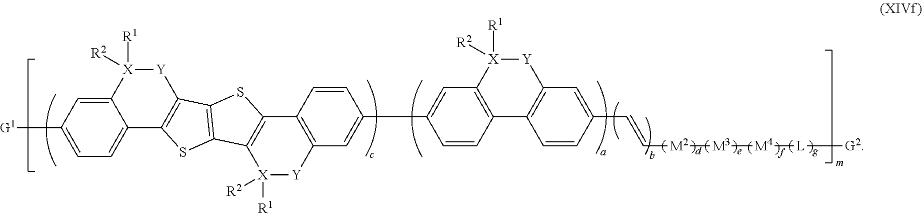

In some cases, the disclosure provides a polymer dye comprising a structure of Formula (XIVg):

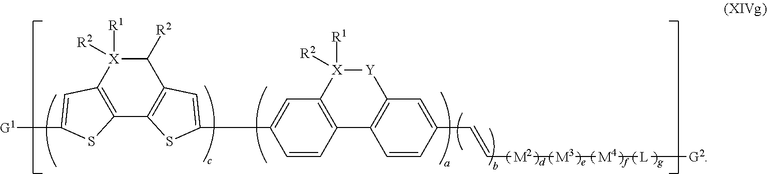

In some cases, the disclosure provides a polymer dye comprising a structure of any one of Formulas (XVa), XVb), (XVc), (XVd), (XVe), (XVf), and (XVg):

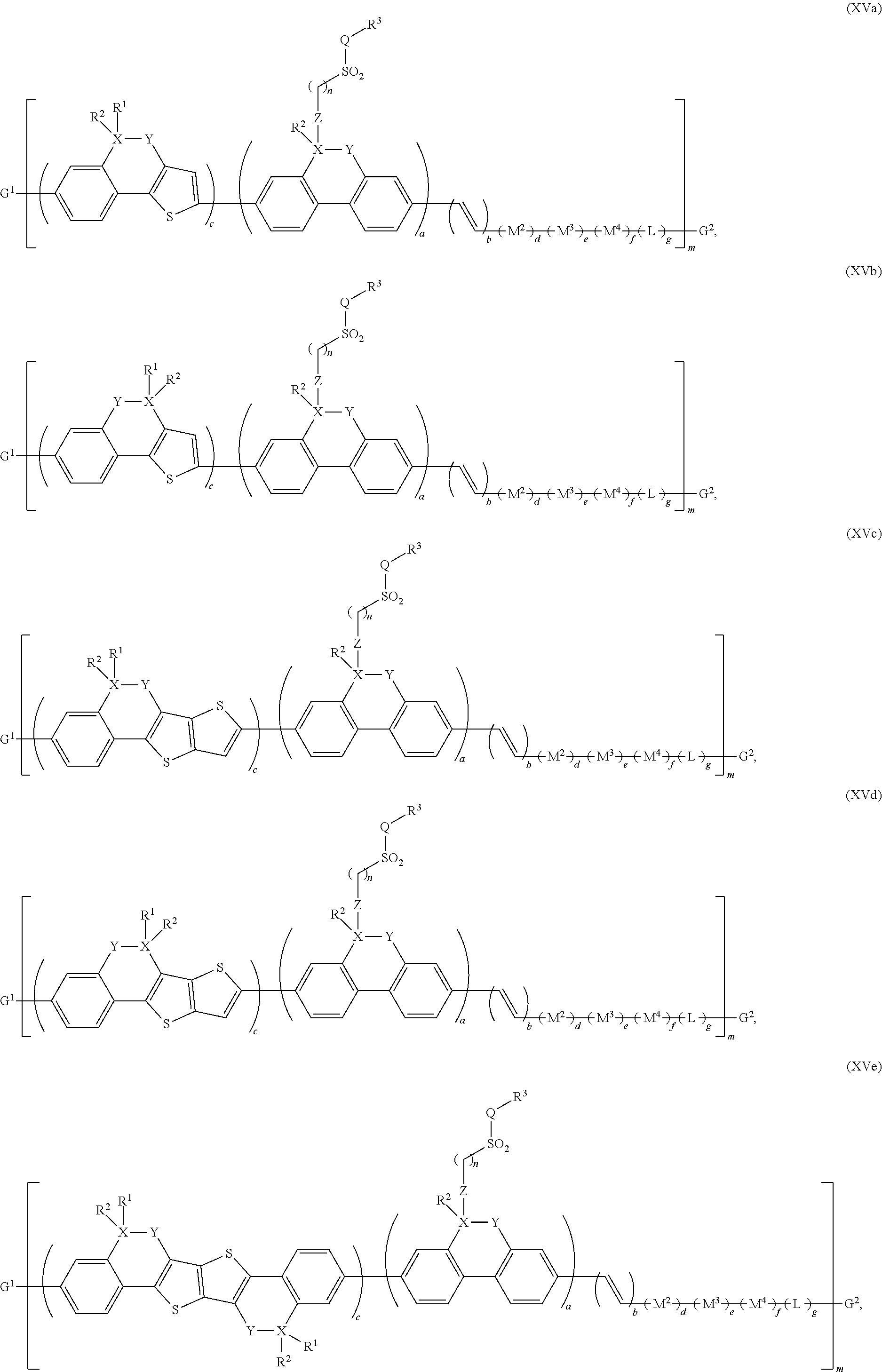

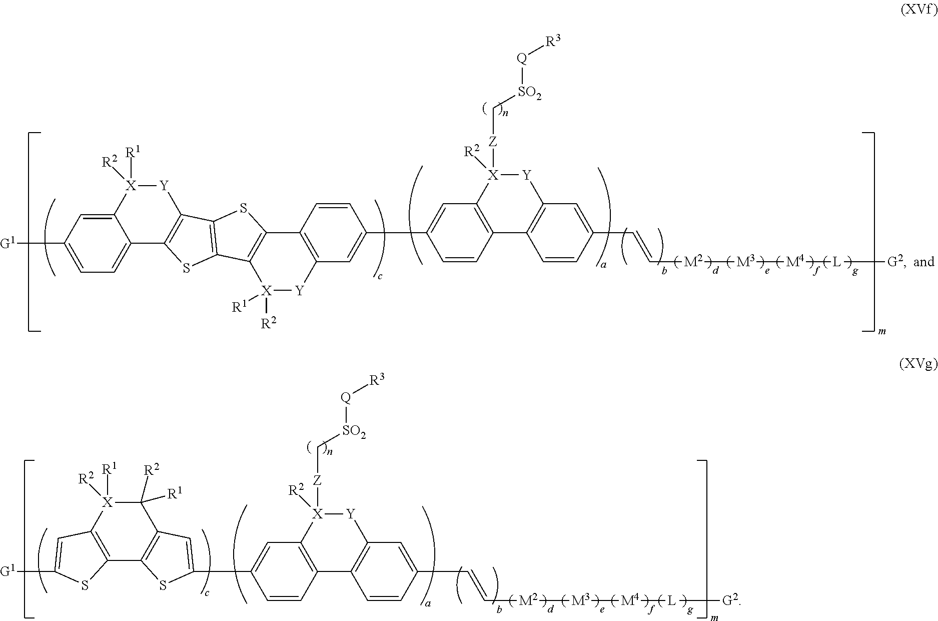

In some cases, the disclosure provides a polymer dye comprising structure of any one of Formulas (XVIa), (XVIb), XVIc), and (XVId):

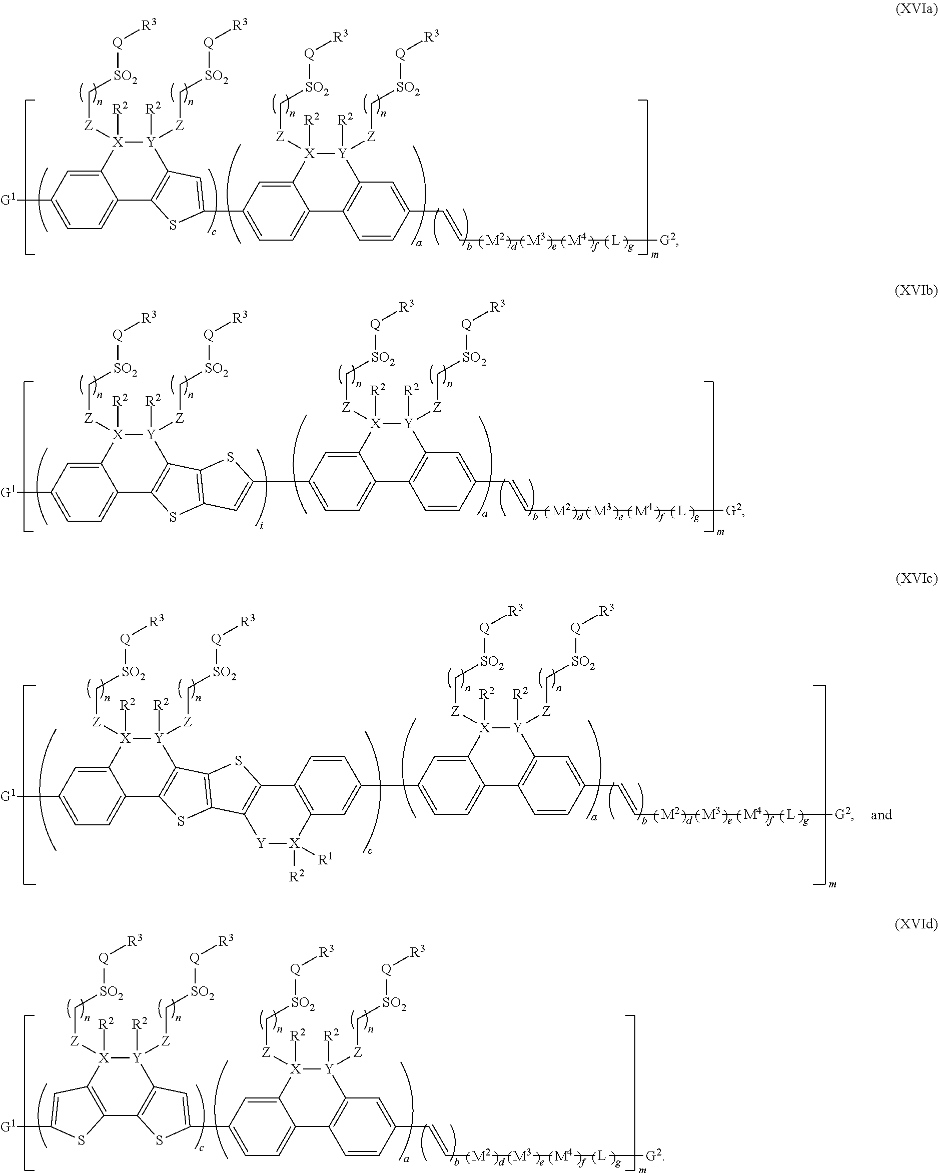

In some cases, the disclosure provides a polymer dye comprising a structure of any one of Formulas (XVIIa), (XVIIb), (XVIIc), and (XVIId):

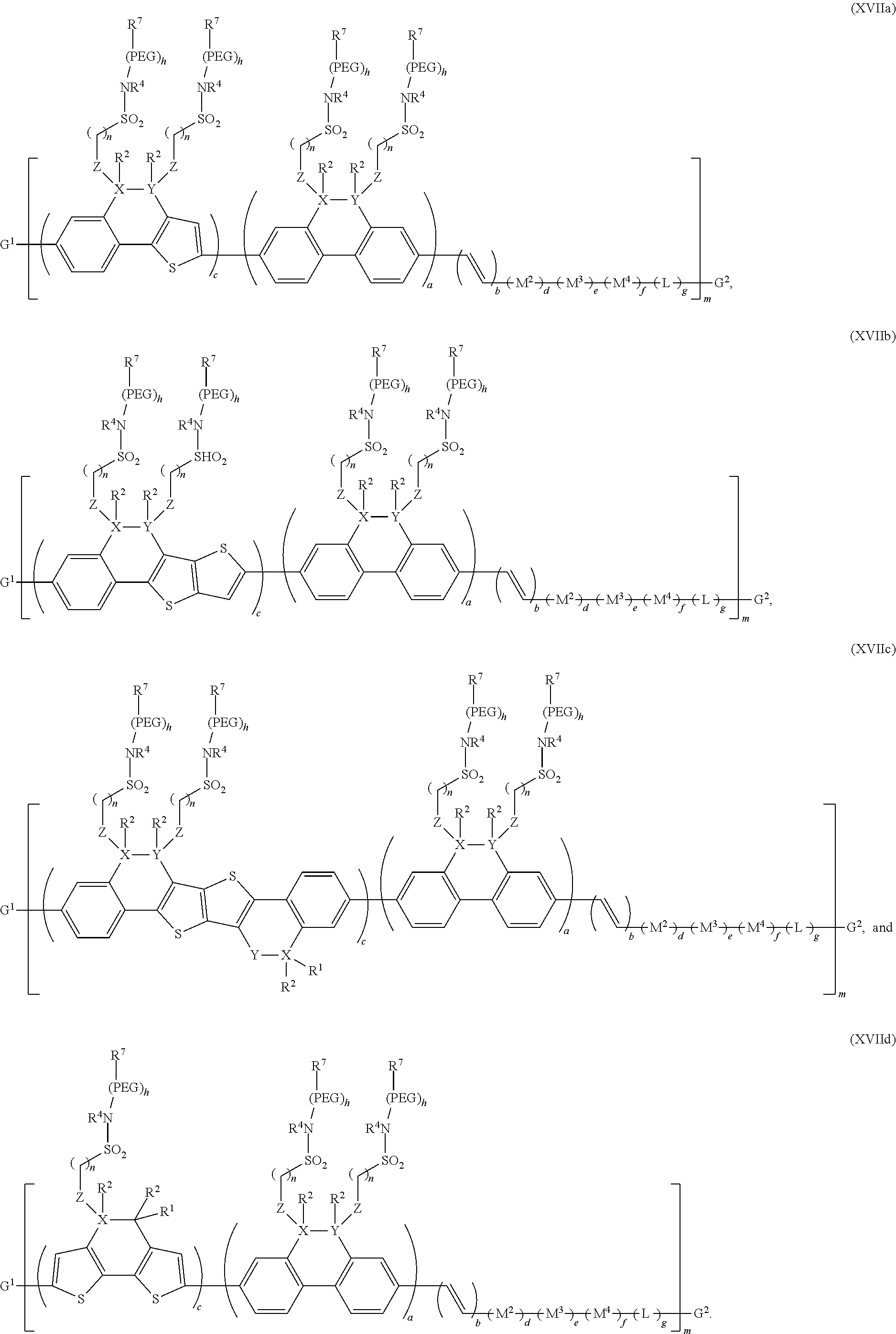

In each of the polymer dyes, (IIIa) to (IIIg), (IV), (V), (VI), (VIIa) to (VIIe), (VIIIa), (VIIIb), (IXa) to (IXc), (Xa), (Xb), (XI), (XII), (XIIIa) to (XIIIe), (XIVa) to (XIVg), (XVa) to (XVg), (XVIa) to (XVId), and (XVIIa) to (XVIId), each M¹, M², M³, M⁴, Ar¹, Ar², X, Y, E, G¹, G², J, Z, Q, W, W¹, L, L¹, L², L³, R¹, R², R³, R⁴, R⁵, R⁷, R⁹, a, b, c, d, e, f, g, h, i, k, m, n, s, and t are as previously defined herein, and at least one of R¹, R², R³, or R⁴ comprises a water-solubilizing moiety.

The fluorescent polymers of the present disclosure can contain polymer modifying units, represented as M², M³, or M⁴, that are capable of altering the polymer band gap. Each M², M³, or M⁴ can be evenly or randomly distributed along the polymer main chain. Each M², M³, or M⁴ is optional. In some cases, the disclosure provides a polymer dye comprising a structure as provided herein, wherein each optional M², M³ or M⁴ is independently selected from the group consisting of:

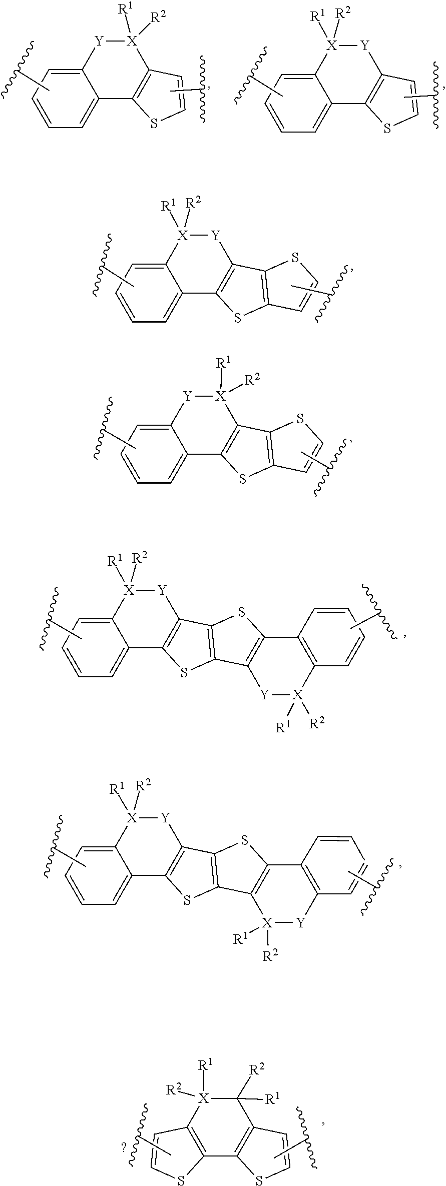

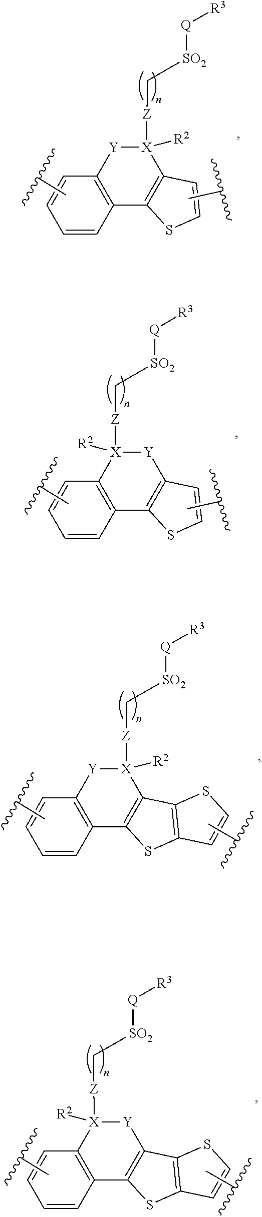

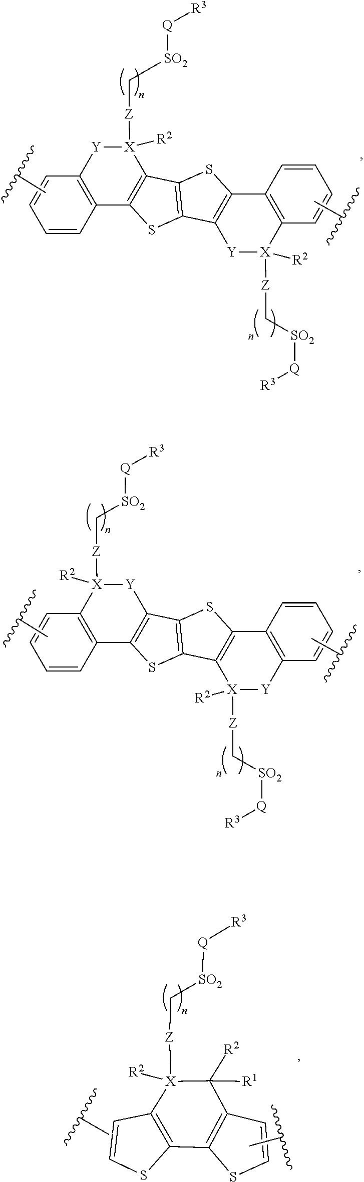

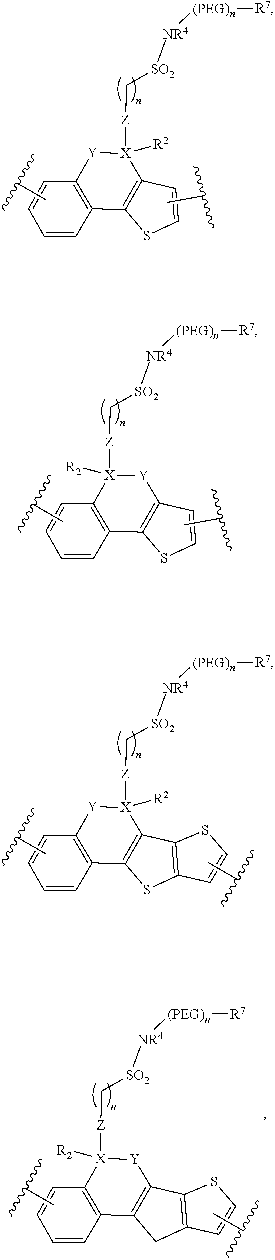

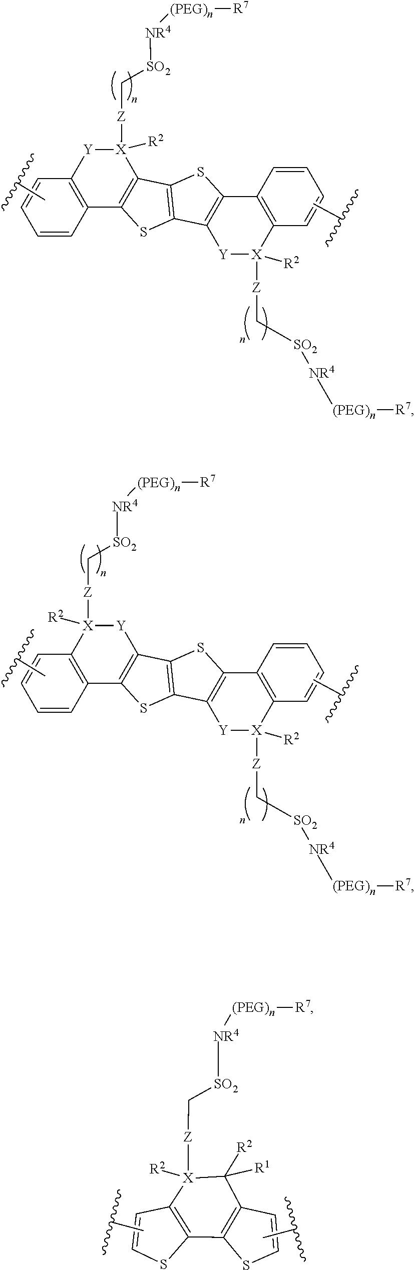

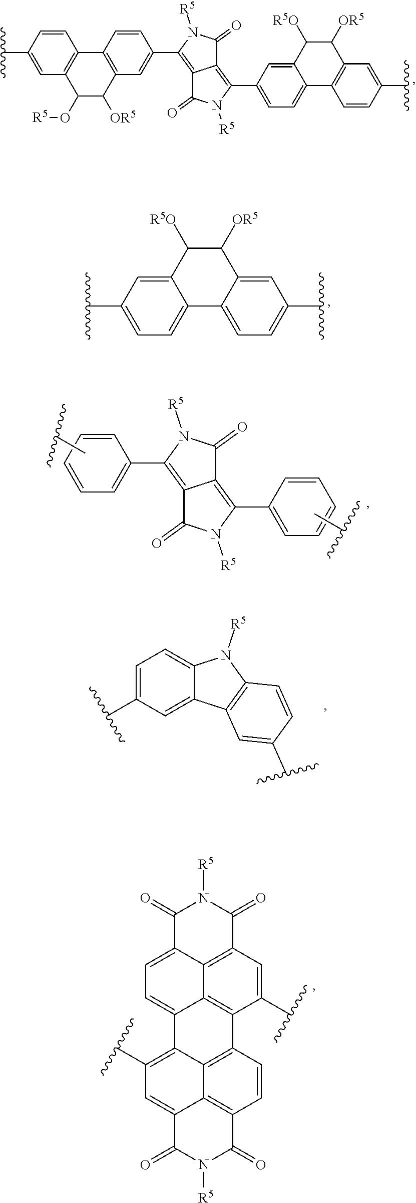

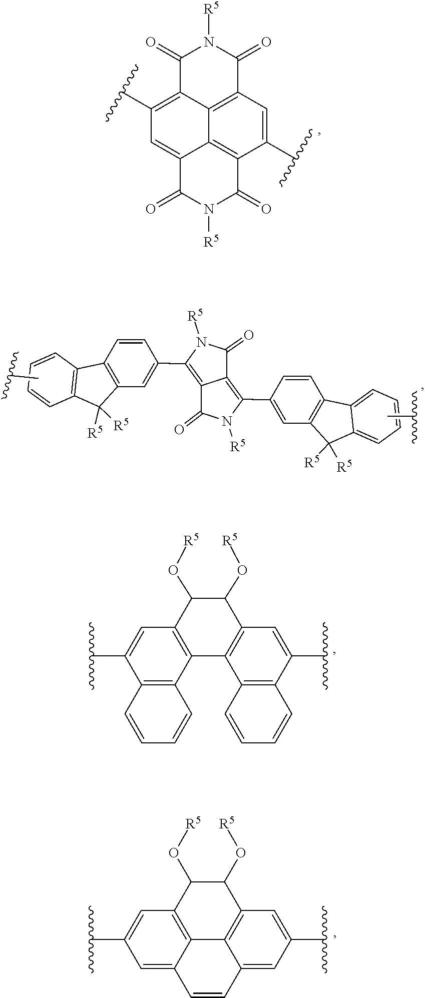

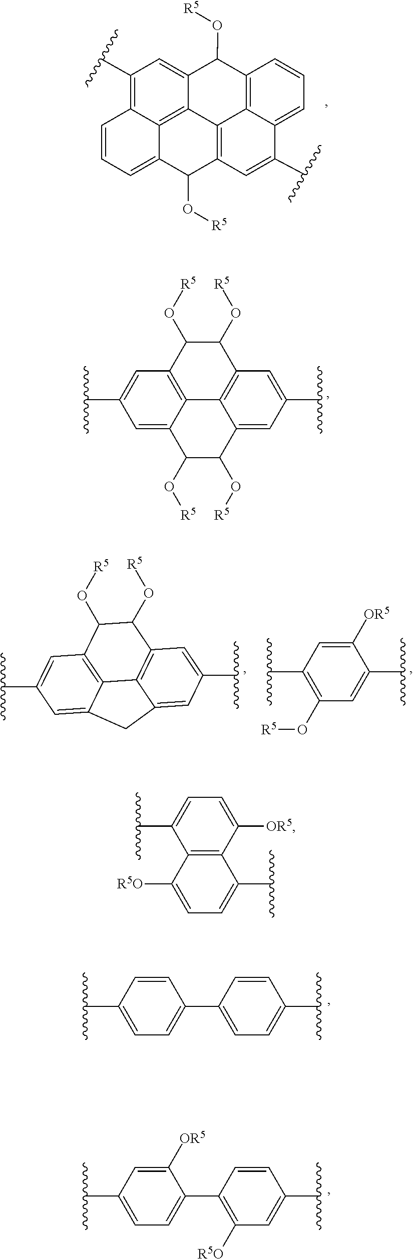

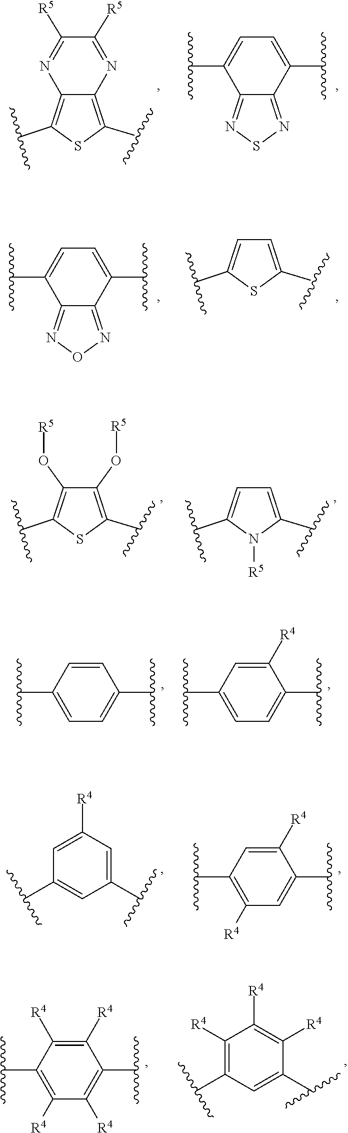

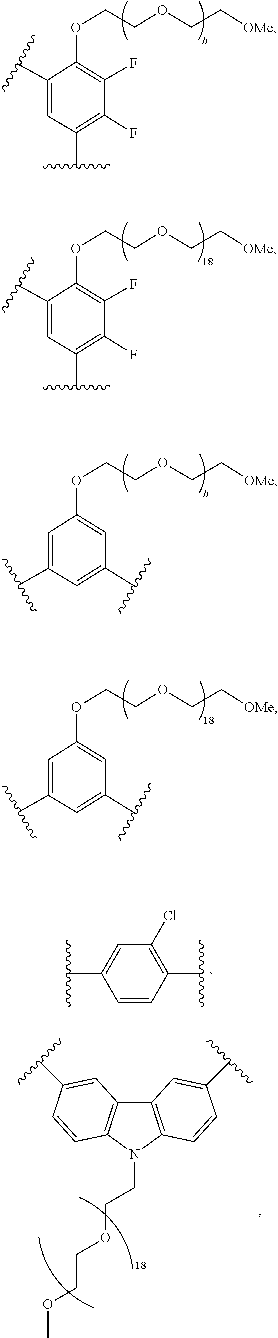

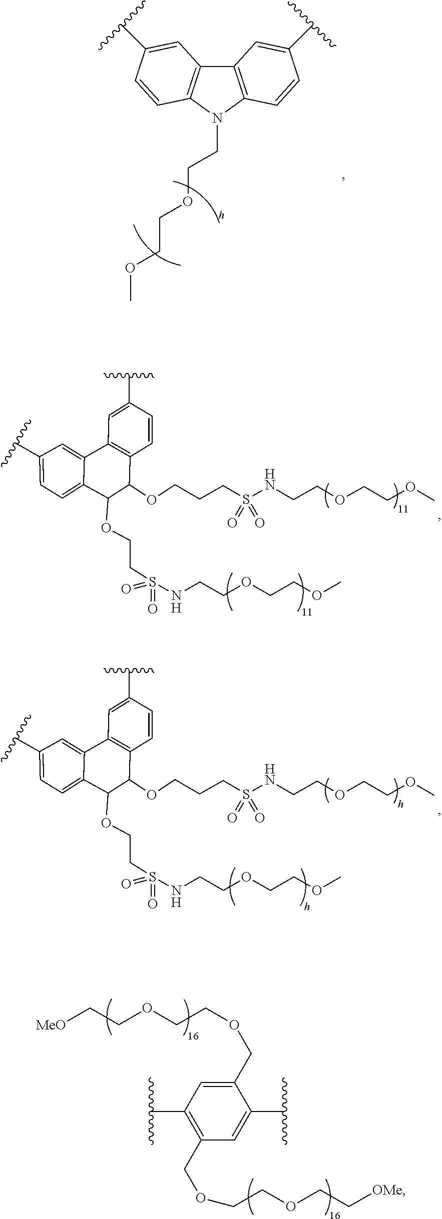

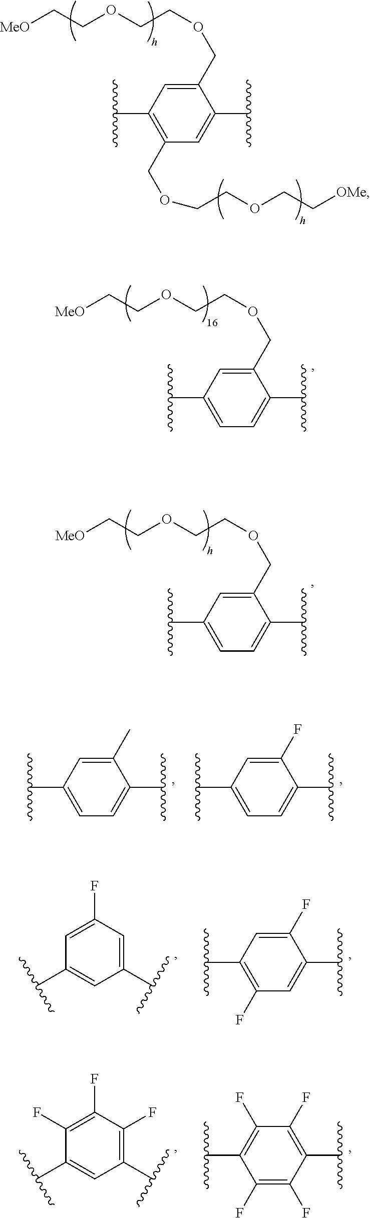

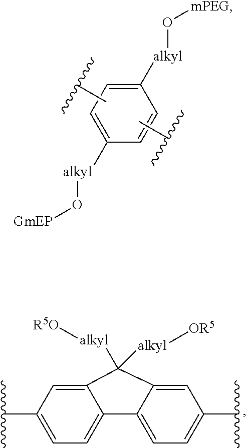

an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substitute, wherein
each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50, optionally conjugated to E.

R⁵ can be halogen. R⁵ can be hydroxyl. R⁵ can be C₁-C₁₂ alkyl. R⁵ can be C₂-C₁₂ alkene. R⁵ can be C₂-C₁₂ alkyne. R⁵ can be C₃-C₁₂ cycloalkyl. R⁵ can be C₁-C₁₂haloalkyl. R⁵ can be C₁-C₁₂ alkoxy. R⁵ can be C₂-C₁₈ (hetero)aryloxy. R⁵ can be C₂-C₁₈ (hetero)arylamino. R⁵ can be carboxylic acid. R⁵ can be carboxylate ester. R⁵ can be (CH₂)_{x′}(OCH₂—CH₂)_{y′}OCH₃. R⁵ can be C₂₋₁₈ (hetero)aryl group. x′ can be an integer from 0-20. x′ can be an integer from 0-10. x′ can be an integer from 1-4. y′ can be an integer from 0-50. y′ can be an integer from 0-40. y′ can be an integer from 0-30. y′ can be an integer from 0-20. y′ can be an integer from 0-10. y′ can be an integer from 1-4.

In some cases, R⁴ can be H. R⁴ can be alkyl. R⁴ can be PEG. R⁴ can be a water-solubilizing moiety. R⁴ can be a linker moiety. R⁴ can be a chromophore. R⁴ can be carboxylic amine. R⁴ can be amine. R⁴ can be carbamate. R⁴ can be carboxylic acid. R⁴ can be carboxylate. R⁴ can be maleimide.

In some embodiments, at least one of R¹, R², R³, or R⁴ comprises a water-solubilizing moiety.

The polymer according to the disclosure may be a water-soluble fluorescent polymer dye.

The water-soluble fluorescent polymer dye according to the disclosure may comprise one or more water-soluble moieties. The water solubilizing moieties can each independently selected from the group consisting of a PEG group, linked PEG group, carboxylic acid, carboxylate, polyvinyl alcohol, glycol, peptide, polyphosphate, polyalcohol, sulfonate, phosphonate, boronate, amine, ammonium, sulfonium, phosphonium, alcohol, polyol, oxazoline, zwitterionic derivative, carbohydrate, nucleotide, polynucleotide, substituted PEG group, substituted carboxy group, substituted carboxylic acid, substituted carboxylate, substituted glycol, substituted peptide, substituted polyphosphate, substituted polyalcohol, substituted sulfonate, substituted phosphonate, substituted boronate, substituted amine, substituted ammonium, substituted sulfoniums substituted phosphonium, substituted zwitterionic derivative, substituted carbohydrate, substituted nucleotide, and substituted polynucleotide, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, phosphonate,

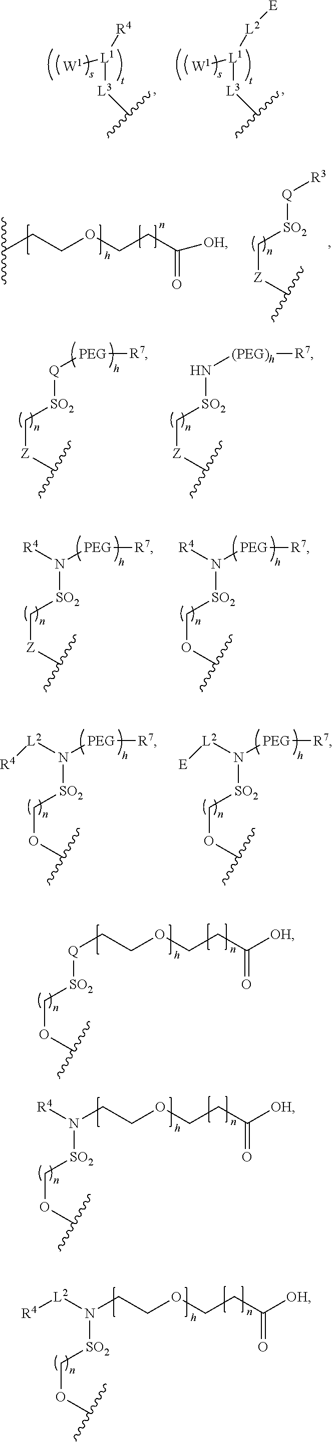

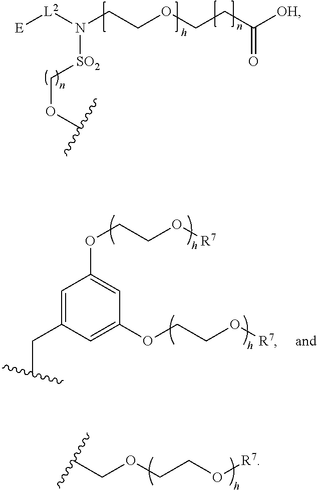

Each of X, Y, Z, W, R¹, R², R³, R⁴, R⁵, R⁶, R⁷, M, L, L¹, L², L³, J, G¹, G², Q a, b, c, d, e, f, h, k, m, and n are as described above.

The disclosure provides a polymer tandem dye comprising a polymer dye according to the present disclosure; and a signaling chromophore covalently linked to the polymer dye in energy-receiving proximity therewith. In some embodiments, the fluorescent polymer tandem can be water-soluble. In some embodiments, the fluorescent tandem polymer may comprise a specific binding partner covalently linked to the polymer.

The disclosure provides a labeled specific binding partner, comprising a polymer dye according to the disclosure; and a specific binding partner covalently linked to the polymer dye, wherein the specific binding partner is capable of specific binding to a target analyte. The specific binding partner may be selected from the group consisting of a protein, peptide, affinity ligand, antibody, antibody fragment, carbohydrate, lipid, nucleic acid, and an aptamer. The specific binding partner may be an antibody. In some embodiments, the specific binding partner may be covalently linked to a G¹ and/or G² moiety of the polymer dye.

In some embodiments, the fluorescent polymer of the present disclosure can be a fluorescent copolymer comprising a combination of polymers, wherein at least one or more of two or more of, or three or more of the fluorescent polymers has a structure selected from the group consisting of Formula (Ib), Formula (IIIa), Formula (IIIb), Formula (IV), Formula (V), Formula (VI), Formula (VIIa), Formula (VIIb), Formula (VIIc), Formula (VIId), Formula (VIIe), Formula (VIIIa), Formula (VIIIb), Formula (IXa), Formula (IXb), Formula (IXc), Formula (Xa), Formula (Xb), Formula (XI), Formula (XII), Formula (XIIIa), Formula (XIIIb), Formula (XIIIc), Formula (XIIId). Formula (XIIIe), Formula (XIVa), Formula (XIVb), Formula (XIVc), Formula (XIVd), Formula (XIVe), Formula (XIVf), Formula (XIVg), Formula (XVa), Formula (XVb), Formula (XVc), Formula (XVd), Formula (XVe), Formula (XVf), Formula (XVg), Formula (XVIa), Formula (XVIb), Formula (XVIc), Formula (XVId), Formula (XVIIa), Formula (XVIIb), Formula (XVIIc), and Formula (XVIId).

In some embodiments, the fluorescent polymers as described herein are characterized by a minimum number average molecular weight, Mn, of greater than 5,000 g/mol, greater than 10,000 g/mol, greater than 15,000 g/mol, greater than 20,000 g/mol, greater than 25,000 g/mol, greater than 30,000 g/mol, greater than 40,000 g/mol, greater than 50,000 g/mol, greater than 60,000 g/mol, greater than 70,000 g/mol, greater than 80,000 g/mol, greater than 90,000 g/mol, or greater than 100,000 g/mol.

In some embodiments, polymers as described herein are characterized by a minimum weight average molecular weight, Mw, of greater than 5,000 g/mol, greater than 10,000 g/mol, greater than 15,000 g/mol, greater than 20,000 g/mol, greater than 25,000 g/mol, greater than 30,000 g/mol, greater than 40,000 g/mol, greater than 50,000 g/mol, greater than 60,000 g/mol, greater than 70,000 g/mol, greater than 80,000 g/mol, greater than 90,000 g/mol, or greater than 100,000 g/mol. Number average and weight average molecular weight values can be determined by gel permeation chromatography (GPC) using polymeric standards (e.g., polystyrene or like material).

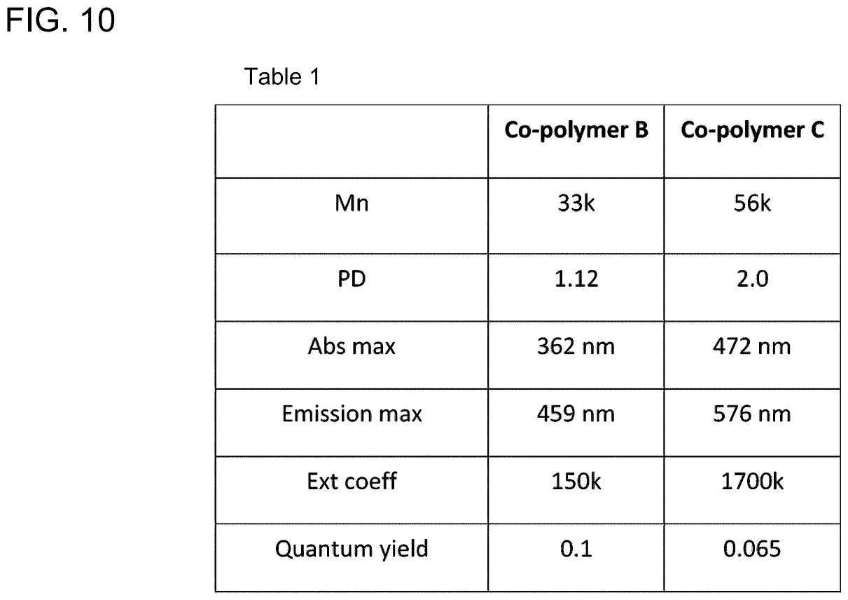

It is believed that the fluorescent polymers of the present disclosure possess certain physical and chemical characteristics of absorption, fluorescence, brightness, molecular weight, polydispersity, dye to protein ratio when conjugated to a binding partner (e.g., antibody etc.). In some instances, the ranges of these parameters are those shown in FIG. 10, and Table 2.

TABLE 2
Photophysical properties of fluorescent co-
polymer containing DHNT, DHNTT, or DHBDT monomers
according to the present disclosure.

| | |
|---|---|
| Number Averaged Molecular Weight | 10K to 200K, 30K to 100K (e.g., |
| (Mn, Dalton) | 33K or 56K) |
| Polydispersity | 1-2.2, 1.1-2 (e.g., 1.12) |
| Extinction Coefficient at 375 nm | 90K to 4,000K, 100k-3,000k (e.g., |
| (M⁻¹cm⁻¹) | 1,700K) |
| QY factor | 0.01-1.0, 0.05-0.5 (e.g., 0.1) |
| Brightness | 0.5K to 2, 200K, 10K to 1,000K |
| | (e.g., 15K, 110K) |
| Absorption/Emission Max | Abs λₘₐₓ340-600 nm; 360-475 nm |
| | Em λₑₘ 380-700 nm; 440-580 nm |

### Monomers

The present disclosure provides monomers for making the fluorescent polymers and co-polymers described herein. Non-limiting examples of the monomers or co-monomers (or monomeric units) may comprise a structure selected from the group consisting of Formula (Ia), (IIa), (IIb), (IIc), (IId), (IIe), (IIf) and (IIg):

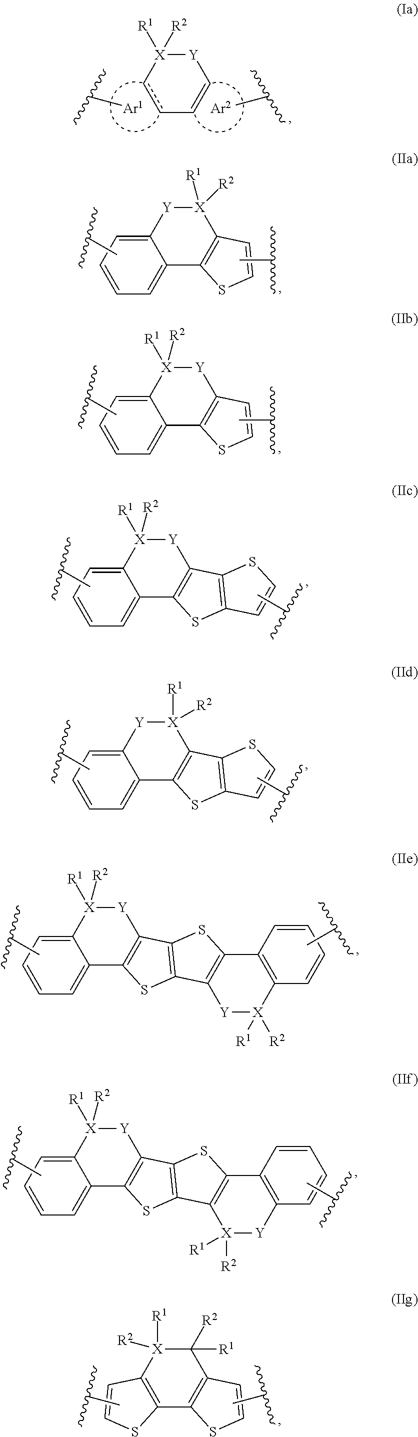

wherein,
one or both terminal ends [symbol] of the monomers of the present disclosure is independently a halogen atom, such as Br, boronic ester or boronic acid, silyl, diazonium salt, triflate, acetyloxy, sulfonate, or phosphate which can undergo Pd or Nickel salt catalyzed polymerization reactions.

In some case, the monomers or co-monomers (or monomeric units) may comprise a structure selected from the group consisting of Formula (IIaa), (IIbb), (IIcc), (IIdd), (IIee), (IIff), and (IIgg). In some case, the monomers or co-monomers (or monomeric units) may comprise a structure selected from the group consisting of Formula (IIaaa), (IIbbb), (IIccc), (IIddd), (IIeee), (IIfff), and (IIggg).

In some cases, one or both [symbol] in the monomer or co-monomer is a site for covalent attachment to the unsaturated backbone of the fluorescent polymer.

Each Ar¹, Ar², X, Y, R¹, R², R³, Z, Q, R⁴, W¹, L¹, L², L³, E, R⁵, R⁶, R⁷, h, n, s, t is described herein. Each h is independently an integer from 0 to 50, 10 to 20, or 11 to 18. Each n is independently an integer from 1 to 20, 2 to 18, 3 to 15, 4 to 12, or 5 to 10. Each s is independently 1 or 2. Each t is independently 0, 1, 2, or 3. In some examples, at least one of, or at least two of R¹, R², R³, or R⁴ comprises a water-solubilizing moiety.

In some embodiments, the monomer or co-monomer of the present disclosure is water soluble.

### Monomer Synthesis

In some embodiments, the present disclosure provides methods, processes, and synthetic routes for the monomers, fluorescent polymers, and fluorescent copolymers described herein.

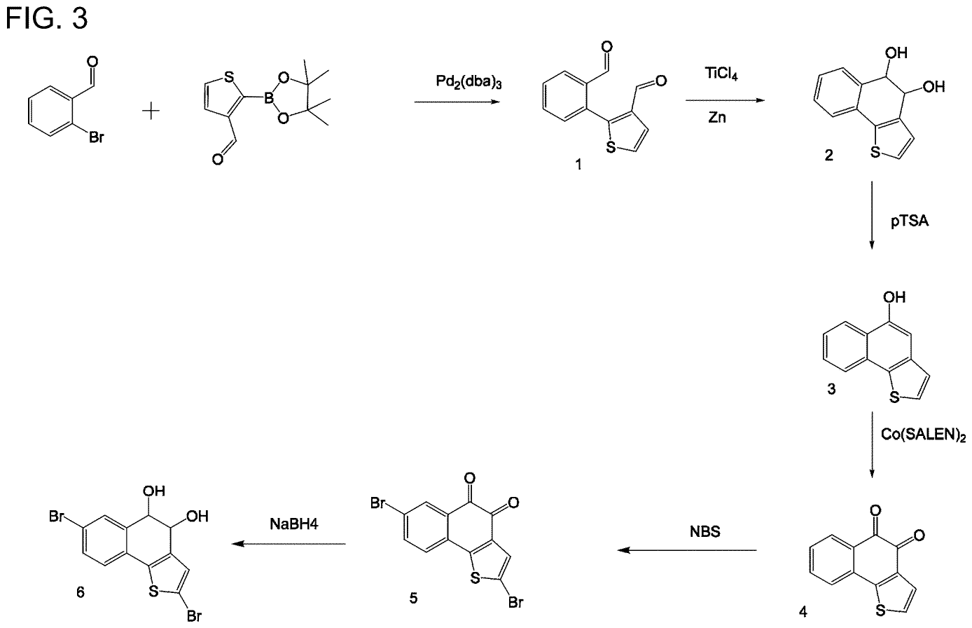

In one example, a dibromo DHNT diol monomer (compound 6) can be prepared according to the synthetic scheme shown in FIG. 3.

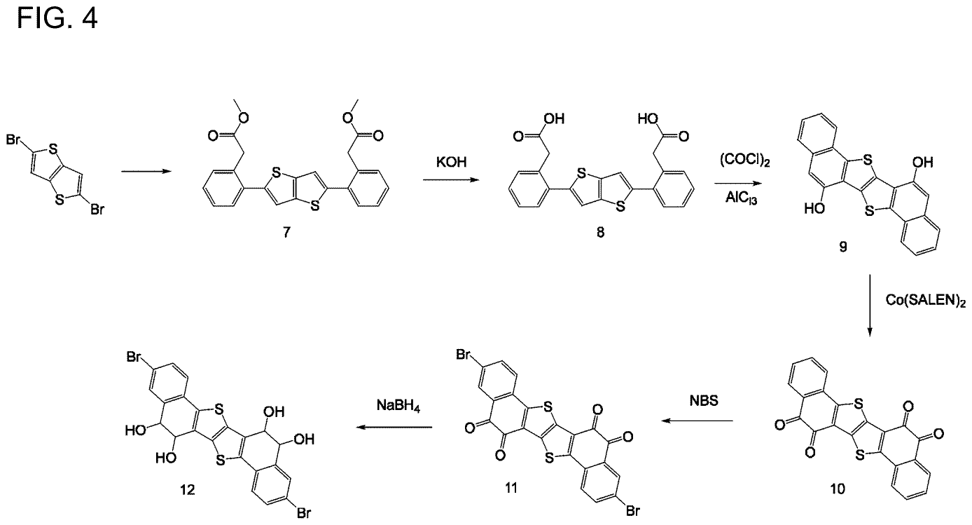

In another example, a dibromo DHNTT tetraol monomer (compound 12) can be prepared according to the synthetic scheme shown in FIG. 4.

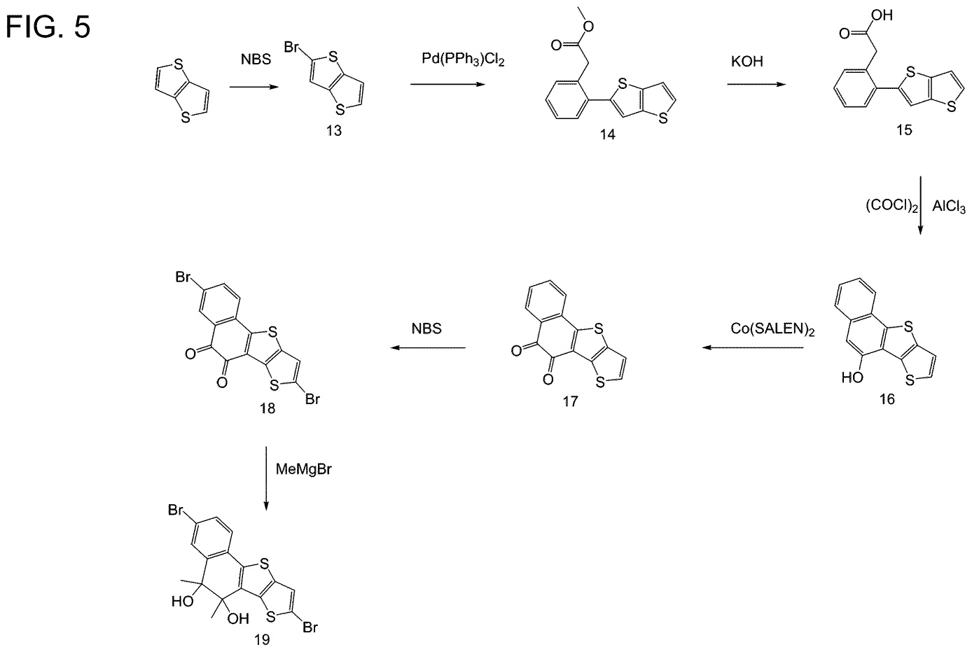

In another example, a dibromo DHNTT diol monomer (compound 19) can be prepared according to the synthetic scheme shown in FIG. 5.

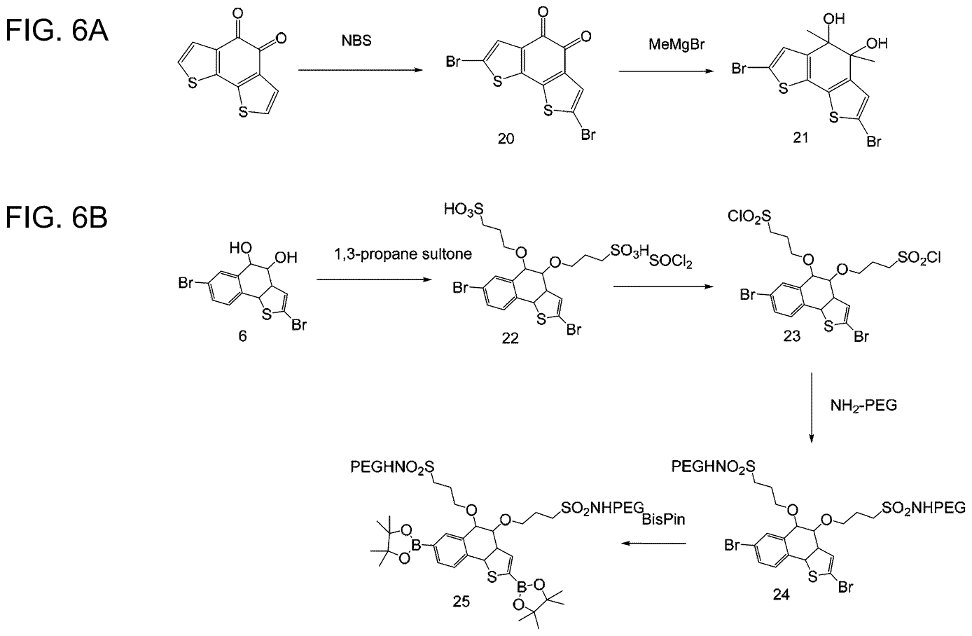

In another example, a dibromo DHBDT diol monomer (compound 21) can be prepared according to the synthetic scheme shown in FIG. 6A.

### Polymerization

The compounds described in the above embodiments may be made using procedures known in the art. In some embodiments, the fluorescent polymers can be made from dihydronaphthothiophene (DHNT), dihydronapthothienothiophene (DHNTT), or dihydrobenzodithiophene (DHBDT) monomers, optionally combined with modifying units or linker units. In some instances, the linker or modifying units may be electron rich. In some instances, the linker or modifying units may be electron poor. In some embodiments, bright fluorescent polymeric dyes can be made from co-polymerization of DHNT, DHNTT, and/or DHBDT monomers or with DHP, fluorene, and/or phenyl co-monomers. The DHP, phenyl, and/or fluorene co-monomers can be optionally substituted DHP monomers, optionally substituted fluorene monomers, and/or optionally substituted phenyl monomers. DHP and fluorene monomers and methods for making them are disclosed in WO 2017/180998. Optionally substituted phenyl monomers are known and commercially available, for example, from Sigma Aldrich.

Synthesis of diboronic ester derivatives from a dihalide monomer can be accomplished via Suzuki coupling with bis(pinacolato) diboron according to FIG. 6B, as described in the present examples. For example, the di-boronic ester DHNT monomer compound 25 can be prepared by reacting the di-bromo DENT monomer compound 24 with 3 equivalents of bispinacolatodiboron under nitrogen, in the presence KOAc and Pd(dppf)Cl₂ as catalyst.

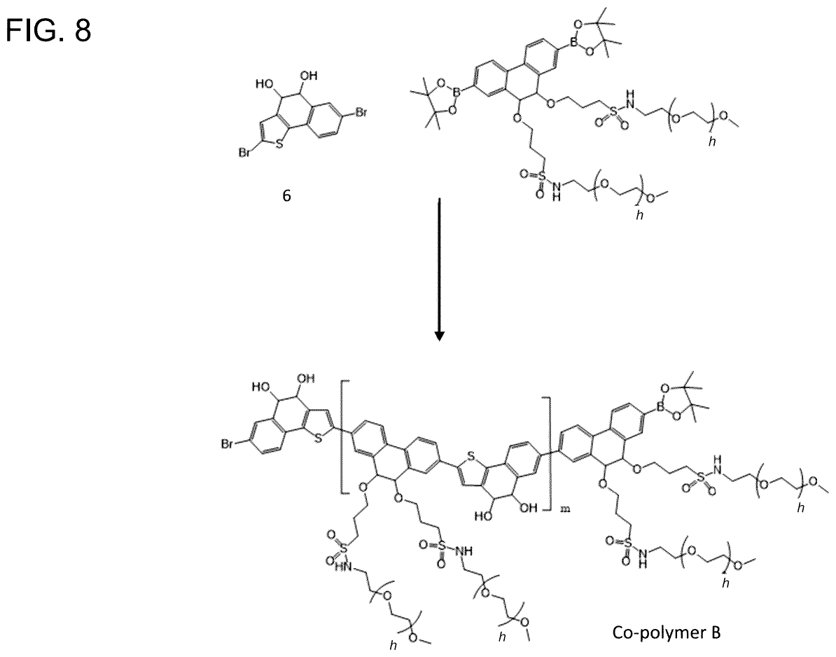

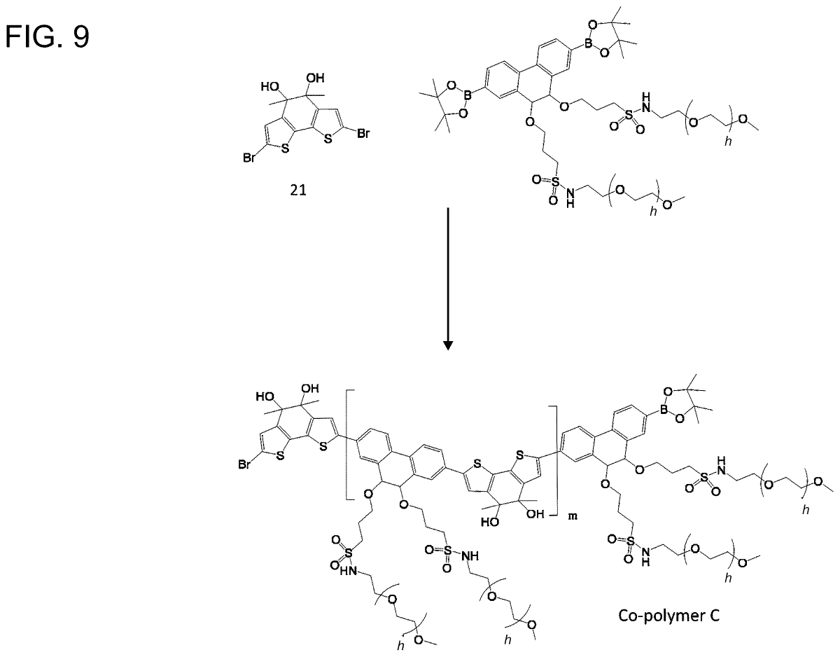

Generally, polymerization of monomer units described above can be accomplished using polymerization techniques known to those of skill in the art or using methods known in the art in combination with methods described herein. For example, polymerization can be achieved via Suzuki coupling according to FIG. 8 and FIG. 9.

A general polymerization protocol can be employed as follows. The bromo and boronic monomers are mixed in (DMF-water) under nitrogen, CsF and 10% Pd(OAc)₂ are added, and the mixture is heated to ~80° C. Polymerization may be monitored using UV-Vis spectroscopy and SEC chromatography.

A capping agent (selected from G¹) containing an appropriate functional group can be added to the reaction mixture and ~3 hours later a second capping agent (selected from G²) can be added to the reaction mixture. After the reaction is complete, the crude reaction mixture is evaporated and passed through a gel filtration column to remove small organic molecules and low MW oligomers.

### Capping Units

Linkers and capping units can be conjugated to a fluorescent polymer backbone of this disclosure via similar mechanisms as described previously. For example, bromo- and boronic esters of capping units can be used to append one or both ends of a polymer. Utilizing both bromo- and boronic esters of capping units will append both ends of polymer. Utilizing only one form, either a bromo- or boronic ester of a capping unit, will append only those ends terminated with its respective complement and for symmetric polymerizations can be used to statistically modify only one end of a polymer. For asymmetric polymers this approach is used to chemically ensure the polymers are only modified at a single chain terminus. Capping units can also be appended asymmetrically by first reacting a bromo-capping unit with a polymer with a boronic ester end and subsequently reacting the polymer with a boronic ester capping unit.

For example, capping agents of the present disclosure can be made as shown in scheme (I):

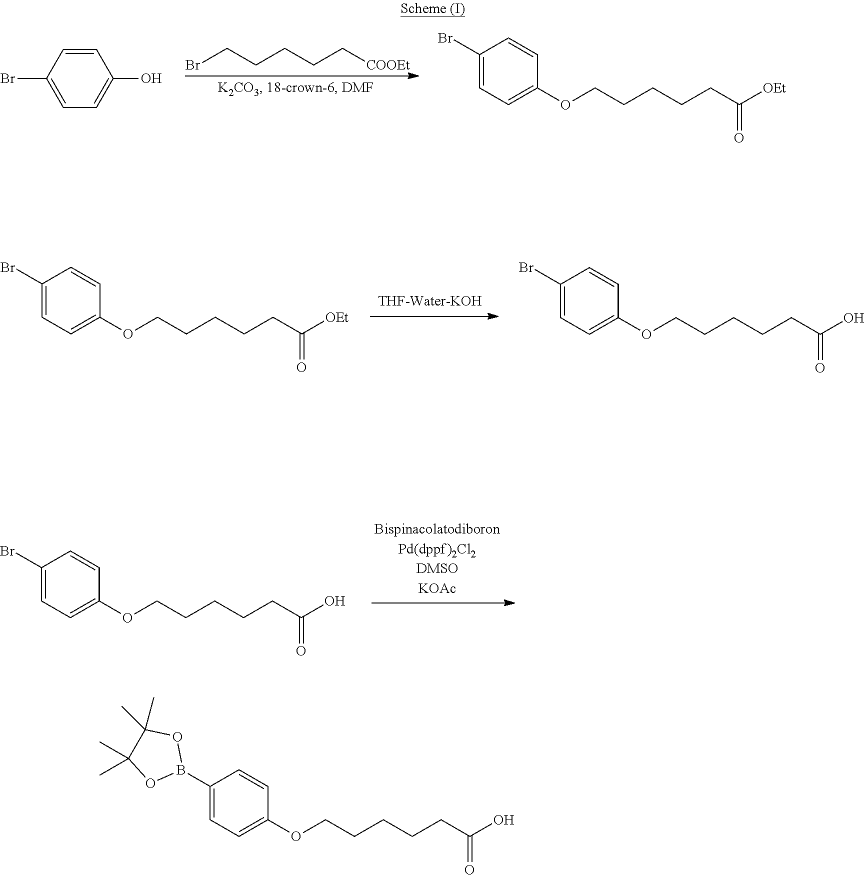

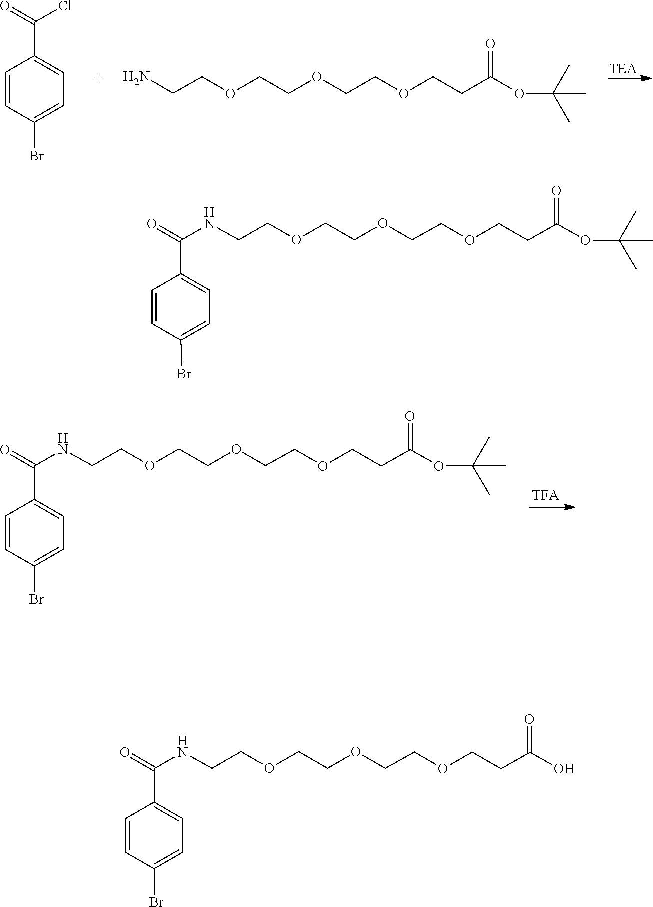 [SMILES: O=C(Cl)c1ccc(Br)cc1 ; CC(C)(C)OC(=O)CCOCCOCCOCCN ; CC(C)(C)OC(=O)CCOCCOCCOCCNC(=O)c1ccc(Br)cc1 ; CC(C)(C)OC(=O)CCOCCOCCOCCNC(=O)c1ccc(Br)cc1 ; O=C(O)CCOCCOCCOCCNC(=O)c1ccc(Br)cc1]

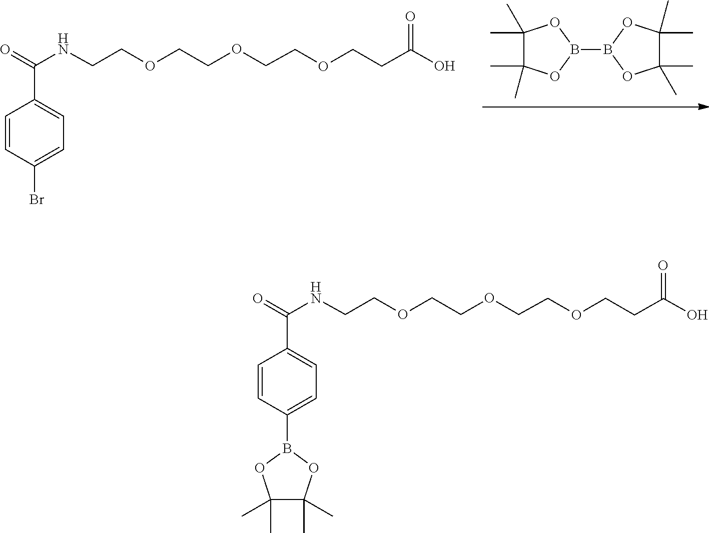

### Binding Partners

A “binding partner” or “specific binding partner” of the present disclosure can be any molecule or complex of molecules capable of specifically binding to a target analyte. A binding partner of this disclosure includes, for example, proteins, small organic molecules, carbohydrates (including polysaccharides), oligonucleotides, polynucleotides, lipids, affinity ligand, antibody, antibody fragment, an aptamer and the like. In some embodiments, the binding partner is an antibody or fragment thereof. Specific binding in the context of the present disclosure refers to a binding reaction which is determinative of the presence of a target analyte in the presence of a heterogeneous population. Thus, under designated assay conditions, the specified binding partners bind preferentially to a particular protein or isoform of the particular protein and do not bind in a significant amount to other proteins or other isoforms present in the sample.

When the binding partners are antibodies, they may be monoclonal or polyclonal antibodies. The term antibody as used herein refers to immunoglobulin molecules and immunologically active portions of immunoglobulin (Ig) molecules. Such antibodies include, but are not limited to, polyclonal, monoclonal, mono-specific polyclonal antibodies, antibody mimics, chimeric, single chain, Fab, Fab′ and F(ab′)₂ fragments, Fv, and a Fab expression library.

### Complexes

In general, water-soluble fluorescent polymers of the present disclosure can be conjugated to binding partners to form a conjugated water-soluble fluorescent polymer complex (also referred to as a “labeled specific binding partner”) using techniques known to those of skill in the art or using methods known in the art in combination with methods described herein.

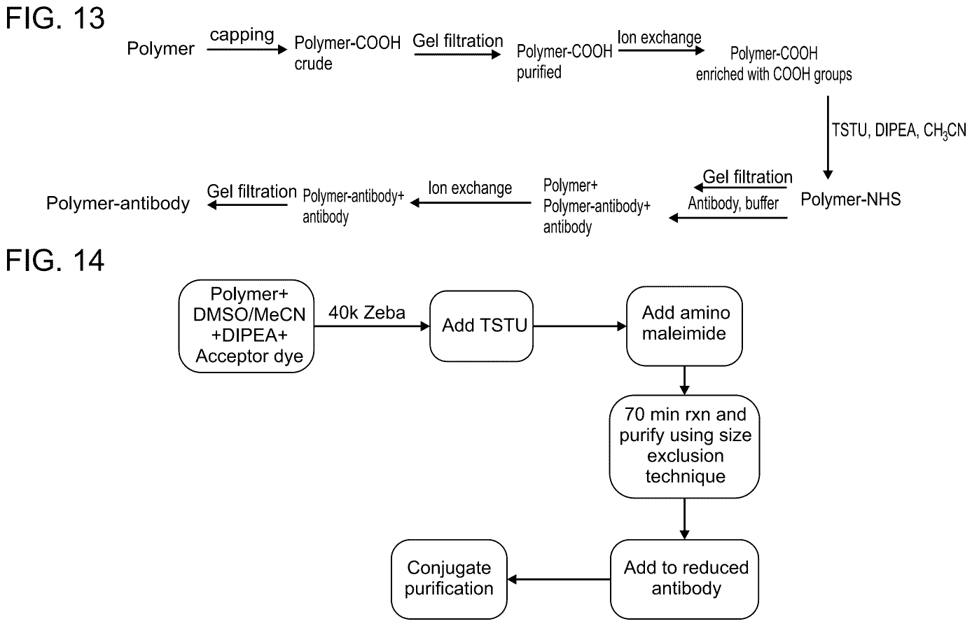

In some embodiments, water-soluble fluorescent polymers of the present disclosure can be conjugated to specific binding partners using the method of direct modification of core polymers described in US2020/0190253, which is incorporated herein by reference in its entirety. For example, a polymer-antibody complex can be prepared according to the general scheme as shown in FIG. 13.

For example, preparation of polymer NHS ester can proceed as follows. Using a clean vial, dissolve 5 mg of polymer in 1 mL dry CH₃CN. To this, add 15 mg N,N,N′,N′-tetramethyl-O—(N-succinimidyl)uranium tetrafluoroborate (TSTU) and stir for 2 more minutes. To this, add 100 uL N,N-diisopropylethylamine (DIPEA) and continue stirring overnight with the cap sealed with parafilm. Later evaporate off the organic solvents in the reaction mixture. Dissolve the crude NHS in about 750 uL of 1×BBS buffer (pH 8.8) by a quick vortex and transfer it to a Zeba column 40K MWCO. Spin down the sample at 2200 RPM for 2 min and use this polymer NHS immediately.

Conjugation of polymer NHS with CD4 can proceed as follows. Take the polymer NHS in 1×BBS (~800 uL), spin down, add to 0.6 mg of CD4 and mix with 100 μL of 0.5M Borate buffer (pH 9.0). Vortex quickly for 30 seconds and allow to mix for 3-4 hours in the coulter mix.

Purification of polymer-antibody conjugate through Histrap HP column can proceed as follows. Approach 1: After the crude reaction purify the conjugate using a Histrap HP column. Load the sample using 1×PBS buffer and collect the unbound fraction. This can be done using 20 CV of buffer. Later change the buffer to wash the bound fraction which has both conjugate and free antibody. This can be done using 1×PBS with 0.25M imidazole running for 10 CV.

Approach 2: SP Sepharose FF column. Equilibrate the column and load the sample using 20 mM Citrate buffer pH 3.5 and collect the unbound fraction. This can be done using 20 CV of buffer. Later change the buffer to elute the bound fraction which has both conjugate and free antibody. This can be done using 20 mM Tris buffer pH 8.5 running for 20 CV.

Approach 3: Load the crude conjugate in a Tangential flow filtration system equipped with a 300K MWCO membrane. The conjugate is washed using 1×PBS until the filtrate show no absorption at 405 nm or 355 nm. Later the compound is concentrated.

Purification of polymer-antibody conjugate through SEC column can proceed as follows. Load the crude conjugate containing free antibody to the Size Exclusion Column, using 1×PBS. Pool the tubes after checking the absorption spectra and concentrate in an Amicon Ultra-15 having a 30 KDa MWCO centrifugal concentrator.

Purification of polymer-antibody conjugate through a Nuvia HR-S column (Bio-Rad Laboratories, Inc.) can proceed as follows. Load crude polymer-antibody conjugate mixture to the Nuvia HR-S column using a biological buffer having a pH between about 2 to about 14 and a conductivity less than 3 mS/cm. Due to charge-charge interactions between the matrix and biomolecules, the polymer antibody conjugate will bind to the resin while free polymer dye will not interact with the resin and will flow through. Disrupt the charge-charge interactions between the matrix and polymer antibody conjugate by using a salt (e.g., NaCl, KCl, phosphate etc.) at a concentration ranging from about 0.1 to 2 M. The salt concentration can be reduced by adjusting the pH of the elution buffer. For example, the conjugate can be eluted using a biological buffer and gradient of salt concentration (e.g., NaCl, KCl) between about 100 to 1000 mM at a pH of between about 6 to about 10.

Purification of conjugate through a Nuvia cPrime column (Bio-Rad Laboratories, Inc.) can proceed as follows. Load crude polymer-antibody conjugate mixture to the Nuvia cPrime column using a biological buffer having a pH between about 2 to about 14 and a salt concentration (e.g., NaCl. KCl) ranging from 0 to about 1 M. Unreacted polymers will flow through the column while the polymer-antibody conjugate will bind to the column. Elute the polymer-antibody conjugate by increasing the pH of the elution buffer. For example, the crude polymer antibody conjugate can be loaded into the Nuvia cPrime column using a biological buffer pH 5.0, 5 mM NaCl and eluted with a biological buffer pH 7.0 and gradient of salt concentration 5 to 500 mM.

Purification of conjugate through an Anti-mouse anti-H+L antibody-agarose bead can proceed as follows. Mix crude polymer-antibody conjugate mixture with anti-mouse anti-H+L antibody-agarose bead in a biological buffer having a pH between about 6 to about 8 for about 30 minutes at room temperature. The anti-mouse anti H+L antibody-agarose bead will bind to the polymer antibody conjugate. Remove unreacted polymers by washing with the above-mentioned biological buffer using a benchtop centrifuge with a speed of 300 g for 3 minutes. Repeat the washing process at least three times. To elute the polymer-antibody conjugate, apply an IgG elution buffer with a pH ranging from about 2 to about 4 to the washed antibody-agarose bead and incubate for about 10 to 15 min. Centrifuge to collect the flow through that contains the polymer antibody conjugate.

### Tandem Polymers

In some embodiments, the fluorescent polymers or water-soluble fluorescent polymers of the disclosure, and conjugates thereof, include acceptor dyes or chromophores attached to the backbone. When a light source excites the polymer backbone, the acceptor dyes or chromophores can absorb energy of an appropriate wavelength and emit or transfer energy. Mechanisms for energy transfer between the polymers and labeled specific binding partners of the present disclosure and a linked acceptor chromophore include, for example, resonant energy transfer (e.g., Forster (or fluorescence) resonant energy transfer, FRET), quantum charge exchange (Dexter energy transfer) and the like. The terms “chromophore” and “fluorophore” are used interchangeably herein.

Any convenient fluorescent dyes may be utilized in the tandem dyes as an acceptor chromophore (acceptor dye). The chromophores and fluorophores may be selected from, for example, coumarins, fluoresceins, rhodamines, cyanines, bodipys, or other polycyclic aromatics. Many fluorophores are commercially available and may be selected from, but are not limited to, for example, any dye available from Beckman Coulter, Inc., including, but not limited to, SuperNova polymer dyes; any dye available from Becton Dickinson Biosciences, including, but not limited to, BD Horizon Brilliant™ polymer dyes; any dye available from ThermoFisher Scientific, including, but not limited to, Super Bright polymer dyes, and Alexa Fluor dyes, including, but not limited to, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680; ATTO 390, ATTO 465, ATTO 488, ATTO 495, ATTO 514, ATTO 532, ATTO 550, ATTO 565, ATTO 590, ATTO 594, ATTO 610, ATTO 620, ATTO 633, ATTO 647, ATTO 647N, ATTO 655, ATTO 665, ATTO 680, ATTO 700, ATTO 725, ATTO 740, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-ROX, 6-TAMRA, 6-Carboxyrhodamine 6G, 6-CR6G, 6-JOE, 6-FAM, 6-ROX, Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy Fl, Bodipy R6G, Bodipy TMR, Bodipy TR, CF 488A, CF 555, CF 568, CF 594ST, CF 633, CF 640R, CF 647, CF 660C, CF 680, CF680R, CF 750, CF 770, CF 790, CL-NERF, CMFDA, Cy2, Cy3, Cy3.5, Cy5, Cy5.5, Cy7, DDAO, DiA, DiD, DiI, DyLight 488, DyLight 550, DyLight 594, DyLight 633, DyLight 650, DyLight 680, DyLight 755, DyLight 800, DiO, DiR, DM-NERF, DsRed, DTAF, DY-490, DY-495, DY-505, DY-530, DY-547, DY-548, DY-549, DY-549P1, DY-550, DY-554, DY-555, DY-556, DY-560, DY-590, DY-591, DY-594, DY-605, DY-610, DY-615, DY-630, DY-631, DY-632, DY-633, DY-634, DY-635, DY-636, DY-647, DY-648, DY-649, DY-649P1, DY-650, DY-651, DY-652, DY-654, DY-675, DY-676, DY-677, DY-678, DY-679, DY-679P1, DY-680, DY-681, DY-682, DY-700, DY-701, DY-703, DY-704, DY-730, DY-731, DY-732, DY-734, DY-749, DY-750, DY-751, DY-752, DY-754, DY-776, DY-777, DY-778, DY-780, DY-781, DY-782, DY-800, DY-831, Eosin, Erythrosin, FITC, Fluo-3, Fluo-4, Fluor-Ruby, FluorX, FM 1-43, FM 1-46, iFluor 488, iFluor 555, iFluor 594, iFluor 647, iFluor 680, iFluor 700, iFluor 750, iFluor 780, Lyso Tracker Green, Lyso Tracker Yellow, Mitotracker Green, Mitotracker Orange, Mitotracker Red, NBD, Oregon Green 488, Oregon Green 514, PKH26, PKH67, Resorufin, RH 414, Rhod-2, Rhodamine, Rhodamine 110, Rhodamine 123, Rhodamine 6G, Rhodamine B, Rhodamine Green, Rhodamine Red, Rose Bengal, Spectrum Green, Spectrum Orange, Spectrum Red, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, Texas Red, Tide Fluor 2 (TF2), Tide Fluor 2WS (TF2WS), Tide Fluor 3 (TF3), Tide Fluor 3WS(TF3WS), Tide Fluor 4 (TF4), Tide Fluor 5WS (TF5WS), Tide Fluor 6WS (TF6WS), Tide Fluor 7WS (TF7WS), Tide Fluor 8WS (TF8WS), TRITC, and XTRITC. Acceptor dyes useful in the disclosure may include, for example, a cyanine dye, a xanthene dye, a coumarin dye, a thiazine dye, an acridine dye, FITC, CY3B, Cy55, Alexa 488, Texas red, Cy5, Cy7, Alexa 750, Cy55, Cy3B, Cy3.5, Alexa 750, 800 CW, Biotium CF 555, diethyl coumarin, DY705 (Dyomics), DY431, DY485XL, DY500XL, DY610, DY640, DY654, DY 682, DY 700, DY 701, DY 704, DY 730, DY 731, DY732, DY 734, DY 752, DY 778, DY 782, DY 800, DY 831 and 800CW.

The acceptor dye may be a pendant acceptor dye.

The tandem dye may be a co-polymer comprising DHNT, DHNTT, and/or DHBDT monomer units with DHP and/or fluorene co-monomer units according to the present disclosure comprising one or more, two or more, three or more, 1-30, 2-20, or 2.5-10 acceptor dye moieties. In some embodiments, the acceptor dye moieties may be attached to the polymer or co-polymer via a linker moiety.

For example, an acceptor dye attached to the water-soluble fluorescent polymer or water-soluble fluorescent polymer conjugate backbone can be as shown in Scheme (II):

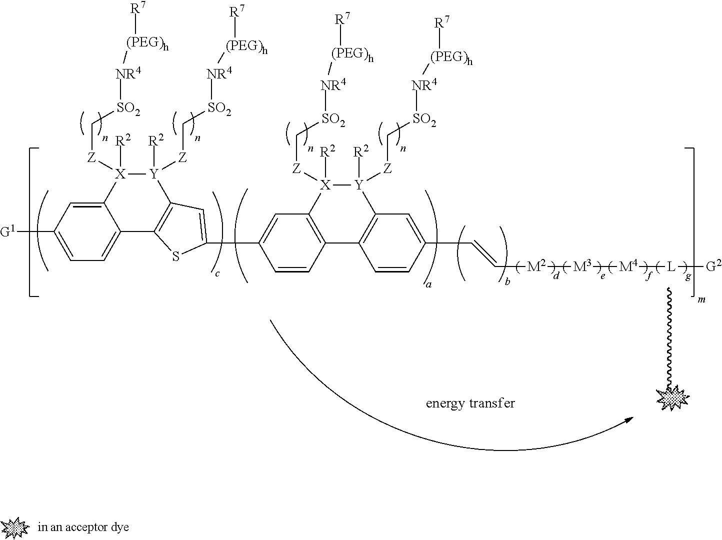

Water-soluble fluorescent tandem polymers or water-soluble fluorescent tandem polymer complexes can be prepared using techniques known to those of skill in the art or using methods known in the art in combination with methods described herein, for example, as outlined in FIG. 14.

For example, a polymer-acceptor dye may be prepared as follows. The polymer according to the disclosure may be dissolved with sonication in anhydrous DMSO optionally with warming in a water bath, e.g., >30 deg C. to about 50 deg C. Diisopropylethylamine (DIPEA) in acetonitrile can be added. A solution of about 1 to 12 equivalents, or about 2 to about 10 equivalents, or about 4 to about 8 equivalents of an NHS-derivatized acceptor dye (e.g., near-IR absorbing Dy752NHS; Dyomics GmbH) dissolved in anhydrous DMSO may be added to the reaction mixture which then may be allowed to stir for about 1 to 3 hrs at ambient room temperature protected from light to obtain a polymer-acceptor dye.

For conjugation to a specific binding partner such as an antibody, a polymer-acceptor dye maleimide may be prepared as follows. After stirring from about 1 to about 3 hours, or about 2 hours, TSTU dissolved in acetonitrile may be added to the polymer-acceptor dye mixture and the activation process may be carried out for 30 min at RT by constant stirring, protect from light. A 40 K Zeba spin column may be equilibrated with 20 mM borate pH 8.8 buffer, proceeded as described by the manufacturer. At the same time, a solution of N-(2-aminoethyl)maleimide trifluoroacetate salt may be prepared using 20 μL anhydrous DMSO and kept in the Zeba collection tube.

The activated tandem polymer may be dissolved in 20 mM borate pH 8.8 buffer and combined with the maleimide using the equilibrated Zeba spin column. The polymer amount after the Zeba column step may be estimated by measuring the UV 414 of tandem polymer. The resulting mixture of tandem polymer-maleimide may be incubated at RT for ~1-2 hours. During the incubation period, a 30% ethanol water and a 50 mM MOPS, 100 mM sodium perchlorate, 4 mM EDTA pH 7.0 (MOPS buffer) may be prepared. After the reaction, the tandem polymer-maleimide may be washed using a 30 or 50 kDa MWCO Amicon concentrator with at least 30-40 mL 30% ethanol water, followed by buffer exchange to MOPS buffer using at least 30-40 mL of MOPS buffer. This mixture containing maleimide-functionalized tandem dye polymer may be stored at 4° C. overnight before further usage.

The polymer-antibody conjugate may be prepared as follows. A 0.5 mL 40 K Zeba column may be equilibrated using 1×PBS and 1 mg binding partner (e.g., CD4 mAb) may be passed through the equilibrated Zeba column. To the buffer exchanged mAb in 1×PBS excess DTT may be added and the resulting mixture incubated at RT for ~30 min. A 50 mM MES, 0.1 M sodium perchlorate, 4 mM EDTA pH 5.8 (MES buffer) may be prepared and kept aside in the dark. After 30 min, the reduced mAb may be diluted and passed through a 40 K Zeba column pre-equilibrated with the MES buffer to remove excess DTT. The reduced binding partner (e.g., reduced CD4 mAb in MES) may be mixed with the polymer-maleimide (brought to RT before mixing) and incubated for about 3 hours by rolling at RT, protected from light, to form tandem polymer-antibody conjugate.

A polymer tandem dye is provided comprising the polymer dye according to the present disclosure; and a signaling chromophore covalently linked to the polymer dye in energy-receiving proximity therewith.

In some embodiments, instead of being attached to a ligand in the polymer backbone, acceptor dyes, chromophores, fluorophores, functional moieties and binding partners can be attached to polymers of the present disclosure through a linker moiety using the method of direct modification of core polymers described in US2020/0190253, which is incorporated herein by reference in its entirety.

### Methods of Detecting an Analyte in a Sample

A method is provided for detecting an analyte in a sample, the method comprising providing a sample that is suspected of containing a target analyte; and contacting the sample with a binding partner conjugated to a fluorescent polymer or tandem polymer of the present disclosure, wherein the binding partner is capable of specifically binding to the analyte.

A method is provided for detecting a target analyte in a sample comprising providing a sample that is suspected of containing the target analyte; and contacting the sample with a labeled specific binding partner according to the present disclosure to form a fluorescent polymer dye conjugate complex with the target analyte if the target analyte is present; applying a light source to the sample that can excite the at least one fluorescent polymer dye or tandem polymer dye conjugate complex; and detecting light emitted from the fluorescent polymer dye conjugate complex.

A light source is applied to the sample that can excite the water-soluble fluorescent polymer or tandem polymer; and light emitted from the conjugated water-soluble fluorescent polymer complex is detected. The emitted light may have a wavelength greater than about 380 nm, or within a range of about 380 nm and about 1000 nm, or about 380 nm and about 800 nm. In the typical assay, water-soluble fluorescent polymers of the present disclosure are excitable with a light having wavelength between about 350 nm and about 500 nm and the emitted light is typically between about 430 nm and about 600 nm. Alternatively, excitation light can have a wavelength between about 360 nm and about 480 nm and the emitted light can have a wavelength between about 450 nm and about 580 nm. While the fluorescent polymers of the present disclosure can have a near UV, violet, or blue excitation spectrum, one of skill in the art will understand that the spectrum can be tuned to another laser if the polymers are copolymerized with appropriate modifying units.

In the method of the present disclosure, the fluorescent polymer can be any water-soluble fluorescent polymer of the present disclosure as disclosed herein. The fluorescent polymer can have a structure selected from the group consisting of Formula (Ib), Formula (IIIa), Formula (IIIb), Formula (IV), Formula (V), Formula (VI), Formula (VIIa), Formula (VIIb), Formula (VIIc), Formula (VIId), Formula (VIIe), Formula (VIIIa), Formula (VIIIb), Formula (IXa), Formula (IXb), Formula (IXc), Formula (Xa), Formula (Xb), Formula (XI), Formula (XII), Formula (XIIIa), Formula (XIIIb), Formula (XIIIc), Formula (XIIId), Formula (XIIIe), Formula (XIVa), Formula (XIVb), Formula (XIVc), Formula (XIVd), Formula (XIVe), Formula (XIVf), Formula (XIVg), Formula (XVa), Formula (XVb), Formula (XVc), Formula (XVd), Formula (XVe), Formula (XVf), Formula (XVg), Formula (XVIa), Formula (XVIb), Formula (XVIc), Formula (XVId), Formula (XVIIa), Formula (XVIIb), Formula (XVIIc), and Formula (XVIId). The water-soluble fluorescent polymer can comprise at least one co-monomer M¹ having a structure according to Formula (Ia), (IIa), (IIb), (IIc), (IId), (IIe), (IIf), (IIg), (IIaa), (IIbb), (IIcc), (IIdd), (IIee), (IIff), (IIgg), (IIaaa), (IIbbb), (IIccc), (IIddd), (IIeee), (IIfff), and (IIggg).

In the method of the present disclosure, the fluorescent polymer can also be fluorescent copolymer comprising: (i) at least one fluorescent polymer having a structure selected from the group consisting of Formula (Ib), Formula (IIIa), Formula (IIIb), Formula (IV), Formula (V), Formula (VI), Formula (VIIa), Formula (VIIb), Formula (VIIc), Formula (VIId), Formula (VIIe), Formula (VIIIa), Formula (VIIIb), Formula (IXa), Formula (IXb), Formula (IXc), Formula (Xa), Formula (Xb), Formula (XI), Formula (XII), Formula (XIIIa), Formula (XIIIb), Formula (XIIIc), Formula (XIIId), Formula (XIIIe), Formula (XIVa), Formula (XIVb), Formula (XIVc), Formula (XIVd), Formula (XIVe), Formula (XIVf), Formula (XIVg), Formula (XVa), Formula (XVb), Formula (XVc), Formula (XVd), Formula (XVe), Formula (XVf), Formula (XVg), Formula (XVIa), Formula (XVIb), Formula (XVIc), Formula (XVId), Formula (XVIIa), Formula (XVIIb), Formula (XVIIc), and Formula (XVIId), and (ii) a π-conjugated co-monomer in the repeating unit. The π-substituted comonomer can be an optionally substituted fluorene monomer. The π-substituted comonomer can be an optionally substituted dihydrophenanthrene (DHP) monomer. The π-substituted comonomer can be an optionally substituted tetrahydropyrene (THP) monomer. The π-substituted comonomer can be an optionally substituted benzene monomer. The optionally substituted fluorene structures can be those disclosed in WO 2017/180998A2. The π-substituted comonomer can be an optionally substituted fused DHP monomers as disclosed in U.S. application Ser. No. 18/207,983.

### Samples

The sample in the methods of the present disclosure can be, for example, blood, bone marrow, spleen cells, lymph cells, bone marrow aspirates (or any cells obtained from bone marrow), urine (lavage), serum, saliva, cerebral spinal fluid, urine, amniotic fluid, interstitial fluid, feces, mucus, or tissue (e.g., tumor samples, disaggregated tissue, disaggregated solid tumor). In certain embodiments, the sample is a blood sample. In some embodiments, the blood sample is whole blood. The whole blood can be obtained from the subject using standard clinical procedures. In some embodiments, the sample is a subset of one or more cells of whole blood (e.g., erythrocyte, leukocyte, lymphocyte (e.g., T cells, B cells or NK cells), phagocyte, monocyte, macrophage, granulocyte, basophil, neutrophil, eosinophil, platelet, or any cell with one or more detectable markers). In some embodiments, the sample can be from a cell culture.

The subject can be a human (e.g., a patient suffering from a disease), a commercially significant mammal, including, for example, a monkey, cow, or horse. Samples can also be obtained from household pets, including, for example, a dog or cat. In some embodiments, the subject is a laboratory animal used as an animal model of disease or for drug screening, for example, a mouse, a rat, a rabbit, or guinea pig.

### Analytes

An “analyte” also known as a “target analyte” as used herein, refers to a substance, e.g., molecule, whose abundance/concentration is determined by some analytical procedure. For example, in the present disclosure, an analyte can be a protein, peptide, nucleic acid, lipid, carbohydrate small molecule, or a target-associated biomolecule.

The target analyte may be, for example, nucleic acids (DNA, RNA, mRNA, tRNA, or rRNA), peptides, polypeptides, proteins, lipids, ions, monosaccharides, oligosaccharides, polysaccharides, lipoproteins, glycoproteins, glycolipids, or fragments thereof. In some embodiments, the target analyte is a protein and can be, for example, a structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, ion channels and the like. The protein can be an activatable protein or a protein differentially expressed or activated in diseased or aberrant cells. including but not limited to transcription factors, DNA and/or RNA-binding and modifying proteins, nuclear import and export receptors, regulators of apoptosis or survival and the like.

### Assays

The polymer dyes, tandem dyes, and labeled specific binding partners according to the disclosure may find use in a variety of applications, including diagnostic and research applications, in which the labelling, detection and/or analysis of a target of interest is desirable. Such applications include methodologies such as, for example, cytometry, microscopy, immunoassays (e.g., competitive or non-competitive), fluorescence in situ hybridization (FISH), cell tracing, receptor labeling, fluorescence spectroscopy, assessment of a free analyte, assessment of receptor bound ligand, and so forth. The polymer dyes, tandem polymer dyes, and labeled specific binding partners described herein may be useful in analysis of any of a number of samples, including but not limited to, biological fluids, cell culture samples, and tissue samples. In certain aspects, the compositions, system and methods described herein may find use in methods where analytes are detected in a sample, if present, using fluorescent labels, such as in fluorescent activated cell sorting or analysis, immunoassays, immunostaining, and the like. In certain instances, the polymer dyes, tandem polymer dyes, and labeled specific binding partners find use in applications where the evaluation of a sample for the presence of a target analyte is of interest. In some cases, the polymer dyes, tandem polymer dyes, and labeled specific binding partners find use in any assay format where the detection and/or analysis of a target from a sample is of interest, including but not limited to, flow cytometry, fluorescence microscopy, in-situ hybridization, enzyme-linked immunosorbent assays (ELISAs), western blot analysis, magnetic cell separation assays and fluorochrome purification chromatography. In certain instances, the polymer dyes, tandem polymer dyes, and labeled specific binding partners find use in any application where the fluorescent labelling of a target molecule is of interest. The subject polymer dyes, tandem polymer dyes, and labeled specific binding partners may be adapted for use in any convenient applications where pairs of specific binding members find use, such as biotin-streptavidin and hapten-anti-hapten antibody.

Assay systems utilizing a binding partner and a fluorescent label to quantify bound molecules are well known. Examples of such systems include flow cytometers, scanning cytometers, imaging cytometers, fluorescence microscopes, and confocal fluorescent microscopes.

In some embodiments, flow cytometry is used to detect fluorescence. A number of devices suitable for this use are available and known to those skilled in the art. Examples include BCI Navios, Gallios, Aquios, and CytoFLEX flow cytometers.

In other embodiments, an assay is used. The assay can be an immunoassay. Examples of immunoassays useful in the disclosure include, but are not limited to, fluoroluminescence assay (FLA), and the like. The assays can also be carried out on protein arrays.

When the binding partners are antibodies, antibody or multiple antibody sandwich assays can also be used. A sandwich assay refers to the use of successive recognition events to build up layers of various binding partners and reporting elements to signal the presence of a particular analyte. Examples of sandwich assays are disclosed in U.S. Pat. No. 4,486,530 and in the references noted therein.

### Kits

A kit is provided comprising at least one polymer dye, polymer tandem dye, or labeled specific binding partner according to the present disclosure, wherein the at least one polymer dye, polymer tandem dye, or labeled specific binding partner optionally comprises a conjugation tag. A kit is provided comprising at least one polymer dye according to the disclosure. A kit is provided comprising at least one polymer tandem dye according to the disclosure. A kit is provided comprising at least one labeled specific binding partner according to the present disclosure.

The disclosure provides kits for use in practicing the subject methods. A kit can include a polymer, labeled specific binding partner, or tandem dye as described herein and a container. Any convenient containers can be utilized, such as tubes, bottles, or wells in a multi-well strip or plate, a box, a bag, an insulated container, and the like. In some instances, the subject kits can include one or more components selected from a polymer, labeled specific binding partner, or tandem dye according to the present disclosure, a fluorophore, a chromophore, a specific binding member, a specific binding member conjugate, a support bound specific binding member, a cell, a support, a biocompatible aqueous elution buffer, and/or instructions for use. In some embodiments of the kit, the DHP bridged compound, or tandem dye according to the present disclosure is covalently linked to a specific binding partner.

In some instances, the subject kits can be “labeling kits” that include a polymer or tandem dye according to the present disclosure comprising a sidechain chemoselective functional group (also referred to as a “conjugation tag”) such as, for example, a NHS ester of a polymer or tandem dye according to the disclosure and the like, to which any convenient target moiety of interest (e.g., an acceptor dye, fluorophore, chromophore, a specific binding partner, a support) can be conjugated. The chemoselective functional group may include a reactive group (e.g., biotin) that targets specific functional groups on biomolecules (e.g., streptavidin, proteins, or antibodies), such as, for example, primary amines, sulfhydryls, carboxyls, or carbohydrates. The chemoselective functional group (“conjugation tag”) can be one used in “click chemistry” reactions.

In certain instances, the conjugation tag includes a maleimide functional group and the target moiety includes a thiol functional group, or vice versa. In some instances, the conjugation tag includes an alkyne functional group (e.g., a cyclooctyne group) and the target moiety includes an azide functional group, or vice versa, which can be conjugated via Click chemistry. In certain instances, the conjugation tag includes an alkene functional group (e.g., a cyclooctene group) and the target moiety includes a tetrazine functional group, or vice versa, which can be conjugated via inverse-demand Diels-Alder cycloaddition reaction. In some instances, the conjugation tag includes an amine-reactive chemical group, such as, for example, a NHS ester (N-hydroxysuccinimde esters) or imidoester functional group and the target moiety includes a NH₂ functional group, or vice versa. In some instances, the conjugation tag includes a biotin-binding protein (e.g., Avidin, Streptavidin, or NeutrAvidin) and the target moiety includes a biotin molecule, or vice versa, which can non-covalently interact.

### EXAMPLES

### Example 1. Synthesis of DHNT Monomer Molecule 6: 2,7-dibromo-4,5-dihydronaphtho[1,2-b]thiophene-4,5-diol

Synthesis of DHNT molecule 6 (2,7-dibromo-4,5-dihydronaphtho[1,2-b]thiophene-4,5-diol) is illustrated in FIG. 3, and is briefly described as follows.

Intermediate compound 1: 2-(2-formylphenyl)thiophene-3-carbaldehyde was prepared as follows. In a round bottom flask methyl 2-(2-(4,4,5,5-tetramethyl-1,3,2-dioxaborolan-2-yl)thiophen-3-carbaldehyde (1.46 mmoles, 0.35 g) and 2-bromo-benzaldehyde (1.61 mmmoles, 0.29 g) were dissolved in THF (14 mL) and CsF (5.84 mmoles, 0.89 g) was added. The mixture was purged with bubbling Nitrogen for 5 minutes and then Pd₂(dba)₃ (0.112 mmoles, 0.11 g) and P(tBu)₃ (0.31 mmol) added. The reaction was heated to 45° C. for 20 hours. After that the reaction was cooled down to room temperature, diluted with ethyl acetate and filtered over a silica pad. The collected mixture was concentrated and purified using automated column chromatography using silica gel as stationary phase and hexanes/ethyl acetate as mobile phase to afford the desired compound 1 (0.3 g, 93%).

Intermediate compound 2: 4,5-dihydronaphtho[1,2-b]thiophene-4,5-diol was prepared as follows. Intermediate compound 1 (4.62 mmoles, 1 g) was dissolved in THF (80 mL) and the mixture was cooled to −70° C. with a dry ice/acetone bath under Nitrogen atmosphere. Then 6.94 mL of 1M TiCl₄ were slowly added (6.94 mmoles). The reaction was stirred for 30 minutes at −70° C. and then Zn powder (13.9 mmoles, 0.91) was added. The reaction mixture was allowed to warm up to 0° C. and left to stir for an additional 3 hours. After that, the mixture was diluted with Ethyl Acetate (150 mL) and washed with diluted brine. The organic layer was collected, dried over Mg₂SO₄ and concentrated. The residue was purified by automated column chromatography using silica gel as stationary phase and hexanes/ethyl acetate as mobile phase to afford compound 2 (0.46 g, 46%).

Intermediate compound 3: naphtho[1,2-b]thiophen-5-ol was prepared as follows. Intermediate compound 2 (2.11 mmoles, 0.46 g) was dissolved in acetone (50 mL) and p-toluensulfonic acid added (0.74 mmoles, 0.14 g). The mixture was stirred at 70° C. for 16 hours. After that the solvent was evaporated, the residue dissolved in Ethyl Acetate and washed with diluted brine. The organic layer was collected, dried over Mg₂SO₄ and concentrated. The residue was purified by automated column chromatography using silica gel as stationary phase and hexanes/ethyl acetate as mobile phase to afford compound 3 (0.42 g, 99%).

Intermediate compound 4: naphtho[1,2-b]thiophene-4,5-dione was prepared as follows. Intermediate compound 3 (2.4 mmoles, 0.48 g) was dissolved in 3 mL of DMF and Co(SALEN)₂ (0.24 mmoles, 78 mg) added. The reaction mixture was left to react uncapped for 16 hours and then poured into H₂O (100 mL). The mixture was filtered and washed with more H₂O to afford compound 4 as a red-orange solid (0.45 g, 88%).

Intermediate compound 5: 2,7-dibromonaphtho[1,2-b]thiophene-4,5-dione was prepared as follows. Intermediate compound 4 (0.91 mmoles, 0.195 g) was dissolved in sulfuric acid and the mixture cooled down to 0° C. NBS (2.28 mmoles, 0.4 g) was added portion wise and the mixture left to react for 16 hours in the ice bath. The mixture was then poured into cold water and filtered. The solid was purified by automated column chromatography using silica gel as stationary phase and hexanes/ethyl acetate as mobile phase to afford a fraction containing the product 5 as bright red solid (0.14 g, 41%).

Compound 6: 2,7-dibromo-4,5-dihydronaphtho[1,2-b]thiophene-4,5-diol was prepared as follows. Intermediate compound 5 (0.134 mmoles, 50 mg) was dissolved in 13 mL of Ethanol then 2 mL of H₂O were added. Then, NaBH₄ (1.324 mmoles, 51 mg) was added. After 24 hours the mixture was quenched by adding 4 mL of saturated NH₄Cl and 0.5 ML HCL 1M. The mixture was then extracted with Ethyl Acetate, organic layer was collected, dried over Mg₂SO₄ and concentrated. The residue was purified by automated column chromatography using silica gel as stationary phase and hexanes/ethyl acetate as mobile phase to afford compound 6 (10 mg, 20%).

### Example 2. Synthesis of DHNTT Monomer Molecule 12: (3,10-dibromo-5,6,12,13-tetrahydronaphtho[1,2-b]naphtho[2′,1′:4,5]thieno[2,3-d]thiophene-5,6,12,13-tetraol)

Synthesis of DHNTT molecule 12 (3,10-dibromo-5,6,12,13-tetrahydronaphtho[1,2-b]naphtho[2′,1′:4,5]thieno[2,3-d]thiophene-5,6,12,13-tetraol) is illustrated in FIG. 4, and is briefly described as follows.

Compound 7: dimethyl 2,2′-(thieno[3,2-b]thiophene-2,5-diylbis(2,1-phenylene))diacetate was prepared as follows. In a round bottom flask methyl methyl 2-(2-(4,4,5,5-tetramethyl-1,3,2-dioxaborolan-2-yl)phenyl)acetate (3.77 mmoles, 1.04 g) and 2,5-dibromothieno[3,2-b]thiophene (1.71 mmoles, 0.51 g) were dissolved in DME (40 mL) and KF (25.7 mmoles, 1.49 g) dissolved in 13 mL H₂O was added. The mixture was purged with bubbling Nitrogen for 10 minutes and then Pd(PPh₃)₂Cl₂ (0.257 mmoles, 0.18 g) added. The reaction was heated to 80° C. for 22 hours. After that the reaction was cooled down to room temperature, 50 mL of aqueous NH₄Cl added and extracted with Ethyl Acetate. The residue was precipitated from EtOAc/Hexanes 1/1 to afford product 7 (0.55 g, 74%).

Compound 8: 2,2′-(thieno[3,2-b]thiophene-2,5-diylbis(2,1-phenylene))diacetic acid was prepared as follows. Compound 7 (1.26 mmoles, 0.55 g) was dissolved in THF (20 mL) and 11 mL of 12M KOH were added. The reaction mixture was heated to 75° C. for 16 hours and then neutralized with HCL conc (13 mL). The mixture was extracted with Ethyl Acetate, the organic layer dried over Mg₂SO₄ and concentrated to afford compound 8 as a yellow solid (0.373 g, 81%).

Compound 9: naphtho[1,2-b]naphtho[2′,1′:4,5]thieno[2,3-d]thiophene-6,13-diol was prepared as follows. Compound 8 (0.277 mmoles, 0.11 g) was dissolved in THF (6 mL) and DMF (100 μL) added. The reaction mixture was cooled with an ice bath and then (COCl)₂ added (1.38 mmoles, 0.69 ml of 2M solution in dichloromethane). The reaction was stirred for 2 hours and then the solvents evaporated. The residue was dissolved in 1,2-dichloroethane (10 mL), AlCl₃ (0.78 mmoles, 0.104 g) added and the mixture heated to 67° C. After 16 hours the reaction mixture was diluted with dichloromethane and washed with water. The organic layer was separated, and the solvents evaporated. The impure product was purified by automated column chromatography using silica gel as stationary phase and hexanes/ethyl acetate as mobile phase to afford compound 9 (33 mg, 32%).

Compound 10: naphtho[1,2-b]naphtho[2′,1′:4,5]thieno[2,3-d]thiophene-5,6,12,13-tetraone was prepared as follows. Compound 9 (0.078 mmoles, 29 mg) was dissolved in 0.6 mL of DMF and Co(SALEN)₂ (0.008 mmoles, 3 mg) added. The reaction mixture was left to react uncapped for 72 hours and then poured into H₂O (10 mL), the mixture was filtered and washed with more H₂O to afford compound 10 as a red solid (10 mg, 32%).

Compound 11: 3,10-dibromonaphtho[1,2-b]naphtho[2′,1′:4,5]thieno[2,3-d]thiophene-5,6,12,13-tetraone can be prepared by reacting Compound 10 with 2,2 equivalents of NBS in H₂SO₄. When reaction is completed water can be added and the mixture filtered to afford compound 11.

Compound 12: 3,10-dibromo-5,6,12,13-tetrahydronaphtho[1,2-b]naphtho[2′,1′:4,5]thieno[2,3-d]thiophene-5,6,12,13-tetraol can be made by dissolving compound 11 in dry THF and reacting with 4 equivalents NaBH₄. When the reaction is completed, the reaction mixture can be quenched by adding saturated NH₄Cl and extracting with Ethyl Acetate. The organic layer can be purified by automated column chromatography using silica gel as stationary phase to afford purified compound 12.

### Example 3. Synthesis of DHNTT Monomer Molecule 19: (3,8-dibromo-5,6-dimethyl-5,6-dihydronaphtho[1,2-b]thieno[2,3-d]thiophene-5,6-diol)

Synthesis of DHNTT molecule 19 is illustrated in FIG. 5, and is briefly described as follows.

Compound 13: 2-bromothieno[3,2-b]thiophene was prepared as follows. In a round bottom flask, thieno[3,2-b]thiophene (7.13 mmoles, 1 g) was dissolved in dichloromethane (80-mL) and NBS (7.5 mmoles, 1.33 g) was added in three portions. The reaction mixture was left to stir at room temperature and in the dark for 16 hours. After that, the mixture was concentrated to about 2 mL and 50 mL of hexanes added. The resulting mixture was concentrated to about 10 mL using a rotavapor and then cooled to about 4° C. to induce precipitation. The precipitate was discarded and the supernatant collected and concentrated to afford a mix of product in which approximately 75%, by HPLC analysis, was compound 13. This product was used as is the next step.

Compound 14: methyl 2-(2-(thieno[3,2-b]thiophen-2-yl)phenyl)acetate was prepared as follows. In a round bottom flask methyl methyl 2-(2-(4,4,5,5-tetramethyl-1,3,2-dioxaborolan-2-yl)phenyl)acetate (0.427 mmoles, 118 mg) and compound 13 (0.47 mmmoles, 0.11 g) were dissolved in DME (5 mL) and KF (3.42 mmoles, 0.2 g) dissolved in 2 mL H₂O was added. The mixture was purged with bubbling Nitrogen for 10 minutes and then Pd(PPh₃)₂Cl₂ (0.04 mmoles, 30 mg) added. The reaction was heated to 80° C. for 22 hours. After that the reaction was cooled down to room temperature, 5 mL of aqueous NH₄Cl added and extracted with Ethyl Acetate. The residue was purified by automated column chromatography using silica gel as stationary phase and hexanes/ethyl acetate as mobile phase to afford compound 14 (55 mg, 45%).

Compound 15: 2-(2-(thieno[3,2-b]thiophen-2-yl)phenyl)acetic acid was prepared as follows. Compound 14 (0.174 mmoles, 50 mg) was dissolved in THF (5 mL) and 0.7 mL of 12M KOH were added. The reaction mixture was heated to 75° C. for 16 hours and then neutralized with HCL conc (1 mL). the mixture was extracted with Ethyl Acetate, the organic layer dried over Mg₂SO₄ and concentrated to afford compound 15 (43 mg, 90%).

Compound 16: naphtho[1,2-b]thieno[2,3-d]thiophen-6-ol was prepared as follows. Compound 15 (0.42 mmoles, 115 mg) was dissolved in THF (3 mL) and DMF (20 μL) added. The reaction mixture was cooled with an ice bath and then (COCl)₂ added (1.05 mmoles, 0.5 ml of 2M solution in dichloromethane). The reaction was stirred for 2 hours and then the solvents evaporated. The residue was dissolved in 1,2-dichloroethane (6 mL), AlCl₃ (0.59 mmoles, 78 mg) added, and the mixture heated to 67° C. After 16 hours the reaction mixture was diluted with dichloromethane and washed with water. The organic layer was separated, and the solvents evaporated. The impure product was purified by automated column chromatography using silica gel as stationary phase and hexanes/ethyl acetate as mobile phase to afford compound 16 (102 mg, 95%).

Compound 17: naphtho[1,2-b]thieno[2,3-d]thiophene-5,6-dione was prepared as follows. Compound 16 (0.43 mmoles, 110 mg) was dissolved in 2 mL of DMF and Co(SALEN)₂ (0.04 mmoles, 15 mg) added. The reaction mixture was left to react uncapped for 16 hours and then poured into H₂O (10 mL), the mixture was filtered and washed with more H₂O to afford compound 17 as a red solid (109 mg, 94%).

Compound 18: 3,8-dibromonaphtho[1,2-b]thieno[2,3-d]thiophene-5,6-dione was prepared as follows. Compound 17 (0.056 mmoles, 15 mg) was dissolved in sulfuric acid and the mixture cooled down to 0° C. NBS (0.13 mmoles, 28 mg) was added portion wise, and the mixture left to react for 4 hours in the ice bath and then at ambient temperature for 14 hours. The mixture was then poured into cold water and filtered. HPLC analysis showed that the solid contained the product 18 (20 mg, 83%).

Compound 19, 3,8-dibromo-5,6-dimethyl-5,6-dihydronaphtho[1,2-b]thieno[2,3-d]thiophene-5,6-diol, can be made by dissolving compound 18 in dry THF and reacting with MeMgBr, When the reaction is completed, the reaction mixture can be quenched by adding saturated NH₄Cl and extracting with Ethyl Acetate. The organic layer can be purified by automated column chromatography using silica gel as stationary phase to afford purified compound 19.

### Example 4. Synthesis of DHBDT Monomer Molecule 21: 2,7-dibromo-4,5-dimethyl-4,5-dihydrobenzo[2,1-b:3,4-b′]dithiophene-4,5-diol

Synthesis of DHBDT molecule 21 (2,7-dibromo-4,5-dimethyl-4,5-dihydrobenzo[2,1-b:3,4-b′]dithiophene-4,5-diol) is illustrated in FIG. 6A, and is briefly described as follows.

Compound 20: 2,7-dibromobenzo[2,1-b:3,4-b′]dithiophene-4,5-dione was prepared as follows. Starting benzo[2,1-b:3,4-b′]dithiophene-4,5-dione (1.14 mmoles, 250 mg) was dissolved in 6 mL acetic acid and the mixture stirred. NBS (2.61 mmoles, 465 mg) was added portion wise, and the mixture left to react at ambient temperature for 48 hours. The mixture was then poured into cold water and filtered to afford product 20 (221 mg, 52%).

Compound 21: 2,7-dibromo-4,5-dimethyl-4,5-dihydrobenzo[2,1-b:3,4-b′]dithiophene-4,5-diol was prepared as follows. Compound 20 was dissolved in dry 2 mL THF and the mixture cooled with an ice bath under nitrogen atmosphere. 0.16 mL 3M MeMgBr were slowly added, and the mixture left to react for 12 hours. After that the reaction mixture was quenched by adding saturated NH₄Cl and HCl 1M. The mixture was extracted with Ethyl Acetate and the solvents evaporated. The residue was purified by automated column chromatography using silica gel as stationary phase and hexanes/ethyl acetate as mobile phase to afford compound 21 (8 mg, 43%).

### Example 5. Pegylated Monomer Compounds and Boronic Esters

The DHNT diol monomer 6 can be PEGylated and boronic esters can be prepared as outlined in FIG. 6B.

Compound 22 can be prepared according to the following procedure: In a 2 neck round bottom flask diol Compound 6 and 18-Crown-6 in THF are added. The solution is purged with nitrogen. In another round bottom flask, 1,3 propane sultone is added in THF and purged with nitrogen. This sultone solution is added to the solution containing Compound 6 by addition funnel over a period of 20-30 minutes. The reaction is stirred at RT for 4-5 hours. After that, the solvent is evaporated off and the precipitate dissolved in water. Acetone is added to induce precipitation of the product as a disodium salt. The precipitate is filtered and re-dissolved in water (minimal amount), neutralized with HCl, and precipitated again in acetone to provide Compound 22.

Compound 23 can be prepared by reacting a DMF solution of Compound 22 with SOCl₂. When the reaction is complete, the mixture is poured into water, filtered, and the precipitate dried to obtain Compound 23.

Compound 24 can be prepared by reacting Compound 23 with 2.2 equivalent of PEG amine in a dichloromethane/TEA mixture. The reaction can be sonicated for 3 hours or stirred overnight. After that the reaction mixture can be washed with an acidified diluted brine solution extracted in dichloromethane. The pure product PEGylated dibromo compound 24 can be purified by column chromatography (silica gel, MeOH—CHCl₃).

Compound 25 can be prepared by reacting for 5-6 hours at 80° C. di-bromo compound 24 with 3 equivalents of bispinacolatodiboron in DMSO under nitrogen, in the presence of KOAc and Pd(dppf)Cl₂ as catalyst. The reaction mixture can then be diluted with CHCl₃ and washed with water. The organic layer can be filtered over celite to remove leftover catalyst and afford the PEGylated di-boronic ester Compound 25.

The same synthetic approach as described for compound 6 can be adopted to introduce PEG chains and to synthetize corresponding boronic esters from compounds 12, 19 and 21.

### Example 6. Polymerization Reactions

A polymerization method to provide DHP-DHNT co-polymer B from DHNT dibromo monomer molecule 6 (2,7-dibromo-4,5-dihydronaphtho[1,2-b]thiophene-4,5-diol) and a diboronic ester of DHP-sulfonamide PEG was performed as shown in FIG. 8. Diboronic ester of DHP-sulfonamide PEG can be prepared according to the method of WO 2017/180998, which is incorporated herein by reference.

A polymerization method to provide DHP-DHBDT co-polymer C from DHBDT dibromo monomer molecule 21 (2,7-dibromo-4,5-dimethyl-4,5-dihydrobenzo[2,1-b:3,4-b′]dithiophene-4,5-diol) and a diboronic ester of DHP-sulfonamide PEG was performed as shown in FIG. 9. As mentioned above, the diboronic ester of DHP-sulfonamide PEG can be prepared according to the method of WO 2017/180998, which is incorporated herein by reference.

A general polymerization protocol was employed as follows. In a round bottom flask, both the bromo and boronic monomers were added in (DMF-water) mixture and purged with nitrogen for 10 minutes. Under nitrogen, about 20 equivalent of CsF and 10% of Pd(OAc)₂ were mixed and heated at 80° C. Polymerization was monitored using UV-Vis spectroscopy and SEC chromatography. Later, a capping agent (selected from G¹) containing an appropriate functional group was added to the reaction mixture and 3 hours later a second capping agent (selected from G²) was added to the reaction mixture. After the reaction was complete, the crude reaction mixture was evaporated and passed through a gel filtration column to remove small organic molecules and low MW oligomers.

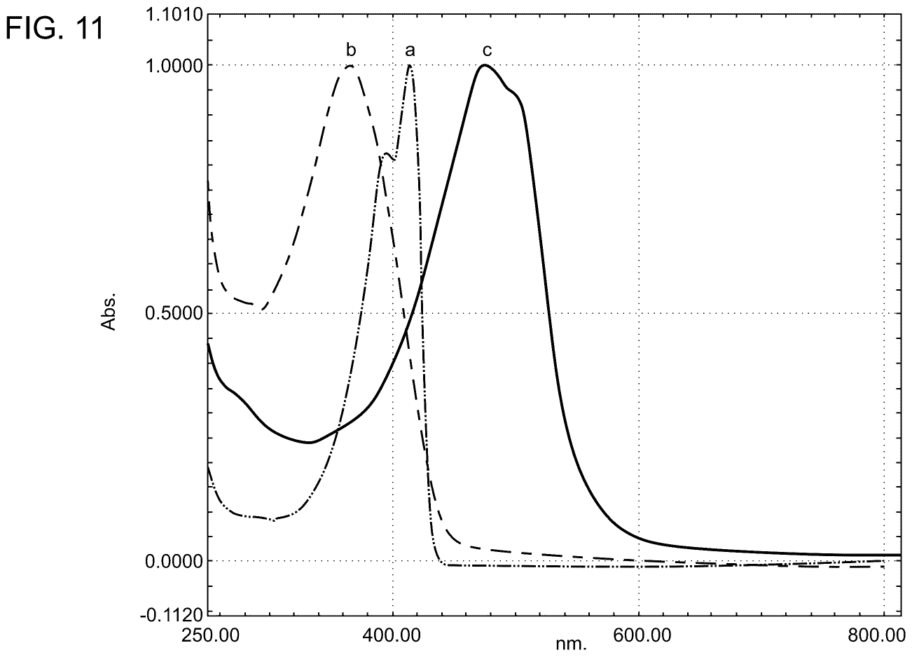

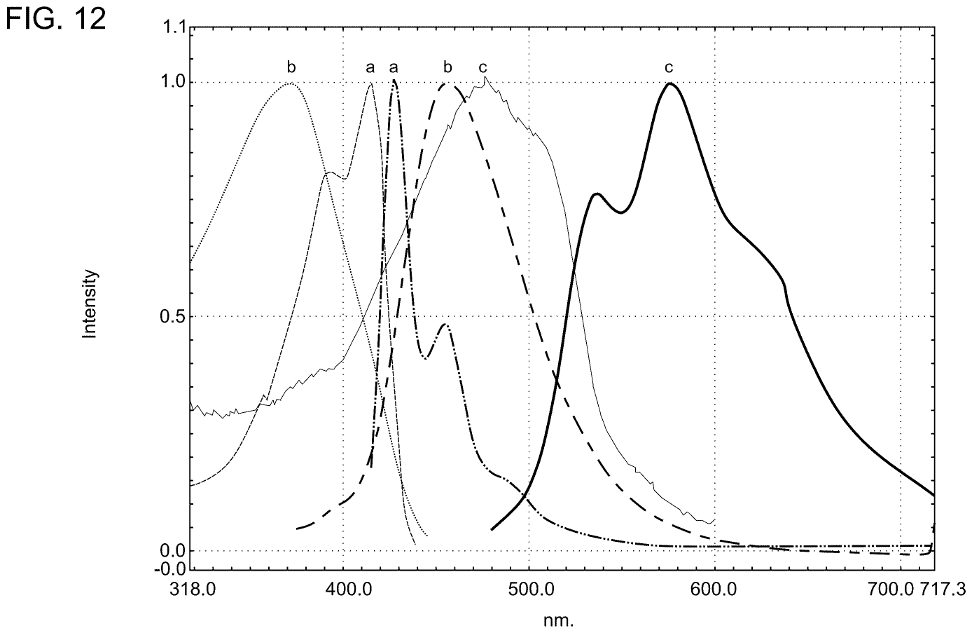

Photophysical data of DHP-DHNT co-polymer B and DHP-DHBDT co-polymer C, including Mn, polydispersity PD, Abs max, emission max, extinction coefficient, and quantum yield, are shown in FIG. 10. UV-Vis absorbance spectra are shown in FIG. 11. Fluorescence excitation and emission spectra are shown in FIG. 12.

As shown in FIG. 12, DHP-DHNT co-polymer B (b) and DHP-DHBDT co-polymer C (c) exhibit longer Stokes shifts than control violet DHP-DHP-polymer SN427 (a). Specifically, DHP-DHNT co-polymer B exhibited excitation absorbance maxima of ~362 nm and fluorescence emission max of ~459 nm (Stokes shift of ~97 nm), and DHP-DHBDT co-polymer C exhibited excitation absorbance max of 472 nm and emission max of ~576 nm (Stokes shift ~104 nm). In contrast, DHP-DHP control polymer SN427 exhibited excitation abs max at about 414 nm and fluorescence emission max of 427 nm, (Stokes shift ~13 nm).

### Example 7. Comparison of Fluorescence Emission Spectra

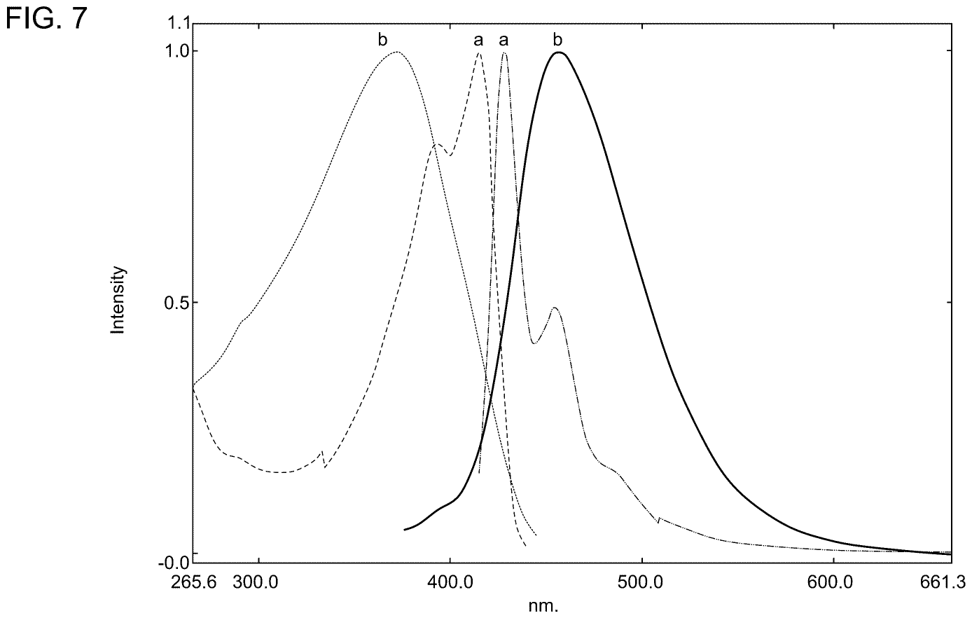

A comparison of fluorescence excitation/emission spectra of dihydrophenanthrene (DHP-DHP) control polymer SN427 compared to DHP-DHNT co-polymer B and DHP-DHBDT co-polymer C was performed. DHP-DHP control polymer exhibited excitation maxima at about 414 nm and fluorescence emission maxima of about 427 nm. DHP-DHNT co-polymer B exhibited excitation maxima of about 362 nm and fluorescence emission maxima of about 459 nm. DHP-DHBDT co-polymer C exhibited excitation Abs max of about 472 nm and an emission max of about 576 nm. The data is shown at FIGS. 7, 10 and 12.

As shown in FIG. 12, the co-polymers of the present disclosure exhibit red shifted emission spectra compared to control DHP polymer. Specifically, DHP-DHNT co-polymer B (em max of about 459 nm) and DHP-DHBDT co-polymer C (em max 576 nm) exhibit red shifted emission spectra compared to control violet DHP-DHP-polymer SN427 (em max 427 nm).

### Example 8. Comparison of UV-Vis Absorbance Spectra

A comparison of UV-Vis absorbance spectra of dihydrophenanthrene (DHP-DHP) control polymer SN427 compared to DHP-DHNT co-polymer B and DHP-DHBDT co-polymer was performed. Normalized UV-Vis spectra of violet polymer (a, black), DHP-DHNT co-polymer B (b, blue) and DHP-DHBDT co-polymer C (c, red) in PBS 1×, are shown in FIG. 11.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A water-soluble polymer dye comprising at least one co-monomer M¹ having a structure according to Formula (Ia): 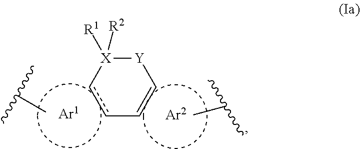
wherein
Ar¹ and Ar² are each independently substituted or unsubstituted aryl or heteroaryl groups and at least one of Ar¹ and Ar² comprises at least one fused thienyl ring;
each X is independently selected from the group consisting of C and Si;
each Y is independently selected from the group consisting of a bond, CR¹R², CHR¹, CHR², SiHR², SiHR¹, and SiR¹R², and when Y is a bond X is directly bonded to both rings, and when Y is a bond, Ar¹ and Ar² are not both thienyl;
each R¹ is independently selected from the group consisting of a water-solubilizing moiety, a linker moiety, alkyl, alkene, alkyne, cycloalkyl, hydroxy, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, phosphonate, 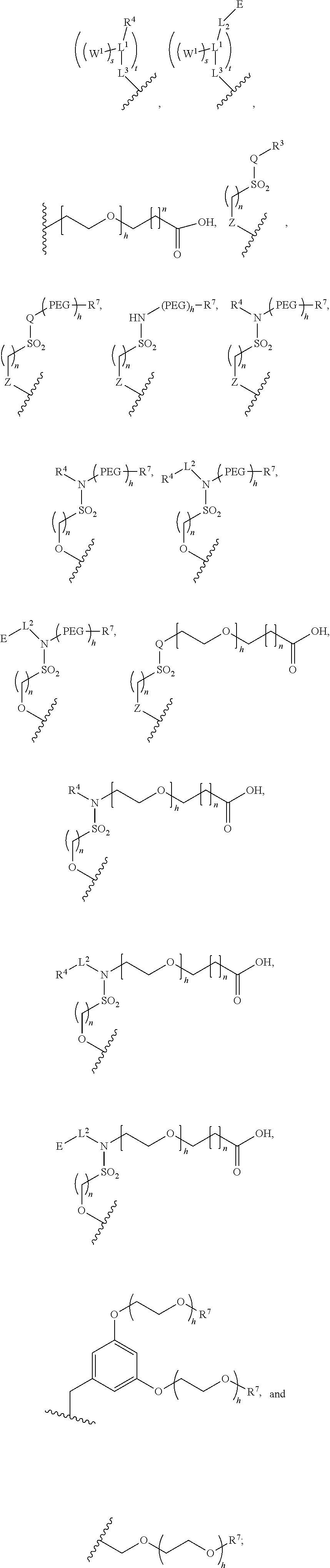
each R² is independently selected from the group consisting of a water-solubilizing moiety, a linker moiety, H, alkyl, alkene, alkyne, cycloalkyl, halogen, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, phosphonate, 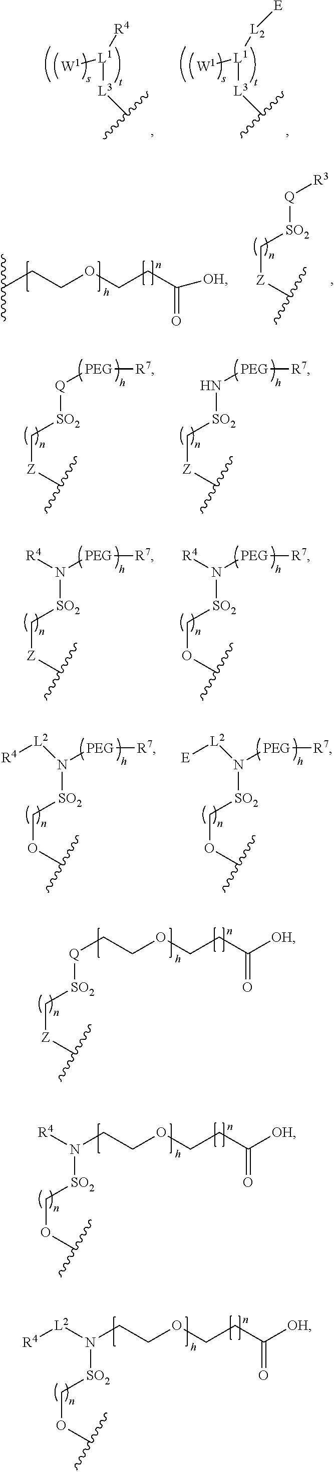 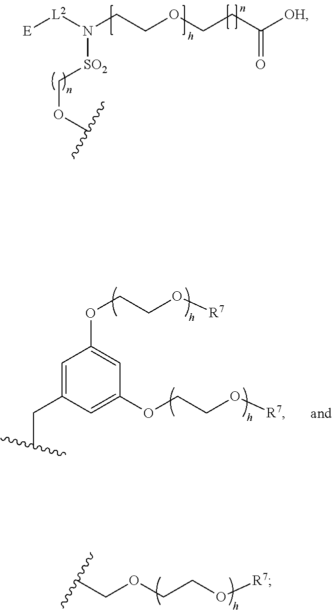
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, and a PEG group;
each Z is independently selected from the group consisting of CH₂, CHR⁴, O, NR⁴, and NH;
each Q is independently selected from the group consisting of a bond, NH, NR⁴, C₁-C₁₂ alkylene, CHR⁴, and CH₂;
each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, an amine, hydroxyl, alkylamino, substituted or unsubstituted C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₈ alkyl, x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof;
each W¹ is independently a water-solubilizing moiety;
L¹, L², and L³ are each independently selected linker moieties;
each E is independently selected from the group consisting of a chromophore, a functional moiety, a substrate, and a binding partner;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester and —OC₁-C₁₂ hydroxy;
at least one of R¹, R², R³, or R⁴ comprises a water-solubilizing moiety;
each h is independently an integer from 0 to 50;
each n is independently an integer from 1 to 20;
each s is independently 1 or 2; and
each t is independently 0, 1, 2, or 3.

2. The water-soluble polymer dye according to claim 1, wherein the at least one co-monomer M¹ comprises a structure selected from the group consisting of Formula (IIa), (IIb), (IIc), (IId), (IIe), (IIf), and (IIg):

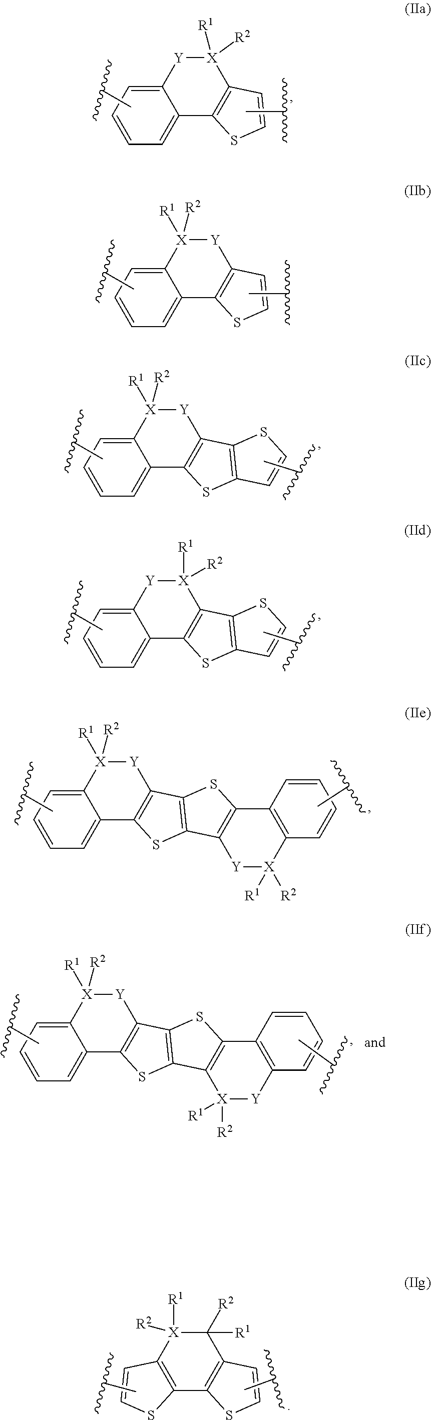

3. The polymer dye according to claim 2, wherein the at least one co-monomer M¹ has a structure selected from the group consisting of Formula (IIaa), (IIbb), (IIcc), (IIdd), (IIee), (IIff), (IIgg), (IIaaa), (IIbbb), (IIccc), (IIddd), (IIeee), (IIfff), and (IIggg):

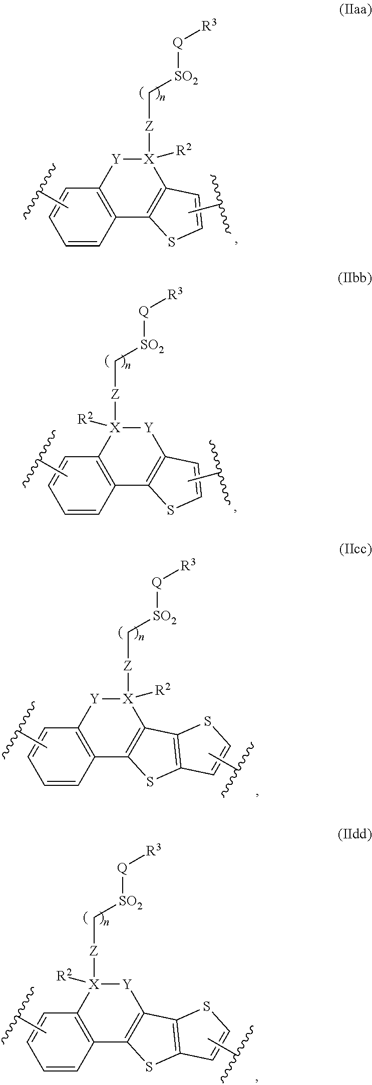

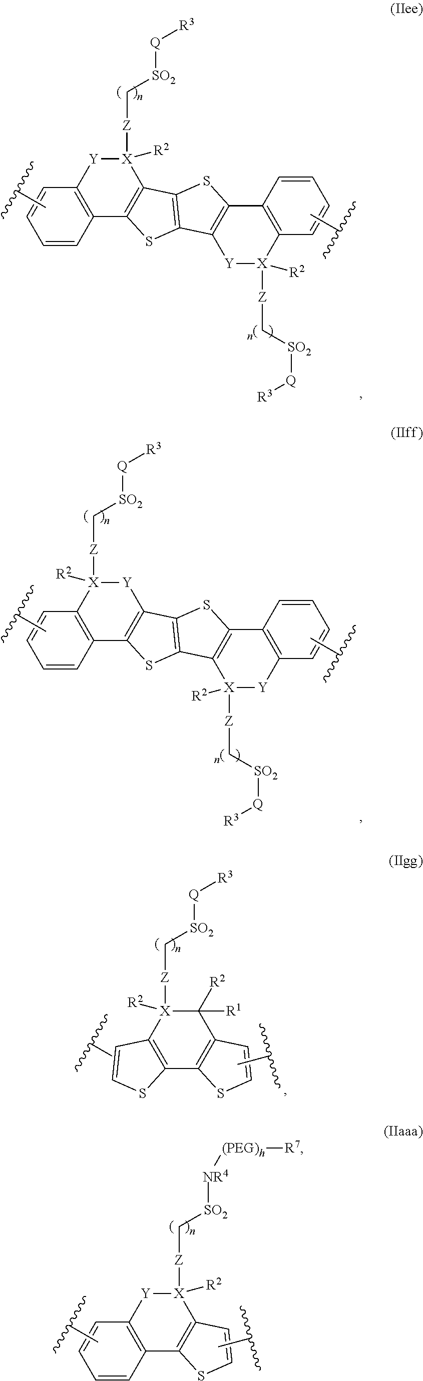

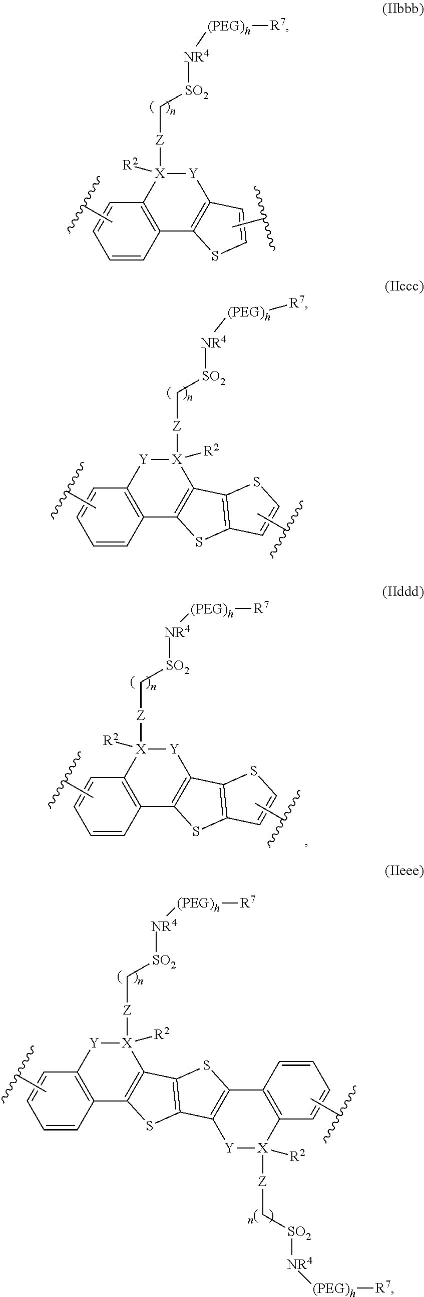

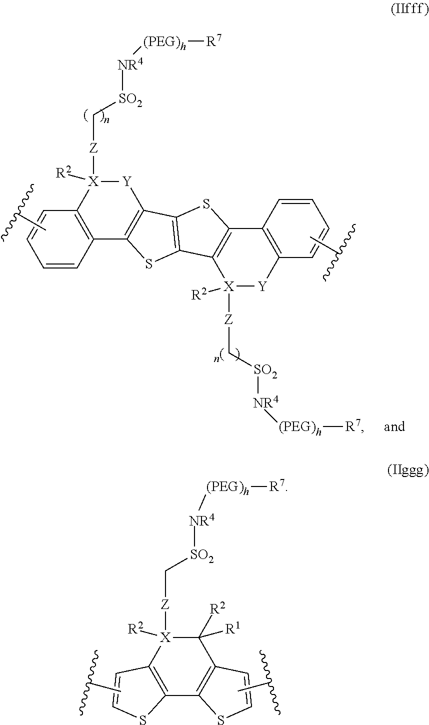

4. The polymer dye according to claim 3, comprising the structure of Formula (Ib): 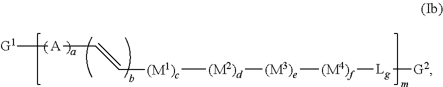
wherein
each A is selected from a substituted or unsubstituted aryl or heteroaryl group;
each optional linker L is independently a linker moiety;
G¹ and G² are each independently selected from the group consisting of an unmodified polymer terminus and a modified polymer terminus, optionally conjugated to E;
a, c, d, e, f and g define the mol % of each unit within the structure which each can be evenly or randomly repeated along the polymer main chain and where a is a mol % from 10 to 100%, c is a mol % from >0 to 90%, each d, e, and f is a mol % from 0 to 90%, and each g is a mol % from 0 to 25%;
each b is independently 0 or 1;
m is an integer from 1 to about 10,000;
each M¹ is a co-monomer having a structure independently selected from the group consisting of Formulas (Ia), (IIa), (IIb), (IIc), (IId), (IIe), (IIf), (IIg), (IIaa), (IIbb), (IIcc), (IIdd), (IIee), (IIff), (IIgg), (IIaaa), (IIbbb), (IIccc), (IIddd), (IIeee), (IIfff), and (IIggg);
each optional M², M³, and M⁴ is independently selected from the group consisting of an aryl moiety, heteroaryl moiety, or co-monomer M¹ having a structure selected from the group consisting of Formulas (Ia), (IIa), (IIb), (IIc), (IId), (IIe), (IIf), (IIg), (IIaa), (IIbb), (IIcc), (IIdd), (IIee), (IIff), (IIgg), (IIaaa), (IIbbb), (IIccc), (IIddd), (IIeee), (IIfff), and (IIggg), wherein
M¹, M², M³, and M⁴ are evenly or randomly distributed along the polymer main chain, and
each M¹ or optional M², M³, or M⁴ is independently optionally substituted, and optionally terminated with a functional group selected from amine, carbamate, carboxylic acid, carboxylate, alkyl carboxylate, carboxylic amine, carbamate, carboxylate ester, maleimide, halogenated maleimide, activated ester, N-hydroxysuccinimidyl, imido ester, halogen, boronic ester, boronic acid, hydrozonyl, hydrazine, hydrazide, hydrazone, azide, alkyne, cyclooctyne, aldehyde, tetrazine, alkene, cyclooctene, dienes, dienophiles, thiol, amide, sulfonamide, alkyl sulfonate, ether, thioether, thiocarbamate, sulfonyl fluoride, hydroxyl, iodoacetyl, iodoacetamide, hydrazido, hydrazino, aldehyde, ketone, phosphine, epoxide, urea, thiourea, thioester, imine, disulfides, and protected groups thereof, each optionally conjugated to a substrate, chromophore, or binding partner.

5. The polymer according to claim 4, wherein A is selected from the group consisting of: 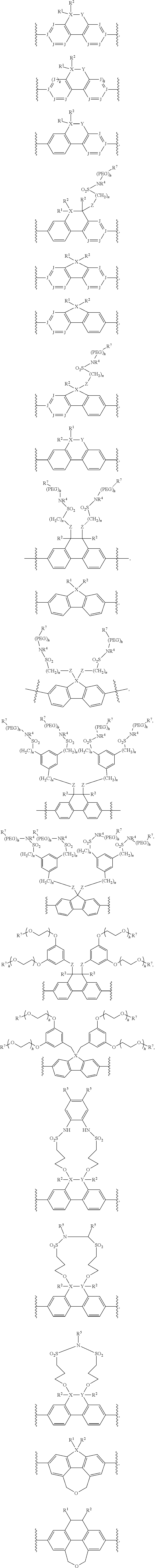 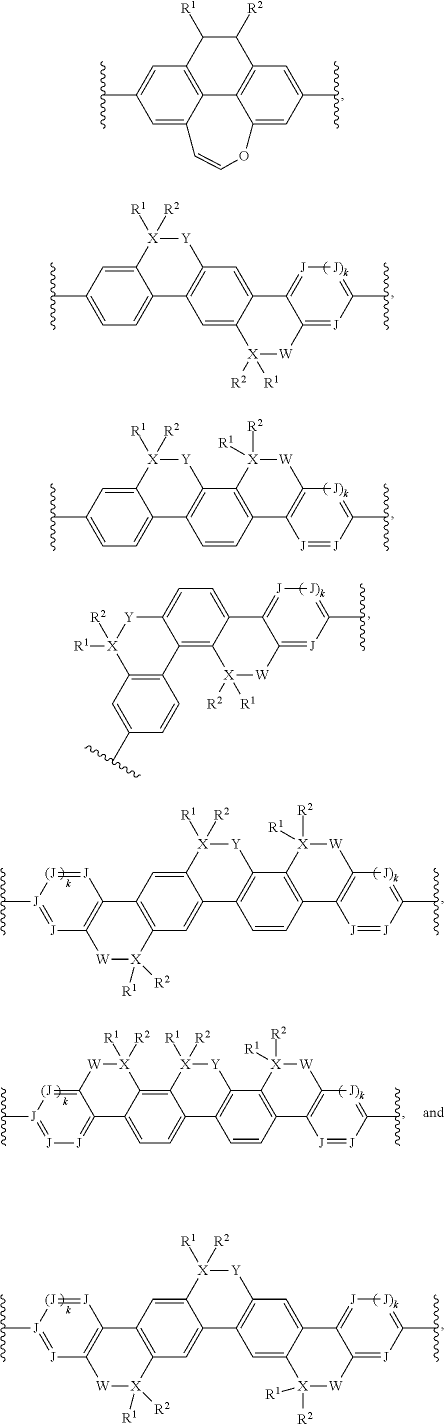
wherein
each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}—OCH₃, and (CH₂)_{x′}(OCH₂—CH₂)_{y′}OCF₃ where each x′ is independently an integer from 0-20 and each y′ is independently an integer from 0-50;
each J is independently selected from the group consisting of C, C(R²), N, N(R²), P, S, Se, O, Se, and Si(R²); and
each W is independently selected from the group consisting of a bond and Y; when W is a bond X is directly bonded to both rings; and
each k is independently selected from 0, 1, or 2.

6. The polymer dye according to claim 4, wherein the dye comprises a structure selected from the group consisting of Formula (IIIa), (IIIb), (IV), (V), (VI), (VIIa), (VIIb), (VIIc), (VIId), (VIIe), (VIIIa), (VIIIb), (XII), (XIIIa), (XIIIb), (XIIIc), (XIIId), and (XIIIe):

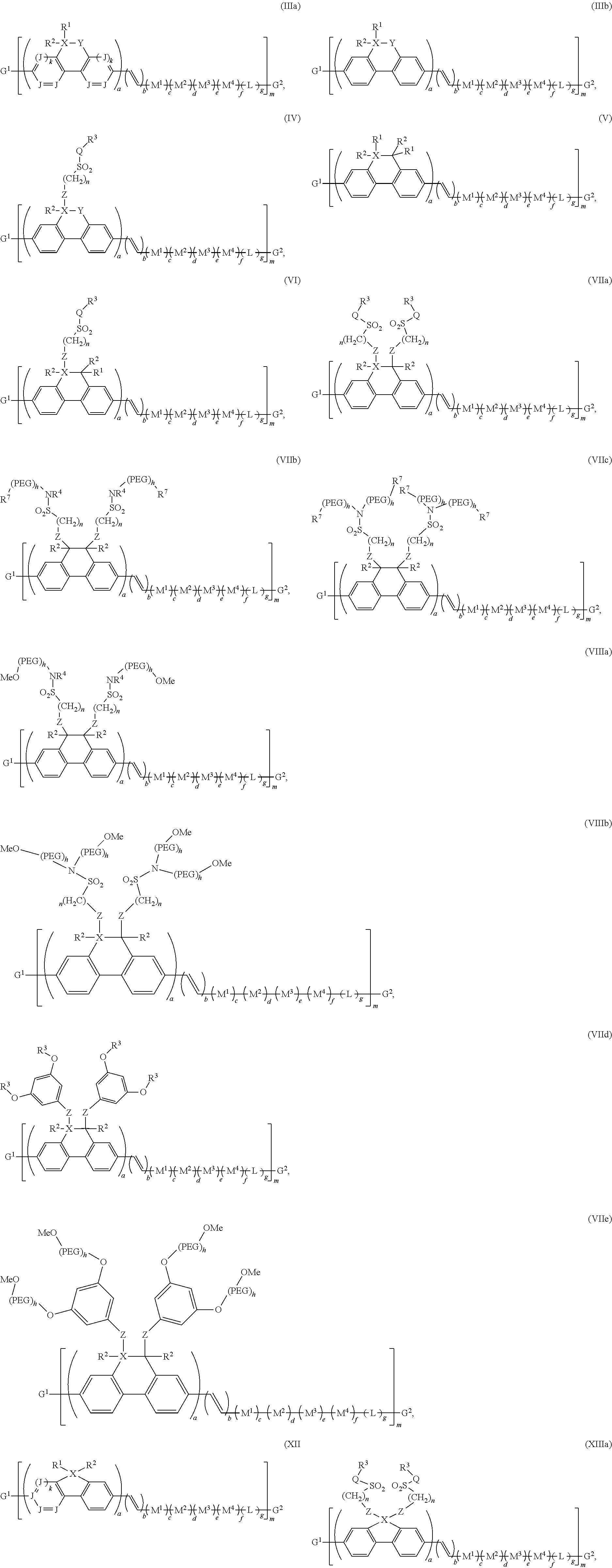

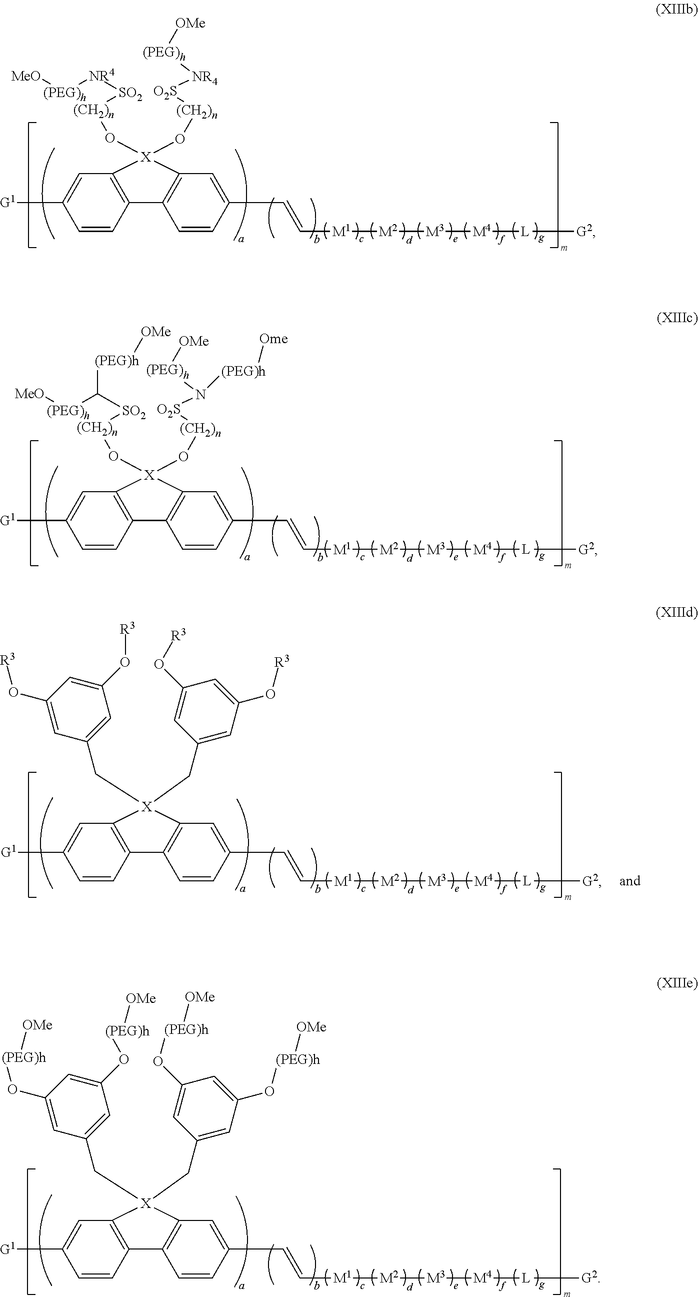

7. The polymer dye according to claim 5, wherein the dye comprises a structure selected from the group consisting of Formula (IXa), (IXb), (IXc), (Xa), (Xb), and (XI): 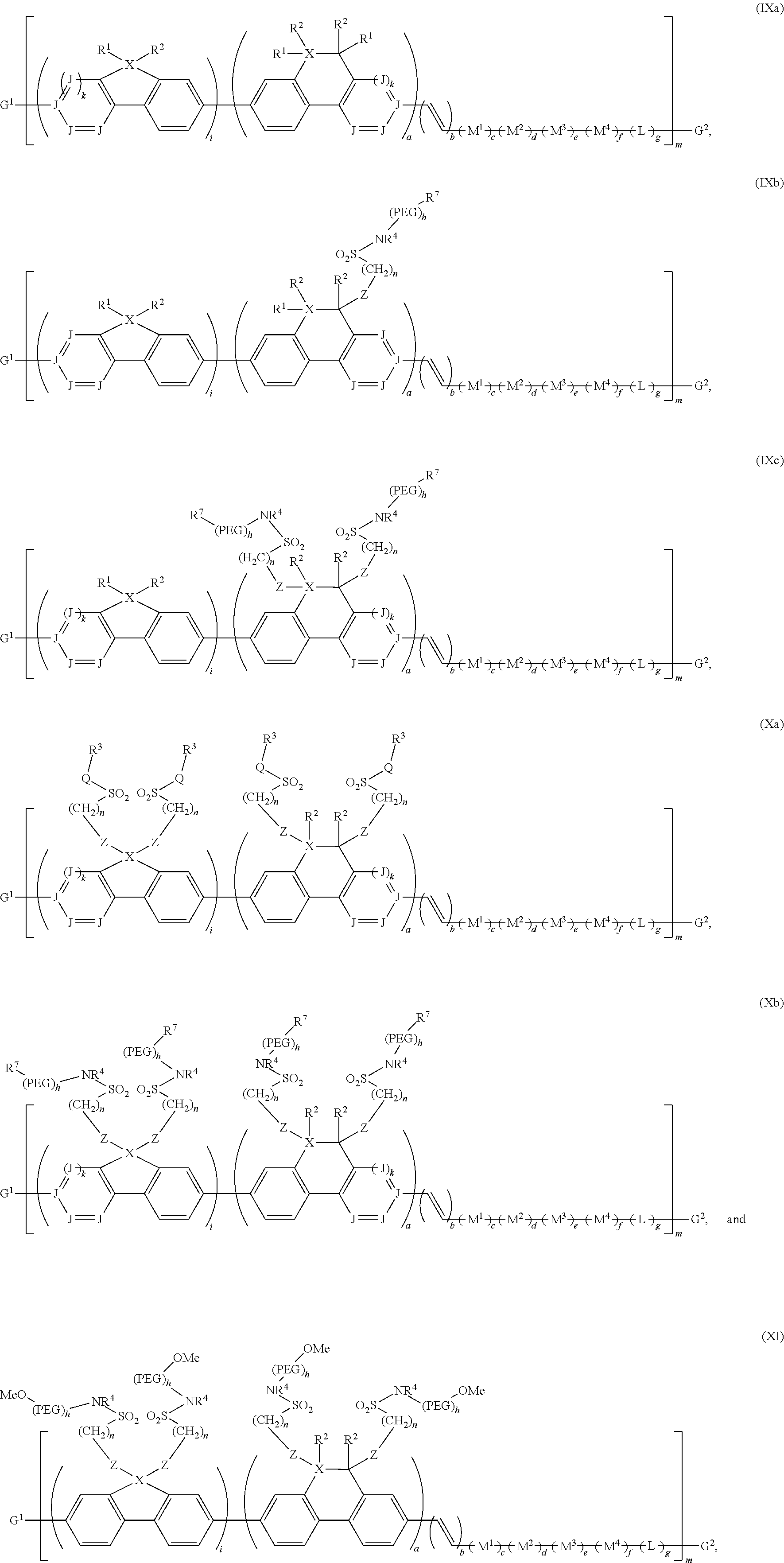
wherein
a and i together is a mol % from 10 to 100%.

8. The polymer dye according to claim 4, comprising a structure selected from the group consisting of Formula (XIVa), (XIVb), (XIVc), (XIVd), (XIVe), (XIVf), and (XIVg):

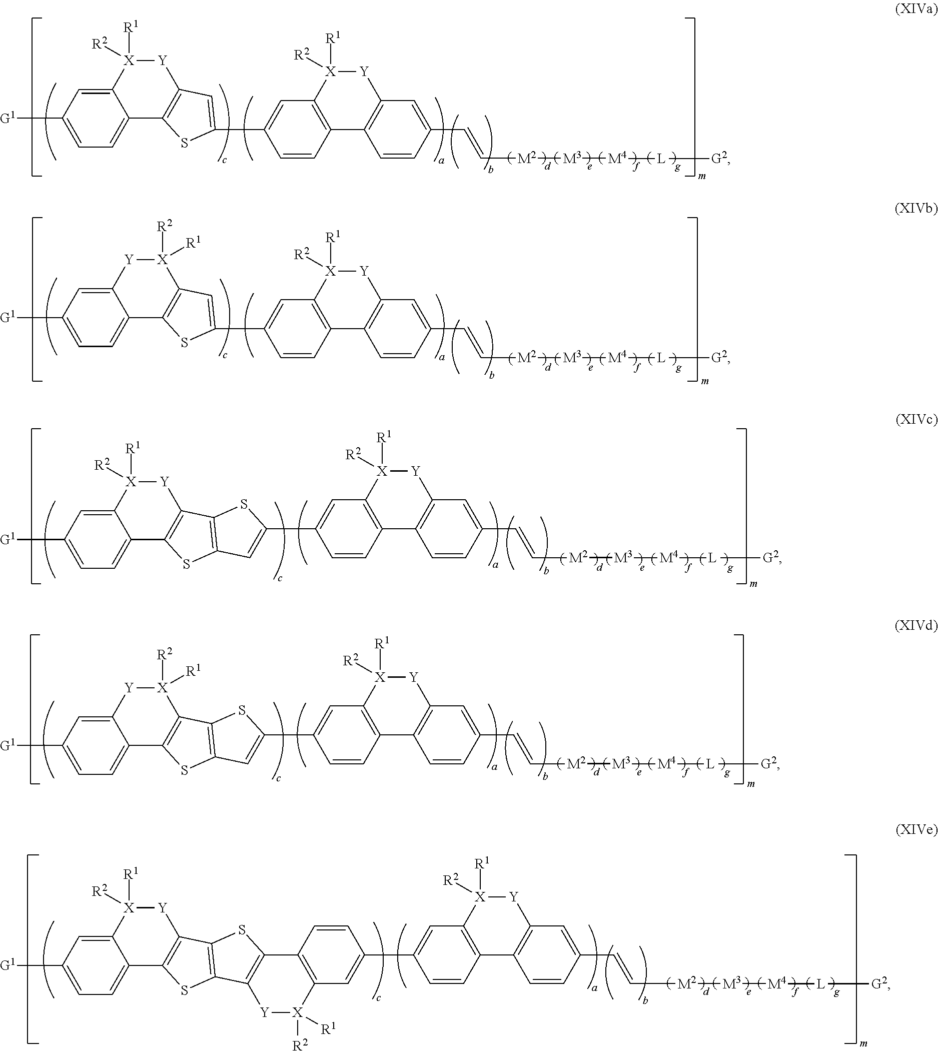

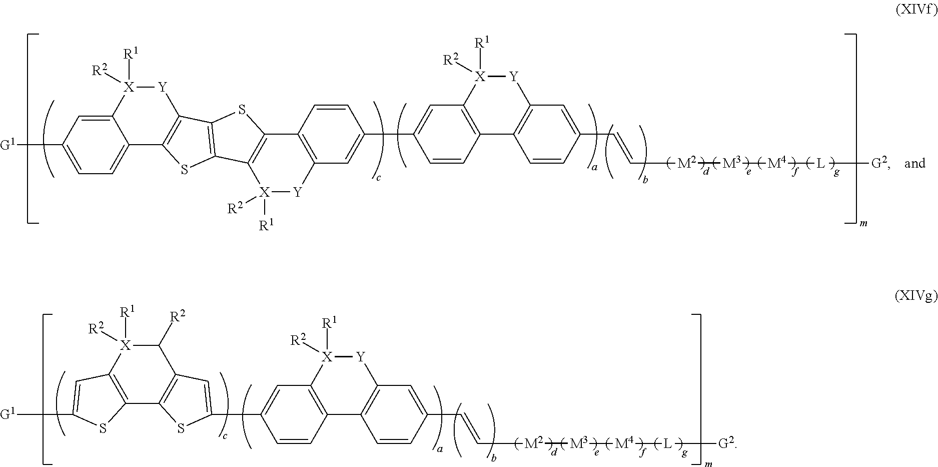

9. The polymer dye according to claim 8, wherein the dye comprises a structure selected from the group consisting of Formula (XVa), (XVb), (XVc), (XVd), (XVe), (XVf), (XVg), (XVIa), (XVIb), (XVIc), (XVId), (XVIIa), (XVIIb), (XVIIc), and (XVIId):

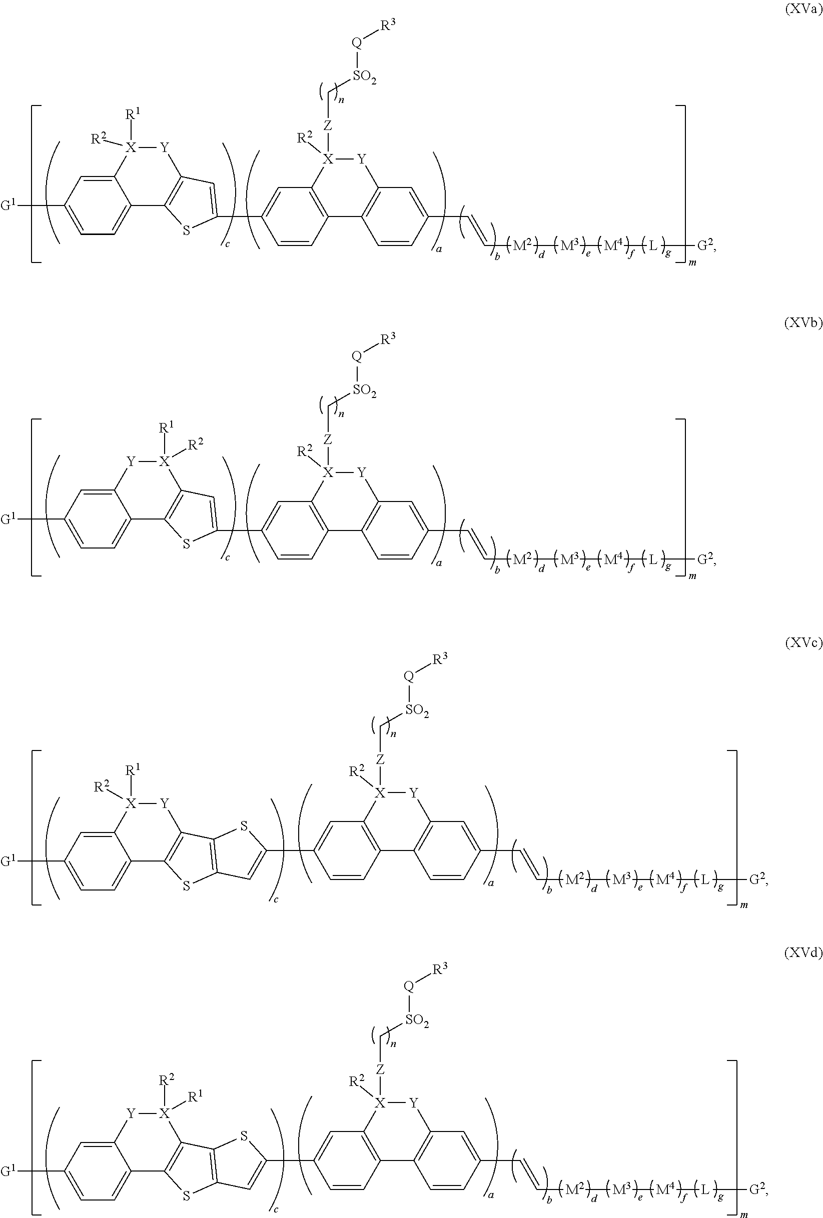

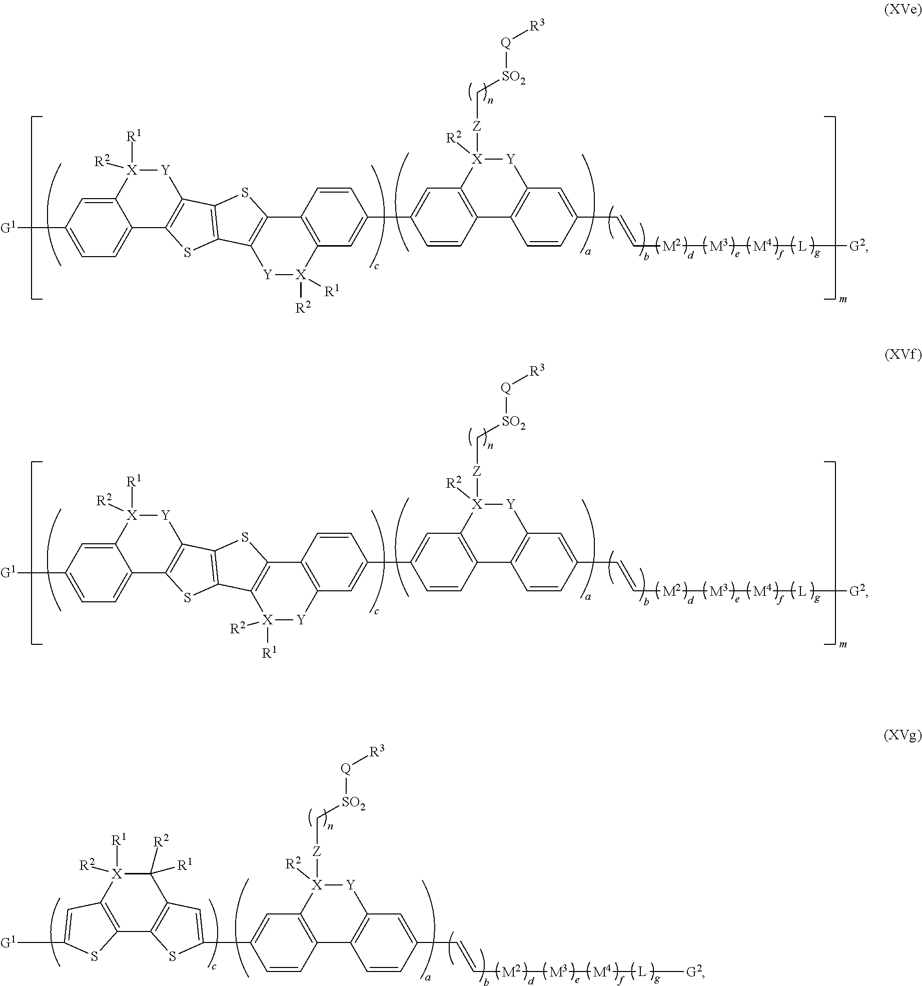

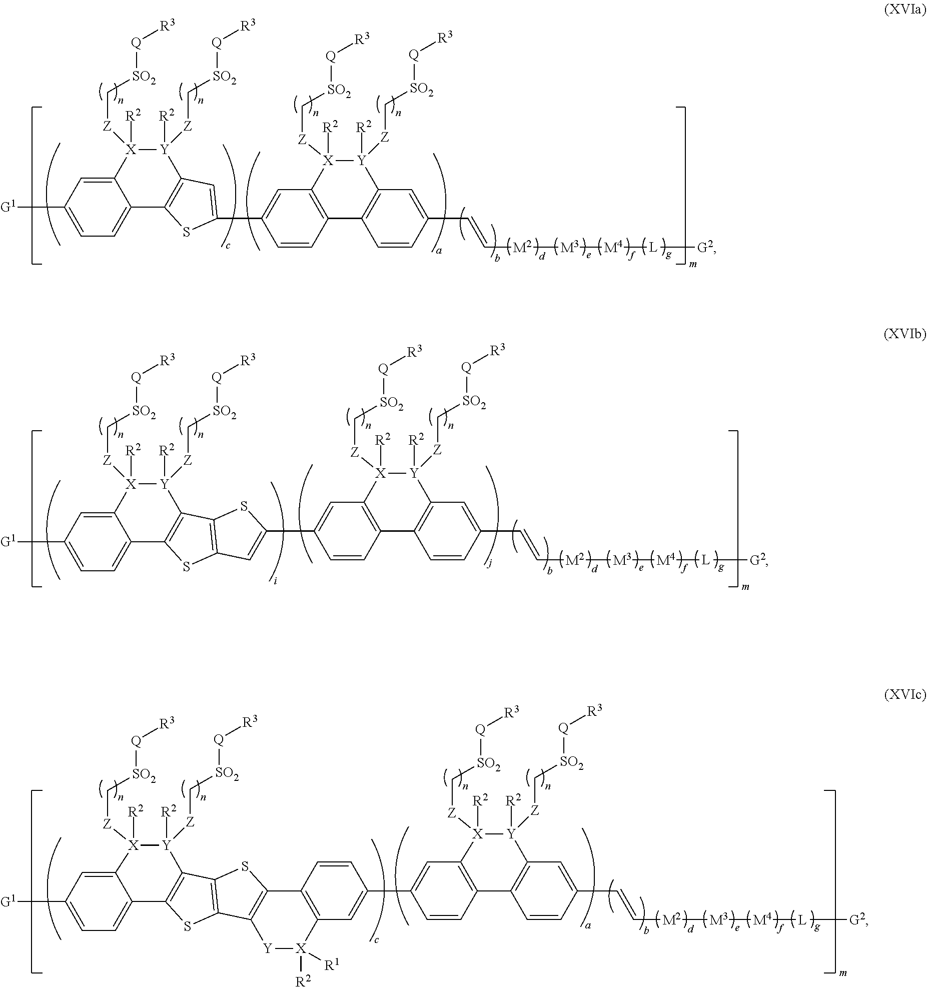

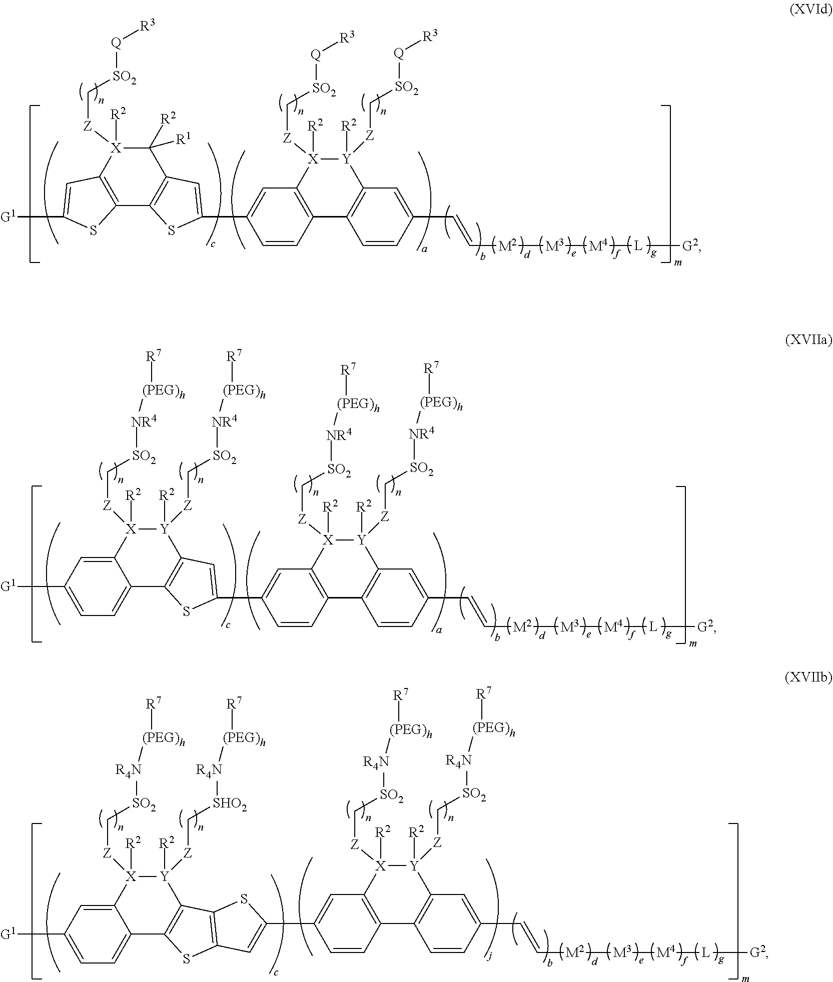

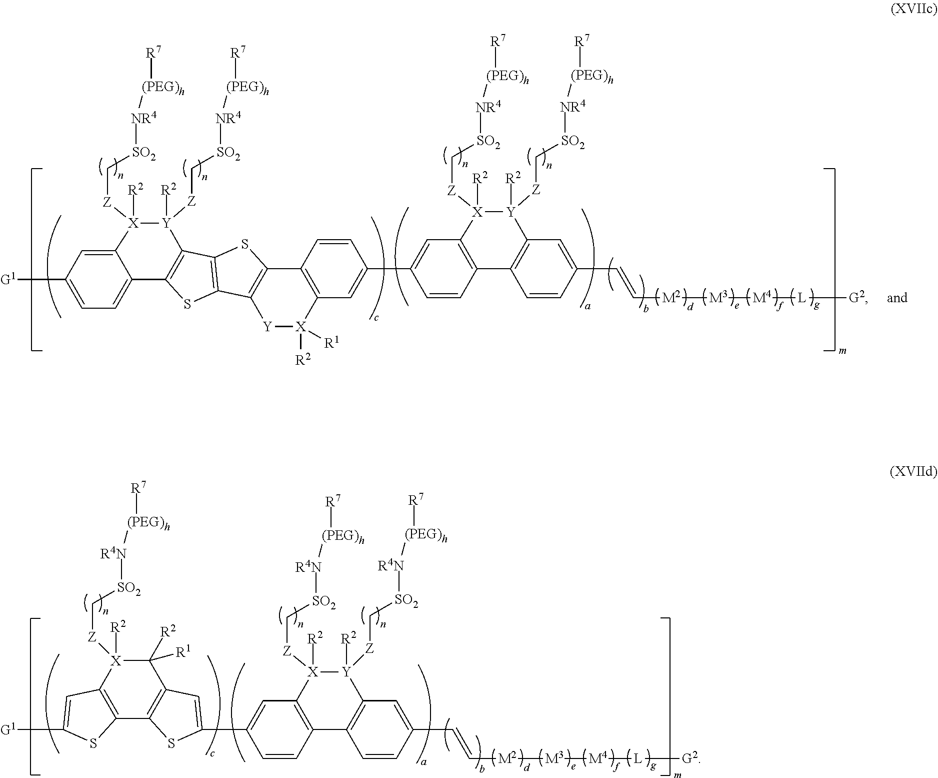

10. The polymer dye according to claim 4, wherein each optional M², M³ or M⁴ is co-monomer independently selected from the group consisting of: 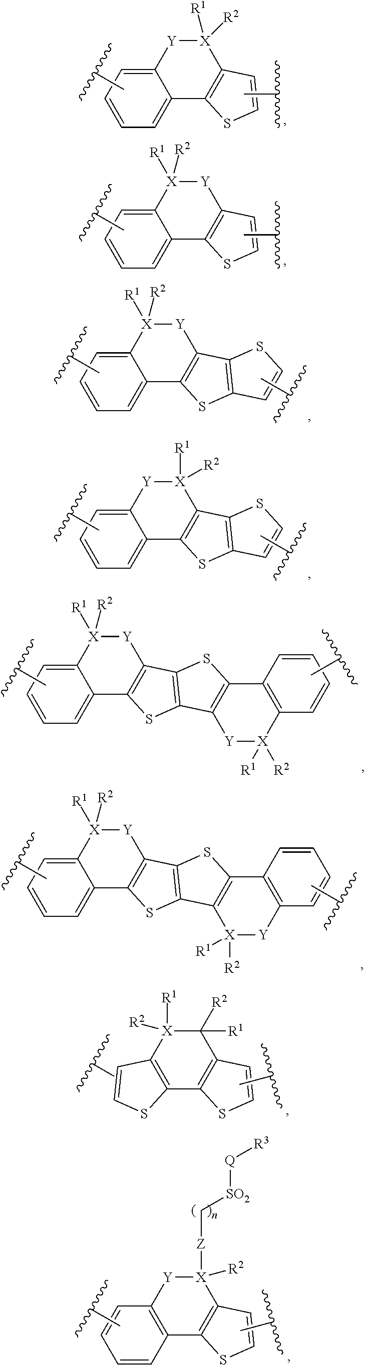 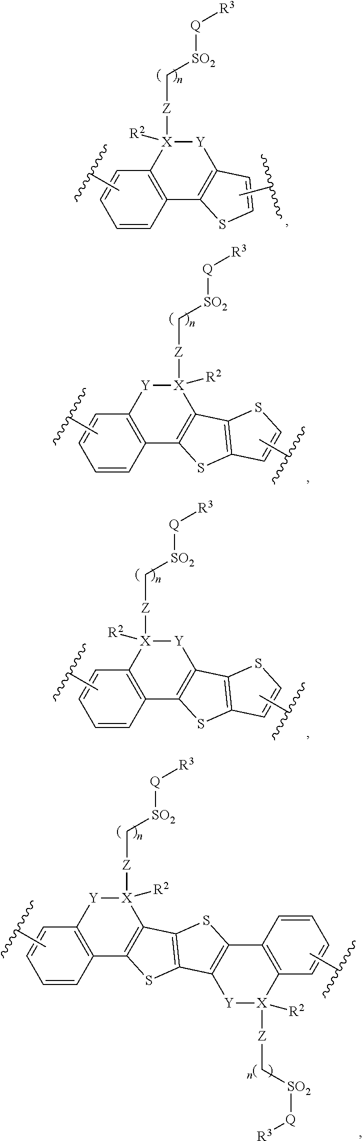 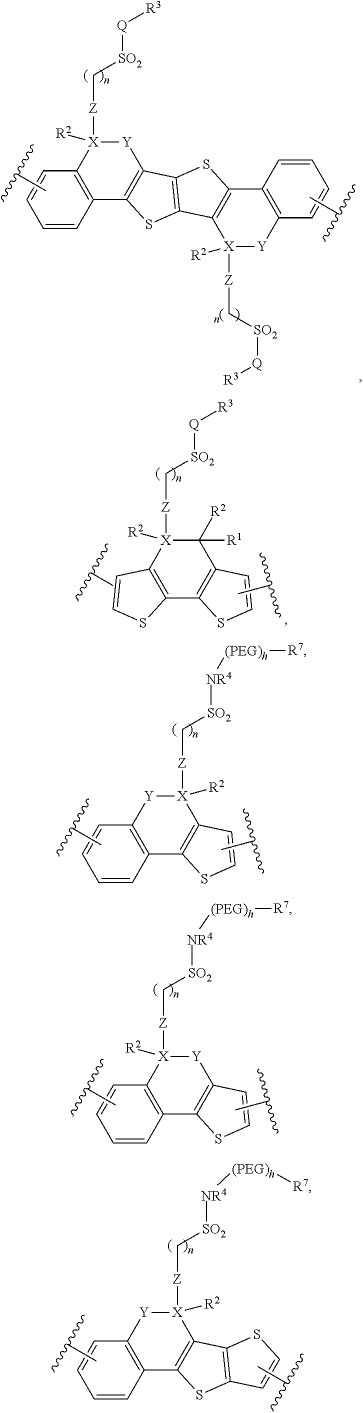 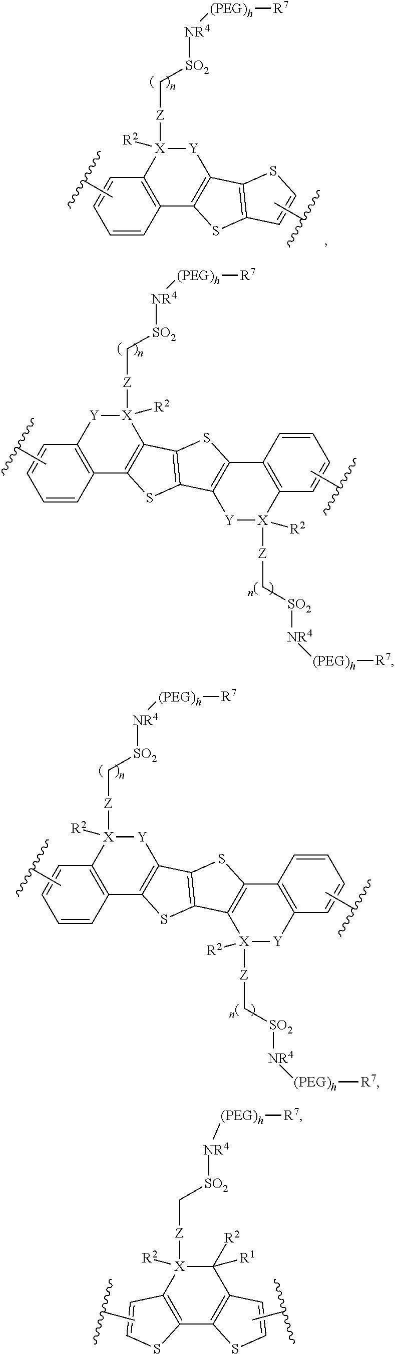 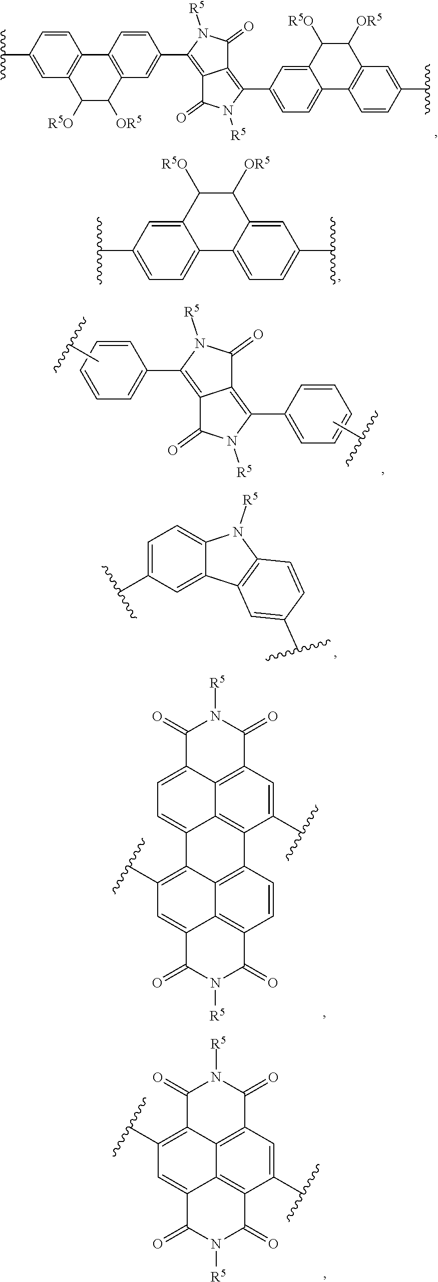 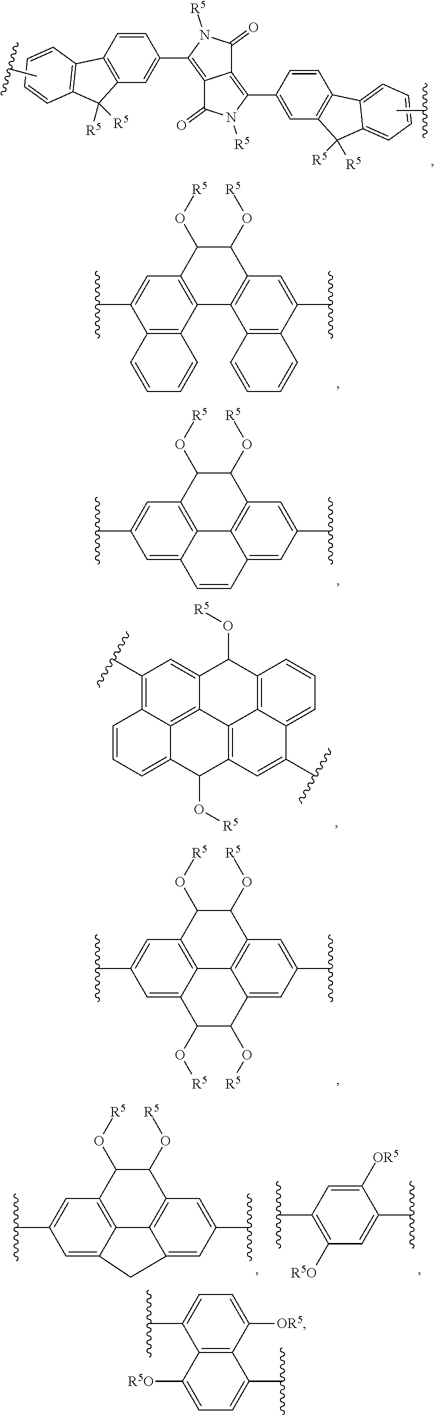 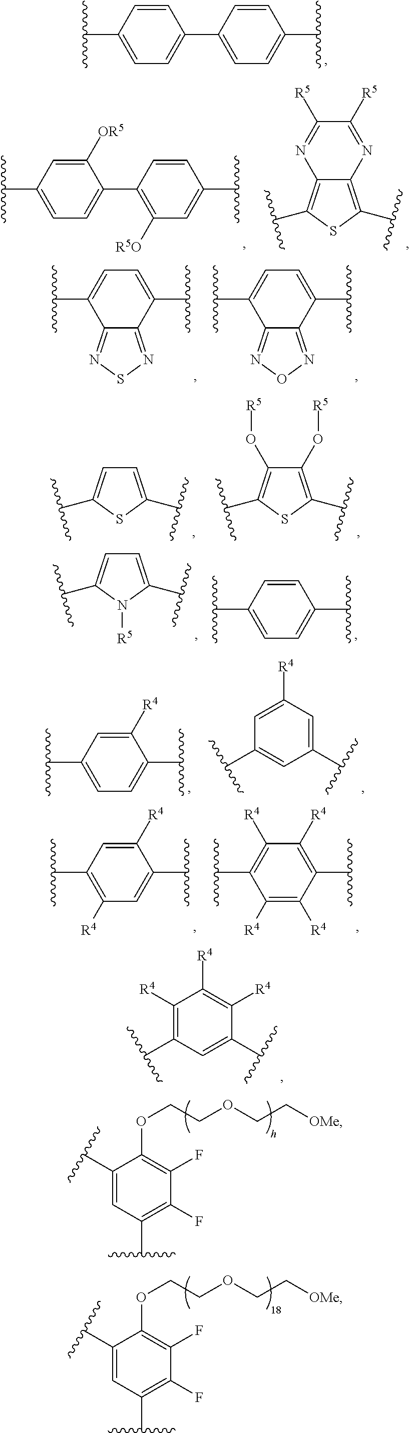 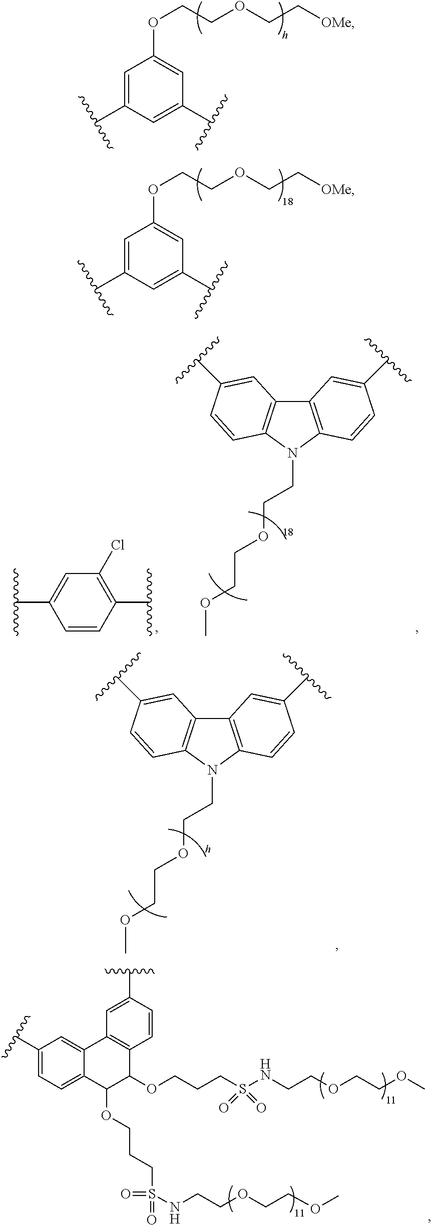 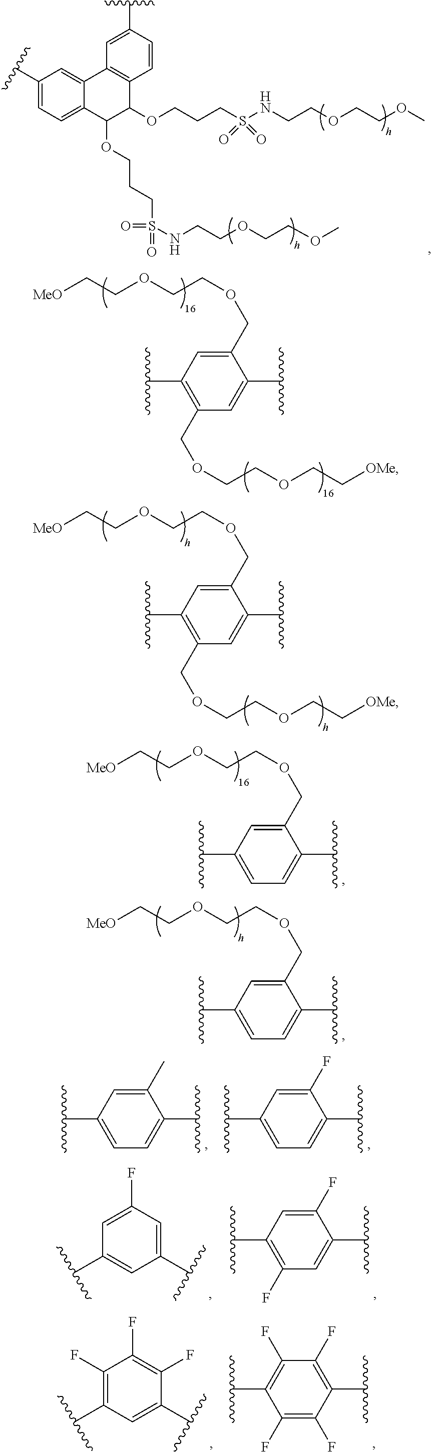 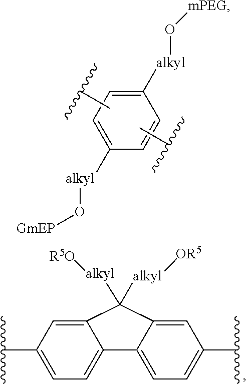
an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substitute, wherein
each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50, optionally conjugated to E.

11. The polymer dye according to claim 4, wherein each optional linker L is independently selected from the group consisting of an aryl or heteroaryl group evenly or randomly distributed along the polymer main chain and that is substituted with one or more pendant chains terminated with a functional group selected from amine, carbamate, carboxylic acid, carboxylate, alkyl carboxylate, carboxylic amine, carbamate, carboxylate ester, maleimide, halogenated maleimide, activated ester, N-hydroxysuccinimidyl, imido ester, halogen, boronic ester, boronic acid, hydrozonyl, hydrazine, hydrazide, hydrazone, azide, alkyne, cyclooctyne, aldehyde, tetrazine, alkene, cyclooctene, dienes, dienophiles, thiol, amide, sulfonamide, alkyl sulfonates, ether, thioether, thiocarbamate, sulfonyl fluoride, hydroxyl, iodoacetyl, iodoacetamide, hydrazido, hydrazino, aldehyde, ketone, phosphine, epoxide, urea, thiourea, thioester, imine, disulfides, and protected groups thereof, each optionally conjugated to E, optionally wherein
each optional linker L is independently selected from the group consisting of: 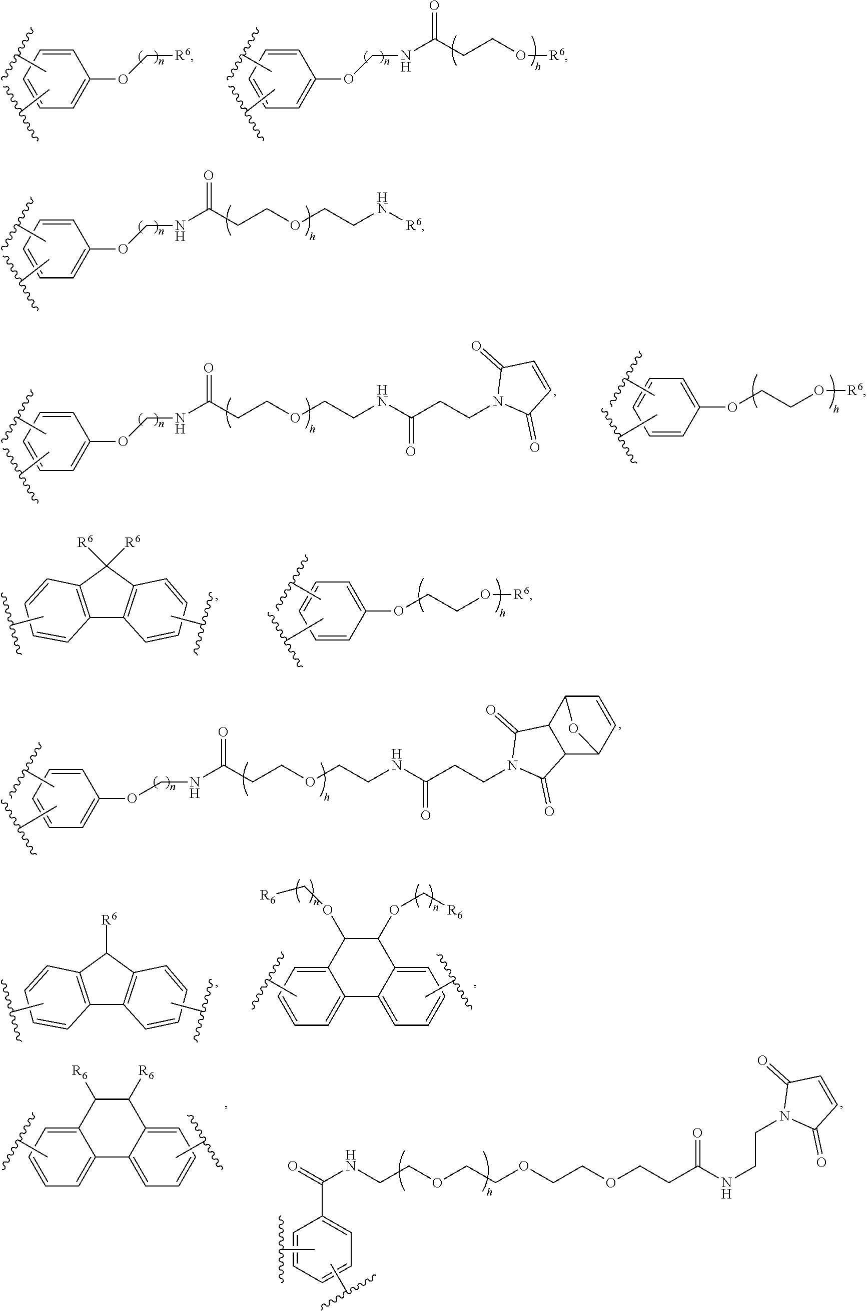 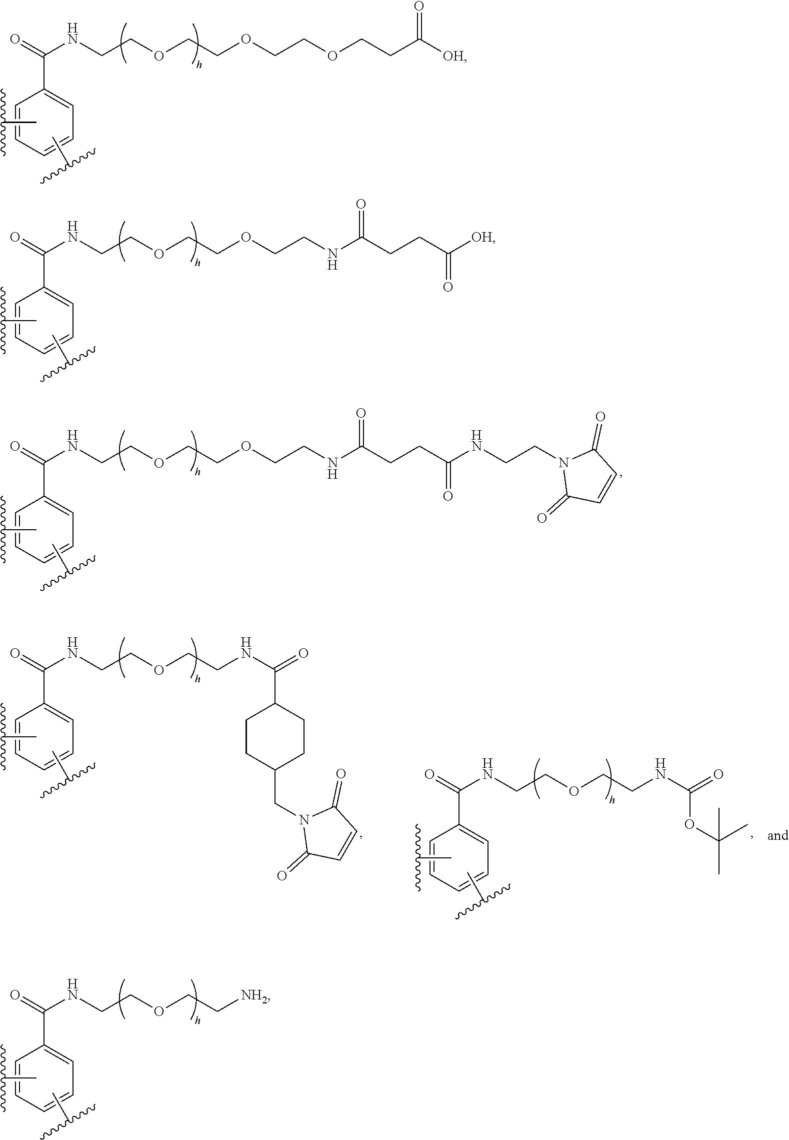
wherein
each R⁶ is independently selected from the group consisting of H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino, benzyl optionally substituted with one or more halogen, hydroxyl, C₁-C₁₂ alkoxy, or (OCH₂CH₂)_{f}OCH₃,

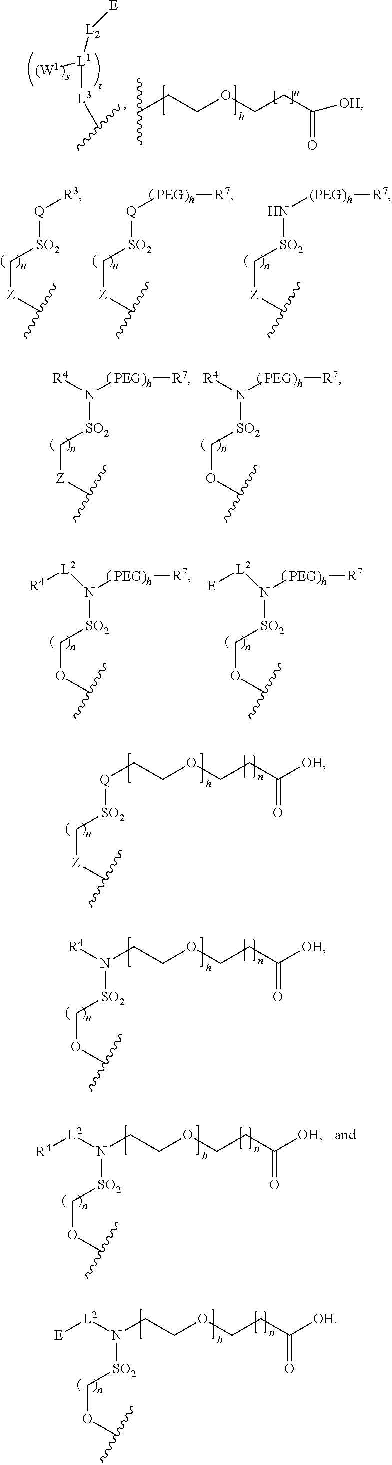

12. The polymer dye according to claim 4, wherein G¹ and G² are each independently selected from the group consisting of hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), and optionally substituted fluorene, optionally substituted tetrahydropyrene (THP), wherein the substituted aryl, heteroaryl, fluorene, DHP or THP are substituted with one or more pendant chains terminated with a functional group, optionally conjugated to E, optionally wherein
G¹ and G² are each independently selected from the group consisting of: 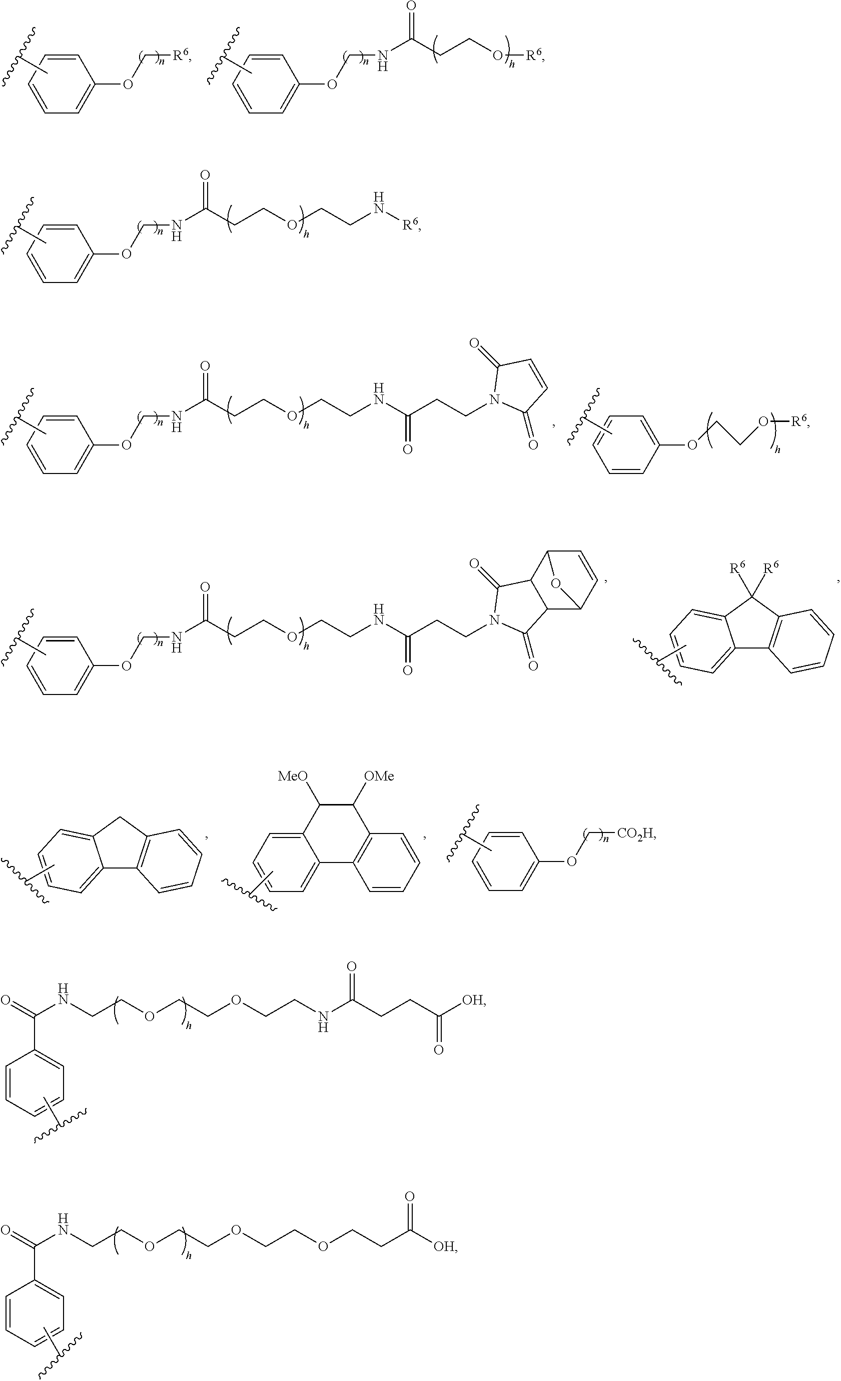 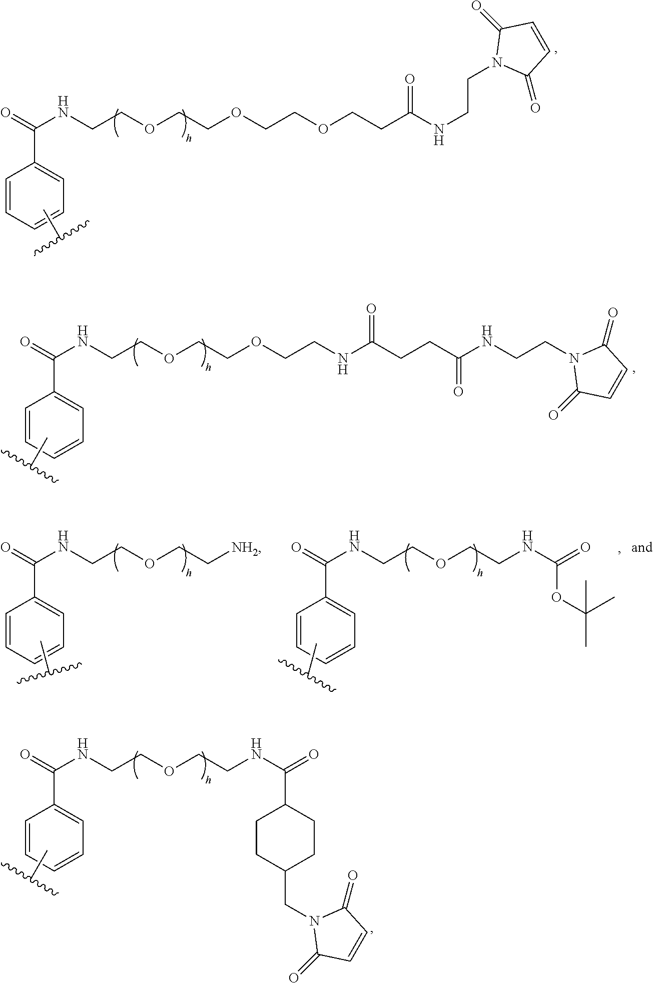
wherein
each R⁶ is independently selected from the group consisting of H, OH, SH, NHCOO-t-butyl, (CH₂)ₙCOOH, (CH₂)ₙCOOCH₃, (CH₂)ₙ(CH₂CH₂O)_{f}COOH, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂ halo alkyl, C₁-C₁₂ (hetero)aryl, C₁-C₁₂ (hetero)arylamino, 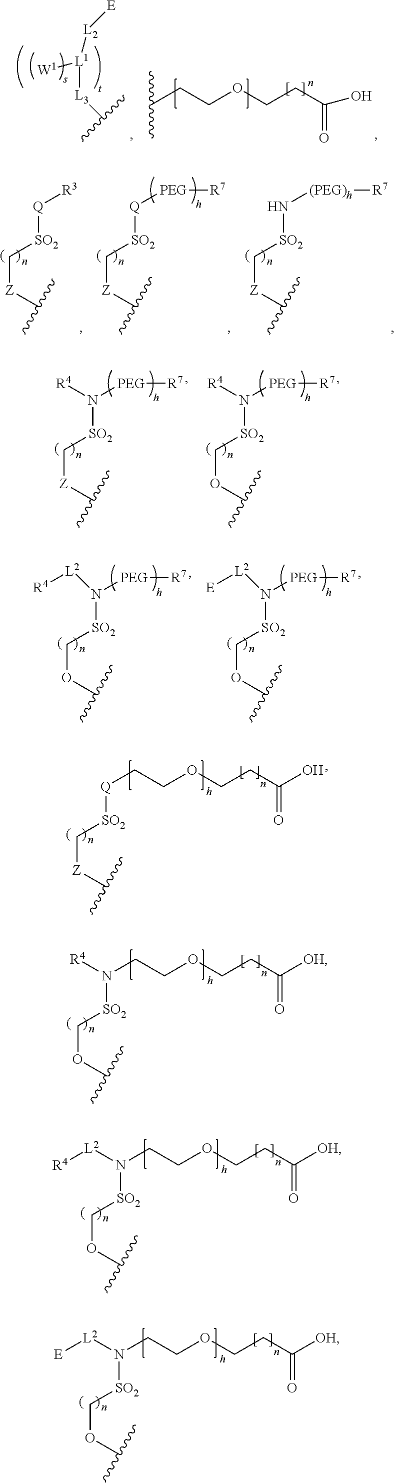
benzyl optionally substituted with one or more halogen, hydroxyl, C₁-C₁₂ alkoxy, or (OCH₂CH₂)_{f}OCH₃, and one or more pendant chains terminated with a functional group, optionally conjugated to E.

13. The polymer dye according to claim 1, wherein the polymer dye is a water-soluble fluorescent polymer dye comprising one or more water-soluble moieties, optionally wherein the water-solubilizing moieties are each independently selected from the group consisting of a PEG group, carboxylic acid, carboxylate, polyvinyl alcohol, glycol, peptide, polyphosphate, polyalcohol, sulfonate, phosphonate, boronate, amine, ammonium, sulfonium, phosphonium, alcohol, polyol, oxazoline, zwitterionic derivative, carbohydrate, nucleotide, polynucleotide, substituted PEG group, substituted carboxy group, substituted carboxylic acid, substituted carboxylate, substituted glycol, substituted peptide, substituted polyphosphate, substituted polyalcohol, substituted sulfonate, substituted phosphonate, substituted boronate, substituted amine, substituted ammonium, substituted sulfoniums substituted phosphonium, substituted zwitterionic derivative, substituted carbohydrate, substituted nucleotide, and substituted polynucleotide, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide,

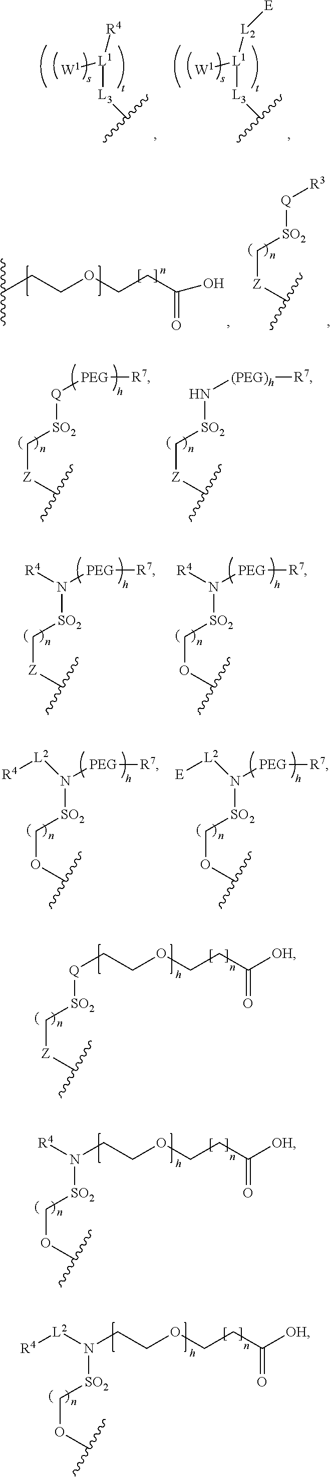

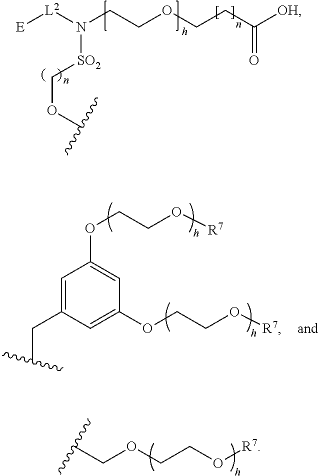

14. A polymer tandem dye comprising:
the polymer dye according to claim 1; and
at least one signaling chromophore covalently linked to the polymer dye in energy-receiving proximity therewith.

15. A labeled specific binding partner, comprising
the polymer dye according to claim 1; and
a specific binding partner capable of specific binding to a target analyte covalently linked to the polymer dye.

16. A method for detecting a target analyte in a sample comprising:
providing a sample that is suspected of containing the target analyte; and
contacting the sample with a labeled specific binding partner according to claim to form a fluorescent polymer dye conjugate complex with the target analyte if the target analyte is present;
applying a light source to the sample that can excite the at least one fluorescent polymer dye conjugate complex; and
detecting light emitted from the fluorescent polymer dye conjugate complex, optionally
wherein the sample is a biological sample selected from the group consisting of blood, bone marrow, spleen cells, lymph cells, bone marrow aspirates, urine, serum, saliva, cerebral spinal fluid, urine, amniotic fluid, interstitial fluid, feces, mucus, tissue, or is from a cell culture.

17. A kit comprising at least one polymer dye, polymer tandem dye, or labeled specific binding partner according to claim 1.

18. The kit according to claim 17, wherein the at least one polymer dye, polymer tandem dye, or labeled specific binding partner comprises a conjugation tag.

19. The labeled specific binding partner according to claim 15, wherein the specific binding partner is selected from the group consisting of a protein, peptide, affinity ligand, antibody, antibody fragment, carbohydrate, lipid, nucleic acid, and an aptamer.

20. The labeled specific binding partner according to claim 15, wherein the specific binding partner is covalently linked to a G¹ and/or G² moiety of the polymer dye.
